SEQUENCE LISTING (173 sequences)

SEQ ID NO: 1 - protein, 7 aa, Artificial Sequence
  source: /note=“Description of Artificial Sequence Synthetic peptide”
DYKDDDK

SEQ ID NO: 2 - protein, 22 aa, Artificial Sequence
  source: /note=“Description of Artificial Sequence Synthetic peptide”
DYKDHDGDYKDHDIDYKDDDDK

SEQ ID NO: 3 - protein, 7 aa, Artificial Sequence
  source: /note=“Description of Artificial Sequence Synthetic peptide”
PKKKRKV

SEQ ID NO: 4 - protein, 9 aa, Artificial Sequence
  source: /note=“Description of Artificial Sequence Synthetic peptide”
PAAKRVKLD

SEQ ID NO: 5 - protein, 10 aa, Artificial Sequence
  source: /note=“Description of Artificial Sequence Synthetic peptide”
EGAPPAKRAR

SEQ ID NO: 6 - protein, 8 aa, Artificial Sequence
  source: /note=“Description of Artificial Sequence Synthetic peptide”
VSRKRPRP

SEQ ID NO: 7 - protein, 18 aa, Artificial Sequence
  source: /note=“Description of Artificial Sequence Synthetic peptide”
KRPAATKKAGQAKKKKLD

SEQ ID NO: 8 - protein, 38 aa, Artificial Sequence
  source: /note=“Description of Artificial Sequence Synthetic polypeptide”
NQSSNFGPMKGGNFGGRSSGPYGGGGQYFAKPRNQGGY

SEQ ID NO: 9 - protein, 17 aa, Artificial Sequence
  source: /note=“Description of Artificial Sequence Synthetic peptide”
PKTRRRPRRSQRKRPPT

SEQ ID NO: 10 - DNA, 130 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 321 bp, Artificial Sequence
  source: /note=“Description of Artificial Sequence Synthetic polynucleotide”
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 12 - DNA, 393 bp, Artificial Sequence
  source: /note=“Description of Artificial Sequence Synthetic polynucleotide”
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 13 - DNA, 48 bp, Artificial Sequence
  source: /note=“Description of Artificial Sequence Synthetic oligonucleotide”
cttgttctttttgcagaagctcagaataaacgctcaactttggcagat

SEQ ID NO: 14 - DNA, 133 bp, Artificial Sequence
  source: /note=“Description of Artificial Sequence Synthetic polynucleotide”
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 15 - DNA, 66 bp, Artificial Sequence
  source: /note=“Description of Artificial Sequence Synthetic oligonucleotide”
gactacaaagaccatgacggtgattataaagatcatgacatcgattacaaggatgacgatgacaag

SEQ ID NO: 16 - DNA, 66 bp, Artificial Sequence
  source: /note=“Description of Artificial Sequence Synthetic oligonucleotide”
gattataaagatcatgacggggactataaggatcacgacatagactacaaagacgatgatgacaaa

SEQ ID NO: 17 - DNA, 1143 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 18 - DNA, 1143 bp, Artificial Sequence
  source: /note=“Description of Artificial Sequence Synthetic polynucleotide”
gattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 19 - DNA, 1143 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 20 - DNA, 1143 bp, Artificial Sequence
  source: /note=“Description of Artificial Sequence Synthetic polynucleotide”
gattacaaagaccacgacggagactacaaggaccatgatattgactacaaagacgatgatgataagatggcacccaaaaagaagagaaaagtgggaatccacggtgtaccggccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 21 - DNA, 1143 bp, Artificial Sequence
  source: /note=“Description of Artificial Sequence Synthetic polynucleotide”
gactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 22 - DNA, 1143 bp, Artificial Sequence
  source: /note=“Description of Artificial Sequence Synthetic polynucleotide”
gattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 23 - DNA, 1143 bp, Artificial Sequence
  source: /note=“Description of Artificial Sequence Synthetic polynucleotide”
gactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 24 - DNA, 63 bp, Artificial Sequence
  source: /note=“Description of Artificial Sequence Synthetic oligonucleotide”
ggcagcggagagggcagaggaagcctgctcacctgcggtgacgtggaggaaaaccctggccct

SEQ ID NO: 25 - DNA, 1221 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 26 - DNA, 1221 bp, Artificial Sequence
  source: /note=“Description of Artificial Sequence Synthetic polynucleotide”
gattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 27 - DNA, 1221 bp, Artificial Sequence
  source: /note=“Description of Artificial Sequence Synthetic polynucleotide”
gattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 28 - DNA, 1221 bp, Artificial Sequence
  source: /note=“Description of Artificial Sequence Synthetic polynucleotide”
gattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 29 - DNA, 1221 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 30 - DNA, 1221 bp, Artificial Sequence
  source: /note=“Description of Artificial Sequence Synthetic polynucleotide”
gattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 31 - DNA, 1221 bp, Artificial Sequence
  source: /note=“Description of Artificial Sequence Synthetic polynucleotide”
gactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 32 - DNA, 592 bp, Artificial Sequence
  source: /note=“Description of Artificial Sequence Synthetic polynucleotide”
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 33 - DNA, 223 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctat

SEQ ID NO: 34 - DNA, 108 bp, Artificial Sequence
  source: /note=“Description of Artificial Sequence Synthetic polynucleotide”
aggaacccctagtgatggagttggccactccctctctgcgcgctcgctcgctcactgaggccgcccgggctttgcccgggcggcctcagtgagcgagcgagcgcgcag

SEQ ID NO: 35 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 36 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 37 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 38 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 39 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 40 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 41 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatcttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 42 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 43 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 44 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 45 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 46 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 47 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 48 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagttgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 49 - DNA, 4491 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttgagctcttcgaaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcctaggtgcttgttctttttgcagaagctcagaataaacgctcaactttggcagatactagtcaggtaagtatcaaggttacaagacaggtttaaggagaccaatagaaactgggcttgtcgagacagagaagactcttgcgtttctgataggcacctattggtcttactgacatccactttgcctttctctccacaggaccggtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttctgataactcgagtctagaaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctggctagcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgcggccgcgtcgagcgcaggaacccctagtgatggagttggccactccctctctgcgcgctcgctcgctcactgaggccgcccgggctttgcccgggcggcctcagtgagcgagcgagcgcgcag

SEQ ID NO: 50 - DNA, 66 bp, Artificial Sequence
  source: /note=“Description of Artificial Sequence Synthetic oligonucleotide”
gattataaagaccatgatggtgattacaaggaccatgacatcgattataaagacgacgacgacaaa

SEQ ID NO: 51 - DNA, 66 bp, Artificial Sequence
  source: /note=“Description of Artificial Sequence Synthetic oligonucleotide”
gattataaggatcatgatggagactataaggatcatgacatagattacaaagatgacgatgacaag

SEQ ID NO: 52 - DNA, 66 bp, Artificial Sequence
  source: /note=“Description of Artificial Sequence Synthetic oligonucleotide”
gactacaaagatcatgatggcgactacaaagatcatgatatagattacaaagacgatgacgacaaa

SEQ ID NO: 53 - DNA, 66 bp, Artificial Sequence
  source: /note=“Description of Artificial Sequence Synthetic oligonucleotide”
gattacaaagaccatgatggcgactataaagaccatgacatcgactacaaggatgatgatgataaa

SEQ ID NO: 54 - DNA, 66 bp, Artificial Sequence
  source: /note=“Description of Artificial Sequence Synthetic oligonucleotide”
gactacaaggaccacgacggagactataaagaccatgatatagattacaaggacgatgacgataaa

SEQ ID NO: 55 - DNA, 66 bp, Artificial Sequence
  source: /note=“Description of Artificial Sequence Synthetic oligonucleotide”
gactacaaggaccacgacggtgactacaaagaccacgatatagactataaagatgacgatgataag

SEQ ID NO: 56 - DNA, 66 bp, Artificial Sequence
  source: /note=“Description of Artificial Sequence Synthetic oligonucleotide”
gactataaagaccacgatggcgactacaaagaccacgacatcgattacaaggacgatgatgacaaa

SEQ ID NO: 57 - DNA, 66 bp, Artificial Sequence
  source: /note=“Description of Artificial Sequence Synthetic oligonucleotide”
gattataaggaccatgacggagactataaagaccatgatattgactacaaagacgacgatgataag

SEQ ID NO: 58 - DNA, 66 bp, Artificial Sequence
  source: /note=“Description of Artificial Sequence Synthetic oligonucleotide”
gactataaggaccatgatggagactataaagatcacgatattgactataaagatgatgatgataag

SEQ ID NO: 59 - DNA, 30 bp, Artificial Sequence
  source: /note=“Description of Artificial Sequence Synthetic oligonucleotide”
cctaaaaagaaacgaaaagtgggcattcac

SEQ ID NO: 60 - DNA, 30 bp, Artificial Sequence
  source: /note=“Description of Artificial Sequence Synthetic oligonucleotide”
cccaagaagaagaggaaggtcggcattcat

SEQ ID NO: 61 - DNA, 30 bp, Artificial Sequence
  source: /note=“Description of Artificial Sequence Synthetic oligonucleotide”
cctaagaaaaagagaaaagtcggaatccac

SEQ ID NO: 62 - DNA, 30 bp, Artificial Sequence
  source: /note=“Description of Artificial Sequence Synthetic oligonucleotide”
cccaagaagaaaagaaaagtaggaattcac

SEQ ID NO: 63 - DNA, 30 bp, Artificial Sequence
  source: /note=“Description of Artificial Sequence Synthetic oligonucleotide”
ccaaaaaaaaaacgcaaggttggaatacac

SEQ ID NO: 64 - DNA, 30 bp, Artificial Sequence
  source: /note=“Description of Artificial Sequence Synthetic oligonucleotide”
ccaaagaaaaagaggaaggtgggaatacat

SEQ ID NO: 65 - DNA, 30 bp, Artificial Sequence
  source: /note=“Description of Artificial Sequence Synthetic oligonucleotide”
cccaaaaagaaaagaaaggtgggtattcac

SEQ ID NO: 66 - DNA, 30 bp, Artificial Sequence
  source: /note=“Description of Artificial Sequence Synthetic oligonucleotide”
ccaaagaagaaaagaaaagtggggatccat

SEQ ID NO: 67 - DNA, 30 bp, Artificial Sequence
  source: /note=“Description of Artificial Sequence Synthetic oligonucleotide”
cctaaaaaaaagcggaaagtgggaattcac

SEQ ID NO: 68 - DNA, 30 bp, Artificial Sequence
  source: /note=“Description of Artificial Sequence Synthetic oligonucleotide”
cctaagaagaagagaaaagttggaatacat

SEQ ID NO: 69 - DNA, 30 bp, Artificial Sequence
  source: /note=“Description of Artificial Sequence Synthetic oligonucleotide”
cccaagaagaaacgaaaagtaggaatccat

SEQ ID NO: 70 - DNA, 30 bp, Artificial Sequence
  source: /note=“Description of Artificial Sequence Synthetic oligonucleotide”
cctaagaagaaaagaaaggtcggcattcat

SEQ ID NO: 71 - DNA, 1032 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 72 - DNA, 1032 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 73 - DNA, 1032 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 74 - DNA, 1032 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 75 - DNA, 1032 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 76 - DNA, 1032 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 77 - DNA, 1032 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 78 - DNA, 1110 bp, Artificial Sequence
  source: /note=“Description of Artificial Sequence Synthetic polynucleotide”
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 79 - DNA, 1110 bp, Artificial Sequence
  source: /note=“Description of Artificial Sequence Synthetic polynucleotide”
gctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 80 - DNA, 1110 bp, Artificial Sequence
  source: /note=“Description of Artificial Sequence Synthetic polynucleotide”
gctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 81 - DNA, 1110 bp, Artificial Sequence
  source: /note=“Description of Artificial Sequence Synthetic polynucleotide”
gccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 82 - DNA, 1110 bp, Artificial Sequence
  source: /note=“Description of Artificial Sequence Synthetic polynucleotide”
gccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 83 - DNA, 1110 bp, Artificial Sequence
  source: /note=“Description of Artificial Sequence Synthetic polynucleotide”
gcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 84 - DNA, 1110 bp, Artificial Sequence
  source: /note=“Description of Artificial Sequence Synthetic polynucleotide”
gctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 85 - DNA, 2439 bp, Artificial Sequence
  source: /note=“Description of Artificial Sequence Synthetic polynucleotide”
gattataaagatcatgacggggactataaggatcacgacatagactacaaagacgatgatgacaaaatggcgcctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 86 - DNA, 2439 bp, Artificial Sequence
  source: /note=“Description of Artificial Sequence Synthetic polynucleotide”
gattataaagaccatgatggtgattacaaggaccatgacatcgattataaagacgacgacgacaaaatggcccctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 87 - DNA, 2439 bp, Artificial Sequence
  source: /note=“Description of Artificial Sequence Synthetic polynucleotide”
gattataaggatcatgatggagactataaggatcatgacatagattacaaagatgacgatgacaagatggcacccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 88 - DNA, 2439 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 89 - DNA, 2439 bp, Artificial Sequence
  source: /note=“Description of Artificial Sequence Synthetic polynucleotide”
gattacaaagaccatgatggcgactataaagaccatgacatcgactacaaggatgatgatgataaaatggctccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 90 - DNA, 2439 bp, Artificial Sequence
  source: /note=“Description of Artificial Sequence Synthetic polynucleotide”
gactacaaggaccacgacggagactataaagaccatgatatagattacaaggacgatgacgataaaatggcacccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 91 - DNA, 2439 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 92 - DNA, 2439 bp, Artificial Sequence
  source: /note=“Description of Artificial Sequence Synthetic polynucleotide”
gattacaaagatcacgacggagattacaaagatcacgacattgactataaggacgacgacgataaaatggctccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 93 - DNA, 2439 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 94 - DNA, 2439 bp, Artificial Sequence
  source: /note=“Description of Artificial Sequence Synthetic polynucleotide”
gactataaagaccacgatggcgactacaaagaccacgacatcgattacaaggacgatgatgacaaaatggcacctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 95 - DNA, 2439 bp, Artificial Sequence
  source: /note=“Description of Artificial Sequence Synthetic polynucleotide”
gattataaggaccatgacggagactataaagaccatgatattgactacaaagacgacgatgataagatggcccccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 96 - DNA, 2439 bp, Artificial Sequence
  source: /note=“Description of Artificial Sequence Synthetic polynucleotide”
gactataaggaccatgatggagactataaagatcacgatattgactataaagatgatgatgataagatggcacctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 97 - DNA, 2439 bp, Artificial Sequence
  source: /note=“Description of Artificial Sequence Synthetic polynucleotide”
gactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctacgcgtgccatggactacaaagaccatgacggtgattataaagatcatgacatcgattacaaggatgacgatgacaagatggcccccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 98 - DNA, 2439 bp, Artificial Sequence
  source: /note=“Description of Artificial Sequence Synthetic polynucleotide”
gactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctacgcgtgccatggactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 99 - DNA, 2439 bp, Artificial Sequence
  source: /note=“Description of Artificial Sequence Synthetic polynucleotide”
gactacaaagatcatgatggcgactacaaagatcatgatatagattacaaagacgatgacgacaaaatggctccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctacgcgtgccatggactacaaggaccacgacggtgactacaaagaccacgatatagactataaagatgacgatgataagatggcacctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 100 - DNA, 2211 bp, Artificial Sequence
  source: /note=“Description of Artificial Sequence Synthetic polynucleotide”
gtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaactttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 101 - DNA, 2211 bp, Artificial Sequence
  source: /note=“Description of Artificial Sequence Synthetic polynucleotide”
gtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaattttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 102 - DNA, 2211 bp, Artificial Sequence
  source: /note=“Description of Artificial Sequence Synthetic polynucleotide”
gtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaattttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 103 - DNA, 2211 bp, Artificial Sequence
  source: /note=“Description of Artificial Sequence Synthetic polynucleotide”
gtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 104 - DNA, 2211 bp, Artificial Sequence
  source: /note=“Description of Artificial Sequence Synthetic polynucleotide”
gtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 105 - DNA, 2211 bp, Artificial Sequence
  source: /note=“Description of Artificial Sequence Synthetic polynucleotide”
gttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 106 - DNA, 2211 bp, Artificial Sequence
  source: /note=“Description of Artificial Sequence Synthetic polynucleotide”
gtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttcggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 107 - DNA, 2211 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtctggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 108 - DNA, 2211 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 109 - DNA, 2127 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagtggcagcggagagggcagaggaagcctgctcacctgcggtgacgtggaggaaaaccctggccctgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 110 - DNA, 2211 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 111 - DNA, 2211 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagtggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 112 - DNA, 2211 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctccggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 113 - DNA, 2211 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatctggcagcggagagggcagaggaagcctgctcacctgcggtgacgtggaggaaaaccctggccctgtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 114 - DNA, 2211 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagtggcagcggagagggcagaggaagcctgctcacctgcggtgacgtggaggaaaaccctggccctgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 115 - DNA, 2211 bp, Artificial Sequence
  source: /note=“Description of Artificial Sequence Synthetic polynucleotide”
gtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaactttggcagcggagagggcagaggaagcctgctcacctgcggtgacgtggaggaaaaccctggccctgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 116 - DNA, 432 bp, Artificial Sequence
  source: /note=“Description of Artificial Sequence Synthetic polynucleotide”
gccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcgg

SEQ ID NO: 117 - DNA, 432 bp, Artificial Sequence
  source: /note=“Description of Artificial Sequence Synthetic polynucleotide”
gcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgc

SEQ ID NO: 118 - DNA, 432 bp, Artificial Sequence
  source: /note=“Description of Artificial Sequence Synthetic polynucleotide”
gccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgc

SEQ ID NO: 119 - DNA, 432 bp, Artificial Sequence
  source: /note=“Description of Artificial Sequence Synthetic polynucleotide”
gccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcga

SEQ ID NO: 120 - DNA, 432 bp, Artificial Sequence
  source: /note=“Description of Artificial Sequence Synthetic polynucleotide”
gcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcga

SEQ ID NO: 121 - DNA, 432 bp, Artificial Sequence
  source: /note=“Description of Artificial Sequence Synthetic polynucleotide”
gcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgc

SEQ ID NO: 122 - DNA, 432 bp, Artificial Sequence
  source: /note=“Description of Artificial Sequence Synthetic polynucleotide”
gcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcga

SEQ ID NO: 123 - DNA, 516 bp, Artificial Sequence
  source: /note=“Description of Artificial Sequence Synthetic polynucleotide”
gccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcgg

SEQ ID NO: 124 - DNA, 516 bp, Artificial Sequence
  source: /note=“Description of Artificial Sequence Synthetic polynucleotide”
gctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcaga

SEQ ID NO: 125 - DNA, 516 bp, Artificial Sequence
  source: /note=“Description of Artificial Sequence Synthetic polynucleotide”
gctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcgg

SEQ ID NO: 126 - DNA, 516 bp, Artificial Sequence
  source: /note=“Description of Artificial Sequence Synthetic polynucleotide”
gccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgaga

SEQ ID NO: 127 - DNA, 516 bp, Artificial Sequence
  source: /note=“Description of Artificial Sequence Synthetic polynucleotide”
gccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcaga

SEQ ID NO: 128 - DNA, 516 bp, Artificial Sequence
  source: /note=“Description of Artificial Sequence Synthetic polynucleotide”
gcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcgg

SEQ ID NO: 129 - DNA, 516 bp, Artificial Sequence
  source: /note=“Description of Artificial Sequence Synthetic polynucleotide”
gctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgaga

SEQ ID NO: 130 - protein, 344 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 131 - protein, 370 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 132 - protein, 813 aa, Artificial Sequence
  source: /note=“Description of Artificial Sequence Synthetic polypeptide”
DYKDHDGDYKDHDIDYKDDDDKMAPKKKRKVGIHGVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFGSGEGRGSLLTCGDVEENPGPTRAMDYKDHDGDYKDHDIDYKDDDDKMAPKKKRKVGIHGVPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 133 - protein, 813 aa, Artificial Sequence
  source: /note=“Description of Artificial Sequence Synthetic polypeptide”
DYKDHDGDYKDHDIDYKDDDDKMAPKKKRKVGIHGVPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRSGSGEGRGSLLTCGDVEENPGPTRAMDYKDHDGDYKDHDIDYKDDDDKMAPKKKRKVGIHGVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 134 - protein, 735 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFGSGEGRGSLLTCGDVEENPGPAAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 135 - protein, 737 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRSGSGEGRGSLLTCGDVEENPGPVPAAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 136 - protein, 144 aa, Artificial Sequence
  source: /note=“Description of Artificial Sequence Synthetic polypeptide”
AAMAERPFQCRICMQNFSQSGNLARHIRTHTGEKPFACDICGRKFALKQNLCMHTKIHTGEKPFQCRICMQKFAWQSNLQNHTKIHTGEKPFQCRICMRNFSTSGNLTRHIRTHTGEKPFACDICGRKFARRSHLTSHTKIHLR

SEQ ID NO: 137 - protein, 172 aa, Artificial Sequence
  source: /note=“Description of Artificial Sequence Synthetic polypeptide”
AAMAERPFQCRICMRNFSQSSDLSRHIRTHTGEKPFACDICGRKFALKHNLLTHTKIHTGEKPFQCRICMQNFSDQSNLRAHIRTHTGEKPFACDICGRKFARNFSLTMHTKIHTGERGFQCRICMRNFSLRHDLERHIRTHTGEKPFACDICGRKFAHRSNLNKHTKIHLR

SEQ ID NO: 138 - protein, 21 aa, Artificial Sequence
  source: /note=“Description of Artificial Sequence Synthetic peptide”
GSGEGRGSLLTCGDVEENPGP

SEQ ID NO: 139 - DNA, 1071 bp, Artificial Sequence
  source: /note=“Description of Artificial Sequence Synthetic polynucleotide”
cccaagaagaagaggaaggtcggcattcatggggtacccgccgctatggctgagaggcccttccagtgtcgaatctgcatgcagaacttcagtcagtccggcaacctggcccgccacatccgcacccacaccggcgagaagccttttgcctgtgacatttgtgggaggaaatttgccctgaagcagaacctgtgtatgcataccaagatacacacgggcgagaagcccttccagtgtcgaatctgcatgcagaagtttgcctggcagtccaacctgcagaaccataccaagatacacacgggcgagaagcccttccagtgtcgaatctgcatgcgtaacttcagtacctccggcaacctgacccgccacatccgcacccacaccggcgagaagccttttgcctgtgacatttgtgggaggaaatttgcccgccgctcccacctgacctcccataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 140 - DNA, 1071 bp, Artificial Sequence
  source: /note=“Description of Artificial Sequence Synthetic polynucleotide”
ccaaagaagaaaagaaaagtggggatccatggtgtacccgcagcaatggccgaacgacccttccaatgcagaatatgtatgcagaatttttctcagagcgggaacctggcgaggcacataagaacccatacaggagagaagccattcgcatgcgatatttgcggtagaaaatttgcactcaaacaaaatctctgtatgcacactaaaatccatacaggtgaaaagccttttcagtgcaggatttgtatgcaaaaatttgcttggcaaagtaacttgcagaaccacacaaagatacacacaggagagaaacccttccaatgccgaatctgtatgcgcaacttcagtacatccggaaatttgactagacatattaggacccacaccggcgagaagccatttgcctgcgatatttgtggacggaaattcgcacgacgcagccatctgaccagtcatactaagattcatctccgcggcagccagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 141 - DNA, 1071 bp, Artificial Sequence
  source: /note=“Description of Artificial Sequence Synthetic polynucleotide”
cctaaaaaaaagcggaaagtgggaattcacggcgtgcccgccgccatggcagagagaccctttcaatgtagaatctgtatgcaaaatttctctcagagtggtaaccttgcaagacacatcagaactcatacaggtgagaagccgtttgcatgtgacatttgcggtaggaaatttgccttgaaacagaatctttgtatgcacacaaaaatccatactggtgaaaagccattccaatgccgcatctgtatgcaaaaattcgcgtggcagtccaatttgcagaaccataccaagattcacacgggagaaaaaccatttcagtgccgcatctgcatgcgcaacttttctacatcaggaaaccttacacgacatattcggacgcacactggagaaaaaccatttgcttgtgacatatgcggccgaaaatttgccagacgctctcatctcacctcacatactaagattcatttgcgcggaagtcagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 142 - DNA, 1071 bp, Artificial Sequence
  source: /note=“Description of Artificial Sequence Synthetic polynucleotide”
cccaaaaagaagagaaaagtgggaatccacggtgtaccggccgcgatggcagagagaccatttcagtgtagaatctgtatgcagaacttttcccaatcaggaaacctggcacgacacattagaacccatactggagaaaagccgttcgcttgcgacatttgcggtagaaaatttgctttgaaacagaacttgtgtatgcataccaagattcataccggcgaaaaaccatttcaatgcaggatttgtatgcagaagttcgcctggcaatccaatttgcagaatcatactaaaattcataccggagaaaaaccattccaatgccgcatttgtatgagaaacttttctacctctggcaatctcaccagacatatcagaacacacacaggcgagaaaccgttcgcatgcgatatctgtgggcgaaagtttgccagaagatcccatctcacatcacatactaaaatacatttgcgaggaagtcaactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 143 - DNA, 1071 bp, Artificial Sequence
  source: /note=“Description of Artificial Sequence Synthetic polynucleotide”
cctaagaagaagagaaaagttggaatacatggagtccccgcagcaatggccgagagaccttttcagtgcaggatttgtatgcaaaacttctctcagtccggtaacctggcccggcacatacgaacacataccggcgaaaaaccctttgcttgcgacatctgcggaagaaagttcgctcttaaacagaacctgtgcatgcatacaaaaattcatacaggtgagaagccattccaatgcagaatatgtatgcagaaattcgcctggcaaagcaacctgcaaaaccacactaagatccacacaggggaaaagccttttcaatgtagaatctgtatgagaaactttagtacatccggaaatctcacacgacatatcagaacccacactggagaaaaaccttttgcctgcgacatctgcggaagaaaattcgcccgaaggtcccacttgactagtcataccaaaatccacttgcgaggctcacagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 144 - DNA, 1071 bp, Artificial Sequence
  source: /note=“Description of Artificial Sequence Synthetic polynucleotide”
cccaagaagaaacgaaaagtaggaatccatggcgtgcctgcagcaatggcagagagaccatttcagtgcagaatatgtatgcaaaacttctcccagagcggtaatctggctaggcatattagaacacacaccggggaaaaacctttcgcttgcgatatatgtggtagaaagttcgccctcaaacagaatctgtgcatgcacactaaaatccatacaggagaaaagccctttcagtgtagaatttgtatgcagaaatttgcttggcagtcaaatttgcaaaatcacaccaaaatacacacaggagaaaaaccatttcagtgtagaatatgtatgagaaatttttccacttccggaaatctgaccagacatatacggacacacactggggaaaagcccttcgcttgcgacatctgcggaagaaagttcgctagacggtcccacttgacatcccacactaagatacatcttcgcggtagccaactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 145 - DNA, 1071 bp, Artificial Sequence
  source: /note=“Description of Artificial Sequence Synthetic polynucleotide”
cctaagaagaaaagaaaggtcggcattcatggtgtgcctgcagccatggccgaacgcccatttcaatgtagaatttgtatgcagaatttttcacaatcaggaaacctggctagacatatcagaacacatactggagaaaagccctttgcttgtgatatctgtggaaggaaattcgccctgaaacaaaacctctgtatgcacacaaagatccacaccggcgaaaagcctttccagtgtaggatatgcatgcaaaaattcgcctggcagtccaatctgcagaaccataccaaaattcatactggtgaaaagccatttcagtgcagaatatgtatgagaaactttagcacttcaggaaatctcacaagacatataagaacacatacaggggaaaaaccttttgcttgcgatatctgcggcaggaaattcgctcggagaagtcatctcacaagccatacaaaaatccacctgcgaggaagccagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 146 - DNA, 1149 bp, Artificial Sequence
  source: /note=“Description of Artificial Sequence Synthetic polynucleotide”
cccaagaagaagaggaaggtcggcattcatggggtacccgccgctatggctgagaggcccttccagtgtcgaatctgcatgcgtaacttcagtcagtcctccgacctgtcccgccacatccgcacccacaccggcgagaagccttttgcctgtgacatttgtgggaggaaatttgccctgaagcacaacctgctgacccataccaagatacacacgggcgagaagcccttccagtgtcgaatctgcatgcagaacttcagtgaccagtccaacctgcgcgcccacatccgcacccacaccggcgagaagccttttgcctgtgacatttgtgggaggaaatttgcccgcaacttctccctgaccatgcataccaagatacacaccggagagcgcggcttccagtgtcgaatctgcatgcgtaacttcagtctgcgccacgacctggagcgccacatccgcacccacaccggcgagaagccttttgcctgtgacatttgtgggaggaaatttgcccaccgctccaacctgaacaagcataccaagatacacctgcggggatcccagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 147 - DNA, 1149 bp, Artificial Sequence
  source: /note=“Description of Artificial Sequence Synthetic polynucleotide”
cctaaaaagaaacgaaaagtgggcattcacggcgtacctgctgctatggctgaaagaccttttcaatgtcgaatctgcatgaggaattttagtcagtcatccgacctgagcagacacattcgaacccatactggtgaaaagccatttgcttgcgatatatgtgggagaaaatttgcgttgaaacacaatctgctgacccataccaagattcataccggagaaaaaccattccaatgccgcatttgtatgcagaactttagtgaccagtcaaatctccgcgctcacattcgaacccacactggcgaaaaaccctttgcttgtgacatttgcggtcggaagtttgcccgaaatttttctctgacaatgcacacaaaaatccacaccggggaacgcggctttcaatgtaggatctgtatgagaaattttagccttagacatgatttggaacgacatatcaggacccatacaggcgagaaaccatttgcgtgcgatatttgtggcaggaaattcgcacatagaagtaatctgaacaagcatacaaaaattcatctcagaggaagtcagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 148 - DNA, 1149 bp, Artificial Sequence
  source: /note=“Description of Artificial Sequence Synthetic polynucleotide”
cctaagaaaaagagaaaagtcggaatccacggtgtcccagctgccatggccgagagaccatttcaatgtcggatttgcatgcgcaatttttcccagtcctctgaccttagccggcatattcggacacacacaggtgaaaaacccttcgcatgcgacatttgcggaagaaaattcgctctgaaacacaacctgcttacccatacaaagatccacaccggcgagaaaccgtttcaatgccgaatctgtatgcaaaattttagtgatcaaagtaatctgagagcacatattaggactcacacgggcgagaagccatttgcgtgtgatatctgcggccgaaaattcgcccggaatttctctctgacaatgcacaccaaaatccacactggggaacgaggctttcaatgtagaatatgtatgcggaatttcagtctgaggcacgacctggagcggcacatcagaactcacaccggagaaaaaccattcgcttgtgatatttgcgggaggaagttcgcccataggagcaatctcaataaacacaccaaaatacatcttcggggttctcaactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 149 - DNA, 1149 bp, Artificial Sequence
  source: /note=“Description of Artificial Sequence Synthetic polynucleotide”
cccaagaagaaaagaaaagtaggaattcacggagtccctgccgccatggccgagcgccccttccaatgccgcatatgcatgagaaatttcagccaaagtagcgacctgtcacgacacattagaactcatacgggggagaagccatttgcttgcgatatttgtggcagaaaattcgcactcaaacacaacctgctcacacacaccaagatacacacgggagagaagcccttccaatgtagaatatgtatgcaaaatttcagcgaccaaagtaatttgagagcgcatattcgaactcacaccggcgaaaaaccatttgcctgcgatatttgtgggaggaaatttgccaggaatttttcactcaccatgcacactaagatccacactggcgagcgcggcttccaatgcagaatctgtatgcgaaacttcagtctgcggcatgacctggaaagacatataagaacccacaccggagaaaaaccctttgcctgcgacatatgtggtagaaaattcgcacatcggagtaaccttaacaaacatacaaagatccacttgagaggcagtcagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 150 - DNA, 1149 bp, Artificial Sequence
  source: /note=“Description of Artificial Sequence Synthetic polynucleotide”
ccaaaaaaaaaacgcaaggttggaatacacggtgtacctgccgctatggctgaaagacctttccagtgtaggatttgcatgagaaatttttcccaatcatccgacctttcaaggcatattaggacacacaccggggaaaagccatttgcttgtgatatctgcgggcgcaaatttgctcttaagcacaatcttcttacccacaccaaaattcatacaggagaaaaaccttttcaatgtagaatctgcatgcaaaacttttccgatcagtcaaatcttagagctcatatcagaacccataccggggagaaaccctttgcctgcgacatatgcggaagaaaatttgctaggaactttagtctgaccatgcataccaaaattcataccggcgaacgcggtttccagtgcaggatttgtatgagaaatttctcactgcggcatgatcttgaaagacacatacgaactcataccggagaaaagccattcgcttgcgatatttgtggtagaaaatttgcccacaggtctaaccttaataagcacaccaagattcatctcagaggatctcagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 151 - DNA, 1149 bp, Artificial Sequence
  source: /note=“Description of Artificial Sequence Synthetic polynucleotide”
ccaaagaaaaagaggaaggtgggaatacatggagtaccagcagctatggccgaacgcccttttcaatgcagaatatgtatgcgaaacttctcccaaagctctgatctgtcaaggcacatacggacacacaccggcgaaaaaccctttgcatgtgacatttgtggaagaaaattcgcacttaaacacaatctcctgactcatacaaaaatacatacaggcgaaaaacctttccagtgcagaatctgtatgcagaacttttccgaccaatccaatcttcgcgcccacattagaactcacacaggggagaaacctttcgcttgcgacatatgcggaagaaaatttgccagaaatttttcacttacaatgcacacaaaaatacatactggggaaagagggtttcaatgtcgaatctgtatgagaaatttcagtctgcgccatgatctggagagacatataagaacacacacaggagagaaaccttttgcttgtgacatatgcggccgaaagtttgctcatagatctaatcttaacaaacatacaaagatccatcttcggggttcacaactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 152 - DNA, 1149 bp, Artificial Sequence
  source: /note=“Description of Artificial Sequence Synthetic polynucleotide”
cccaaaaagaaaagaaaggtgggtattcacggagttcccgctgctatggctgagagacctttccaatgtaggatctgtatgcgaaacttctcccagagctccgacctgagtcgccatataagaacccataccggagaaaaaccatttgcttgtgacatttgtggcagaaagttcgctcttaaacacaacctgcttacacatactaaaatacacacaggggagaaaccctttcaatgccggatctgtatgcaaaactttagcgatcaatcaaacttgcgagcccatatccgcactcacaccggcgagaagccttttgcatgcgatatatgtggacggaaatttgctagaaacttctcattgaccatgcatacaaaaatacacaccggggaacgaggatttcaatgtcgaatttgtatgagaaattttagccttaggcacgacttggaacggcacataagaacccacaccggagagaagccttttgcttgtgatatttgcggcagaaagttcgcccatcgcagcaatcttaacaagcacaccaagattcatttgagaggttcccagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 153 - DNA, 66 bp, Artificial Sequence
  source: /note=“Description of Artificial Sequence Synthetic oligonucleotide”
gattacaaagatcacgacggagattacaaagatcacgacattgactataaggacgacgacgataaa

SEQ ID NO: 154 - DNA, 66 bp, Artificial Sequence
  source: /note=“Description of Artificial Sequence Synthetic oligonucleotide”
gattacaaagaccacgacggagactacaaggaccatgatattgactacaaagacgatgatgataag

SEQ ID NO: 155 - DNA, 30 bp, Artificial Sequence
  source: /note=“Description of Artificial Sequence Synthetic oligonucleotide”
cccaaaaagaagagaaaagtgggaatccac

SEQ ID NO: 156 - protein, 10 aa, Artificial Sequence
  source: /note=“Description of Artificial Sequence Synthetic peptide”
PKKKRKVGIH

SEQ ID NO: 157 - DNA, 588 bp, Artificial Sequence
  source: /note=“Description of Artificial Sequence Synthetic polynucleotide”
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgagcatcggccaggccgacgagatgcagagatacgtgaaggagaaccagacccggaataagcacatcaaccccaacgagtggtggaaggtgtaccctagcagcgtgaccgagttcaagttcctgttcgtgagcggccacttcaagggcaactacaaggcccagctgaccaggctgaaccgcaaaaccaactgcaatggcgccgtgctgagcgtggaggagctgctgatcggcggcgagatgatcaaagccggcaccctgacactggaggaggtgcggcgcaagttcaacaacggcgagatcaacttc

SEQ ID NO: 158 - DNA, 588 bp, Artificial Sequence
  source: /note=“Description of Artificial Sequence Synthetic polynucleotide”
cagctggtcaaaagtgaactggaggaaaaaaagagcgaactgagacacaaactgaagtacgtgccacacgaatatattgagctgattgagatcgcgaggaactcaacacaggaccgcattctggagatgaaagtgatggagtttttcatgaaagtatatggatatagaggaaaacaccttgggggtagccgaaagccggacggggcgatctacactgtggggtcaccaattgattatggcgtaattgtcgataccaaagcctacagtggggggtacaatctgagtataggacaggctgatgaaatgcaacgatacgttaaggagaatcagactaggaataaacatatcaatccaaatgaatggtggaaagtctatcccagcagcgtgacagaatttaaatttttgtttgtcagtggacacttcaagggaaattataaggcccagctgactagactgaataggaaaaccaattgtaatggcgcagtgctttcagtggaggaactgctcattggaggtgagatgatcaaggctggaaccctgacgctggaggaggtgcggaggaagtttaacaatggagaaattaacttt

SEQ ID NO: 159 - DNA, 588 bp, Artificial Sequence
  source: /note=“Description of Artificial Sequence Synthetic polynucleotide”
caactggtgaaatccgaactggaagaaaagaaatcagaattgcggcataaactgaagtatgtgccccatgagtacatagaactgatcgagatcgcaaggaactctacccaggacagaatacttgaaatgaaggtcatggaattttttatgaaagtgtacggctacagaggaaaacatttgggaggcagtcgaaaaccagatggcgcaatctatacagtcgggtcccccatagattacggagtgattgtcgacacaaaagcctattccggaggatataaccttagtatcggccaggccgacgagatgcaacgctatgtgaaagaaaaccaaacaagaaataaacatatcaatccaaacgagtggtggaaggtatatccaagcagtgtcacagaattcaaattcctcttcgtgagtgggcactttaaaggcaactacaaagctcaattgaccaggctcaatcggaaaactaattgcaatggcgcagtccttagcgtcgaagaattgctgattggcggggaaatgattaaagcaggaactttgaccttggaggaagtacggagaaagtttaacaacggcgagattaatttt

SEQ ID NO: 160 - DNA, 588 bp, Artificial Sequence
  source: /note=“Description of Artificial Sequence Synthetic polynucleotide”
cagctggtgaaatctgagctggaagagaagaaatctgaactgcgacataaattgaagtacgtcccacacgagtacatcgagttgatcgaaattgcccggaatagcacccaggatagaatattggaaatgaaagtaatggagttttttatgaaggtttatggttacagaggcaagcaccttggaggaagcaggaaaccagatggggcgatttacaccgttgggagtcccatcgattacggagtcatcgtggacacaaaggcctattccggaggctacaacctcagtatcgggcaagccgatgagatgcagagatatgttaaagaaaatcagacgcgaaacaagcacattaacccaaacgaatggtggaaagtttaccctagctcagtgacagaatttaagtttctgtttgtcagcggccacttcaaggggaattataaagcacaactgacccgcctgaaccgaaaaaccaactgtaacggtgctgtgctgagtgtcgaagagttgcttatcggaggagagatgataaaggccggcacactgacgcttgaagaggtacggcgaaaattcaataacggagagattaatttt

SEQ ID NO: 161 - DNA, 588 bp, Artificial Sequence
  source: /note=“Description of Artificial Sequence Synthetic polynucleotide”
cagctggtcaaatcagaacttgaagagaaaaaaagcgaactgagacataaactgaagtacgtgcctcatgaatacatagagctcattgaaatagctaggaatagtacacaggacaggatacttgaaatgaaggtaatggaatttttcatgaaggtttatggataccgggggaaacatctcgggggcagcagaaaaccagacggagcaatttatactgtcgggagtcctatagattatggcgttatcgtcgatacaaaggcctattccggtgggtacaacctctcaattggtcaggctgatgagatgcaaagatacgtcaaagaaaaccaaaccagaaataaacatataaatcccaatgaatggtggaaagtatacccaagttccgtgactgaattcaagttccttttcgtgtctggccactttaaaggaaattataaagcacaattgactagactgaatagaaaaacaaactgtaacggcgcagtgctgtcagtggaagaactgctcataggtggagagatgatcaaggccgggacacttactcttgaggaagttagaaggaagttcaacaacggcgaaatcaacttt

SEQ ID NO: 162 - DNA, 588 bp, Artificial Sequence
  source: /note=“Description of Artificial Sequence Synthetic polynucleotide”
caactggtcaagtcagaattggaagagaaaaaatctgagctgaggcacaaattgaaatacgttcctcacgagtatattgaacttatcgagatagcccgcaatagtacacaagatagaatcttggagatgaaagttatggaattctttatgaaagtctatggctataggggaaaacacctggggggtagcaggaaacctgatggagctatctataccgtaggatcacctattgattatggagtaattgtggacactaaggcatattccggaggatataatttgagtattggtcaggccgacgaaatgcaacgatacgtgaaggaaaatcagacccgcaacaaacacattaatcccaatgaatggtggaaggtataccctagtagcgttacagagtttaaattccttttcgtcagcggccactttaaaggaaattataaagcacaactcaccagacttaatcgaaaaactaactgtaacggcgccgtactgtcagtggaggagctgctcattggaggcgagatgatcaaggccggtactctcacactggaagaagttagaagaaagttcaacaacggggaaattaatttc

SEQ ID NO: 163 - DNA, 588 bp, Artificial Sequence
  source: /note=“Description of Artificial Sequence Synthetic polynucleotide”
cagctggtcaaaagcgaacttgaagaaaagaaatccgagcttagacacaaactgaaatacgtgcctcacgagtatattgagctgattgaaatagcaaggaattcaacacaagacaggatcctcgaaatgaaggttatggagtttttcatgaaagtttacggctacagagggaagcatctgggcggatcaagaaaaccagacggcgcaatctacacagttggatccccaatagattacggagtgattgttgacaccaaggcttattcaggaggttacaatctgtccattggtcaggccgatgaaatgcaaagatatgttaaggaaaatcaaactcgaaacaaacacattaatccaaacgaatggtggaaagtatatccaagctccgtcactgaatttaaatttttgtttgtatccggacattttaagggcaactataaggctcaactgaccagactgaataggaagaccaattgtaacggagctgtactcagcgtggaagaactgcttattggaggcgaaatgattaaggctggcacacttacactcgaagaagttagaagaaaattcaacaatggtgagataaacttc

SEQ ID NO: 164 - DNA, 594 bp, Artificial Sequence
  source: /note=“Description of Artificial Sequence Synthetic polynucleotide”
cagctggtgaagagcgagctggaggagaagaagtccgagctgcggcacaagctgaagtacgtgccccacgagtacatcgagctgatcgagatcgccaggaacagcacccaggaccgcatcctggagatgaaggtgatggagttcttcatgaaggtgtacggctacaggggaaagcacctgggcggaagcagaaagcctgacggcgccatctatacagtgggcagccccatcgattacggcgtgatcgtggacacaaaggcctacagcggcggctacaatctgcctatcggccaggccgacgagatggagagatacgtggaggagaaccagacccgggataagcacctcaaccccaacgagtggtggaaggtgtaccctagcagcgtgaccgagttcaagttcctgttcgtgagcggccacttcaagggcaactacaaggcccagctgaccaggctgaaccacatcaccaactgcgacggcgccgtgctgagcgtggaggagctgctgatcggcggcgagatgatcaaagccggcaccctgacactggaggaggtgcggcgcaagttcaacaacggcgagatcaacttcagatct

SEQ ID NO: 165 - DNA, 594 bp, Artificial Sequence
  source: /note=“Description of Artificial Sequence Synthetic polynucleotide”
cagcttgtgaagtccgaactggaggaaaagaagagcgaactgcgccacaaattgaaatacgttccgcatgagtacatagagctcattgaaatcgctagaaactctacccaagacaggatactggaaatgaaagtgatggaatttttcatgaaagtttatggttataggggcaaacatctgggtggctctcgcaagcccgatggggccatttatactgtcggctcacctatcgactatggcgtcattgtggataccaaggcttattctggaggatacaacctgcccatcggacaagcagacgaaatggaaagatacgtcgaggagaatcaaacccgagacaagcatctgaacccaaacgagtggtggaaagtgtacccgagcagcgttactgagttcaaatttctctttgtaagcggacattttaaagggaattacaaagcacaactgactaggctgaaccatataaccaactgtgacggggccgtattgagtgtggaagagcttctgattggaggagagatgattaaggctggcacactgactctcgaagaagtgaggcgcaaattcaataacggtgaaatcaacttccggtct

SEQ ID NO: 166 - DNA, 594 bp, Artificial Sequence
  source: /note=“Description of Artificial Sequence Synthetic polynucleotide”
cagctggtgaagagtgaattggaagaaaaaaagtcagagctgagacacaaactgaaatatgttccacacgagtacatcgagcttatcgagatagcaagaaactccacccaggacagaattttggaaatgaaagttatggaattctttatgaaagtgtatggctacaggggtaaacatctggggggatcaagaaagcctgatggtgcaatttacacagtgggctctcctatcgactacggtgtgatcgtggatacaaaggcctactctggaggatataatttgcctattggacaagccgatgaaatggaaagatatgtggaggaaaaccagactcgcgataagcacctgaacccaaatgaatggtggaaagtgtacccttcatctgttaccgaatttaaatttttgttcgtttccgggcatttcaaggggaactacaaggcacagctgacgagactgaatcacatcacgaactgcgacggcgctgtactgtccgtggaagagcttttgatcgggggcgaaatgattaaggccggcacactgacgctggaggaggtgcggcgaaaatttaataatggcgagatcaattttaggagt

SEQ ID NO: 167 - DNA, 594 bp, Artificial Sequence
  source: /note=“Description of Artificial Sequence Synthetic polynucleotide”
caactggtcaagtccgaactggaggaaaaaaaaagtgagctgcgacacaagttgaagtacgtaccacacgaatacatcgagctgattgagatagcacggaactctacccaggatagaatactggagatgaaagttatggaattctttatgaaggtgtacggatacagggggaagcatcttggcgggagccggaaaccagacggagcaatctataccgtcgggtcacctatagactatggagttattgtcgatacaaaggcctattcaggaggttataatctgccaatcggccaagccgacgagatggagaggtacgtggaggaaaatcagaccagagacaagcacctgaaccctaatgaatggtggaaagtgtaccctagcagcgtcactgagttcaaattcctgttcgtcagcggtcattttaaaggaaattataaagcccagctcactagactcaaccatattacaaactgcgacggagccgtacttagcgttgaagagttgcttatcggaggagagatgatcaaagccggaaccctcacacttgaagaagtgcgaagaaaattcaataacggagagataaattttaggagt

SEQ ID NO: 168 - DNA, 594 bp, Artificial Sequence
  source: /note=“Description of Artificial Sequence Synthetic polynucleotide”
cagctggttaaatccgaacttgaagaaaaaaaaagtgaactgcggcataaactgaagtatgtcccccatgaatatatcgaactgatagaaatcgcccgaaatagcacccaagatagaatcctcgaaatgaaggttatggaatttttcatgaaggtctatggatataggggcaagcaccttggcggatcccggaaacctgatggagctatctacacagtgggctcaccaatagactatggagttatcgtcgatacaaaagcatacagcggaggatacaatttgccaataggtcaagcagatgagatggaaagatacgtggaggaaaaccaaacaagagataagcatctgaaccccaacgaatggtggaaagtgtaccccagttctgtaaccgaatttaagttcttgttcgtttcaggtcacttcaagggtaattacaaggctcaactgactagactcaaccatattacaaattgcgatggtgctgtgctttccgtggaagaattgctgattggtggagagatgataaaagctggtaccctcaccttggaagaagtgcgcagaaaattcaataatggcgagatcaacttccgaagt

SEQ ID NO: 169 - DNA, 594 bp, Artificial Sequence
  source: /note=“Description of Artificial Sequence Synthetic polynucleotide”
caactggtgaaaagtgaactggaggaaaaaaaatctgagctgagacataaactgaaatacgtaccacatgaatacatagaacttatagaaatagctaggaactccacccaggacagaatacttgaaatgaaggtcatggagttttttatgaaagtttacggatacaggggcaaacaccttggagggtctcggaagcctgatggcgcaatttataccgtgggtagccctatagattatggagtgattgtggatacaaaggcttacagtggcggctataatttgcctatcggacaggccgatgagatggaaagatacgttgaagaaaaccaaacacgagataagcatctgaaccccaatgaatggtggaaagtgtatccttcaagcgttaccgagtttaagttcctcttcgtttctgggcatttcaagggcaactacaaagctcagcttacaagactcaaccacataaccaattgtgatggagcagtcctcagcgtggaagaactccttattgggggtgagatgattaaagcagggacccttactcttgaagaggttagaagaaaattcaataacggagagattaattttagaagt

SEQ ID NO: 170 - DNA, 594 bp, Artificial Sequence
  source: /note=“Description of Artificial Sequence Synthetic polynucleotide”
cagctggtcaagtctgaactggaagaaaaaaaaagcgaactgcggcataaactcaaatacgtcccacatgaatacattgagctcatcgaaattgctagaaactctactcaagataggatattggagatgaaggtaatggaattcttcatgaaggtttatggatatagaggaaaacatcttggaggcagtaggaaacccgatggcgctatctacaccgtagggagtccaatcgactacggcgtgattgttgacaccaaagcctattctggagggtataatctcccaattggacaggcagatgagatggaaagatatgtagaagaaaatcagacaagagataagcaccttaaccctaacgagtggtggaaagtgtacccaagcagtgttactgaatttaaatttctttttgtatcaggacactttaaaggcaattacaaagcacaactgaccagactcaatcacattaccaattgcgacggagccgtactgagcgtggaggagttgctgatcggaggcgaaatgattaaagctggcactctgaccctggaagaagtaagaagaaagttcaataatggagaaataaactttcgctcc

SEQ ID NO: 171 - protein, 196 aa, Artificial Sequence
  source: /note=“Description of Artificial Sequence Synthetic polypeptide”
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLSIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 172 - protein, 198 aa, Artificial Sequence
  source: /note=“Description of Artificial Sequence Synthetic polypeptide”
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCDGAVLSVEELLIGGEMIKAGTLTLEEVRRKFNNGEINFRS

SEQ ID NO: 173 - protein, 4 aa, Artificial Sequence
  source: /note=“Description of Artificial Sequence Synthetic peptide”
NPGP

(19) United States
(12) Patent Application Publication
(10) Pub. No.: US 2023/0048224 A1
(43) Pub. Date: Feb. 16, 2023

(54) ZINC FINGER NUCLEASE VARIANTS FOR TREATING OR PREVENTING LYSOSOMAL STORAGE DISEASES

(71) Applicant: Sangamo Therapeutics, Inc., Richmond, CA (US)

(72) Inventors: Gustavo De Alencastro, Richmond, CA (US); Marshall Huston, Richmond, CA (US); David Shivak, Richmond, CA (US)

(21) Appl. No.: 17/773,507

(22) PCT Filed: Oct. 30, 2020
(86) PCT No.: PCT/US2020/058361

§ 371 Date: Apr. 29, 2022

Related U.S. Application Data
(60) Provisional application No. 62/929,523, filed on Nov. 1, 2019.

(51) Int. Cl. C12N 9/22; C12N 15/90

(52) U.S. Cl. CPC C12N 9/22; C12N 15/907; A61K 38/00

(57) ABSTRACT

The present disclosure provides 2-in-1 zinc finger nuclease variants and methods of treating and/or preventing a lysosomal storage disorder using said zinc finger nuclease variants.

### CROSS-REFERENCE TO RELATED APPLICATIONS

This application claims priority and benefit from U.S. Provisional Application No. 62/929,523, filed Nov. 1, 2019, the contents of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 27, 2020, is named 000222-0008-WO1_SL.txt and is 342,670 bytes in size.

### BACKGROUND

Lysosomal storage disorders (LSDs) are a group of over 70 rare inherited diseases that are characterized by an accumulation of waste products in the lysosomes due to lysosomal dysfunction. The lysosome is the key cellular hub for macromolecule catabolism, recycling and signaling. Defects that impair any of these functions cause the accumulation of undigested or partially digested macromolecules in lysosomes (i.e., storage) or impair the transport of molecules resulting in cellular damage. Most of the disorders are inherited as autosomal recessive traits. Although individually rare, LSDs as a group have a frequency of about 1/8000 live births, making this disease group a major challenge for the health care system.

LSDs have a broad spectrum of clinical phenotypes. The symptoms vary markedly depending on the particular disorder and other variables such as the age of onset. The symptoms can range from mild to severe. Most LSDs have a progressive neurodegenerative clinical course, including developmental delay, movement disorders, seizures, dementia, deafness, and/or blindness. Symptoms in other organ systems including enlarged livers or spleens, pulmonary and cardiac problems, and bones that grow abnormally are also frequently observed.

There is currently no cure for LSDs, and treatment is directed at alleviating symptoms. Current therapies include bone marrow transplantation, enzyme replacement therapy (ERT), umbilical cord blood transplantation, substrate reduction therapy, and chaperone therapy. However, none is a cure.

Accordingly, there exists a continuing need for new therapies for lysosomal storage disease, which can be effective as a stand-alone therapy or as adjunct therapy to current therapies.

### SUMMARY

The present disclosure provides methods and compositions for treating and/or preventing a lysosomal storage disease in a subject. Thus, a first aspect of the disclosure provides a method for treating or preventing a lysosomal storage disorder in a subject, the method comprising modifying a target sequence in the genome of a cell of said subject by introducing into the cell a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; or a vector comprising said nucleic acid encoding a 2-in-1 zinc finger nuclease variant; wherein the polynucleotide encoding the 2A self-cleaving peptide is positioned between the polynucleotide encoding the first zinc finger nuclease and the polynucleotide encoding the second zinc finger nuclease.

A second aspect of the disclosure provides a method for correcting a lysosomal storage disease-causing mutation in the genome of a cell, the method comprising modifying a target sequence in the genome of the cell by introducing into the cell a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; or a vector comprising said nucleic acid encoding a 2-in-1 zinc finger nuclease variant; wherein the polynucleotide encoding the 2A self-cleaving peptide is positioned between the polynucleotide encoding the first zinc finger nuclease and the polynucleotide encoding the second zinc finger nuclease.

A third aspect of the disclosure provides a method for modifying the genome of a cell comprising a mutation in a gene associated with a lysosomal storage disease, the method comprising introducing into a cell a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; or a vector comprising said nucleic acid encoding a 2-in-1 zinc finger nuclease variant; wherein the polynucleotide encoding the 2A self-cleaving peptide is positioned between the polynucleotide encoding the first zinc finger nuclease and the polynucleotide encoding the second zinc finger nuclease.

A fourth aspect of the disclosure provides a method for integrating an exogenous nucleotide sequence into a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; or a vector comprising said nucleic acid encoding a 2-in-1 zinc finger nuclease variant; wherein the polynucleotide encoding the 2A self-cleaving peptide is positioned between the polynucleotide encoding the first zinc finger nuclease and the polynucleotide encoding the second zinc finger nuclease.

A fifth aspect of the disclosure provides a method for disrupting a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; or a vector comprising said nucleic acid encoding a 2-in-1 zinc finger nuclease variant; wherein the polynucleotide encoding the 2A self-cleaving peptide is positioned between the polynucleotide encoding the first zinc finger nuclease and the polynucleotide encoding the second zinc finger nuclease.

In some embodiments, the methods disclosed herein further comprise introducing into the cell a donor nucleic acid or a vector comprising said donor nucleic acid, wherein said donor nucleic acid comprises a polynucleotide encoding a corrective lysosomal storage disease-associated protein or enzyme or portion thereof. In some embodiments, the donor nucleic acid used in the methods of the disclosure is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.

In some embodiments, the corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.

In some embodiments, the nucleic acid encoding a 2-in-1 zinc finger nuclease variant used in the methods of the disclosure, further comprises a polynucleotide sequence selected from one or more of: 1) a polynucleotide sequence encoding a nuclear localization sequence; 2) a 5′ITR polynucleotide sequence; 3) an enhancer polynucleotide sequence; 4) a promoter polynucleotide sequence; 5) a 5′UTR polynucleotide sequence; 6) a chimeric intron polynucleotide sequence; 7) a polynucleotide sequences encoding an epitope tag; 8) a polynucleotide sequence encoding a Fok I cleavage domain; 9) a post-transcriptional regulatory element polynucleotide sequence; 10) a polyadenylation signal sequence; and 11) a 3′ITR polynucleotide sequence.

In some embodiments, the nucleic acid encoding a 2-in-1 zinc finger nuclease variant used in the methods of the disclosure comprises two independent polynucleotide sequences encoding two nuclear localization sequences.

In some embodiments, the nucleic acid encoding a 2-in-1 zinc finger nuclease variant used in the methods of the disclosure comprises two or more independent polynucleotide sequences encoding two or more epitope tags.

In some embodiments, the nucleic acid encoding a 2-in-1 zinc finger nuclease variant used in the methods of the disclosure comprises two or more independent polynucleotide sequences encoding two or more Fok I cleavage domains.

In some embodiments, the polynucleotide encoding the first zinc finger nuclease used in the methods of the disclosure is codon diversified. In some embodiments, the polynucleotide encoding the second zinc finger nuclease used in the methods of the disclosure is codon diversified. In some embodiments, the polynucleotide encoding the first zinc finger nuclease used in the methods of the disclosure is codon diversified and the polynucleotide encoding the second zinc finger nuclease used in the methods of the disclosure is codon diversified. In some embodiments, the polynucleotide encoding the first zinc finger nuclease used in the methods of the disclosure comprises the nucleotide sequence of any one of SEQ ID NOs: 116-129. In some embodiments, the polynucleotide encoding the second zinc finger nuclease used in the methods of the disclosure comprises the nucleotide sequence of any one of SEQ ID NOs: 116-129. In some embodiments, the polynucleotide encoding the first zinc finger nuclease used in the methods of the disclosure comprises a nucleotide sequence encoding the amino acid sequence of SEQ ID NOs: 136 or 137. In some embodiments, the polynucleotide encoding the second zinc finger nuclease used in the methods of the disclosure comprises a nucleotide sequence encoding the amino acid sequence of SEQ ID NOs: 136 or 137. In some embodiments, the polynucleotide sequence encoding the first zinc finger nuclease used in the methods of the disclosure comprises the nucleotide sequence of any one of SEQ ID NOs: 71-84. In some embodiments, the polynucleotide sequence encoding the second zinc finger nuclease used in the methods of the disclosure comprises the nucleotide sequence of any one of SEQ ID NOs: 71-84. In some embodiments, the polynucleotide encoding the first zinc finger nuclease used in the methods of the disclosure comprises a nucleotide sequence encoding the amino acid sequence of SEQ ID NOs: 130 or 131. In some embodiments, the polynucleotide encoding the second zinc finger nuclease used in the methods of the disclosure comprises a nucleotide sequence encoding the amino sequence of SEQ ID NOs: 130 or 131. In some embodiments, the polynucleotide sequence encoding the first zinc finger nuclease used in the methods of the disclosure comprises the nucleotide sequence of any one of SEQ ID NOs: 139-152. In some embodiments, the polynucleotide sequence encoding the second zinc finger nuclease comprises the nucleotide sequence of any one of SEQ ID NOs: 139-152. In some embodiments, the polynucleotide sequence encoding the first zinc finger nuclease used in the methods of the disclosure comprises the nucleotide sequence of any one of SEQ ID NOs: 17-23 and 25-31. In some embodiments, the polynucleotide sequence encoding the second zinc finger nuclease used in the methods of the disclosure comprises the nucleotide sequence of any one of SEQ ID NOs: 17-23 and 25-31.

In some embodiments, the nucleic acid encoding a 2-in-1 zinc finger nuclease variant used in the methods of the disclosure comprises a nucleotide sequence selected from any one of SEQ ID NO: 85-115. In some embodiments, the nucleic acid encoding a 2-in-1 zinc finger nuclease variant used in the methods of the disclosure comprises a nucleotide sequence selected from any one of SEQ ID NO: 35-49.

In some embodiments, the vector used in the methods of the disclosure is an AAV vector.

A sixth aspect of the disclosure provides a method for treating or preventing a lysosomal storage disorder in a subject, the method comprising modifying a target sequence in the genome of a cell of said subject by introducing into the cell a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and the second zinc finger nuclease.

A seventh aspect of the disclosure provides a method for correcting a lysosomal storage disease-causing mutation in the genome of a cell, the method comprising modifying a target sequence in the genome of the cell by introducing into the cell a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and second zinc finger nuclease.

An eighth aspect of the disclosure provides a method for modifying the genome of a cell comprising a mutation in a gene associated with a lysosomal storage disease, the method comprising introducing into a cell a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and second zinc finger nuclease.

A ninth aspect of the disclosure provides a method for integrating an exogenous nucleotide sequence into a target nucleotide sequence in a gene of a cell, wherein said gene comprises a mutation associated with a lysosomal storage disease, the method comprising introducing into the cell a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and second zinc finger nuclease.

A tenth aspect of the disclosure provides a method for disrupting a target nucleotide sequence in a gene of a cell, wherein said gene comprises a mutation associated with a lysosomal storage disease, the method comprising introducing into the cell a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and second zinc finger nuclease.

In some embodiments, the methods of the disclosure further comprise introducing into the cell a donor nucleic acid or a vector comprising said donor nucleic acid, wherein said donor nucleic acid comprises a polynucleotide encoding a corrective lysosomal storage disease-associated protein or enzyme or portion thereof.

In some embodiments, the donor nucleic acid used in the methods of the disclosure is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.

In some embodiments, the corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.

In some embodiments, the 2-in-1 zinc finger nuclease variant used in the methods of the disclosure further comprises one or more of: 1) a nuclear localization sequence; 2) an epitope tag; and 3) a Fok I cleavage domain. In some embodiments, the 2-in-1 zinc finger nuclease variant used in the methods of the disclosure comprises two independent nuclear localization sequences. In some embodiments, the 2-in-1 zinc finger nuclease variant used in the methods of the disclosure comprises two or more independent epitope tags. In some embodiments, the 2-in-1 zinc finger nuclease variant used in the methods of the disclosure comprises two or more independent Fok I cleavage domains. In some embodiments, the first zinc finger nuclease used in the methods of the disclosure is codon diversified. In some embodiments, the second zinc finger nuclease used in the methods of the disclosure is codon diversified. In some embodiments, the first zinc finger nuclease used in the methods of the disclosure is codon diversified and the second zinc finger nuclease used in the methods of the disclosure is codon diversified.

In some embodiments, the first zinc finger nuclease used in the methods of the disclosure is encoded by a polynucleotide comprising the nucleotide sequence of any one of SEQ ID NOs: 116-129. In some embodiments, the polynucleotide encoding the second zinc finger nuclease used in the methods of the disclosure is encoded by a polynucleotide comprising the nucleotide sequence of any one of SEQ ID NOs: 116-129. In some embodiments, the first zinc finger nuclease used in the methods of the disclosure comprises the amino acid sequence of SEQ ID NOs: 136 or 137. In some embodiments, the second zinc finger nuclease used in the methods of the disclosure comprises the amino acid sequence of SEQ ID NOs: 136 or 137. In some embodiments, the first zinc finger nuclease used in the methods of the disclosure is encoded by a polynucleotide sequence comprising the nucleotide sequence of any one of SEQ ID NOs: 71-84. In some embodiments the second zinc finger nuclease used in the methods of the disclosure is encoded by a polynucleotide sequence comprising the nucleotide sequence of any one of SEQ ID NOs: 71-84. In some embodiments, the first zinc finger nuclease used in the methods of the disclosure comprises the amino acid sequence of SEQ ID NOs: 130 or 131. In some embodiments, the second zinc finger nuclease used in the methods of the disclosure comprises the amino sequence of SEQ ID NOs: 130 or 131. In some embodiments, the first zinc finger nuclease used in the methods of the disclosure is encoded by a polynucleotide comprising the nucleotide sequence of any one of SEQ ID NOs: 139-152. In some embodiments, the second zinc finger nuclease used in the methods of the disclosure is encoded by a polynucleotide comprising the nucleotide sequence of any one of SEQ ID NOs: 139-152. In some embodiments, the first zinc finger nuclease used in the methods of the disclosure is encoded by a polynucleotide comprising the nucleotide sequence of any one of SEQ ID NOs: 17-23 and 25-31. In some embodiments, the second zinc finger nuclease used in the methods of the disclosure is encoded by a polynucleotide comprising the nucleotide sequence of any one of SEQ ID NOs: 17-23 and 25-31. In some embodiments, the 2-in-1 zinc finger nuclease variant used in the methods of the disclosure is encoded by a nucleotide sequence selected from any one of SEQ ID NO: 85-115. In some embodiments, the 2-in-1 zinc finger nuclease variant used in the methods of the disclosure is encoded by a nucleotide sequence selected from any one of SEQ ID NO: 35-49.

In some embodiments, the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease. In some embodiments, the lysosomal storage disease is selected from the group consisting of MPS I and MPS II. In some embodiments, the lysosomal storage disease is MPSI. In some embodiments, the lysosomal storage disease is MPS I—Hurler Syndrome, MPS I—Scheie Syndrome, or MPS I Hurler-Scheie Syndrome. In In some embodiments, the lysosomal storage disease is MPSII. In some embodiments, the lysosomal storage disease is MPS II Hunter Syndrome.

An eleventh aspect of the disclosure provides a nucleic acid encoding a 2-in-1 zinc finger nuclease variant comprising: 1) a polynucleotide encoding a first zinc finger nuclease; 2) a polynucleotide encoding a second zinc finger nuclease; and 3) a polynucleotide encoding a 2A self-cleaving peptide; wherein the polynucleotide encoding the 2A self-cleaving peptide is positioned between the polynucleotide encoding the first zinc finger nuclease and the polynucleotide encoding the second zinc finger nuclease. In some embodiments, the nucleic acid encoding a 2-in-1 zinc finger nuclease variant further comprises a polynucleotide sequence selected from one or more of: 1) a polynucleotide sequence encoding a nuclear localization sequence; 2) a 5′ITR polynucleotide sequence; 3) an enhancer polynucleotide sequence; 4) a promoter polynucleotide sequence; 5) a 5′UTR polynucleotide sequence; 6) a chimeric intron polynucleotide sequence; 7) a polynucleotide sequences encoding an epitope tag; 8) a polynucleotide sequence encoding a Fok I cleavage domain; 9) a post-transcriptional regulatory element polynucleotide sequence; 10) a polyadenylation signal sequence; and 11) a 3′ITR polynucleotide sequence.

In some embodiments, the nucleic acid encoding a 2-in-1 zinc finger nuclease variant comprises two independent polynucleotide sequences encoding two nuclear localization sequences. In some embodiments, the nucleic acid encoding a 2-in-1 zinc finger nuclease variant comprises two or more independent polynucleotide sequences encoding two or more epitope tags. In some embodiments, the nucleic acid encoding a 2-in-1 zinc finger nuclease variant comprises two or more independent polynucleotide sequences encoding two or more Fok I cleavage domains. In some embodiments, the polynucleotide encoding the first zinc finger nuclease is codon diversified.

In some embodiments, the polynucleotide encoding the second zinc finger nuclease is codon diversified. In some embodiments, the polynucleotide encoding the first zinc finger nuclease is codon diversified and the polynucleotide encoding the second zinc finger nuclease is codon diversified. In some embodiments, the polynucleotide encoding the first zinc finger nuclease comprises the nucleotide sequence of any one of SEQ ID NOs: 116-129. In some embodiments, the polynucleotide encoding the second zinc finger nuclease comprises the nucleotide sequence of any one of SEQ ID NOs: 116-129. In some embodiments, the polynucleotide encoding the first zinc finger nuclease comprises a nucleotide sequence encoding the amino acid sequence of SEQ ID NOs: 136 or 137. In some embodiments, the polynucleotide encoding the second zinc finger nuclease comprises a nucleotide sequence encoding the amino acid sequence of SEQ ID NOs: 136 or 137. In some embodiments, the polynucleotide sequence encoding the first zinc finger nuclease comprises the nucleotide sequence of any one of SEQ ID NOs: 71-84. In some embodiments, the polynucleotide sequence encoding the second zinc finger nuclease comprises the nucleotide sequence of any one of SEQ ID NOs: 71-84. In some embodiments, the polynucleotide encoding the first zinc finger nuclease comprises a nucleotide sequence encoding the amino acid sequence of SEQ ID NOs: 130 or 131. In some embodiments, the polynucleotide encoding the second zinc finger nuclease comprises a nucleotide sequence encoding the amino sequence of SEQ ID NOs: 130 or 131. In some embodiments, the polynucleotide sequence encoding the first zinc finger nuclease comprises the nucleotide sequence of any one of SEQ ID NOs: 139-152. In some embodiments, the polynucleotide sequence encoding the second zinc finger nuclease comprises the nucleotide sequence of any one of SEQ ID NOs: 139-152. In some embodiments, the polynucleotide sequence encoding the first zinc finger nuclease comprises the nucleotide sequence of any one of SEQ ID NOs: 17-23 and 25-31. In some embodiments, the polynucleotide sequence encoding the second zinc finger nuclease comprises the nucleotide sequence of any one of SEQ ID NOs: 17-23 and 25-31. In some embodiments, the nucleic acid encoding a 2-in-1 zinc finger nuclease variant comprises a nucleotide sequence selected from any one of SEQ ID NO: 85-115. In some embodiments, the nucleic acid encoding a 2-in-1 zinc finger nuclease variant comprises a nucleotide sequence selected from any one of SEQ ID NO: 35-49.

A twelfth aspect of the disclosure provides a 2-in-1 zinc finger nuclease variant comprising: 1) a first zinc finger nuclease; 2) a second zinc finger nuclease; and 3) a 2A self-cleaving peptide; wherein the 2A self-cleaving peptide is positioned between the first zinc finger nuclease and second zinc finger nuclease.

In some embodiments, The 2-in-1 zinc finger nuclease variant, further comprises one or more of: 1) a nuclear localization sequence; 2) an epitope tag; and 3) a Fok I cleavage domain. In some embodiments, the 2-in-1 zinc finger nuclease variant comprises two independent nuclear localization sequences. In some embodiments, the 2-in-1 zinc finger nuclease variant comprises two or more independent epitope tags. In some embodiments, the 2-in-1 zinc finger nuclease variant comprises two or more independent Fok I cleavage domains. In some embodiments, the first zinc finger nuclease is codon diversified.

In some embodiments, the second zinc finger nuclease is codon diversified. In some embodiments, the first zinc finger nuclease is codon diversified and the second zinc finger nuclease is codon diversified. In some embodiments, the first zinc finger nuclease is encoded by a polynucleotide comprising the nucleotide sequence of any one of SEQ ID NOs: 116-129. In some embodiments, the second zinc finger nuclease is encoded by a polynucleotide comprising the nucleotide sequence of any one of SEQ ID NOs: 116-129. In some embodiments, the first zinc finger nuclease comprises the amino acid sequence of SEQ ID NOs: 136 or 137. In some embodiments, the second zinc finger nuclease comprises the amino acid sequence of SEQ ID NOs: 136 or 137. In some embodiments, the first zinc finger nuclease is encoded by a polynucleotide sequence comprising the nucleotide sequence of any one of SEQ ID NOs: 71-84. In some embodiments, the second zinc finger nuclease is encoded by a polynucleotide sequence comprising the nucleotide sequence of any one of SEQ ID NOs: 71-84. In some embodiments, the first zinc finger nuclease comprises the amino acid sequence of SEQ ID NOs: 130 or 131. In some embodiments, the second zinc finger nuclease comprises the amino sequence of SEQ ID NOs: 130 or 131. In some embodiments, the first zinc finger nuclease is encoded by a polynucleotide comprising the nucleotide sequence of any one of SEQ ID NOs: 139-152. In some embodiments, the second zinc finger nuclease is encoded by a polynucleotide comprising the nucleotide sequence of any one of SEQ ID NOs: 139-152. In some embodiments, the first zinc finger nuclease is encoded by a polynucleotide comprising the nucleotide sequence of any one of SEQ ID NOs: 17-23 and 25-31. In some embodiments, the second zinc finger nuclease is encoded by a polynucleotide comprising the nucleotide sequence of any one of SEQ ID NOs: 17-23 and 25-31. In some embodiments, the 2-in-1 zinc finger nuclease variant is encoded by a nucleotide sequence selected from any one of SEQ ID NO: 85-115.

A thirteenth aspect of the disclosure provides a vector comprising a nucleic acid of the disclosure.

A fourteenth aspect of the disclosure provides a cell comprising the nucleic acid or the vector of the disclosure.

A fifteenth aspect of the disclosure provides a pharmaceutical composition comprising a nucleic acid, a vector or a 2-in-1 zinc finger nuclease variant of the disclosure. In some embodiments, the pharmaceutical composition, further comprises a donor nucleic acid.

A sixteenth aspect of the disclosure provides a nucleic acid of the disclosure, for use in treating or preventing a lysosomal storage disorder.

A seventeenth aspect of the disclosure provides a 2-in-1 zinc finger nuclease variant of the disclosure, for use in treating or preventing a lysosomal storage disorder.

An eighteenth aspect of the disclosure provides a vector of the disclosure, for use in treating or preventing a lysosomal storage disorder.

A nineteenth aspect of the disclosure provides a cell of the disclosure, for use in treating or preventing a lysosomal storage disorder.

A twentieth aspect of the disclosure provides a nucleic acid of the disclosure, for use in correcting a lysosomal storage disease-causing mutation in the genome of a cell.

A twenty-first aspect of the disclosure provides a 2-in-1 zinc finger nuclease variant of the disclosure, for use in correcting a lysosomal storage disease-causing mutation in the genome of a cell.

A twenty-second aspect of the disclosure provides a vector of the disclosure, for use in correcting a lysosomal storage disease-causing mutation in the genome of a cell.

A twenty-third aspect of the disclosure provides a cell of the disclosure, for use in correcting a lysosomal storage disease-causing mutation in the genome of a cell.

A twenty-fourth aspect of the disclosure provides a nucleic acid of the disclosure, for use in integrating an exogenous nucleotide sequence into a target nucleotide sequence in a gene of a cell.

A twenty-fifth aspect of the disclosure provides a 2-in-1 zinc finger nuclease variant of the disclosure, for use in integrating an exogenous nucleotide sequence into a target nucleotide sequence in a gene of a cell.

A twenty-sixth aspect of the disclosure provides a vector of the disclosure, for use in integrating an exogenous nucleotide sequence into a target nucleotide sequence in a gene of a cell.

A twenty-seventh aspect of the disclosure provides a cell of the disclosure, for use in integrating an exogenous nucleotide sequence into a target nucleotide sequence in a gene of a cell.

A twenty-eighth aspect of the disclosure provides a nucleic acid of the disclosure, for use in disrupting a target nucleotide sequence in a gene of a cell, wherein said gene comprises a mutation associated with a lysosomal storage disease.

A twenty-ninth aspect of the disclosure provides a 2-in-1 zinc finger nuclease variant of the disclosure, for use in disrupting a target nucleotide sequence in a gene of a cell, wherein said gene comprises a mutation associated with a lysosomal storage disease.

A thirtieth aspect of the disclosure provides a vector of the disclosure, for use in disrupting a target nucleotide sequence in a gene of a cell, wherein said gene comprises a mutation associated with a lysosomal storage disease.

A thirty-first aspect of the disclosure provides a cell of the disclosure, for use in disrupting a target nucleotide sequence in a gene of a cell, wherein said gene comprises a mutation associated with a lysosomal storage disease.

A thirty-first aspect of the disclosure provides a use of a nucleic acid of the disclosure, for the preparation of a medicament for treating or preventing a lysosomal storage disorder.

A thirty-second aspect of the disclosure provides a use of a 2-in-1 zinc finger nuclease variant of the disclosure, for the preparation of a medicament for treating or preventing a lysosomal storage disorder.

A thirty-third aspect of the disclosure provides a use of a vector of the disclosure, for the preparation of a medicament for treating or preventing a lysosomal storage disorder.

A thirty-fourth aspect of the disclosure provides a use of a cell of the disclosure, for the preparation of a medicament for treating or preventing a lysosomal storage disorder.

A thirty-fifth aspect of the disclosure provides a use of a nucleic acid of the disclosure, for the preparation of a medicament for correcting a lysosomal storage disease-causing mutation in the genome of a cell.

A thirty-sixth aspect of the disclosure provides a use of a 2-in-1 zinc finger nuclease variant of the disclosure, for the preparation of a medicament for correcting a lysosomal storage disease-causing mutation in the genome of a cell.

A thirty-seventh aspect of the disclosure provides a use of a vector of the disclosure, for the preparation of a medicament for correcting a lysosomal storage disease-causing mutation in the genome of a cell.

A thirty-eighth aspect of the disclosure provides a use of a cell of the disclosure, for the preparation of a medicament for correcting a lysosomal storage disease-causing mutation in the genome of a cell.

A thirty-ninth aspect of the disclosure provides a use of a nucleic acid of the disclosure, for the preparation of a medicament for integrating an exogenous nucleotide sequence into a target nucleotide sequence in a gene of a cell.

A fortieth aspect of the disclosure provides a use of a 2-in-1 zinc finger nuclease variant of the disclosure, for the preparation of a medicament for integrating an exogenous nucleotide sequence into a target nucleotide sequence in a gene of a cell.

A forty-first aspect of the disclosure provides a use of a vector of the disclosure, for the preparation of a medicament for integrating an exogenous nucleotide sequence into a target nucleotide sequence in a gene of a cell.

A forty-second aspect of the disclosure provides a use of a cell of the disclosure, for the preparation of a medicament for integrating an exogenous nucleotide sequence into a target nucleotide sequence in a gene of a cell.

A forty-third aspect of the disclosure provides a use of a nucleic acid of the disclosure, for the preparation of a medicament for disrupting a target nucleotide sequence in a gene of a cell, wherein said gene comprises a mutation associated with a lysosomal storage disease.

A forty-fourth aspect of the disclosure provides a use of a 2-in-1 zinc finger nuclease variant of the disclosure, for the preparation of a medicament for disrupting a target nucleotide sequence in a gene of a cell, wherein said gene comprises a mutation associated with a lysosomal storage disease.

A forty-fifth aspect of the disclosure provides a use of a vector of the disclosure, for the preparation of a medicament for disrupting a target nucleotide sequence in a gene of a cell, wherein said gene comprises a mutation associated with a lysosomal storage disease.

A forty-sixth aspect of the disclosure provides a use of a cell of the disclosure, for the preparation of a medicament for disrupting a target nucleotide sequence in a gene of a cell, wherein said gene comprises a mutation associated with a lysosomal storage disease.

### BRIEF DESCRIPTION OF THE DRAWINGS

FIG. 1 shows a schematic of translation outcomes using 2A peptide linkage between two zinc finger nucleases (ZFN-L and ZFN-R). 2A peptide sequences allow 2 proteins on the same transcript to be separated via ribosome skipping. Translation of the transcript will have three possible outcomes which comprise ribosome skipping, ribosome fall-off, or ribosome read-through. The most likely outcome is ribosome skipping. “NPGP” (SEQ ID NO: 173).

FIG. 2 shows schematics of exemplary constructs encoding left and right ZFNs of a ZFN pair separated by a 2A (T2A) self-cleaving sequence. “APOE” and “hAAT” refer, respectively, to an ApoE enhancer and human α1-anti-trypsin (hAAT) promoter (Miao C H et al. (2000) Mol. Ther. 1(6):522-532); “β-globin UTR2” refers to a 5′ Xenopus β-globin UTR (see Krieg and Melton (1994) Nuc Acid Res 12(18):7057); “HBB-IGG intron” refers to a human β-globin-IgG chimeric intron; “3×FLAG” refers to 3 copies of a FLAG peptide sequence (see U.S. Pat. No. 6,379,903); “Nuclear Localization or NLS” refers to a nuclear localization signal; “ZFP-L” refers to the left ZFP of a ZFN pair (e.g., ZFP 71557); “ZFP-R” refers to the right ZFP of a ZFN pair (e.g., 71728); “T2A” refers to a self-cleaving T2A sequence; “FokI DNA Cleavage” refers to the FokI cleavage domain of the ZFN; “WPREmut6” refers to a mutated WPRE sequence (see, e.g., Zanta-Boussif et al. (2009) Gene Ther 16(5):605-619); “bGH” refers to the bovine Growth Hormone polyadenylation signal sequence (see Woychik et al. (1984) Proc Natl Acad Sci 81(13):3944-8). The ZFN2-in-1 undiversified construct contains undiversified left and right ZFNs (Panel A). The ZFN2-in-1 “left” diversified construct contains a diversified left ZFNs and an undiversified right ZFN (Panel B). The ZFN2-in-1 “right” diversified construct contains a diversified right ZFNs and an undiversified left ZFN (Panel C). The ZFN2-in-1 double-diversified construct contains diversified left and right ZFNs (Panel D).

FIG. 3 shows an alkaline agarose gel of DNA from ZFN 2-in-1 constructs in AAV2/6-HEK293 cells. The expected band size is approximately 4.5 kb. G173 and G174 are constructs containing a single ZFN, in which G173 contains a left ZFN and G174 contains a right ZFN (G173 and G174 are referred together as ZFN2.0). GUS130, GUS131, GUS132, GUS133, GUS134, GUS140, GUS141, GUS143, GUS144, and GUS145 ZFN2-in-1 constructs have single-diversified ZFNs. GUS136 and GUS146 ZFN2-in-1 constructs have undiversified ZFNs. GUS150 and GUS151 ZFN2-in-1 constructs have double-diversified ZFNs. GUS001 construct is a control AAV vector. The arrows (between 3 and 4 kb) indicate that the non-codon diversified or undiversified 2-in-1 DNA produced a recombination product.

FIGS. 4A and 4B shows exemplary deletion plots obtained following Nextera sequencing for exemplary undiversified 2-in-1 constructs. Deletions (solid arrow) are shown in darker shading and correspond to the region of the construct shown below the plot (position in the vector map). The lighter shaded region (nonskips) indicates expected sequence coverage without deletions (dashed arrow). FIGS. 4A-4B show results for two undiversified 2-in-1 constructs, GUS146 and GUS136 and shows deletions in regions encoding the left ZFN, the 2A peptide and the right ZFN.

FIGS. 5A, 5B, 5C, 5D and 5E shows exemplary deletion plots obtained following Nextera sequencing for exemplary ZFN2-in-1 constructs with diversified left ZFNs and undiversified right. Deletions (solid arrow) are shown in darker shading and correspond to the region of the construct shown below the plot (position in the vector map). The lighter shaded region (nonskips) indicates expected sequence coverage without deletions (dashed arrow). FIGS. 5A-5E show results for exemplary ZFN2-in-1 constructs, GUS140, GUS141, GUS143, GUS 144 and GUS145, respectively, having diversified left ZFNs and undiversified right ZFNs.

FIGS. 6A, 6B, 6C and 6D shows exemplary deletion plots obtained following Nextera sequencing for exemplary ZFN 2-in-1 constructs with diversified right ZFNs and undiversified left ZFNs. Deletions (solid arrow) are shown in darker shading and correspond to the region of the construct shown below the plot (position in the vector map). The lighter shaded region (nonskips) indicates expected sequence coverage without deletions (dashed arrow). FIGS. 6A-6D show results for exemplary ZFN2-in-1 constructs with diversified right ZFNs and undiversified left ZFNs. Panels A-E show results for exemplary ZFN2-in-1 constructs GUS130, GUS131, GUS132, and GUS133, respectively, having diversified left ZFNs and undiversified right ZFNs.

FIGS. 7A and 7B shows exemplary deletion plots obtained following Nextera sequencing for exemplary ZFN2-in-1 constructs with double-diversified ZFNs. Deletions (solid arrow) are shown in darker shading and correspond to the region of the construct shown below the plot (position in the vector map). The lighter shaded region (nonskips) indicates expected sequence coverage without deletions (dashed arrow). FIGS. 7A-7B show results for exemplary ZFN2-in-1 constructs with double-diversified ZFNs, GUS151 and 150, respectively.

FIG. 8 shows indels in HepG2-AAVR cells (Panel A) and primary hepatocytes (348 cells, Corning) (Panel B) following transduction with the indicated AAV ZFN vectors. In Panel A, for each construct, the bar on the left shows results at AAV doses of 100,000 vg/cell and the bar of the right shows results at AAV doses of 300,000 vg/cell. In Panel B, for each construct, the bar on the left shows results at AAV doses of 20,000 vg/cell and the bar of the right shows results at AAV doses of 200,000 vg/cell.

FIG. 9 shows the activity of the various ZFN constructs for on-target (ALB) or off-target (MICU2 and PACSIN1) genes in 348A primary hepatocytes following transduction with a total AAV dose of 20,000 vg/cell or 200,000 vg/cell of the indicated ZFN constructs. “ALB” refers to albumin. “PACSIN” refers to Protein Kinase C and Casein Kinase Substrate in Neurons 1. “MICU2” refers to Mitochondrial Calcium Uptake 2.

FIG. 10 shows a Western Blot showing ZFN expression in HepG2-AAVR cells with the indicated ZFN constructs. The expected molecular weight for ZFNs is approximately 45-50 kDa. For all 2-in-1 constructs, “L” indicates the cells were transduced with a low viral dose of 100,000 vg/cell and “H” indicates the cells were transduced with a high viral dose of 300,000 vg/cell. For the separate ZFN constructs (G173/G174), “L” indicates the cells were transduced with a low viral dose of 50,000 vg/cell of each construct and “H” indicates the cells were transduced with a high viral dose of 150,000 vg/cell of each construct. GUS130, GUS131, GUS132, GUS133, GUS134, GUS135, GUS140, GUS141, GUS143, GUS144, and GUS145 ZFN2-in-1 constructs have single-diversified ZFNs. GUS136 and GUS146 ZFN2-in-1 constructs have undiversified ZFNs. GUS150 and GUS151 ZFN2-in-1 constructs have double-diversified ZFNs. G173 and G174 are constructs containing a single ZFN, in which G173 contains a left ZFN and G174 contains a right ZFN.

### DETAILED DESCRIPTION

The present disclosure provides methods and compositions for treating and/or preventing a lysosomal storage disease in a subject. The disclosure also provides methods of editing or modifying the genome of a cell by either integrating an exogenous sequence or by disrupting or deleting an undesired sequence. The methods include introducing into a cell in a subject 2-in-1 zinc finger nuclease (ZFN) variants having improved targeting and integration efficiency. More specifically, the zinc finger nuclease (ZFN) variants comprise a first zinc finger nuclease, a second zinc finger nuclease and a 2A self-cleaving peptide positioned between the first zinc finger nuclease and the second zinc finger nuclease. These zinc finger nuclease variants are referred to herein as “2-in-1” ZFN variants.

The present disclosure also provides nucleic acids encoding the 2-in-1 zinc finger nuclease variants which are capable of integrating an exogenous nucleotide sequence with high precision and targeting integration efficiency; 2-in-1 zinc finger nuclease variants, vectors, cell and pharmaceutical compositions;

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components.

Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “chromosome,” as used herein, refers to a chromatin complex comprising all or a portion of the genome of a cell. The genome of a cell is often characterized by its karyotype, which is the collection of all the chromosomes that comprise the genome of the cell. The genome of a cell can comprise one or more chromosomes.

“Chromatin,” as used herein, refers to a nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) that extends between nucleosome cores. A molecule of histone H1 is generally associated with the linker DNA. For the purposes of the present disclosure, the term “chromatin” is meant to encompass all types of cellular nucleoprotein, both eukaryotic and prokaryotic. Cellular chromatin includes both chromosomal and episomal chromatin.

An “episome,” as used herein, refers to a replicating nucleic acid, nucleoprotein complex or other structure comprising a nucleic acid that is not part of the chromosomal karyotype of a cell. It is capable of existing and replicating either autonomously in a cell or as part of a host cell chromosome. Examples of episomes include plasmids and certain viral genomes.

The term “cleavage,” as used herein, refers to the breakage of the covalent backbone of a nucleic acid (e.g. DNA) molecule or polypeptide (e.g., protein) molecule. Cleavage can be initiated by a variety of methods including, but not limited to, enzymatic or chemical hydrolysis (e.g., hydrolysis of a phosphodiester bond in a nucleic acid molecule). With respect to nucleic acid molecules, both single-stranded cleavage and double-stranded cleavage are possible, and double-stranded cleavage can occur as a result of two distinct single-stranded cleavage events. Nucleic acid cleavage can result in the production of either blunt ends or staggered ends. In certain embodiments, fusion polypeptides are used for targeted double-stranded DNA cleavage. With respect to polypeptides, cleavage includes proteolytic cleavage which includes a breaking of the peptide bond between amino acids.

A “cleavage half-domain,” as used herein, refers to a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain,” as used herein, refers to a cleavage half-domain that has been modified so as to form obligate heterodimers with another cleavage half-domain (e.g., another engineered cleavage half-domain). See, U.S. Pat. Nos. 7,888,121; 7,914,796; 8,034,598 and 8,823,618, incorporated herein by reference in their entireties.

The term “binding,” as used herein, refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on-target sequence.

A “binding protein,” as used herein, refers to a protein that is able to bind non-covalently to another molecule. A binding protein can bind to, for example, a DNA molecule (a DNA-binding protein), an RNA molecule (an RNA-binding protein) and/or a polypeptide or protein molecule (a protein-binding protein). In the case of a polypeptide- or protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “DNA binding molecule,” as used herein, refers to a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g. the zinc finger domain).

A “DNA binding protein” or “binding domain,” as used herein, refers to a protein, or a domain within a larger protein, that binds DNA in a sequence-specific manner, for example through one or more zinc fingers or through interaction with one or more Repeat Variable Diresidue (RVDs) in a zinc finger protein or TALE, respectively.

An “exogenous” molecule (e.g. nucleic acid sequence or protein) is a molecule that is not normally present in a cell, but can be introduced into a cell by one or more delivery methods. An exogenous molecule can comprise a therapeutic gene, a plasmid or episome introduced into a cell, a viral genome or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

As used herein, the term “product of an exogenous nucleic acid” includes both polynucleotide and polypeptide products, for example, transcription products (polynucleotides such as RNA) and translation products (polypeptides).

An “endogenous” molecule or sequence is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

“Eukaryotic” cells include, but are not limited to, fungal cells (such as yeast), plant cells, animal cells, mammalian cells and human cells (e.g., T-cells), including stem cells (pluripotent and multipotent).

A “fusion” molecule or any variation thereof is a molecule in which two or more subunit molecules are linked, preferably covalently. The subunit molecules can be the same chemical type of molecule or can be different chemical types of molecules. Examples of fusion molecules include, but are not limited to, fusion proteins (for example, a fusion between a zinc-finger DNA binding domain and a cleavage domain) and fusion nucleic acids (for example, a nucleic acid encoding the fusion protein). Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” as used herein, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression,” as used herein, refers to the conversion of the information contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

A “region of interest,” as used herein, refers to any region of cellular chromatin, such as, for example, a gene or a non-coding sequence, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “codon diversified”, as used herein, refers to any nucleotide sequence in which the codon usage is altered as compared to the original “undiversified” or “non-codon diversified” sequence (e.g., the original designed or selected nuclease or wild-type or mutant donor). Codon diversified sequences may be obtained using any program, (e.g., GeneGPS (ATUM), rdrr.io/HVoltB/Kodonz; see also Komatsurbara et al., nature.com/scientific reports; 5:13283, pp. 1-10 (2015)) and may result in sequences that recombine at a different rate than undiversified sequences and/or result in coding sequences that express higher levels of the encoded polypeptide as compared to undiversified sequence. DNA synthesis providers (such as ATUM and Blueheron) also have their internal algorithms for codon diversification.

A “TALE DNA binding domain” or “TALE” (Transcription activator-like effector), as used herein, refers to a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. Nos. 8,586,526 and 9,458,205. The term “TALEN” (Transcription activator-like effector nuclease) refers to one TALEN or a pair of TALENs (the members of the pair are referred to as “left and right” or “first and second” or “pair”) that dimerize to cleave the target gene. Zinc finger and TALE binding domains can be “engineered” to bind to a predetermined nucleotide sequence, for example, via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,568,526; 6,140,081; 6,453,242; and 6,534,261; see also International Patent Publication Nos. WO 98/53058; WO 98/53059; WO 98/53060; WO 02/016536; and WO 03/016496.

“Recombination,” as used herein, refers to a process of exchanging genetic information between two polynucleotides. For the purposes of this disclosure, “homologous recombination (HR)”, as used herein, refers to a specialized form of such exchange that takes place, for example, during repair of double-strand breaks in cells via homology-directed repair mechanisms. This process requires nucleotide sequence homology, and uses a “donor” molecule (i.e., exogenous DNA) as a template to repair a “target” molecule (i.e., a molecule with a double-stranded break), and is also referred to as “non-crossover gene conversion” or “short tract gene conversion,” because it leads to the transfer of genetic information from the donor to the target molecule. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first target sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

The term “heterologous” means derived from a genotypically distinct entity from that of the rest of the entity to which it is being compared. For example, a polynucleotide introduced by genetic engineering techniques into a plasmid or vector derived from a different species is a heterologous polynucleotide.

The term “% Indel”, as used herein, refers to the percentage of insertions or deletions of several nucleotides in the target sequence of the genome.

“Modulation” (or variants thereof) of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE or CRISPR/Cas system as described herein. Thus, gene inactivation may be partial or complete.

The terms “operative linkage” and “operatively linked” (or “operably linked”) or variations thereof, as used herein, are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous. For example, a linker sequence can be located between both sequences. With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

A “functional” protein, polypeptide, polynucleotide or nucleic acid refers to any protein, polypeptide, polynucleotide or nucleic acid that provides the same function as the wild-type protein, polypeptide, polynucleotide or nucleic acid. A “functional fragment” of a protein, polypeptide, polynucleotide or nucleic acid is a protein, polypeptide, polynucleotide or nucleic acid whose sequence is not identical to the full-length protein, polypeptide or nucleic acid, yet retains the same function as the full-length protein, polypeptide, polynucleotide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Patent Publication No. WO 98/44350.

The term “safe-harbor locus or site,” as used herein, is a genomic locus where genes or other genetic elements can be safely inserted and expressed, because they are known to be tolerant to genetic modification without any undesired effects.

The term “sequence” refers to a nucleotide sequence of any length, which can be DNA or RNA; can be linear, circular or branched and can be either single-stranded or double-stranded. The term “sequence” also refers to an amino acid sequence of any length. The term “transgene” or “donor gene” refers to a nucleotide sequence that is inserted into a genome. A transgene can be of any length, for example between 2 and 100,000,000 nucleotides in length (or any integer value therebetween or thereabove), between about 100 and 100,000 nucleotides in length (or any integer therebetween), between about 2000 and 20,000 nucleotides in length (or any value therebetween) or between about 5 and 15 kb (or any value therebetween).

The term “specificity” (or variations thereof), as used herein, refers to the nuclease being able to bind the target sequence in a specific location with precision. The terms “specificity” and “precision” are used interchangeably.

The terms “subject” and “patient” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject to which the polynucleotides and polypeptides of the invention can be administered.

A “disease associated gene or protein” is one that is defective in some manner in a genetic (e.g., monogenic) disease. Non-limiting examples of genetic diseases include severe combined immunodeficiency, cystic fibrosis, lysosomal storage diseases (e.g., MPS I (Hurler Syndrome), MPS II (Hunter Syndrome), Fabry disease, Pompe disease, PKU, Tay-Sach's, Gaucher, Niemann-Pick Type A and B, GM1 Gangliosidosis, MPS4 A (Morquio syndrome) MPSI (Sly disease), Multiple sulfatase deficiency, Galactosialidosis, Sialidosis, Sialic acid storage disease, Mucolipidosis type II, Farber disease, Cholesterol Ester Storage disease, Wolman disease, or the like), sickle cell anemia, and thalassemia.

The term “target nucleotide sequence” or “target site,” as used herein, refers to a nucleotide sequence located in the genome of a cell which is specifically recognized by a zinc finger nucleotide binding domain of the zinc finger nuclease protein of the disclosure.

The terms “treating” and “treatment” or variations thereof, as used herein, refer to reduction in severity and/or frequency of symptoms, elimination of symptoms and/or underlying cause, prevention of the occurrence of symptoms and/or their underlying cause, delaying the occurrence of symptoms and/or their underlying cause, and improvement or remediation of damage. The treatment may help decrease the dose of one or more other medications required to treat the disease, and/or improve the quality of life.

An “effective dose” or “effective amount,” as used herein, refers to a dose and/or amount of the composition given to a subject as disclosed herein, that can help treat or prevent the occurrence of symptoms.

A polynucleotide “vector” or “construct” is capable of transferring gene sequences to target cells. Typically, “vector construct,” “expression vector,” “expression construct,” “expression cassette,” and “gene transfer vector,” mean any nucleic acid construct capable of directing the expression of a gene of interest and which can transfer gene sequences to target cells. Thus, the term includes cloning, and expression vehicles, as well as integrating vectors.

As used herein, the term “variant” refers to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 86%, about 87%, about 88% about 89%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “zinc-finger DNA binding protein” or “zinc-finger nucleotide binding domain,” as used herein, refers to a protein, or a domain within a larger protein, that binds DNA in a sequence-specific manner through one or more zinc fingers, which are regions of amino acid sequence within the binding domain whose structure is stabilized through coordination of one or more zinc ions. The term zinc finger DNA binding protein is abbreviated as zinc finger protein or ZFP.

The term “zinc-finger nuclease protein” or “zinc-finger nuclease”, as used herein, refers to a protein comprising a zinc-finger DNA binding domain (ZFP) directly or indirectly linked to a DNA cleavage domain (e.g., a Fok I DNA cleavage domain). The term zinc-finger nuclease protein is abbreviated as zinc finger nuclease or ZFN. The cleavage domain may be connected directly to the ZFP. Alternatively, the cleavage domain is connected to the ZFP by way of a linker. The linker region is a sequence which comprises about 1-150 amino acids. Alternatively, the linker region is a sequence which comprises about 6-50 nucleotides. The term includes one ZFN as well as a pair of ZFNs (the members of the pair are referred to as “left and right” or “first and second” or “pair”) that dimerize to cleave the target gene. A pair of ZFNs can be referred to as “left and right”, “first and second” or “pair” and can dimerize to cleave a target gene.

The term “zinc finger nuclease variant” as used herein, refers to a 2-in-1 zinc finger nuclease variant.

“Secretory cell,” as used herein, refers to cells, which are typically derived from epithelium, that secrete molecules (e.g., metabolic byproducts and hormones) into a lumen. Secretory tissues comprise such secretory cells. Examples of secretory tissues include, but are not limited to the gut lining, pancreas, gallbladder, liver, tissues associated with the eye and mucous membranes such as salivary glands, mammary glands, prostate gland, pituitary gland and other members of the endocrine system.

As used herein, “delaying” or “slowing” the progression of an LSD refers to preventing, deferring, hindering, slowing, retarding, stabilizing, and/or postponing development of the disease. This delay can be of varying lengths of time, depending on the history of the disease and/or individual being treated.

The term “supportive surgery,” as used herein, refers to surgical procedures that may be performed on a subject to alleviate symptoms that may be associated with a disease. For subjects with a LSD, such supportive surgeries may include heart valve replacement surgery, tonsillectomy and adenoidectomy, placement of ventilating tubes, repair of abdominal hernias, cervical decompression, treatment of carpal tunnel syndrome, surgical decompression of the median nerve, instrumented fusion (to stabilize and strengthen the spine), arthroscopy, hip or knee replacement, and correction of the lower limb axis, and tracheostomy (see Wraith et al. (2008) Eur J Pediatr. 167(3):267-277; and Scarpa et al. (2011) Orphanet Journal of Rare Diseases 6:72).

“Wheelchair dependent,” as used herein, refers to a subject that is unable to walk due to injury or illness and must rely on a wheelchair to move around.

The term “mechanical ventilator” or “medical ventilator,” as used herein, refers to a device that improves the exchange of air into and out of the lungs. A subject using a mechanical ventilator will be able to maintain adequate levels of oxygen in the blood.

A “symptom,” as used herein, refers to a phenomenon or feeling of departure from normal function, sensation, or structure that is experienced by a subject. For example, a subject with LSD may have symptoms including but not limited to decline in functional abilities, neurologic deterioration, joint stiffness, immobility leading to wheelchair dependency, and difficulty breathing leading to required use of a mechanical ventilator. These symptoms can lead to a shortened life span.

### Methods of Using 2-In-Zinc Finger Nuclease Variants for Lysosomal Storage Disease

The present disclosure relates to a method of treating or preventing a lysosomal storage disease in a subject in need thereof by introducing into the cell of the subject a 2-in-1 zinc finger nuclease variant as disclosed herein. The present disclosure relates to a method of treating or preventing a lysosomal storage disease in a subject in need thereof by introducing into the cell of the subject a nucleic acid encoding a 2-in-1 zinc finger nuclease variant as disclosed herein.

In one aspect, the disclosure provides a method for treating a lysosomal storage disease in a subject, the method comprising modifying a target sequence in the genome of a cell of said subject. In some embodiments, the disclosure provides a method for treating a lysosomal storage disease in a subject, the method comprising modifying a target sequence in the genome of a cell of said subject by introducing into the cell a 2-in-1 zinc finger nuclease variant of the disclosure. In some embodiments, the disclosure provides a method for treating a lysosomal storage disease in a subject, the method comprising modifying a target sequence in the genome of a cell of said subject by introducing into the cell a nucleic acid encoding a 2-in-1 zinc finger nuclease variant of the disclosure. In some embodiments, the disclosure provides a method for treating a lysosomal storage disease in a subject, the method comprising modifying a target sequence in the genome of a cell of said subject by introducing into the cell a vector of the disclosure. In some embodiments, the disclosure provides a method for treating a lysosomal storage disease in a subject, the method comprising administering to the subject a pharmaceutical composition of the disclosure.

In some embodiments, the disclosure provides a method for treating a lysosomal storage disease in a subject, the method comprising modifying a target sequence in the genome of a cell of said subject by contacting the cell with a 2-in-1 zinc finger nuclease variant of the disclosure. In some embodiments, the disclosure provides a method for treating a lysosomal storage disease in a subject, the method comprising modifying a target sequence in the genome of a cell of said subject by contacting the cell with a nucleic acid encoding a 2-in-1 zinc finger nuclease variant of the disclosure. In some embodiments, the disclosure provides a method for treating a lysosomal storage disease in a subject, the method comprising modifying a target sequence in the genome of a cell of said subject by contacting the cell with a vector of the disclosure. In some embodiments, the disclosure provides a method for treating a lysosomal storage disease in a subject, the method comprising modifying a target sequence in the genome of a cell of said subject by contacting the cell with a pharmaceutical of the disclosure.

In some embodiments, the disclosure provides a method for treating a lysosomal storage disease in a subject, the method comprising administering to said subject a 2-in-1 zinc finger nuclease variant of the disclosure. In some embodiments, the disclosure provides a method for treating a lysosomal storage disease in a subject, the method comprising the method comprising administering to said subject a nucleic acid encoding a 2-in-1 zinc finger nuclease variant of the disclosure. In some embodiments, the disclosure provides a method for treating a lysosomal storage disease in a subject, the method comprising administering to said subject a vector of the disclosure. In some embodiments, the disclosure provides a method for treating a lysosomal storage disease in a subject, the method comprising administering to said subject a pharmaceutical composition of the disclosure.

In one aspect, the disclosure provides a method for preventing a lysosomal storage disease in a subject, the method comprising modifying a target sequence in the genome of a cell of said subject. In some embodiments, the disclosure provides a method for preventing a lysosomal storage disease in a subject, the method comprising modifying a target sequence in the genome of a cell of said subject by introducing into the cell the 2-in-1 zinc finger nuclease variant of the disclosure. In some embodiments, the disclosure provides a method for preventing a lysosomal storage disease in a subject, the method comprising modifying a target sequence in the genome of a cell of said subject by introducing into the cell a nucleic acid encoding the 2-in-1 zinc finger nuclease variant of the disclosure. In some embodiments, the disclosure provides a method for preventing a lysosomal storage disease in a subject, the method comprising modifying a target sequence in the genome of a cell of said subject by introducing into the cell a vector of the disclosure. In some embodiments, the disclosure provides a method for preventing a lysosomal storage disease in a subject, the method comprising administering to the subject a pharmaceutical composition of the disclosure.

In some embodiments, the disclosure provides a method for preventing a lysosomal storage disease in a subject, the method comprising modifying a target sequence in the genome of a cell of said subject by contacting the cell with a 2-in-1 zinc finger nuclease variant of the disclosure. In some embodiments, the disclosure provides a method for preventing a lysosomal storage disease in a subject, the method comprising modifying a target sequence in the genome of a cell of said subject by contacting the cell with a nucleic acid encoding a 2-in-1 zinc finger nuclease variant of the disclosure. In some embodiments, the disclosure provides a method for preventing a lysosomal storage disease in a subject, the method comprising modifying a target sequence in the genome of a cell of said subject by contacting the cell with a vector of the disclosure. In some embodiments, the disclosure provides a method for preventing a lysosomal storage disease in a subject, the method comprising modifying a target sequence in the genome of a cell of said subject by contacting the cell with a pharmaceutical of the disclosure.

In some embodiments, the disclosure provides a method for preventing a lysosomal storage disease in a subject, the method comprising administering to said subject a 2-in-1 zinc finger nuclease variant of the disclosure. In some embodiments, the disclosure provides a method for preventing a lysosomal storage disease in a subject, the method comprising the method comprising administering to said subject a nucleic acid encoding a 2-in-1 zinc finger nuclease variant of the disclosure. In some embodiments, the disclosure provides a method for preventing a lysosomal storage disease in a subject, the method comprising administering to said subject a vector of the disclosure. In some embodiments, the disclosure provides a method for preventing a lysosomal storage disease in a subject, the method comprising administering to said subject a pharmaceutical composition of the disclosure.

In some embodiments, the method of treating or preventing a lysosomal storage disease includes improving or maintaining (slowing the decline) of functional ability in a human subject having a LSD. In some embodiments, the method of treating or preventing a lysosomal storage disease includes decreasing the need (dose level or frequency) for enzyme replacement therapy (ERT) in a subject with a LSD. In some embodiments, the method of treating or preventing a lysosomal storage disease includes delaying the need for ERT initiation in a subject with a LSD. In some embodiments, the method of treating or preventing a lysosomal storage disease includes delaying, reducing or eliminating the need for supportive surgery in a subject with a LSD (e.g., MPS II). In some embodiments, the method of treating or preventing a lysosomal storage disease includes delaying, reducing or preventing the need for a bone marrow transplant in a subject with a LSD In some embodiments, the method of treating or preventing a lysosomal storage disease includes improving the functional (delaying decline, maintenance) ability in a subject with a LSD. In some embodiments, the method of treating or preventing a lysosomal storage disease includes suppressing disability progression in a human subject having a LSD. In some embodiments, the method of treating or preventing a lysosomal storage disease includes delaying, reducing or preventing the need for the use of a medical ventilator device in a subject with a LSD. In some embodiments, the method of treating or preventing a lysosomal storage disease includes delaying onset of confirmed disability progression or reducing the risk of confirmed disability progression in a human subject having a LSD. In some embodiments, the method of treating or preventing a lysosomal storage disease includes reducing, stabilizing or maintaining urine GAGs in a subject with a LSD. In some embodiments, the method of treating or preventing a lysosomal storage disease includes extending life expectancy in a subject with a LSD.

In one aspect, the disclosure provides a method for correcting a lysosomal storage disease-causing mutation in the genome of a cell. In some embodiments, the disclosure provides a method for correcting a lysosomal storage disease-causing mutation in the genome of a cell, the method comprising modifying a target sequence in the genome of the cell by introducing into the cell the 2-in-1 zinc finger nuclease variant of the disclosure. In some embodiments, the disclosure provides a method for correcting a lysosomal storage disease-causing mutation in the genome of a cell, the method comprising modifying a target sequence in the genome of the cell by introducing into the cell a nucleic acid encoding the 2-in-1 zinc finger nuclease variant of the disclosure. In some embodiments, the disclosure provides a method for correcting a lysosomal storage disease-causing mutation in the genome of a cell, the method comprising modifying a target sequence in the genome of the cell by introducing into the cell a vector of the disclosure. In some embodiments, the disclosure provides a method for correcting a lysosomal storage disease-causing mutation in the genome of a cell, the method comprising modifying a target sequence in the genome of the cell by introducing into the cell a pharmaceutical composition of the disclosure.

In some embodiments, the disclosure provides a method for correcting a lysosomal storage disease-causing mutation in the genome of a cell, the method comprising modifying a target sequence in the genome of the cell by contacting the with cell the 2-in-1 zinc finger nuclease variant of the disclosure. In some embodiments, the disclosure provides a method for correcting a lysosomal storage disease-causing mutation in the genome of a cell, the method comprising modifying a target sequence in the genome of the cell by contacting the cell with a nucleic acid encoding the 2-in-1 zinc finger nuclease variant of the disclosure. In some embodiments, the disclosure provides a method for correcting a lysosomal storage disease-causing mutation in the genome of a cell, the method comprising modifying a target sequence in the genome of the cell by contacting the cell with a vector of the disclosure. In some embodiments, the disclosure provides a method for correcting a lysosomal storage disease-causing mutation in the genome of a cell, the method comprising contacting the cell with pharmaceutical composition of the disclosure.

In one aspect, the disclosure provides a method for improving or maintaining (slowing the decline) of functional ability in a subject having a lysosomal storage disease. In some embodiments, the disclosure provides a method for improving or maintaining (slowing the decline) of functional ability in a subject having a lysosomal storage disease, the method comprising modifying a target sequence in the genome of a cell of the subject by introducing into the cell the 2-in-1 zinc finger nuclease variant of the disclosure. In some embodiments, the disclosure provides a method for improving or maintaining (slowing the decline) of functional ability in a subject having a lysosomal storage disease, the method comprising modifying a target sequence in the genome of a cell of the subject by introducing into the cell a nucleic acid encoding the 2-in-1 zinc finger nuclease variant of the disclosure. In some embodiments, the disclosure provides a method for improving or maintaining (slowing the decline) of functional ability in a subject having a lysosomal storage disease, the method comprising modifying a target sequence in the genome of a cell of the subject by introducing into the cell a vector of the disclosure. In some embodiments, the disclosure provides a method for improving or maintaining (slowing the decline) of functional ability in a subject having a lysosomal storage disease, the method comprising administering to the subject a pharmaceutical composition of the disclosure.

In another aspect, the disclosure provides a method of decreasing the need (dose level or frequency) for enzyme replacement therapy (ERT) in a subject with a LSD. In some embodiments, the disclosure provides a method of decreasing the need (dose level or frequency) for ERT in a subject with a LSD, the method comprising modifying a target sequence in the genome of a cell of said subject by introducing into the cell a 2-in-1 zinc finger nuclease variant of the disclosure. In some embodiments, the disclosure provides a method of decreasing the need (dose level or frequency) for ERT in a subject with a LSD, the method comprising modifying a target sequence in the genome of a cell of said subject by introducing into the cell a nucleic acid encoding a 2-in-1 zinc finger nuclease variant of the disclosure. In some embodiments, the disclosure provides a method of decreasing the need (dose level or frequency) for ERT in a subject with a LSD, the method comprising modifying a target sequence in the genome of a cell of said subject by introducing into the cell a vector of the disclosure. In some embodiments, the disclosure provides a method of decreasing the need (dose level or frequency) for ERT in a subject with a LSD, the method comprising administering to the subject a pharmaceutical composition of the disclosure.

In some embodiments of the method for treating or preventing a lysosomal disease or for correcting a lysosomal disease-causing mutation, at least one cell, cell type or tissue comprise a recombination site that is recognized by a zinc finger nucleotide binding domain. This cell(s) is transformed with a donor nucleic acid construct (a “donor construct”) comprising a second recombination sequence and one or more polynucleotides of interest (typically a therapeutic gene). Into the same cell, a 2-in-1 zinc finger nuclease variant of the disclosure or a nucleic acid encoding the 2-in-1 zinc finger nuclease variant of the disclosure is introduced. The 2-in-1 zinc finger nuclease variant specifically recognizes the recombination sequences, under conditions such that the nucleic acid sequence of interest is inserted into the genome via a recombination event between the first and second recombination sites. Subjects treatable using the methods of the invention include both humans and non-human animals.

A variety of lysosomal storage diseases that may be treated by the methods disclosed herein. Exemplary lysosomal storage diseases that may be treated and/or prevented by 2-in-1 zinc finger nuclease variants described herein include, but are not limited to, Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/J/A), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS ME Sanfilippo Syndrome Type B, MPS IIIC Sanfilippo Syndrome Type C, MPSIIII)—Sanfilippo Syndrome Type I), MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, Wolman Disease and the like.

In some embodiments, a subject having MPS II may have attenuated form MPSII or severe MPS II. “Severe MPS II” in subjects is characterized by delayed speech and developmental delay between 18 months to 3 years of age. The disease is characterized in severe MPS II subjects by organomegaly, hyperactivity and aggressiveness, neurologic deterioration, joint stiffness and skeletal deformities (including abnormal spinal bones), coarse facial features with enlarged tongue, heart valve thickening, hearing loss and hernias. The life expectancy of untreated subjects with severe Hunter syndrome is into the mid teenage years with death due to neurologic deterioration and/or cardiorespiratory failure. “Attenuated form MPS II” in subjects are typically diagnosed later than the severe subjects. The somatic clinical features are similar to the severe subjects, but overall disease severity in milder with, in general, slower disease progression with no or only mild cognitive impairment. Death in the untreated attenuated form is often between the ages of 20-30 years from cardiac and respiratory disease.

The proteins associated with the various lysosomal storage diseases include, but are not limited to those set forth in Table 1.

TABLE 1

| LSD | Enzyme or protein | Gene |
|---|---|---|
| Alpha-mannosidosis | Alpha-D-mannosidase | MAN2B1 |
| Aspartylglucosaminuria | N-aspartyl-beta- | AGA |
| | glucosaminidase | |
| Cholesteryl ester storage | Lysosomal acid lipase | LIPA |
| disease | | |
| Cystinosis | Cystinosin | CTNS |
| Danon Disease | Lysosomal associated | LAMP2 |
| | membrane protein 2 | |
| Fabry Disease | Alpha-galactosidase A | GLA |
| Farber Disease | Acid ceramidase | ASAH1 |
| Fucosidosis | Alpha fucosidase | FUCA1 |
| Galactosialidosis | Cathepsin A | CTSA |
| Gaucher Disease Type I | Acid beta-glucocerebrosidase | GBA |
| Gaucher Disease Type II | Acid beta-glucocerebrosidase | GBA |
| Gaucher Disease Type III | Acid beta-glucocerebrosidase | GBA |
| GM1 Gangliosidosis | Beta galactosidase | GLB1 |
| (Types I, II and III) | | |
| GM2 Sandhoff Disease | Beta hexosaminidase A | HEXB |
| (I/J/A) | Beta hexosaminidase B | |
| GM2 Tay-Sachs disease | Beta-hexosaminidase | HEXA |
| GM2 Gangliosidosis AB | GM2 ganglioside activator | GM2A |
| variant | (GM2A) | |
| I-Cell Disease/ | GLcNAc-1-phospho- | GNPTAB |
| Mucolipidosis II | transferase | |
| Krabbe Disease | Beta-galactosylceramidase | GALC |
| Lysosomal acid lipase | Lysosomal acid lipase | LIPA |
| deficiency | | |
| Metachromatic | Arylsulfatase A | ARSA |
| Leukodystrophy | | |
| MPS I—Hurler Syndrome | Alpha-L-iduronidase | IDUA |
| MPS I—Scheie Syndrome | Alpha-L-iduronidase | IDUA |
| MPS I Hurler-Scheie | Alpha-L-iduronidase | IDUA |
| Syndrome | | |
| MPS II Hunter Syndrome | Iduronate-2-sulphatase | IDS |
| MPS IIIA—Sanfilippo | Heparan N-sulfatase | SGSH |
| Syndrome Type A | | |
| MPS IIIB—Sanfilippo | Alpha-N-acetylglucos- | NAGLU |
| Syndrome Type B | aminidase | |
| MPS IIIC—Sanfilippo | acetyl CoA:alpha-glucos- | GSNAT |
| Syndrome Type C | aminide | |
| | acetyltransferase | |
| MPSIIID—Sanfilippo | N-acetyl glucosamine-6- | GNS |
| Syndrome Type D | sulfatase | |
| MPS IV—Morquio Type A | Galactosamine-6-sulfate | GALNS |
| | sulfatase | |
| MPS IV—Morquio Type B | Beta-galactosidase | GLB1 |
| MPS VI—Maroteaux-Lamy | Arylsulfatase B | ARSB |
| MPS VII—Sly Syndrome | Beta-glucuronidase | GUSB |
| MPS IX—Hyaluronidase | Hyaluronidase | HYALl |
| Deficiency | | |
| Mucolipidosis I—Sialidosis | Neuraminidase | NEU1 |
| Mucolipidosis IIIC | GlcNAc-1-phospho- | GNPTG |
| | transferase | |
| Mucolipidosis Type IV | Mucolipin-1 | MCOLN1 |
| Multiple Sulfatase | Formylglycine-generating | SUMF1 |
| Deficiency | enzyme (FGE) | |
| Neuronal Ceroid | Palmitoyl-protein | PPT1 |
| Lipofuscinosis T1 | thioesterase 1 | |
| Neuronal Ceroid | tripeptidyl peptidase 1 | TPP1 |
| Lipofuscinosis T2 | | |
| Neuronal Ceroid | CLN3 protein | CLN3 |
| Lipofuscinosis T3 | | |
| Neuronal Ceroid | Cysteine string protein alpha | DNAJC5 |
| Lipofuscinosis T4 | | |
| Neuronal Ceroid | CLN5 protein | CLN5 |
| Lipofuscinosis T5 | | |
| Neuronal Ceroid | CLN6 protein | CLN6 |
| Lipofuscinosis T6 | | |
| Neuronal Ceroid | CLN7 protein | CLN7 |
| Lipofuscinosis T7 | | |
| Neuronal Ceroid | CLN8 protein | CLN8 |
| Lipofuscinosis T8 | | |
| Niemann-Pick Disease | Acid sphingomyelinase | SMPD1 |
| Type A | | |
| Niemann-Pick Disease | Acid sphingomyelinase | SMPD1 |
| Type B | | |
| Niemann-Pick Disease | NPC 1/NPC2 | NPC1, NPC2 |
| Type C | | |
| Phenylketonuria | Phenylalanine hydroxylase | PAH |
| Pompe Disease | Acid alpha-glucosidase | GAA |
| Pycnodysostosis, | cathepsin K | CTSK |
| Sialic Acid Storage Disease | Sialin (sialic acid transporter) | SLC17A5 |
| Schindler Disease | Alpha-N- | NAGA |
| | acetylgalactosaminidase | |
| Wolman Disease | Lysosomal acid lipase | LIPA |

Thus, in some embodiments, the methods disclosed herein further comprise introducing into the cell a corrective disease-associated protein or enzyme or portion thereof. In some embodiments, the methods disclosed herein further comprise introducing into the cell a nucleic acid molecule encoding a corrective disease-associated protein or enzyme or portion thereof. In some embodiments, the methods disclosed herein comprise introducing into the cell a corrective disease-associated protein or enzyme as set forth in Table 1 or portions thereof. In some embodiments, the methods disclosed herein comprise introducing into the cell a corrective disease-associated gene as set forth in Table 1 or portions thereof.

In some embodiments, the methods disclosed herein comprise inserting one or more corrective disease-associated genes as set forth in Table 1 or portions thereof into a safe harbor locus (e.g. albumin) in a cell for expression of the needed protein(s) (e.g. enzyme(s) in Table 1) and release into the blood stream. Once in the bloodstream, the secreted enzyme may be taken up by cells in the tissues, wherein the enzyme is then taken up by the lysosomes such that the GAGs are broken down. In some embodiments, the inserted transgene encoding the disease associated protein (e.g., IDS, IDUA, GLA, GAA, PAH, etc.) is codon optimized. In some embodiments, the transgene is one in which the relevant epitope is removed without functionally altering the protein. In some embodiments, the methods comprise insertion of an episome expressing the corrective enzyme (or protein)-encoding transgene into a cell for expression of the needed enzyme and release into the blood stream. In some embodiments, the insertion is into a secretory cell, such as a liver cell for release of the product into the blood stream.

The method for treatment or correction of a disease-causing mutation can take place in vivo or ex vivo. By “in vivo” it is meant in the living body of an animal. By “ex vivo” it is meant that cells or organs are modified outside of the body, such cells or organs are typically returned to a living body.

Methods for the therapeutic administration of vectors or constructs including the zinc finger nuclease proteins of the disclosure are well known in the art. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995, all of which are incorporated by reference herein), using viral vectors (Monahan, et al, Gene Therapy 4:40-49, 1997; Onodera, et al, Blood 91:30-36, 1998, all of which are incorporated by reference herein), by uptake of “naked DNA”, and the like. Techniques well known in the art for the transfection of cells (see discussion above) can be used for the ex vivo administration of nucleic acid constructs. The exact formulation, route of administration and dosage can be chosen by the individual physician in view of the patient's condition. (Fingl et al., 1975, in “The Pharmacological Basis of Therapeutics”, Ch. 1 p 1).

As disclosed herein, the zinc finger nuclease protein and methods described herein can be used for gene modification, gene correction, and gene disruption.

The zinc finger nuclease protein and methods described herein can also be applied to stem cell based therapies, including but not limited to editing that results in: correction of somatic cell mutations; disruption of dominant negative alleles; disruption of genes required for the entry or productive infection of pathogens into cells; enhanced tissue engineering, for example, by editing gene activity to promote the differentiation or formation of functional tissues; and/or disrupting gene activity to promote the differentiation or formation of functional tissues; blocking or inducing differentiation, for example, by editing genes that block differentiation to promote stem cells to differentiate down a specific lineage pathway. Cell types for this procedure include but are not limited to, T-cells, B cells, hematopoietic stem cells, and embryonic stem cells. Additionally, induced pluripotent stem cells (iPSC) may be used which would also be generated from a patient's own somatic cells. Therefore, these stem cells or their derivatives (differentiated cell types or tissues) could be potentially engrafted into any person regardless of their origin or histocompatibility.

In some embodiments, the methods and compositions of the invention are used to supply a transgene encoding one or more therapeutics in a hematopoietic stem cell such that mature cells (e.g., RBCs) derived from these cells contain the therapeutic. These stem cells can be differentiated in vitro or in vivo and may be derived from a universal donor type of cell which can be used for all subjects. Additionally, the cells may contain a transmembrane protein to traffic the cells in the body. Treatment can also comprise use of subject cells containing the therapeutic transgene where the cells are developed ex vivo and then introduced back into the subject. For example, HSC containing a suitable transgene may be inserted into a subject via an autologous bone marrow transplant. Alternatively, stem cells such as muscle stem cells or iPSC which have been edited using with the transgene maybe also injected into muscle tissue.

Thus, this technology may be of use in a condition where a subject is deficient in some protein due to problems (e.g., problems in expression level or problems with the protein expressed as sub- or non-functioning

By way of non-limiting examples, production of the defective or missing proteins is accomplished and used to treat LSD. Nucleic acid donors encoding the proteins may be inserted into a safe harbor locus (e.g. albumin) and expressed either using an exogenous promoter or using the promoter present at the safe harbor. Alternatively, donors can be used to correct the defective gene in situ. The desired transgene may be inserted into a CD34+ stem cell and returned to a subject during a bone marrow transplant. Finally, the nucleic acid donor maybe be inserted into a CD34+ stem cell at a beta globin locus such that the mature red blood cell derived from this cell has a high concentration of the biologic encoded by the nucleic acid donor. The biologic-containing RBC can then be targeted to the correct tissue via transmembrane proteins (e.g. receptor or antibody). Additionally, the RBCs may be sensitized ex vivo via electrosensitization to make them more susceptible to disruption following exposure to an energy source (see International Patent Publication No. WO 2002/007752).

In addition to therapeutic applications, the zinc finger nuclease protein and methods described herein can be used for cell line engineering and the construction of disease models.

In one aspect, provided herein is a nucleic acid encoding a 2-in-1 zinc finger variant as disclosed herein, for use in treating or preventing a lysosomal storage disorder.

In one aspect, provided herein is a 2-in-1 zinc finger nuclease variant as disclosed herein, for use in treating or preventing a lysosomal storage disorder.

In one aspect, provided herein is a vector as disclosed herein, for use in treating or preventing a lysosomal storage disorder.

In one aspect, provided herein is a cell as disclosed herein, for use in treating or preventing a lysosomal storage disorder.

In one aspect provided herein is a nucleic acid encoding a 2-in-1 zinc finger variant as disclosed herein, for use in correcting a lysosomal storage disease-causing mutation in the genome of a cell.

In one aspect, provided herein is a 2-in-1 zinc finger nuclease variant as disclosed herein, for use in correcting a lysosomal storage disease-causing mutation in the genome of a cell.

In one aspect, provided herein is a vector as disclosed herein, for use in correcting a lysosomal storage disease-causing mutation in the genome of a cell.

In one aspect, provided herein is a cell as disclosed herein, for use in correcting a lysosomal storage disease-causing mutation in the genome of a cell.

In one aspect provided herein is a nucleic acid encoding a 2-in-1 zinc finger variant as disclosed herein, for use in integrating an exogenous nucleotide sequence into a target nucleotide sequence in a gene of a cell.

In one aspect, provided herein is a 2-in-1 zinc finger nuclease variant as disclosed herein, for use in integrating an exogenous nucleotide sequence into a target nucleotide sequence in a gene of a cell.

In one aspect, provided herein is a vector as disclosed herein, for use in integrating an exogenous nucleotide sequence into a target nucleotide sequence in a gene of a cell.

In one aspect, provided herein is a cell as disclosed herein, for use in integrating an exogenous nucleotide sequence into a target nucleotide sequence in a gene of a cell.

In one aspect provided herein is a nucleic acid encoding a 2-in-1 zinc finger variant as disclosed herein, for use in disrupting a target nucleotide sequence in a gene of a cell, wherein said gene comprises a mutation associated with a lysosomal storage disease.

In one aspect, provided herein is a 2-in-1 zinc finger nuclease variant as disclosed herein, for use in disrupting a target nucleotide sequence in a gene of a cell, wherein said gene comprises a mutation associated with a lysosomal storage disease.

In one aspect, provided herein is a vector as disclosed herein, for use in disrupting a target nucleotide sequence in a gene of a cell, wherein said gene comprises a mutation associated with a lysosomal storage disease.

In one aspect, provided herein is a cell as disclosed herein, for use in disrupting a target nucleotide sequence in a gene of a cell, wherein said gene comprises a mutation associated with a lysosomal storage disease.

### Zinc Finger Nuclease Variant Nucleic Acids

In one aspect, disclosed herein is a nucleic acid encoding a 2-in-1 zinc finger nuclease variant. In some embodiments, the nucleic acid encoding a 2-in-1 zinc finger nuclease variant comprises: a) a polynucleotide encoding a first zinc finger nuclease; b) a polynucleotide encoding a second zinc finger nuclease; and c) a polynucleotide encoding a 2A self-cleaving peptide; wherein the polynucleotide encoding the 2A self-cleaving peptide is positioned between the polynucleotide encoding the first zinc finger nuclease and the polynucleotide encoding the second zinc finger nuclease. In some embodiments, the polynucleotide encoding the first zinc finger nuclease is codon diversified. In some embodiments, the polynucleotide encoding the first zinc finger nuclease is not codon diversified. In some embodiments the polynucleotide encoding the second zinc finger nuclease is codon diversified. In some embodiments the polynucleotide encoding the second zinc finger nuclease is not codon diversified. In some embodiments, the polynucleotide encoding the first zinc finger nuclease and the polynucleotide encoding the second zinc finger nuclease are each independently codon diversified. In some embodiments, neither the polynucleotide encoding the first zinc finger nuclease nor the polynucleotide encoding the second zinc finger nuclease is codon diversified.

In some embodiments, the nucleic acid encoding the 2-in-1 zinc finger nuclease variant further comprises a nucleic acid sequence selected from one or more of: a) one or more polynucleotide sequences encoding a nuclear localization sequence; b) a 5′ITR polynucleotide sequence; c) an enhancer polynucleotide sequence; d) a promoter polynucleotide sequence; e) a 5′UTR polynucleotide sequence; f) a chimeric intron polynucleotide sequence; g) one or more polynucleotide sequences encoding an epitope tag; h) one or more cleavage domains; i) a post-transcriptional regulatory element polynucleotide sequence; j) a polyadenylation signal sequence; k) a 3′ UTR polynucleotide sequence; and l) a 3′ITR polynucleotide sequence.

In some embodiments, the polynucleotide sequence encoding the first zinc finger nuclease comprises the nucleotide sequence of any one of SEQ ID NOs: 116-129. In some embodiments, the polynucleotide sequence encoding the first zinc finger nuclease comprises the nucleotide sequence of SEQ ID NO: 116. In some embodiments, the polynucleotide sequence encoding the first zinc finger nuclease comprises the nucleotide sequence of SEQ ID NO: 117. In some embodiments, the polynucleotide sequence encoding the first zinc finger nuclease comprises the nucleotide sequence of SEQ ID NO: 118. In some embodiments, the polynucleotide sequence encoding the first zinc finger nuclease comprises the nucleotide sequence of SEQ ID NO: 119. In some embodiments, the polynucleotide sequence encoding the first zinc finger nuclease comprises the nucleotide sequence of SEQ ID NO: 120. In some embodiments, the polynucleotide sequence encoding the first zinc finger nuclease comprises the nucleotide sequence of SEQ ID NO: 121. In some embodiments, the polynucleotide sequence encoding the first zinc finger nuclease comprises the nucleotide sequence of SEQ ID NO: 122. In some embodiments, the polynucleotide sequence encoding the first zinc finger nuclease comprises the nucleotide sequence of SEQ ID NO: 123. In some embodiments, the polynucleotide sequence encoding the first zinc finger nuclease comprises the nucleotide sequence of SEQ ID NO: 124. In some embodiments, the polynucleotide sequence encoding the first zinc finger nuclease comprises the nucleotide sequence of SEQ ID NO: 125. In some embodiments, the polynucleotide sequence encoding the first zinc finger nuclease comprises the nucleotide sequence of SEQ ID NO: 126. In some embodiments, the polynucleotide sequence encoding the first zinc finger nuclease comprises the nucleotide sequence of SEQ ID NO: 127. In some embodiments, the polynucleotide sequence encoding the first zinc finger nuclease comprises the nucleotide sequence of SEQ ID NO: 128. In some embodiments, the polynucleotide sequence encoding the first zinc finger nuclease comprises the nucleotide sequence of SEQ ID NO:129.

In some embodiments, the polynucleotide sequence encoding the first zinc finger nuclease comprises a nucleotide sequence encoding the amino acid sequence of any one of SEQ ID NOs: 136-137. In some embodiments, the polynucleotide sequence encoding the first zinc finger nuclease comprises a nucleotide sequence encoding the amino acid sequence of SEQ ID NOs: 136. In some embodiments, the polynucleotide sequence encoding the first zinc finger nuclease comprises a nucleotide sequence encoding the amino acid sequence of SEQ ID NOs: 137.

In some embodiments, the polynucleotide sequence encoding the second zinc finger nuclease comprises the nucleotide sequence of any one of SEQ ID NOs: 116-129. In some embodiments, the polynucleotide sequence encoding the second zinc finger nuclease comprises the nucleotide sequence of SEQ ID NO: 116. In some embodiments, the polynucleotide sequence encoding the second zinc finger nuclease comprises the nucleotide sequence of SEQ ID NO: 117. In some embodiments, the polynucleotide sequence encoding the second zinc finger nuclease comprises the nucleotide sequence of SEQ ID NO: 118. In some embodiments, the polynucleotide sequence encoding the second zinc finger nuclease comprises the nucleotide sequence of SEQ ID NO: 119. In some embodiments, the polynucleotide sequence encoding the second zinc finger nuclease comprises the nucleotide sequence of SEQ ID NO: 120. In some embodiments, the polynucleotide sequence encoding the second zinc finger nuclease comprises the nucleotide sequence of SEQ ID NO: 121. In some embodiments, the polynucleotide sequence encoding the second zinc finger nuclease comprises the nucleotide sequence of SEQ ID NO: 122. In some embodiments, the polynucleotide sequence encoding the second zinc finger nuclease comprises the nucleotide sequence of SEQ ID NO: 123. In some embodiments, the polynucleotide sequence encoding the second zinc finger nuclease comprises the nucleotide sequence of SEQ ID NO: 124. In some embodiments, the polynucleotide sequence encoding the second zinc finger nuclease comprises the nucleotide sequence of SEQ ID NO: 125. In some embodiments, the polynucleotide sequence encoding the second zinc finger nuclease comprises the nucleotide sequence of SEQ ID NO: 126. In some embodiments, the polynucleotide sequence encoding the second zinc finger nuclease comprises the nucleotide sequence of SEQ ID NO: 127. In some embodiments, the polynucleotide sequence encoding the second zinc finger nuclease comprises the nucleotide sequence of SEQ ID NO: 128. In some embodiments, the polynucleotide sequence encoding the second zinc finger nuclease comprises the nucleotide sequence of SEQ ID NO:129.

In some embodiments, the polynucleotide sequence encoding the second zinc finger nuclease comprises a nucleotide sequence encoding the amino acid sequence of any one of SEQ ID NOs: 136-137. In some embodiments, the polynucleotide sequence encoding the second zinc finger nuclease comprises a nucleotide sequence encoding the amino acid sequence of SEQ ID NOs: 136. In some embodiments, the polynucleotide sequence encoding the second zinc finger nuclease comprises a nucleotide sequence encoding the amino acid sequence of SEQ ID NOs: 137.

In some embodiments, the nucleic acid encoding the 2-in-1 zinc finger nuclease variant further comprises one or more polynucleotide sequences encoding one or more cleavage domains. Any suitable cleavage domain can be associated with (e.g., operatively linked) to a zinc finger DNA-binding domain (e.g., ZFP). In some embodiments, the two or more cleavage domains are the same. In some embodiments, the two or more cleavage domains have the same amino acid sequence. In some embodiments, the two or more cleavage domains have different amino acid sequences. In some embodiments, the two or more cleavage domains are encoded by a polynucleotide having the same nucleotide sequence. In some embodiments, the two or more cleavage domains are encoded by a polynucleotide having different nucleotide sequences. In some embodiments, the cleavage domain comprises a Fok I cleavage domain, which is active as a dimer. In some embodiments the polynucleotide sequence encoding the one or more Fok I cleavage domain is codon diversified. In some embodiments the polynucleotide sequence encoding the one or more Fok I cleavage domain is not codon diversified. In some embodiments the polynucleotide sequence encoding a first Fok I cleavage domain is operatively linked to the polynucleotide sequence encoding the first zinc finger DNA binding protein (ZFP). In some embodiments the polynucleotide sequence encoding a second Fok I cleavage domain is operatively linked to the polynucleotide sequence encoding the second zinc finger DNA binding protein (ZFP). In some embodiments the polynucleotide sequence encoding a first Fok I cleavage domain is located 3′ to the polynucleotide sequence encoding the first zinc finger DNA binding protein (ZFP). In some embodiments the polynucleotide sequence encoding a second Fok I cleavage domain is located 3′ to the polynucleotide sequence encoding the second zinc finger DNA binding protein (ZFP).

In some embodiments, the cleavage domain comprises one or more engineered cleavage half-domain (also referred to as dimerization domain mutants) that minimize or prevent homodimerization, as described, for example, in U.S. Pat. Nos. 8,772,453; 8,623,618; 8,409,861; 8,034,598; 7,914,796; and 7,888,121, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in some embodiments, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. U.S. Pat. Nos. 7,914,796 and 8,034,598, the disclosures of which are incorporated by reference in their entireties. In some embodiments, the engineered cleavage half-domain comprises mutations at positions 486, 499 and 496 (numbered relative to wild-type Fok I), for instance mutations that replace the wild type Gln (Q) residue at position 486 with a Glu(E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In some embodiments, the engineered cleavage half-domain comprises mutations at positions 490, 538 and 537 (numbered relative to wild-type Fok I), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In some embodiments, the engineered cleavage half-domain comprises mutations at positions 490 and 537 (numbered relative to wild-type Fok I), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. No. 8,772,453. In some embodiments, the engineered cleavage half domain comprises the “Sharkey” and/or “Sharkey mutations” (see Guo et al. (2010) J. Mol. Biol. 400(1):96-107).

Engineered cleavage half-domains described herein can be prepared using any suitable method, for example, by site-directed mutagenesis of wild-type cleavage half-domains (Fok I) as described in U.S. Pat. Nos. 7,888,121; 7,914,796; 8,034,598; and 8,623,618 and U.S. Patent Publication Nos. 2019/0241877 and 2018/0087072.

In some embodiments, the polynucleotide sequence encoding the first zinc finger nuclease comprises the nucleotide sequence of any one of SEQ ID NOs: 71-84. In some embodiments, the polynucleotide sequence encoding the first zinc finger nuclease comprises the nucleotide sequence of SEQ ID NO: 71. In some embodiments, the polynucleotide sequence encoding the first zinc finger nuclease comprises the nucleotide sequence of SEQ ID NO: 72. In some embodiments, the polynucleotide sequence encoding the first zinc finger nuclease comprises the nucleotide sequence of SEQ ID NO: 73. In some embodiments, the polynucleotide sequence encoding the first zinc finger nuclease comprises the nucleotide sequence of SEQ ID NO: 74 In some embodiments, the polynucleotide sequence encoding the first zinc finger nuclease comprises the nucleotide sequence of SEQ ID NO: 75. In some embodiments, the polynucleotide sequence encoding the first zinc finger nuclease comprises the nucleotide sequence of SEQ ID NO: 76. In some embodiments, the polynucleotide sequence encoding the first zinc finger nuclease comprises the nucleotide sequence of SEQ ID NO: 77. In some embodiments, the polynucleotide sequence encoding the first zinc finger nuclease comprises the nucleotide sequence of SEQ ID NO: 78. In some embodiments, the polynucleotide sequence encoding the first zinc finger nuclease comprises the nucleotide sequence of SEQ ID NO: 79. In some embodiments, the polynucleotide sequence encoding the first zinc finger nuclease comprises the nucleotide sequence of SEQ ID NO: 80. In some embodiments, the polynucleotide sequence encoding the first zinc finger nuclease comprises the nucleotide sequence of SEQ ID NO: 81. In some embodiments, the polynucleotide sequence encoding the first zinc finger nuclease comprises the nucleotide sequence of SEQ ID NO: 82. In some embodiments, the polynucleotide sequence encoding the first zinc finger nuclease comprises the nucleotide sequence of SEQ ID NO: 83. In some embodiments, the polynucleotide sequence encoding the first zinc finger nuclease comprises the nucleotide sequence of SEQ ID NO:84.

In some embodiments, the polynucleotide sequence encoding the second zinc finger nuclease comprises the nucleotide sequence of any one of SEQ ID NOs: 71-84. In some embodiments, the polynucleotide sequence encoding the second zinc finger nuclease comprises the nucleotide sequence of SEQ ID NO: 71. In some embodiments, the polynucleotide sequence encoding the second zinc finger nuclease comprises the nucleotide sequence of SEQ ID NO: 72. In some embodiments, the polynucleotide sequence encoding the second zinc finger nuclease comprises the nucleotide sequence of SEQ ID NO: 73. In some embodiments, the polynucleotide sequence encoding the second zinc finger nuclease comprises the nucleotide sequence of SEQ ID NO: 74 In some embodiments, the polynucleotide sequence encoding the second zinc finger nuclease comprises the nucleotide sequence of SEQ ID NO: 75. In some embodiments, the polynucleotide sequence encoding the second zinc finger nuclease comprises the nucleotide sequence of SEQ ID NO: 76. In some embodiments, the polynucleotide sequence encoding the second zinc finger nuclease comprises the nucleotide sequence of SEQ ID NO: 77. In some embodiments, the polynucleotide sequence encoding the second zinc finger nuclease comprises the nucleotide sequence of SEQ ID NO: 78. In some embodiments, the polynucleotide sequence encoding the second zinc finger nuclease comprises the nucleotide sequence of SEQ ID NO: 79. In some embodiments, the polynucleotide sequence encoding the second zinc finger nuclease comprises the nucleotide sequence of SEQ ID NO: 80. In some embodiments, the polynucleotide sequence encoding the second zinc finger nuclease comprises the nucleotide sequence of SEQ ID NO: 81. In some embodiments, the polynucleotide sequence encoding the second zinc finger nuclease comprises the nucleotide sequence of SEQ ID NO: 82. In some embodiments, the polynucleotide sequence encoding the second zinc finger nuclease comprises the nucleotide sequence of SEQ ID NO: 83. In some embodiments, the polynucleotide sequence encoding the second zinc finger nuclease comprises the nucleotide sequence of SEQ ID NO:84.

In some embodiments, the polynucleotide sequence encoding the first zinc finger nuclease comprises a nucleotide sequence encoding the amino acid sequence of any one of SEQ ID NOs: 130-131. In some embodiments, the polynucleotide sequence encoding the first zinc finger nuclease comprises a nucleotide sequence encoding the amino acid sequence of SEQ ID NOs: 130. In some embodiments, the polynucleotide sequence encoding the first zinc finger nuclease comprises a nucleotide sequence encoding the amino acid sequence of SEQ ID NOs: 131.

In some embodiments, the polynucleotide sequence encoding the second zinc finger nuclease comprises a nucleotide sequence encoding the amino acid sequence of any one of SEQ ID NOs: 130-131. In some embodiments, the polynucleotide sequence encoding the second zinc finger nuclease comprises a nucleotide sequence encoding the amino acid sequence of SEQ ID NOs: 130. In some embodiments, the polynucleotide sequence encoding the second zinc finger nuclease comprises a nucleotide sequence encoding the amino acid sequence of SEQ ID NOs: 131.

In some embodiments, the nucleic acid encoding the 2-in-1 zinc finger nuclease variants further comprises one or more nucleotide sequences encoding one or more nuclear localization sequence (NLS). In some embodiments, the nucleic acid encoding the 2-in-1 zinc finger nuclease variant comprises a nucleotide sequence encoding a first nuclear localization sequence (NLS) and a nucleotide sequence encoding a second nuclear localization sequence (NLS), wherein the nucleotide sequence encoding first nuclear localization sequence (NLS) is located 5′ to the nucleotide sequence encoding the first zinc finger DNA binding protein (ZFP) and the nucleotide sequence encoding the second nuclear localization sequence (NLS) is located 5′ to the nucleotide sequence encoding the second zinc finger DNA binding protein (ZFP). In some embodiments, the nucleotide sequence encoding the first NLS is operatively linked to the nucleotide sequence encoding the first ZFP and the nucleotide sequence encoding the second NLS is operatively linked to the nucleotide sequence encoding the second ZFP. In some embodiments, the nucleotide sequence encoding the first NLS is codon diversified. In some embodiments, the nucleotide sequence encoding the first NLS is not codon diversified. In some embodiments, the nucleotide sequence encoding the second NLS is codon diversified. In some embodiments, the nucleotide sequence encoding the second NLS is not codon diversified. In some embodiments, the nucleotide sequence encoding each of the two or more NLS is the same. In some embodiments, the nucleotide sequence encoding each of the two or more NLS is the different. In some embodiments, each of the two or more NLS have the same amino acid sequence. In some embodiments, each of the two or more NLS have different amino acid sequences. In some embodiments, the polynucleotide encoding the first NLS comprises the nucleotide sequence set forth in any one of SEQ ID NO: 59-70 or 155. In some embodiments, the polynucleotide encoding the first NLS comprises the nucleotide sequence set forth in SEQ ID NO: 59. In some embodiments, the polynucleotide encoding the first NLS comprises the nucleotide sequence set forth in SEQ ID NO: 60. In some embodiments, the polynucleotide encoding the first NLS comprises the nucleotide sequence set forth in SEQ ID NO: 61. In some embodiments, the polynucleotide encoding the first NLS comprises the nucleotide sequence set forth in SEQ ID NO: 62. In some embodiments, the polynucleotide encoding the first NLS comprises the nucleotide sequence set forth in SEQ ID NO: 63. In some embodiments, the polynucleotide encoding the first NLS comprises the nucleotide sequence set forth in SEQ ID NO: 64. In some embodiments, the polynucleotide encoding the first NLS comprises the nucleotide sequence set forth in SEQ ID NO: 65. In some embodiments, the polynucleotide encoding the first NLS comprises the nucleotide sequence set forth in SEQ ID NO: 66. In some embodiments, the polynucleotide encoding the first NLS comprises the nucleotide sequence set forth in SEQ ID NO: 67. In some embodiments, the polynucleotide encoding the first NLS comprises the nucleotide sequence set forth in SEQ ID NO: 68. In some embodiments, the polynucleotide encoding the first NLS comprises the nucleotide sequence set forth in SEQ ID NO: 69. In some embodiments, the polynucleotide encoding the first NLS comprises the nucleotide sequence set forth in SEQ ID NO: 70. In some embodiments, the polynucleotide encoding the first NLS comprises the nucleotide sequence set forth in SEQ ID NO: 155. In some embodiments, the polynucleotide encoding the second NLS comprises the nucleotide sequence set forth in any one of SEQ ID NO: 59-70 or 155. In some embodiments, the polynucleotide encoding the second NLS comprises the nucleotide sequence set forth in SEQ ID NO: 59. In some embodiments, the polynucleotide encoding the second NLS comprises the nucleotide sequence set forth in SEQ ID NO: 60. In some embodiments, the polynucleotide encoding the second NLS comprises the nucleotide sequence set forth in SEQ ID NO: 61. In some embodiments, the polynucleotide encoding the second NLS comprises the nucleotide sequence set forth in SEQ ID NO: 62. In some embodiments, the polynucleotide encoding the second NLS comprises the nucleotide sequence set forth in SEQ ID NO: 63. In some embodiments, the polynucleotide encoding the second NLS comprises the nucleotide sequence set forth in SEQ ID NO: 64. In some embodiments, the polynucleotide encoding the second NLS comprises the nucleotide sequence set forth in SEQ ID NO: 65. In some embodiments, the polynucleotide encoding the second NLS comprises the nucleotide sequence set forth in SEQ ID NO: 66. In some embodiments, the polynucleotide encoding the second NLS comprises the nucleotide sequence set forth in SEQ ID NO: 67. In some embodiments, the polynucleotide encoding the second NLS comprises the nucleotide sequence set forth in SEQ ID NO: 68. In some embodiments, the polynucleotide encoding the second NLS comprises the nucleotide sequence set forth in SEQ ID NO: 69. In some embodiments, the polynucleotide encoding the second NLS comprises the nucleotide sequence set forth in SEQ ID NO: 70. In some embodiments, the polynucleotide encoding the first NLS comprises the nucleotide sequence set forth in SEQ ID NO: 155.

In some embodiments, the polynucleotide encoding the first NLS comprises a nucleotide sequence encoding the amino acid sequence set forth in any one of SEQ ID NO: 3-9 and 156. In some embodiments, the polynucleotide encoding the first NLS comprises a nucleotide sequence encoding the amino acid sequence set forth in SEQ ID NO: 3. In some embodiments, the polynucleotide encoding the first NLS comprises a nucleotide sequence encoding the amino acid sequence set forth in SEQ ID NO: 4. In some embodiments, the polynucleotide encoding the first NLS comprises a nucleotide sequence encoding the amino acid sequence set forth in SEQ ID NO:5. In some embodiments, the polynucleotide encoding the first NLS comprises a nucleotide sequence encoding the amino acid sequence set forth in SEQ ID NO: 6. In some embodiments, the polynucleotide encoding the first NLS comprises a nucleotide sequence encoding the amino acid sequence set forth in SEQ ID NO: 7. In some embodiments, the polynucleotide encoding the first NLS comprises a nucleotide sequence encoding the amino acid sequence set forth in SEQ ID NO: 8. In some embodiments, the polynucleotide encoding the first NLS comprises a nucleotide sequence encoding the amino acid sequence set forth in SEQ ID NO: 9. In some embodiments, the polynucleotide encoding the first NLS comprises a nucleotide sequence encoding the amino acid sequence set forth in SEQ ID NO: 156. In some embodiments, the polynucleotide encoding the second NLS comprises a nucleotide sequence encoding the amino acid sequence set forth in any one of SEQ ID NO: 3-9 and 156. In some embodiments, the polynucleotide encoding the second NLS comprises a nucleotide sequence encoding the amino acid sequence set forth in SEQ ID NO: 3. In some embodiments, the polynucleotide encoding the second NLS comprises a nucleotide sequence encoding the amino acid sequence set forth in SEQ ID NO: 4. In some embodiments, the polynucleotide encoding the second NLS comprises a nucleotide sequence encoding the amino acid sequence set forth in SEQ ID NO:5. In some embodiments, the polynucleotide encoding the second NLS comprises a nucleotide sequence encoding the amino acid sequence set forth in SEQ ID NO: 6. In some embodiments, the polynucleotide encoding the second NLS comprises a nucleotide sequence encoding the amino acid sequence set forth in SEQ ID NO: 7. In some embodiments, the polynucleotide encoding the second NLS comprises a nucleotide sequence encoding the amino acid sequence set forth in SEQ ID NO: 8. In some embodiments, the polynucleotide encoding the second NLS comprises a nucleotide sequence encoding the amino acid sequence set forth in SEQ ID NO: 9. In some embodiments, the polynucleotide encoding the second NLS comprises a nucleotide sequence encoding the amino acid sequence set forth in SEQ ID NO: 156.

In some embodiments, the polynucleotide sequence encoding the first zinc finger nuclease comprises the nucleotide sequence of any one of SEQ ID NOs: 139-152. In some embodiments, the polynucleotide sequence encoding the first zinc finger nuclease comprises the nucleotide sequence of SEQ ID NO: 139. In some embodiments, the polynucleotide sequence encoding the first zinc finger nuclease comprises the nucleotide sequence of SEQ ID NO: 140. In some embodiments, the polynucleotide sequence encoding the first zinc finger nuclease comprises the nucleotide sequence of SEQ ID NO: 141. In some embodiments, the polynucleotide sequence encoding the first zinc finger nuclease comprises the nucleotide sequence of SEQ ID NO: 142. In some embodiments, the polynucleotide sequence encoding the first zinc finger nuclease comprises the nucleotide sequence of SEQ ID NO: 143. In some embodiments, the polynucleotide sequence encoding the first zinc finger nuclease comprises the nucleotide sequence of SEQ ID NO: 144. In some embodiments, the polynucleotide sequence encoding the first zinc finger nuclease comprises the nucleotide sequence of SEQ ID NO: 145. In some embodiments, the polynucleotide sequence encoding the first zinc finger nuclease comprises the nucleotide sequence of SEQ ID NO: 146. In some embodiments, the polynucleotide sequence encoding the first zinc finger nuclease comprises the nucleotide sequence of SEQ ID NO: 147. In some embodiments, the polynucleotide sequence encoding the first zinc finger nuclease comprises the nucleotide sequence of SEQ ID NO: 148. In some embodiments, the polynucleotide sequence encoding the first zinc finger nuclease comprises the nucleotide sequence of SEQ ID NO: 149. In some embodiments, the polynucleotide sequence encoding the first zinc finger nuclease comprises the nucleotide sequence of SEQ ID NO: 150. In some embodiments, the polynucleotide sequence encoding the first zinc finger nuclease comprises the nucleotide sequence of SEQ ID NO: 151. In some embodiments, the polynucleotide sequence encoding the first zinc finger nuclease comprises the nucleotide sequence of SEQ ID NO: 152.

In some embodiments, the polynucleotide sequence encoding the second zinc finger nuclease comprises the nucleotide sequence of any one of SEQ ID NOs: 139-152. In some embodiments, the polynucleotide sequence encoding the second zinc finger nuclease comprises the nucleotide sequence of SEQ ID NO: 139. In some embodiments, the polynucleotide sequence encoding the second zinc finger nuclease comprises the nucleotide sequence of SEQ ID NO: 140. In some embodiments, the polynucleotide sequence encoding the second zinc finger nuclease comprises the nucleotide sequence of SEQ ID NO: 141. In some embodiments, the polynucleotide sequence encoding the second zinc finger nuclease comprises the nucleotide sequence of SEQ ID NO: 142. In some embodiments, the polynucleotide sequence encoding the second zinc finger nuclease comprises the nucleotide sequence of SEQ ID NO: 143. In some embodiments, the polynucleotide sequence encoding the second zinc finger nuclease comprises the nucleotide sequence of SEQ ID NO: 144. In some embodiments, the polynucleotide sequence encoding the second zinc finger nuclease comprises the nucleotide sequence of SEQ ID NO: 145. In some embodiments, the polynucleotide sequence encoding the second zinc finger nuclease comprises the nucleotide sequence of SEQ ID NO: 146. In some embodiments, the polynucleotide sequence encoding the second zinc finger nuclease comprises the nucleotide sequence of SEQ ID NO: 147. In some embodiments, the polynucleotide sequence encoding the second zinc finger nuclease comprises the nucleotide sequence of SEQ ID NO: 148. In some embodiments, the polynucleotide sequence encoding the second zinc finger nuclease comprises the nucleotide sequence of SEQ ID NO: 149. In some embodiments, the polynucleotide sequence encoding the second zinc finger nuclease comprises the nucleotide sequence of SEQ ID NO: 150. In some embodiments, the polynucleotide sequence encoding the second zinc finger nuclease comprises the nucleotide sequence of SEQ ID NO: 151. In some embodiments, the polynucleotide sequence encoding the second zinc finger nuclease comprises the nucleotide sequence of SEQ ID NO: 152.

In some embodiments, the nucleic acid encoding the 2-in-1 zinc finger nuclease variant further comprises one or more nucleotide sequences encoding one or more epitope tag. Epitope tags or expression tags refer to a peptide sequence engineered to be positioned 5′ or 3′ to a translated protein. Epitope tags include, for example one or more copies of FLAG, HA, CBP, GST, HBH, MBP, Myc, His, polyHis, S-tag, SUMO, TAP, TAGP, TRX, V5, GFP, RFP, YFP, and the like. “Expression tags” include sequences that encode reporters that may be operably linked to a desired gene sequence in order to monitor expression of the gene of interest.

In some embodiments, the nucleic acid encoding the 2-in-1 zinc finger nuclease variant further comprises one or more nucleotide sequences encoding one or more copies of an epitope tag. In some embodiments, the nucleic acid encoding the 2-in-1 zinc finger nuclease variant further comprise a first nucleotide sequence encoding a first epitope tag and a second nucleotide sequence encoding a second epitope tag. In some embodiments, each of said first epitope tag and second epitope tag is the same. In some embodiments, the first nucleotide sequence encoding the first epitope tag is located 5′ to the nucleotide sequence encoding the first ZFP, and the second nucleotide sequence encoding the second epitope tag is located 5′ to the nucleotide sequence encoding the second ZFP. In some embodiments, the first nucleotide sequence encoding the first epitope tag is located 5′ to the nucleotide sequence encoding the first NLS, and the second nucleotide sequence encoding the second epitope tag is located 5′ to the nucleotide sequence encoding the second NLS. In some embodiments, the first nucleotide sequence encoding the first epitope tag is located 3′ to the nucleotide sequence encoding the first ZFP, and the second nucleotide sequence encoding the second epitope tag is located 3′ to the nucleotide sequence encoding the second ZFP. In some embodiments, the first nucleotide sequence encoding the first epitope tag is located 3′ to the nucleotide sequence encoding the first NLS, and the second nucleotide sequence encoding the second epitope tag is located 3′ to the nucleotide sequence encoding the second NLS. In some embodiments, the first nucleotide sequence encoding the first epitope tag is codon diversified. In some embodiments, the first nucleotide sequence encoding the first epitope tag is not codon diversified. In some embodiments, the second nucleotide sequence encoding the second epitope tag is codon diversified. In some embodiments, the second nucleotide sequence encoding the second epitope tag is not codon diversified. In some embodiments, each of the two or more epitope tags has the same amino acid sequence. In some embodiments, each of the two or more epitope tags has different amino acid sequences. In some embodiments, each of the two or more epitope tags is encoded by a polynucleotide having the same nucleotide sequence. In some embodiments, each of the two or more epitope tags is encoded by a polynucleotide having different nucleotide sequences.

In some embodiments, the nucleic acid encoding the 2-in-1 zinc finger nuclease variant further comprises one or more nucleotide sequences encoding one or more copies of a FLAG tag. In some embodiments, the epitope tag is 3×FLAG. In some embodiments, the nucleic acid encoding the 2-in-1 zinc finger nuclease variant further comprise a first nucleotide sequence encoding a first FLAG tag and a second nucleotide sequence encoding a second FLAG tag. In some embodiments, each of said first FLAG tag and second FLAG tag is 3×FLAG. In some embodiments, the first nucleotide sequence encoding the first FLAG tag is located 5′ to the nucleotide sequence encoding the first ZFP, and the second nucleotide sequence encoding the second FLAG tag is located 5′ to the nucleotide sequence encoding the second ZFP. In some embodiments, the first nucleotide sequence encoding the first FLAG tag is located 5′ to the nucleotide sequence encoding the first NLS, and the second nucleotide sequence encoding the second FLAG tag is located 5′ to the nucleotide sequence encoding the second NLS. In some embodiments, the first nucleotide sequence encoding the first FLAG tag is located 3′ to the nucleotide sequence encoding the first ZFP, and the second nucleotide sequence encoding the second FLAG tag is located 3′ to the nucleotide sequence encoding the second ZFP. In some embodiments, the first nucleotide sequence encoding the first FLAG tag is located 3′ to the nucleotide sequence encoding the first NLS, and the second nucleotide sequence encoding the second FLAG tag is located 3′ to the nucleotide sequence encoding the second NLS. In some embodiments, the first nucleotide sequence encoding the first FLAG tag is codon diversified. In some embodiments, the first nucleotide sequence encoding the first FLAG tag is not codon diversified. In some embodiments, the second nucleotide sequence encoding the second FLAG tag is codon diversified. In some embodiments, the second nucleotide sequence encoding the second FLAG tag is not codon diversified. In some embodiments, each of the two or more FLAG tags has the same amino acid sequence. In some embodiments, each of the two or more FLAG tags has different amino acid sequences. In some embodiments, each of the two or more FLAG tags is encoded by a polynucleotide having the same nucleotide sequence. In some embodiments, each of the two or more FLAG tags is encoded by a polynucleotide having different nucleotide sequences.

In some embodiments, the nucleotide sequence encoding the first FLAG tag comprises the nucleotide sequence set forth in any one of SEQ ID NO: 15-16 or 50-58. In some embodiments, the nucleotide sequence encoding the first FLAG tag comprises the nucleotide sequence set forth in SEQ ID NO: 15. In some embodiments, the nucleotide sequence encoding the first FLAG tag comprises the nucleotide sequence set forth in SEQ ID NO: 16. In some embodiments, the nucleotide sequence encoding the first FLAG tag comprises the nucleotide sequence set forth in SEQ ID NO: 50. In some embodiments, the nucleotide sequence encoding the first FLAG tag comprises the nucleotide sequence set forth in SEQ ID NO: 51. In some embodiments, the nucleotide sequence encoding the first FLAG tag comprises the nucleotide sequence set forth in SEQ ID NO: 52. In some embodiments, the nucleotide sequence encoding the first FLAG tag comprises the nucleotide sequence set forth in SEQ ID NO: 53. In some embodiments, the nucleotide sequence encoding the first FLAG tag comprises the nucleotide sequence set forth in SEQ ID NO: 54. In some embodiments, the nucleotide sequence encoding the first FLAG tag comprises the nucleotide sequence set forth in SEQ ID NO: 55. In some embodiments, the nucleotide sequence encoding the first FLAG tag comprises the nucleotide sequence set forth in SEQ ID NO: 56. In some embodiments, the nucleotide sequence encoding the first FLAG tag comprises the nucleotide sequence set forth in SEQ ID NO: 57. In some embodiments, the nucleotide sequence encoding the first FLAG tag comprises the nucleotide sequence set forth in SEQ ID NO: 58. In some embodiments, the nucleotide sequence encoding the second FLAG tag comprises the nucleotide sequence set forth in any one of SEQ ID NO: 15-16 or 50-58. In some embodiments, the nucleotide sequence encoding the second FLAG tag comprises the nucleotide sequence set forth in SEQ ID NO: 15. In some embodiments, the nucleotide sequence encoding the second FLAG tag comprises the nucleotide sequence set forth in SEQ ID NO: 16. In some embodiments, the nucleotide sequence encoding the second FLAG tag comprises the nucleotide sequence set forth in SEQ ID NO: 50. In some embodiments, the nucleotide sequence encoding the second FLAG tag comprises the nucleotide sequence set forth in SEQ ID NO: 51. In some embodiments, the nucleotide sequence encoding the second FLAG tag comprises the nucleotide sequence set forth in SEQ ID NO: 52. In some embodiments, the nucleotide sequence encoding the second FLAG tag comprises the nucleotide sequence set forth in SEQ ID NO: 53. In some embodiments, the nucleotide sequence encoding the second FLAG tag comprises the nucleotide sequence set forth in SEQ ID NO: 54. In some embodiments, the nucleotide sequence encoding the second FLAG tag comprises the nucleotide sequence set forth in SEQ ID NO: 55. In some embodiments, the nucleotide sequence encoding the second FLAG tag comprises the nucleotide sequence set forth in SEQ ID NO: 56. In some embodiments, the nucleotide sequence encoding the second FLAG tag comprises the nucleotide sequence set forth in SEQ ID NO: 57. In some embodiments, the nucleotide sequence encoding the second FLAG tag comprises the nucleotide sequence set forth in SEQ ID NO: 58. In some embodiments, the nucleotide sequence encoding the first FLAG tag comprises a nucleotide sequence encoding the amino acid sequence set forth in any one of SEQ ID NO: 1-2. In some embodiments, the nucleotide sequence encoding the first FLAG tag comprises an nucleotide sequence encoding the amino acid sequence set forth in SEQ ID NO: 1. In some embodiments, the nucleotide sequence encoding the first FLAG tag comprises a nucleotide sequence encoding the amino acid sequence set forth in SEQ ID NO: 2. In some embodiments, the nucleotide sequence encoding the second FLAG tag comprises the nucleotide sequence encoding the amino acid sequence set forth in any one of SEQ ID NO: 1-2. In some embodiments, the nucleotide sequence encoding the second FLAG tag comprises a nucleotide sequence encoding the amino acid sequence set forth in SEQ ID NO: 1. In some embodiments, the nucleotide sequence encoding the second FLAG tag comprises a nucleotide sequence encoding amino acid sequence set forth in SEQ ID NO: 2.

In some embodiments, the polynucleotide sequence encoding the first zinc finger nuclease comprises the nucleotide sequence of any one of SEQ ID NOs: 17-23 and 25-31. In some embodiments, the polynucleotide sequence encoding the first zinc finger nuclease comprises the nucleotide sequence of SEQ ID NO: 17. In some embodiments, the polynucleotide sequence encoding the first zinc finger nuclease comprises the nucleotide sequence of SEQ ID NO: 18. In some embodiments, the polynucleotide sequence encoding the first zinc finger nuclease comprises the nucleotide sequence of SEQ ID NO: 19. In some embodiments, the polynucleotide sequence encoding the first zinc finger nuclease comprises the nucleotide sequence of SEQ ID NO: 20. In some embodiments, the polynucleotide sequence encoding the first zinc finger nuclease comprises the nucleotide sequence of SEQ ID NO: 21. In some embodiments, the polynucleotide sequence encoding the first zinc finger nuclease comprises the nucleotide sequence of SEQ ID NO: 22. In some embodiments, the polynucleotide sequence encoding the first zinc finger nuclease comprises the nucleotide sequence of SEQ ID NO: 23. In some embodiments, the polynucleotide sequence encoding the first zinc finger nuclease comprises the nucleotide sequence of SEQ ID NO: 25. In some embodiments, the polynucleotide sequence encoding the first zinc finger nuclease comprises the nucleotide sequence of SEQ ID NO: 26. In some embodiments, the polynucleotide sequence encoding the first zinc finger nuclease comprises the nucleotide sequence of SEQ ID NO: 27. In some embodiments, the polynucleotide sequence encoding the first zinc finger nuclease comprises the nucleotide sequence of SEQ ID NO: 28. In some embodiments, the polynucleotide sequence encoding the first zinc finger nuclease comprises the nucleotide sequence of SEQ ID NO: 29. In some embodiments, the polynucleotide sequence encoding the first zinc finger nuclease comprises the nucleotide sequence of SEQ ID NO: 30. In some embodiments, the polynucleotide sequence encoding the first zinc finger nuclease comprises the nucleotide sequence of SEQ ID NO: 31.

In some embodiments, the polynucleotide sequence encoding the second zinc finger nuclease comprises the nucleotide sequence of any one of SEQ ID NOs: 17-23 and 25-31. In some embodiments, the polynucleotide sequence encoding the second zinc finger nuclease comprises the nucleotide sequence of SEQ ID NO: 17. In some embodiments, the polynucleotide sequence encoding the second zinc finger nuclease comprises the nucleotide sequence of SEQ ID NO: 18. In some embodiments, the polynucleotide sequence encoding the second zinc finger nuclease comprises the nucleotide sequence of SEQ ID NO: 19. In some embodiments, the polynucleotide sequence encoding the second zinc finger nuclease comprises the nucleotide sequence of SEQ ID NO: 20. In some embodiments, the polynucleotide sequence encoding the second zinc finger nuclease comprises the nucleotide sequence of SEQ ID NO: 21. In some embodiments, the polynucleotide sequence encoding the second zinc finger nuclease comprises the nucleotide sequence of SEQ ID NO: 22. In some embodiments, the polynucleotide sequence encoding the second zinc finger nuclease comprises the nucleotide sequence of SEQ ID NO: 23. In some embodiments, the polynucleotide sequence encoding the second zinc finger nuclease comprises the nucleotide sequence of SEQ ID NO: 25. In some embodiments, the polynucleotide sequence encoding the second zinc finger nuclease comprises the nucleotide sequence of SEQ ID NO: 26. In some embodiments, the polynucleotide sequence encoding the second zinc finger nuclease comprises the nucleotide sequence of SEQ ID NO: 27. In some embodiments, the polynucleotide sequence encoding the second zinc finger nuclease comprises the nucleotide sequence of SEQ ID NO: 28. In some embodiments, the polynucleotide sequence encoding the second zinc finger nuclease comprises the nucleotide sequence of SEQ ID NO: 29. In some embodiments, the polynucleotide sequence encoding the second zinc finger nuclease comprises the nucleotide sequence of SEQ ID NO: 30. In some embodiments, the polynucleotide sequence encoding the second zinc finger nuclease comprises the nucleotide sequence of SEQ ID NO: 31.

A “2A sequence” or “2A self-cleaving sequence”, as used herein, refers to any sequence that encodes a peptide which can induce the cleaving a recombinant protein in a cell. In some embodiments the nucleotide sequence encoding the 2A self-cleaving sequence encodes a peptide that is between 15 and 25 amino acids. In some embodiments the nucleotide sequence encoding the 2A self-cleaving sequence encodes a peptide that is between 18 and 22 amino acids. Non-limiting examples of 2A self-cleaving peptides include T2A, P2A, E2A and F2A sequences. See, e.g., Donnelly et al. (2001) J. Gen. Virol. 82:1013-1025.

In some embodiments, the nucleotide sequence encoding the 2A self-cleaving sequence comprises the nucleotide sequence of SEQ ID NO:24. In some embodiments the nucleotide sequence encodes a 2A self-cleaving sequence comprising the amino acid sequence of SEQ ID NO: 138.

In some embodiments, the nucleic acid encoding a 2-in-1 zinc finger nuclease variant comprises a nucleotide selected from any one of SEQ ID NO: 85-115. In some embodiments, the nucleic acid encoding a 2-in-1 zinc finger nuclease variant comprises the nucleotide sequence of SEQ ID NO: 85. In some embodiments, the nucleic acid encoding a 2-in-1 zinc finger nuclease variant comprises the nucleotide sequence of SEQ ID NO: 86. In some embodiments, the nucleic acid encoding a 2-in-1 zinc finger nuclease variant comprises the nucleotide sequence of SEQ ID NO: 87. In some embodiments, the nucleic acid encoding a 2-in-1 zinc finger nuclease variant comprises the nucleotide sequence of SEQ ID NO: 88. In some embodiments, the nucleic acid encoding a 2-in-1 zinc finger nuclease variant comprises the nucleotide sequence of SEQ ID NO: 89. In some embodiments, the nucleic acid encoding a 2-in-1 zinc finger nuclease variant comprises the nucleotide sequence of SEQ ID NO: 90. In some embodiments, the nucleic acid encoding a 2-in-1 zinc finger nuclease variant comprises the nucleotide sequence of SEQ ID NO: 91. In some embodiments, the nucleic acid encoding a 2-in-1 zinc finger nuclease variant comprises the nucleotide sequence of SEQ ID NO: 92. In some embodiments, the nucleic acid encoding a 2-in-1 zinc finger nuclease variant comprises the nucleotide sequence of SEQ ID NO: 93. In some embodiments, the nucleic acid encoding a 2-in-1 zinc finger nuclease variant comprises the nucleotide sequence of SEQ ID NO: 94. In some embodiments, the nucleic acid encoding a 2-in-1 zinc finger nuclease variant comprises the nucleotide sequence of SEQ ID NO: 95. In some embodiments, the nucleic acid encoding a 2-in-1 zinc finger nuclease variant comprises the nucleotide sequence of SEQ ID NO: 96. In some embodiments, the nucleic acid encoding a 2-in-1 zinc finger nuclease variant comprises the nucleotide sequence of SEQ ID NO: 97. In some embodiments, the nucleic acid encoding a 2-in-1 zinc finger nuclease variant comprises the nucleotide sequence of SEQ ID NO: 98. In some embodiments, the nucleic acid encoding a 2-in-1 zinc finger nuclease variant comprises the nucleotide sequence of SEQ ID NO: 99. In some embodiments, the nucleic acid encoding a 2-in-1 zinc finger nuclease variant comprises the nucleotide sequence of SEQ ID NO: 100. In some embodiments, the nucleic acid encoding a 2-in-1 zinc finger nuclease variant comprises the nucleotide sequence of SEQ ID NO: 101. In some embodiments, the nucleic acid encoding a 2-in-1 zinc finger nuclease variant comprises the nucleotide sequence of SEQ ID NO: 102. In some embodiments, the nucleic acid encoding a 2-in-1 zinc finger nuclease variant comprises the nucleotide sequence of SEQ ID NO: 103. In some embodiments, the nucleic acid encoding a 2-in-1 zinc finger nuclease variant comprises the nucleotide sequence of SEQ ID NO: 104. In some embodiments, the nucleic acid encoding a 2-in-1 zinc finger nuclease variant comprises the nucleotide sequence of SEQ ID NO: 105. In some embodiments, the nucleic acid encoding a 2-in-1 zinc finger nuclease variant comprises the nucleotide sequence of SEQ ID NO: 106. In some embodiments, the nucleic acid encoding a 2-in-1 zinc finger nuclease variant comprises the nucleotide sequence of SEQ ID NO: 107. In some embodiments, the nucleic acid encoding a 2-in-1 zinc finger nuclease variant comprises the nucleotide sequence of SEQ ID NO: 108. In some embodiments, the nucleic acid encoding a 2-in-1 zinc finger nuclease variant comprises the nucleotide sequence of SEQ ID NO: 109. In some embodiments, the nucleic acid encoding a 2-in-1 zinc finger nuclease variant comprises the nucleotide sequence of SEQ ID NO: 110. In some embodiments, the nucleic acid encoding a 2-in-1 zinc finger nuclease variant comprises the nucleotide sequence of SEQ ID NO: 111. In some embodiments, the nucleic acid encoding a 2-in-1 zinc finger nuclease variant comprises the nucleotide sequence of SEQ ID NO: 112. In some embodiments, the nucleic acid encoding a 2-in-1 zinc finger nuclease variant comprises the nucleotide sequence of SEQ ID NO: 113. In some embodiments, the nucleic acid encoding a 2-in-1 zinc finger nuclease variant comprises the nucleotide sequence of SEQ ID NO: 114. In some embodiments, the nucleic acid encoding a 2-in-1 zinc finger nuclease variant comprises the nucleotide sequence of SEQ ID NO: 115.

In some embodiments, the nucleic acid encoding a 2-in-1 zinc finger nuclease variant comprises a nucleotide sequence selected from any one of SEQ ID NO: 35-49. In some embodiments, the nucleic acid encoding a 2-in-1 zinc finger nuclease variant comprises a nucleotide sequence selected from any one of SEQ ID NO: 35. In some embodiments, the nucleic acid encoding a 2-in-1 zinc finger nuclease variant comprises a nucleotide sequence selected from any one of SEQ ID NO: 36. In some embodiments, the nucleic acid encoding a 2-in-1 zinc finger nuclease variant comprises a nucleotide sequence selected from any one of SEQ ID NO: 37. In some embodiments, the nucleic acid encoding a 2-in-1 zinc finger nuclease variant comprises a nucleotide sequence selected from any one of SEQ ID NO: 35-38. In some embodiments, the nucleic acid encoding a 2-in-1 zinc finger nuclease variant comprises a nucleotide sequence selected from any one of SEQ ID NO: 39. In some embodiments, the nucleic acid encoding a 2-in-1 zinc finger nuclease variant comprises a nucleotide sequence selected from any one of SEQ ID NO: 40. In some embodiments, the nucleic acid encoding a 2-in-1 zinc finger nuclease variant comprises a nucleotide sequence selected from any one of SEQ ID NO: 41. In some embodiments, the nucleic acid encoding a 2-in-1 zinc finger nuclease variant comprises a nucleotide sequence selected from any one of SEQ ID NO: 42. In some embodiments, the nucleic acid encoding a 2-in-1 zinc finger nuclease variant comprises a nucleotide sequence selected from any one of SEQ ID NO: 43. In some embodiments, the nucleic acid encoding a 2-in-1 zinc finger nuclease variant comprises a nucleotide sequence selected from any one of SEQ ID NO: 44. In some embodiments, the nucleic acid encoding a 2-in-1 zinc finger nuclease variant comprises a nucleotide sequence selected from any one of SEQ ID NO: 45. In some embodiments, the nucleic acid encoding a 2-in-1 zinc finger nuclease variant comprises a nucleotide sequence selected from any one of SEQ ID NO: 46. In some embodiments, the nucleic acid encoding a 2-in-1 zinc finger nuclease variant comprises a nucleotide sequence selected from any one of SEQ ID NO: 47. In some embodiments, the nucleic acid encoding a 2-in-1 zinc finger nuclease variant comprises a nucleotide sequence selected from any one of SEQ ID NO: 48. In some embodiments, the nucleic acid encoding a 2-in-1 zinc finger nuclease variant comprises a nucleotide sequence selected from any one of SEQ ID NO: 49.

In some embodiments, the nucleic acid encoding a 2-in-1 zinc finger nuclease variant comprises a nucleotide encoding the amino acid sequence set forth in any one of SEQ ID NO: 132-135. In some embodiments, the nucleic acid encoding a 2-in-1 zinc finger nuclease variant comprises a nucleotide encoding the amino acid sequence set forth in SEQ ID NO: 132. In some embodiments, the nucleic acid encoding a 2-in-1 zinc finger nuclease variant comprises a nucleotide encoding the amino acid sequence set forth in SEQ ID NO: 133. In some embodiments, the nucleic acid encoding a 2-in-1 zinc finger nuclease variant comprises a nucleotide encoding the amino acid sequence set forth in SEQ ID NO: 134. In some embodiments, the nucleic acid encoding a 2-in-1 zinc finger nuclease variant comprises a nucleotide encoding the amino acid sequence set forth in SEQ ID NO: 135.

In some embodiments, the nucleic acid encoding a 2-in-1 zinc finger nuclease variant further comprises one or more 5′ITR, enhancer, promoter, 5′UTR, intron, post-transcriptional regulatory element, polyadenylation signal, or 3′ITR or any combination thereof. Each of the one or more 5′ITR, 3′ITR, enhancer, promoter, 5′UTR, 3′UTR, intron, post-transcriptional regulatory element, polyadenylation signal, and is independently operatively linked to the polynucleotide encoding the first and second ZFPs. Examples of such sequences are in Table 1.

In some embodiments, the nucleic acid encoding a 2-in-1 zinc finger nuclease variant further comprises one or more inverted terminal repeat (ITR) sequences. ITR are comprised of a nucleotide sequence that is followed by its reverse complement. Examples of inverted repeats include direct repeats, tandem repeats and palindromes. The ITR may be 5′ITR, a 3′ITR or both. The ITRs play a role in the integration of the viral construct into the host genome and rescue the viral construct from the host genome.

In some embodiments, the nucleic acid sequence encoding the 2-in-1 zinc finger nuclease variant further comprises a 5′ITR. In some embodiments, the 5′ITR comprises the nucleotide sequence set forth in SEQ ID NO: 10. In some embodiments, the nucleic acid sequence encoding the 2-in-1 zinc finger nuclease variant further comprises a 3′ITR. In some embodiments, the 3′ITR comprise the nucleotide sequence set forth in SEQ ID NO: 34. In some embodiments, the nucleic acid sequence encoding a 2-in-1 zinc finger nuclease variant further comprises an enhancer. In some embodiments, the enhancer is a eukaryotic enhancer. In some embodiments, the enhancer is a liver-specific enhancer. In some embodiments, the enhancer is a prokaryotic enhancer. In some embodiments the enhancer may be a viral enhancer. Exemplary enhancers include alpha 1 microglobulin/bikunin enhancer, SV40, CMV, HBV, and apolipoprotein E (ApoE). An exemplary liver-specific enhancer includes apolipoprotein E (APOE).

In some embodiments, the enhancer comprises a liver-specific enhancer. In some embodiments, the enhancer comprises an APOE enhancer. In some embodiments, the enhancer comprises the nucleotide sequence set forth in SEQ ID NO: 11.

In some embodiments, the nucleic acid sequence encoding the 2-in-1 zinc finger nuclease variant further comprises a promoter. In some embodiments, the promoter is a eukaryotic promoter. In some embodiments, the promoter is a prokaryotic promoter. In some embodiments, the promoter is a viral promoter. In some embodiments, the promoter is a liver-specific promoter. Exemplary promoters include CMV, CMVP, EF1a, CAG, PGK, TRE, U6, UAS, SV40, 5′LTR, polyhedron promoter (PH), TK, RSV, adenoviral E1A, human alpha 1-antitrypsin (hAAT), murine albumin (mAlb), phosphoenolpyruvate carboxykinase (rPECK), rat liver fatty acid binding protein, minimal transthyretin (TTR), thyroxine-binding globulin (TBG), EFla, PGK1, Ubc, human beta-actin, CAG, Ac5, CaMKIIa, GAL1, GAL10, TEF1, GDS, ADH1, CaMV35S, Ubi, H1, U6, HBV and the like. Exemplary viral promoters include CMV, SV40, 5′LTR, PH, TK, RSV, adenoviral ElA, CaMV35S, HBV and the like. Exemplary liver-specific promoters include human alpha 1-antitrypsin (hAAT), murine albumin (mAlb), phosphoenolpyruvate carboxykinase (rPECK), rat liver fatty acid binding protein, minimal transthyretin (TTR), thyroxine-binding globulin (TBG) and the like.

In some embodiments, the promoter comprises a hAAT promoter. In some embodiments, the promoter comprises the nucleotide sequence set forth in SEQ ID NO: 12.

In some embodiments, the nucleic acid sequence encoding the 2-in-1 zinc finger nuclease variant further comprises a UTR sequence. The UTR may be a 5′ UTR, a 3′UTR or both. In some embodiments, the nucleic acid sequence encoding the 2-in-1 zinc finger nuclease variant comprises a 5′UTR. In some embodiments, the nucleic acid sequence encoding the 2-in-1 zinc finger nuclease variant comprises a 3′UTR. In some embodiments, the nucleic acid sequence encoding the 2-in-1 zinc finger nuclease variant comprises a 5′UTR and a 3′UTR. In some embodiments, the 5′UTR comprises the nucleotide sequence set forth in SEQ ID NO: 13.

In some embodiments, the nucleic acid sequence encoding the 2-in-1 zinc finger nuclease variant further comprises a chimeric intron. Chimeric intron refers to an intronic regulatory element engineered into a polynucleotide construct. Chimeric introns have been reported to enhance mRNA processing (i.e. splicing), increase expression levels of downstream open reading frames, increase expression of weak promoters, and increase duration of expression in vivo. Exemplary chimeric intron includes Human β-globin/IgG chimeric intron. In some embodiments, the chimeric intron comprises a Human β-globin/IgG chimeric intron. In some embodiments, the chimeric intron comprises the nucleotide sequence set forth in SEQ ID NO: 14.

In some embodiments, the nucleic acid sequence encoding the 2-in-1 zinc finger nuclease variant further comprises a post-transcriptional regulatory element. Exemplary post-transcriptional regulatory elements include Woodchuck hepatitis virus post-transcriptional regulatory element (WPRE) and hepatitis B post-transcriptional regulatory element (HPRE). WPRE is a 600 bp long tripartite element containing gamma, alpha, and beta elements, in the given order, (Donello et al. (1992) J Virol 72:5085-5092) and contributes to the strong expression of transgenes in AAV systems (Loeb et al. (1999) Hum Gene Ther 10:2295-2305). It also enhances the expression of a transgene lacking introns. In its natural form, WPRE contains a partial open reading frame (ORF) for the WHV-X protein. The fully expressed WHV-X protein, in the context of other viral elements like the WHV (We2) enhancer, has been associated with a higher risk of hepatocarcinoma in woodchucks and mice (Hohne et. al (1990) EMBO J 9(4):1137-45; Flajolet et. al (1998) J Virol 72(7):6175-80). The WHV-X protein does not appear to be directly oncogenic, but some studies suggest that under certain circumstances it can act as a weak cofactor for the generation of liver cancers associated with infection by hepadnaviruses (hepatitis B virus for man; woodchuck hepatitis virus for woodchucks). “Wildtype” WPRE refers to a 591 bp sequence (nucleotides 1094-1684 in GenBank accession number J02442) containing a portion of the WHV X protein open-reading frame (ORF) in its 3′ region. A “mutated” WPRE sequence (i.e. WPREmut6) refers to a WPRE sequence that lacks the transcription of a fragment of the potentially oncogenic woodchuck hepatitis virus-X protein. In this element, there is an initial ATG start codon for WHV-X at position 1502 and a promoter region with the sequence GCTGA at position 1488. In Zanta-Boussif (ibid), a mut6WPRE sequence was disclosed wherein the promoter sequence at position 1488 was modified to ATCAT and the start codon at position 1502 was modified to TTG, effectively prohibiting expression of WHV-X. In the J04514.1 WPRE variant, the ATG WHV X start site is a position 1504, and a mut6 type variant can be made in the this J04514.1 strain. Another WPRE variant is the 247 bp WPRE3 variant comprising only minimal gamma and alpha elements from the wild type WPRE (Choi et al. (2014) Mol Brain 7:17), which lacks the WHV X sequences. A WPRE sequence (e.g., WRPEmut6 variant) from J02442.1 may also be used.

In some embodiments, the nucleic acid sequence encoding the 2-in-1 zinc finger nuclease variant comprises a 3′ WPRE sequence (see U.S. Patent Publication No. 2016/0326548). In some embodiments, the WPRE is a wild type WPRE. In some embodiments, the WPRE element is a mutated in the ‘X’ region to prevent expression of Protein X (see U.S. Pat. No. 7,419,829). In some embodiments, the mutated WPRE element comprises mutations described in Zanta-Boussif et al. (2009) Gene Ther 16(5):605-619, for example a WPREmut6 sequence. In some embodiments, the WPRE is a WPRE3 variant (Choi et al. (2014) Mol Brain 7:17). In some embodiments, the WPRE comprises a WPREmut6. In some embodiments, the WPRE comprises the nucleotide sequence set forth in SEQ ID NO: 32.

In some embodiments, the nucleic acid sequence encoding the 2-in-1 zinc finger nuclease variant further comprises a polyadenylation (poly A) signal. Exemplary polyadenylation signals include bovine Growth Hormone (bGH), human Growth Hormone (hGH), SV40, and rbGlob. In some embodiments, the poly A signal comprises a bGH poly A signal. In some embodiments the poly A signal comprises a hGH poly A signal. In some embodiments, the poly A signal comprises an SV40 poly A signal. In some embodiments, the poly A signal comprises a rbGlob poly A signal. In some embodiments, the poly A signal comprises the nucleotide sequence set forth in SEQ ID NO: 33.

In some embodiments, the 2-in-1 zinc finger nuclease variant nucleic acid sequence of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans.

Thus, in addition to the sequences encoding the components of the paired nuclease, the constructs may include additional coding or non-coding sequences in any order or combination. Constructs include constructs in which the left ZFN coding sequence is 5′ to the right ZFN coding sequence and constructs in which the right ZFN-encoding sequence is 5′ the left ZFN coding sequence. One or both of the left or right ZFN encoding sequences may be codon diversified in any way. The term “single diversified constructs” refers to constructs in which one ZFN (either left or right in any order in the construct) is encoded by a diversified sequence. The term “dual diversified constructs” refers to constructs in which both the left and right ZFNs (in any order in the construct) are codon diversified.

### Zinc Finger Nuclease Variants

In one aspect, disclosed herein is a 2-in-1 zinc finger nuclease variant encoded by any of the polynucleotide sequences disclosed herein. In some embodiments, disclosed herein is a 2-in-1 zinc finger nuclease variant comprising a first zinc finger nuclease and a second zinc finger nuclease separated by a 2A self-cleaving peptide positioned in between the first zinc finger nuclease and the second zinc finger nuclease. In some embodiments, the first zinc finger nuclease is codon diversified. In some embodiments, the first zinc finger nuclease is not codon diversified. In some embodiments the second zinc finger nuclease is codon diversified. In some embodiments the second zinc finger nuclease is not codon diversified. In some embodiments, the first zinc finger nuclease and the second zinc finger nuclease are each independently codon diversified. In some embodiments, neither the first zinc finger nuclease nor the second zinc finger nuclease is codon diversified.

In some embodiments, the 2-in-1 zinc finger nuclease variant further comprises a) one or nuclear localization sequences; b) one or more epitope tag; and c) one or more cleavage domains.

In some embodiments, the first zinc finger nuclease comprises the amino acid sequence of any one of SEQ ID NOs: 136-137. In some embodiments, the first zinc finger nuclease comprises the amino acid sequence of SEQ ID NOs: 136. In some embodiments, the first zinc finger nuclease comprises the amino acid sequence of SEQ ID NOs: 137.

In some embodiments, the first zinc finger nuclease is encoded by a polynucleotide comprising the nucleotide sequence set forth in any one of SEQ ID NOs: 116-129. In some embodiments, the first zinc finger nuclease is encoded by a polynucleotide comprising the nucleotide sequence set forth in SEQ ID NO: 116. In some embodiments, the first zinc finger nuclease is encoded by a polynucleotide comprising the nucleotide sequence set forth in SEQ ID NO: 117. In some embodiments, the first zinc finger nuclease is encoded by a polynucleotide comprising the nucleotide sequence set forth in SEQ ID NO: 118. In some embodiments, the first zinc finger nuclease is encoded by a polynucleotide comprising the nucleotide sequence set forth in SEQ ID NO: 119. In some embodiments, the first zinc finger nuclease is encoded by a polynucleotide comprising the nucleotide sequence set forth in SEQ ID NO: 120. In some embodiments, the first zinc finger nuclease is encoded by a polynucleotide comprising the nucleotide sequence set forth in SEQ ID NO: 121. In some embodiments, the first zinc finger nuclease is encoded by a polynucleotide comprising the nucleotide sequence set forth in SEQ ID NO: 122. In some embodiments, the first zinc finger nuclease is encoded by a polynucleotide comprising the nucleotide sequence set forth in SEQ ID NO: 123. In some embodiments, the first zinc finger nuclease is encoded by a polynucleotide comprising the nucleotide sequence set forth in SEQ ID NO: 124. In some embodiments, the first zinc finger nuclease is encoded by a polynucleotide comprising the nucleotide sequence set forth in SEQ ID NO: 125. In some embodiments, the first zinc finger nuclease is encoded by a polynucleotide comprising the nucleotide sequence set forth in SEQ ID NO: 126. In some embodiments, the first zinc finger nuclease is encoded by a polynucleotide comprising the nucleotide sequence set forth in SEQ ID NO: 127. In some embodiments, the first zinc finger nuclease is encoded by a polynucleotide comprising the nucleotide sequence set forth in SEQ ID NO: 128. In some embodiments, the first zinc finger nuclease is encoded by a polynucleotide comprising the nucleotide sequence set forth in SEQ ID NO: 129.

In some embodiments, the second zinc finger nuclease comprises the amino acid sequence of any one of SEQ ID NOs: 136-137. In some embodiments, the second zinc finger nuclease comprises the amino acid sequence of SEQ ID NOs: 136. In some embodiments, the second zinc finger nuclease comprises the amino acid sequence of SEQ ID NOs: 137.

In some embodiments, the second zinc finger nuclease is encoded by a polynucleotide comprising the nucleotide sequence set forth in any one of SEQ ID NOs: 116-129. In some embodiments, the second zinc finger nuclease is encoded by a polynucleotide comprising the nucleotide sequence set forth in SEQ ID NO: 116. In some embodiments, the second zinc finger nuclease is encoded by a polynucleotide comprising the nucleotide sequence set forth in SEQ ID NO: 117. In some embodiments, the second zinc finger nuclease is encoded by a polynucleotide comprising the nucleotide sequence set forth in SEQ ID NO: 118. In some embodiments, the second zinc finger nuclease is encoded by a polynucleotide comprising the nucleotide sequence set forth in SEQ ID NO: 119. In some embodiments, the second zinc finger nuclease is encoded by a polynucleotide comprising the nucleotide sequence set forth in SEQ ID NO: 120. In some embodiments, the second zinc finger nuclease is encoded by a polynucleotide comprising the nucleotide sequence set forth in SEQ ID NO: 121. In some embodiments, the second zinc finger nuclease is encoded by a polynucleotide comprising the nucleotide sequence set forth in SEQ ID NO: 122. In some embodiments, the second zinc finger nuclease is encoded by a polynucleotide comprising the nucleotide sequence set forth in SEQ ID NO: 123. In some embodiments, the second zinc finger nuclease is encoded by a polynucleotide comprising the nucleotide sequence set forth in SEQ ID NO: 124. In some embodiments, the second zinc finger nuclease is encoded by a polynucleotide comprising the nucleotide sequence set forth in SEQ ID NO: 125. In some embodiments, the second zinc finger nuclease is encoded by a polynucleotide comprising the nucleotide sequence set forth in SEQ ID NO: 126. In some embodiments, the second zinc finger nuclease is encoded by a polynucleotide comprising the nucleotide sequence set forth in SEQ ID NO: 127. In some embodiments, the second zinc finger nuclease is encoded by a polynucleotide comprising the nucleotide sequence set forth in SEQ ID NO: 128. In some embodiments, the second zinc finger nuclease is encoded by a polynucleotide comprising the nucleotide sequence set forth in SEQ ID NO: 129.

In some embodiments, the 2-in-1 zinc finger nuclease variant further comprises one or more cleavage domains. Any suitable cleavage domain can be associated with (e.g., operatively linked) to a zinc finger DNA-binding domain (e.g., ZFP). Each of the cleavage domains may have the same amino acid sequence. Alternatively, they each of the cleavage domains may have a different amino acid sequence. In some embodiments, the cleavage domain comprises a Fok I cleavage domain, which is active as a dimer. In some embodiments the nucleotide sequence encoding the one or more Fok I cleavage domain is codon diversified. In some embodiments the nucleotide sequence encoding the one or more Fok I cleavage domain is not codon diversified. In some embodiments a first Fok I cleavage domain is operatively linked to the first zinc finger DNA binding protein (ZFP). In some embodiments a second Fok I cleavage domain is operatively linked to the second zinc finger DNA binding protein (ZFP). In some embodiments the first Fok I cleavage domain is located 3′ to the first zinc finger DNA binding protein (ZFP). In some embodiments the second Fok I cleavage domain is located 3′ to the second zinc finger DNA binding protein (ZFP).

In some embodiments, the first zinc finger nuclease comprises the amino acid sequence of any one of SEQ ID NOs: 130-131. In some embodiments, the first zinc finger nuclease comprises the amino acid sequence of SEQ ID NOs: 130. In some embodiments, the first zinc finger nuclease comprises the amino acid sequence of SEQ ID NOs: 131.

In some embodiments, the second zinc finger nuclease comprises the amino acid sequence of any one of SEQ ID NOs: 130-131. In some embodiments, the second zinc finger nuclease comprises the amino acid sequence of SEQ ID NOs: 130. In some embodiments, the second zinc finger nuclease comprises a nucleotide sequence encoding the amino acid sequence of SEQ ID NOs: 131.

In some embodiments, the first zinc finger nuclease is encoded by a polynucleotide sequence comprising the nucleotide sequence set forth in any one of SEQ ID NOs: 71-84. In some embodiments, the first zinc finger nuclease is encoded by a polynucleotide comprising the nucleotide sequence as set forth in SEQ ID NO: 71. In some embodiments, the first zinc finger nuclease is encoded by a polynucleotide comprising the nucleotide sequence as set forth in SEQ ID NO: 72. In some embodiments, the first zinc finger nuclease is encoded by a polynucleotide comprising the nucleotide sequence as set forth in SEQ ID NO: 73. In some embodiments, the first zinc finger nuclease is encoded by a polynucleotide comprising the nucleotide sequence as set forth in SEQ ID NO: 74. In some embodiments, the first zinc finger nuclease is encoded by a polynucleotide comprising the nucleotide sequence as set forth in SEQ ID NO: 75. In some embodiments, the first zinc finger nuclease is encoded by a polynucleotide comprising the nucleotide sequence as set forth in SEQ ID NO: 76. In some embodiments, the first zinc finger nuclease is encoded by a polynucleotide comprising the nucleotide sequence as set forth in SEQ ID NO: 77. In some embodiments, the first zinc finger nuclease is encoded by a polynucleotide comprising the nucleotide sequence as set forth in SEQ ID NO: 78. In some embodiments, the first zinc finger nuclease is encoded by a polynucleotide comprising the nucleotide sequence as set forth in SEQ ID NO: 79. In some embodiments, the first zinc finger nuclease is encoded by a polynucleotide comprising the nucleotide sequence as set forth in SEQ ID NO: 80. In some embodiments, the first zinc finger nuclease is encoded by a polynucleotide comprising the nucleotide sequence as set forth in SEQ ID NO: 81. In some embodiments, the first zinc finger nuclease is encoded by a polynucleotide comprising the nucleotide sequence as set forth in SEQ ID NO: 82. In some embodiments, the first zinc finger nuclease is encoded by a polynucleotide comprising the nucleotide sequence as set forth in SEQ ID NO: 83. In some embodiments, the first zinc finger nuclease is encoded by a polynucleotide comprising the nucleotide sequence as set forth in SEQ ID NO: 84.

In some embodiments, the second zinc finger nuclease is encoded by a polynucleotide sequence comprising the nucleotide sequence set forth in any one of SEQ ID NOs: 71-84. In some embodiments, the second zinc finger nuclease is encoded by a polynucleotide comprising the nucleotide sequence as set forth in SEQ ID NO: 71. In some embodiments, the second zinc finger nuclease is encoded by a polynucleotide comprising the nucleotide sequence as set forth in SEQ ID NO: 72. In some embodiments, the second zinc finger nuclease is encoded by a polynucleotide comprising the nucleotide sequence as set forth in SEQ ID NO: 73. In some embodiments, the second zinc finger nuclease is encoded by a polynucleotide comprising the nucleotide sequence as set forth in SEQ ID NO: 74. In some embodiments, the second zinc finger nuclease is encoded by a polynucleotide comprising the nucleotide sequence as set forth in SEQ ID NO: 75. In some embodiments, the second zinc finger nuclease is encoded by a polynucleotide comprising the nucleotide sequence as set forth in SEQ ID NO: 76. In some embodiments, the second zinc finger nuclease is encoded by a polynucleotide comprising the nucleotide sequence as set forth in SEQ ID NO: 77. In some embodiments, the second zinc finger nuclease is encoded by a polynucleotide comprising the nucleotide sequence as set forth in SEQ ID NO: 78. In some embodiments, the second zinc finger nuclease is encoded by a polynucleotide comprising the nucleotide sequence as set forth in SEQ ID NO: 79. In some embodiments, the second zinc finger nuclease is encoded by a polynucleotide comprising the nucleotide sequence as set forth in SEQ ID NO: 80. In some embodiments, the second zinc finger nuclease is encoded by a polynucleotide comprising the nucleotide sequence as set forth in SEQ ID NO: 81. In some embodiments, the second zinc finger nuclease is encoded by a polynucleotide comprising the nucleotide sequence as set forth in SEQ ID NO: 82. In some embodiments, the second zinc finger nuclease is encoded by a polynucleotide comprising the nucleotide sequence as set forth in SEQ ID NO: 83. In some embodiments, the second zinc finger nuclease is encoded by a polynucleotide comprising the nucleotide sequence as set forth in SEQ ID NO: 84.

In some embodiments, the zinc finger nuclease further comprises one or more nuclear localization sequence (NLS). Each of the NLS may have the same amino acid sequence. Alternatively, each NLS may have a different amino acid sequence. In some embodiments, the zinc finger nuclease comprises a first nuclear localization sequence (NLS) and a second nuclear localization sequence (NLS), wherein the first nuclear localization sequence (NLS) is located N-terminal (i.e., upstream) to the first zinc finger DNA binding protein (ZFP) and the second nuclear localization sequence (NLS) is located N-terminal (i.e., upstream) to the second zinc finger DNA binding protein (ZFP). In some embodiments, the first NLS is operatively linked to the first ZFP and the second NLS is operatively linked to the second ZFP. In some embodiments, the first NLS is codon diversified. In some embodiments, the first NLS is not codon diversified. In some embodiments, the second NLS is codon diversified. In some embodiments, the second NLS is not codon diversified.

In some embodiments, the first NLS comprises the amino acid sequence set forth in any one of SEQ ID NO: 3-9 and 156. In some embodiments, the first NLS comprises a nucleotide sequence encoding the amino acid sequence set forth in SEQ ID NO: 3. In some embodiments, the first NLS comprises the amino acid sequence set forth in SEQ ID NO: 4. In some embodiments, the first NLS comprises the amino acid sequence set forth in SEQ ID NO:5. In some embodiments, the first NLS comprises the amino acid sequence set forth in SEQ ID NO: 6. In some embodiments, the first NLS comprises the amino acid sequence set forth in SEQ ID NO: 7. In some embodiments, the first NLS comprises the amino acid sequence set forth in SEQ ID NO: 8. In some embodiments, the first NLS comprises the amino acid sequence set forth in SEQ ID NO: 9. In some embodiments, the first NLS comprises the amino acid sequence set forth in SEQ ID NO: 156. In some embodiments, the second NLS comprises the amino acid sequence set forth in any one of SEQ ID NO: 3-9 and 156. In some embodiments, the second NLS comprises the amino acid sequence set forth in SEQ ID NO: 3. In some embodiments, the second NLS comprises the amino acid sequence set forth in SEQ ID NO: 4. In some embodiments, the second NLS comprises the amino acid sequence set forth in SEQ ID NO:5. In some embodiments, the second NLS comprises the amino acid sequence set forth in SEQ ID NO: 6. In some embodiments, the second NLS comprises the amino acid sequence set forth in SEQ ID NO: 7. In some embodiments, the second NLS comprises the amino acid sequence set forth in SEQ ID NO: 8. In some embodiments, the second NLS comprises the amino acid sequence set forth in SEQ ID NO: 9. In some embodiments, the second NLS comprises the amino acid sequence set forth in SEQ ID NO: 156.

In some embodiments, the first NLS is encoded by the nucleotide sequence set forth in any one of SEQ ID NO: 59-70. In some embodiments, the first NLS is encoded by a nucleotide sequence comprising the nucleotide sequence set forth in SEQ ID NO: 59. In some embodiments, the first NLS is encoded by a nucleotide sequence comprising the nucleotide sequence set forth in SEQ ID NO: 60. In some embodiments, the first NLS is encoded by a nucleotide sequence comprising the nucleotide sequence set forth in SEQ ID NO: 61. In some embodiments, the first NLS is encoded by a nucleotide sequence comprising the nucleotide sequence set forth in SEQ ID NO: 62. In some embodiments, the first NLS is encoded by a nucleotide sequence comprising the nucleotide sequence set forth in SEQ ID NO: 63. In some embodiments, the first NLS is encoded by a nucleotide sequence comprising the nucleotide sequence set forth in SEQ ID NO: 64 In some embodiments, the first NLS is encoded by a nucleotide sequence comprising the nucleotide sequence set forth in SEQ ID NO: 65. In some embodiments, the first NLS is encoded by a nucleotide sequence comprising the nucleotide sequence set forth in SEQ ID NO: 66. In some embodiments, the first NLS is encoded by a nucleotide sequence comprising the nucleotide sequence set forth in SEQ ID NO: 67. In some embodiments, the first NLS is encoded by a nucleotide sequence comprising the nucleotide sequence set forth in SEQ ID NO: 68. In some embodiments, the first NLS is encoded by a nucleotide sequence comprising the nucleotide sequence set forth in SEQ ID NO: 69. In some embodiments, the first NLS is encoded by a nucleotide sequence comprising the nucleotide sequence set forth in SEQ ID NO: 70.

In some embodiments, the second NLS is encoded by the nucleotide sequence set forth in any one of SEQ ID NO: 59-70. In some embodiments, the second NLS is encoded by a nucleotide sequence comprising the nucleotide sequence set forth in SEQ ID NO: 59. In some embodiments, the second NLS is encoded by a nucleotide sequence comprising the nucleotide sequence set forth in SEQ ID NO: 60. In some embodiments, the second NLS is encoded by a nucleotide sequence comprising the nucleotide sequence set forth in SEQ ID NO: 61. In some embodiments, the second NLS is encoded by a nucleotide sequence comprising the nucleotide sequence set forth in SEQ ID NO: 62. In some embodiments, the second NLS is encoded by a nucleotide sequence comprising the nucleotide sequence set forth in SEQ ID NO: 63. In some embodiments, the second NLS is encoded by a nucleotide sequence comprising the nucleotide sequence set forth in SEQ ID NO: 64 In some embodiments, the second NLS is encoded by a nucleotide sequence comprising the nucleotide sequence set forth in SEQ ID NO: 65. In some embodiments, the second NLS is encoded by a nucleotide sequence comprising the nucleotide sequence set forth in SEQ ID NO: 66. In some embodiments, the second NLS is encoded by a nucleotide sequence comprising the nucleotide sequence set forth in SEQ ID NO: 67. In some embodiments, the second NLS is encoded by a nucleotide sequence comprising the nucleotide sequence set forth in SEQ ID NO: 68. In some embodiments, the second NLS is encoded by a nucleotide sequence comprising the nucleotide sequence set forth in SEQ ID NO: 69. In some embodiments, the second NLS is encoded by a nucleotide sequence comprising the nucleotide sequence set forth in SEQ ID NO: 70.

In some embodiments, the 2-in-1 zinc finger nuclease variant further comprises one or more epitope tag. Epitope tags include, for example one or more copies of FLAG, HA, CBP, GST, HBH, MBP, Myc, His, polyHis, S-tag, SUMO, TAP, TAGP, TRX, V5, GFP, RFP, YFP, and the like.

In some embodiments, the 2-in-1 zinc finger nuclease variant further comprises one or one or more copies of a epitope tag. In some embodiments, the 2-in-1 zinc finger nuclease variant comprises a first epitope tag and a second epitope tag. In some embodiments, each of said first epitope tag and second epitope tag is the same. In some embodiments, each of said first epitope tag and second epitope tag are different. In some embodiments, the first epitope tag is located N-terminal to the first ZFP, and the second epitope tag is located N-terminal to the second ZFP. In some embodiments, the first epitope tag is located N-terminal to the first NLS, and the second epitope tag is located N terminal to the second NLS. In some embodiments, the first epitope tag is located C-terminal to the first ZFP, and the second epitope tag is located C-terminal to the second ZFP. In some embodiments, the first epitope tag is located C-terminal to the first NLS, and the second epitope tag is located C-terminal to the second NLS. In some embodiments, the first epitope tag is codon diversified. In some embodiments, the first epitope tag is not codon diversified. In some embodiments, the second epitope tag is codon diversified. In some embodiments, the second epitope tag is not codon diversified.

In some embodiments, the 2-in-1 zinc finger nuclease variant further comprises one or one or more copies of a FLAG tag. In some embodiments, the epitope tag is 3×FLAG. In some embodiments, the 2-in-1 zinc finger nuclease variant comprises a first FLAG tag and a second FLAG tag. In some embodiments, each of said first FLAG tag and second FLAG tag is 3×FLAG. In some embodiments, the first FLAG tag is located N-terminal to the first ZFP, and the second FLAG tag is located N-terminal to the second ZFP. In some embodiments, the first FLAG tag is located N-terminal to the first NLS, and the second FLAG tag is located N terminal to the second NLS. In some embodiments, the first FLAG tag is located C-terminal to the first ZFP, and the second FLAG tag is located C-terminal to the second ZFP. In some embodiments, the first FLAG tag is located C-terminal to the first NLS, and the second FLAG tag is located C-terminal to the second NLS. In some embodiments, the first FLAG tag is codon diversified. In some embodiments, the first FLAG tag is not codon diversified. In some embodiments, the second FLAG tag is codon diversified. In some embodiments, the second FLAG tag is not codon diversified.

In some embodiments, the first FLAG tag comprises the amino acid sequence set forth in any one of SEQ ID NO: 1-2. In some embodiments, the first FLAG tag comprises the amino acid sequence set forth in SEQ ID NO: 1. In some embodiments, the first FLAG tag comprises the amino acid sequence set forth in SEQ ID NO: 2. In some embodiments, the second FLAG tag comprises the amino acid sequence set forth in any one of SEQ ID NO: 1-2. In some embodiments, the second FLAG tag comprises the amino acid sequence set forth in SEQ ID NO: 1. In some embodiments, the second FLAG tag comprises the amino acid sequence set forth in SEQ ID NO: 2.

In some embodiments, the first FLAG tag is encoded by a polynucleotide comprising the nucleotide sequence set forth in any one of SEQ ID NO: 15-16, 50-58, 153 or 154. In some embodiments, the first FLAG tag is encoded by a polynucleotide comprising the nucleotide sequence set forth in any one of SEQ ID NO: 15. In some embodiments, the first FLAG tag is encoded by a polynucleotide comprising the nucleotide sequence set forth in any one of SEQ ID NO: 16. In some embodiments, the first FLAG tag is encoded by a polynucleotide comprising the nucleotide sequence set forth in any one of SEQ ID NO: 50. In some embodiments, the first FLAG tag is encoded by a polynucleotide comprising the nucleotide sequence set forth in any one of SEQ ID NO: 51. In some embodiments, the first FLAG tag is encoded by a polynucleotide comprising the nucleotide sequence set forth in any one of SEQ ID NO: 52. In some embodiments, the first FLAG tag is encoded by a polynucleotide comprising the nucleotide sequence set forth in any one of SEQ ID NO: 53. In some embodiments, the first FLAG tag is encoded by a polynucleotide comprising the nucleotide sequence set forth in any one of SEQ ID NO: 54. In some embodiments, the first FLAG tag is encoded by a polynucleotide comprising the nucleotide sequence set forth in any one of SEQ ID NO: 55. In some embodiments, the first FLAG tag is encoded by a polynucleotide comprising the nucleotide sequence set forth in any one of SEQ ID NO: 56. In some embodiments, the first FLAG tag is encoded by a polynucleotide comprising the nucleotide sequence set forth in any one of SEQ ID NO: 57. In some embodiments, the first FLAG tag is encoded by a polynucleotide comprising the nucleotide sequence set forth in any one of SEQ ID NO: 58. In some embodiments, the second FLAG tag is encoded by a polynucleotide comprising the nucleotide sequence set forth in any one of SEQ ID NO: 153. In some embodiments, the second FLAG tag is encoded by a polynucleotide comprising the nucleotide sequence set forth in any one of SEQ ID NO: 154.

In some embodiments, the second FLAG tag is encoded by a polynucleotide comprising the nucleotide sequence set forth in any one of SEQ ID NO: 15-16, 50-58, 153 or 154. In some embodiments, the second FLAG tag is encoded by a polynucleotide comprising the nucleotide sequence set forth in any one of SEQ ID NO: 15. In some embodiments, the second FLAG tag is encoded by a polynucleotide comprising the nucleotide sequence set forth in any one of SEQ ID NO: 16. In some embodiments, the second FLAG tag is encoded by a polynucleotide comprising the nucleotide sequence set forth in any one of SEQ ID NO: 50. In some embodiments, the second FLAG tag is encoded by a polynucleotide comprising the nucleotide sequence set forth in any one of SEQ ID NO: 51. In some embodiments, the second FLAG tag is encoded by a polynucleotide comprising the nucleotide sequence set forth in any one of SEQ ID NO: 52. In some embodiments, the second FLAG tag is encoded by a polynucleotide comprising the nucleotide sequence set forth in any one of SEQ ID NO: 53. In some embodiments, the second FLAG tag is encoded by a polynucleotide comprising the nucleotide sequence set forth in any one of SEQ ID NO: 54. In some embodiments, the second FLAG tag is encoded by a polynucleotide comprising the nucleotide sequence set forth in any one of SEQ ID NO: 55. In some embodiments, the second FLAG tag is encoded by a polynucleotide comprising the nucleotide sequence set forth in any one of SEQ ID NO: 56. In some embodiments, the second FLAG tag is encoded by a polynucleotide comprising the nucleotide sequence set forth in any one of SEQ ID NO: 57. In some embodiments, the second FLAG tag is encoded by a polynucleotide comprising the nucleotide sequence set forth in any one of SEQ ID NO: 58. In some embodiments, the second FLAG tag is encoded by a polynucleotide comprising the nucleotide sequence set forth in any one of SEQ ID NO: 153. In some embodiments, the second FLAG tag is encoded by a polynucleotide comprising the nucleotide sequence set forth in any one of SEQ ID NO: 154.

In some embodiments, the first zinc finger nuclease is encoded by a polynucleotide comprising the nucleotide sequence of any one of SEQ ID NOs: 17-23 and 25-31. In some embodiments, the first zinc finger nuclease is encoded by a polynucleotide comprising the nucleotide sequence of SEQ ID NO: 17. In some embodiments, the first zinc finger nuclease is encoded by a polynucleotide comprising the nucleotide sequence of SEQ ID NO: 18. In some embodiments, the first zinc finger nuclease is encoded by a polynucleotide comprising the nucleotide sequence of SEQ ID NO: 19. In some embodiments, the first zinc finger nuclease is encoded by a polynucleotide comprising the nucleotide sequence of SEQ ID NO: 20. In some embodiments, the first zinc finger nuclease is encoded by a polynucleotide comprising the nucleotide sequence of SEQ ID NO: 21. In some embodiments, the first zinc finger nuclease is encoded by a polynucleotide comprising the nucleotide sequence of SEQ ID NO: 22. In some embodiments, the first zinc finger nuclease is encoded by a polynucleotide comprising the nucleotide sequence of SEQ ID NO: 23. In some embodiments, the first zinc finger nuclease is encoded by a polynucleotide comprising the nucleotide sequence of SEQ ID NO: 25. In some embodiments, the first zinc finger nuclease is encoded by a polynucleotide comprising the nucleotide sequence of SEQ ID NO: 26. In some embodiments, the first zinc finger nuclease is encoded by a polynucleotide comprising the nucleotide sequence of SEQ ID NO: 27. In some embodiments, the first zinc finger nuclease is encoded by a polynucleotide comprising the nucleotide sequence of SEQ ID NO: 28. In some embodiments, the first zinc finger nuclease is encoded by a polynucleotide comprising the nucleotide sequence of SEQ ID NO: 29. In some embodiments, the first zinc finger nuclease is encoded by a polynucleotide comprising the nucleotide sequence of SEQ ID NO: 30. In some embodiments, the first zinc finger nuclease is encoded by a polynucleotide comprising the nucleotide sequence of SEQ ID NO: 31.

In some embodiments, the second zinc finger nuclease is encoded by a polynucleotide comprising the nucleotide sequence of any one of SEQ ID NOs: 17-23 and 25-31. In some embodiments, the second zinc finger nuclease is encoded by a polynucleotide comprising the nucleotide sequence of SEQ ID NO: 17. In some embodiments, the second zinc finger nuclease is encoded by a polynucleotide comprising the nucleotide sequence of SEQ ID NO: 18. In some embodiments, the second zinc finger nuclease is encoded by a polynucleotide comprising the nucleotide sequence of SEQ ID NO: 19. In some embodiments, the second zinc finger nuclease is encoded by a polynucleotide comprising the nucleotide sequence of SEQ ID NO: 20. In some embodiments, the second zinc finger nuclease is encoded by a polynucleotide comprising the nucleotide sequence of SEQ ID NO: 21. In some embodiments, the second zinc finger nuclease is encoded by a polynucleotide comprising the nucleotide sequence of SEQ ID NO: 22. In some embodiments, the second zinc finger nuclease is encoded by a polynucleotide comprising the nucleotide sequence of SEQ ID NO: 23. In some embodiments, the second zinc finger nuclease is encoded by a polynucleotide comprising the nucleotide sequence of SEQ ID NO: 25. In some embodiments, the second zinc finger nuclease is encoded by a polynucleotide comprising the nucleotide sequence of SEQ ID NO: 26. In some embodiments, the second zinc finger nuclease is encoded by a polynucleotide comprising the nucleotide sequence of SEQ ID NO: 27. In some embodiments, the second zinc finger nuclease is encoded by a polynucleotide comprising the nucleotide sequence of SEQ ID NO: 28. In some embodiments, the second zinc finger nuclease is encoded by a polynucleotide comprising the nucleotide sequence of SEQ ID NO: 29. In some embodiments, the second zinc finger nuclease is encoded by a polynucleotide comprising the nucleotide sequence of SEQ ID NO: 30. In some embodiments, the second zinc finger nuclease is encoded by a polynucleotide comprising the nucleotide sequence of SEQ ID NO: 31.

In some embodiments the 2A self-cleaving peptide is between 15 and 25 amino acids. In some embodiments the 2A self-cleaving peptide is between 18 and 22 amino acids. Non-limiting examples of 2A self-cleaving peptides include T2A, P2A, E2A and F2A sequences. See, e.g., Donnelly et al. (2001) J. Gen. Virol. 82:1013-1025. In some embodiments the 2A self-cleaving sequence comprises the amino acid sequence of SEQ ID NO: 138. In some embodiments, the 2A self-cleaving sequence is encoded by a polynucleotide comprising the nucleotide sequence of SEQ ID NO:24.

In some embodiments, the 2-in-1 zinc finger nuclease variant comprises the amino acid sequence set forth in any one of SEQ ID NO: 132-135. In some embodiments, the 2-in-1 zinc finger nuclease variant comprises the amino acid sequence set forth in SEQ ID NO: 132. In some embodiments, the 2-in-1 zinc finger nuclease variant comprises the amino acid sequence set forth in SEQ ID NO: 133. In some embodiments, the 2-in-1 zinc finger nuclease variant comprises the amino acid sequence set forth in SEQ ID NO: 134. In some embodiments, the 2-in-1 zinc finger nuclease variant comprises the amino acid sequence set forth in SEQ ID NO: 135.

In some embodiments, the 2-in-1 zinc finger nuclease variant is encoded by a nucleic acid comprising a nucleotide sequence selected from any one of SEQ ID NO: 85-115. In some embodiments, the 2-in-1 zinc finger nuclease variant is encoded by a nucleic acid comprising the nucleotide sequence set forth in SEQ ID NO: 85. In some embodiments, the 2-in-1 zinc finger nuclease variant is encoded by a nucleic acid comprising the nucleotide sequence set forth in SEQ ID NO: 86. In some embodiments, the 2-in-1 zinc finger nuclease variant is encoded by a nucleic acid comprising the nucleotide sequence set forth in SEQ ID NO: 87. In some embodiments, the 2-in-1 zinc finger nuclease variant is encoded by a nucleic acid comprising the nucleotide sequence set forth in SEQ ID NO: 88. In some embodiments, the 2-in-1 zinc finger nuclease variant is encoded by a nucleic acid comprising the nucleotide sequence set forth in SEQ ID NO: 89. In some embodiments, the 2-in-1 zinc finger nuclease variant is encoded by a nucleic acid comprising the nucleotide sequence set forth in SEQ ID NO: 90. In some embodiments, the 2-in-1 zinc finger nuclease variant is encoded by a nucleic acid comprising the nucleotide sequence set forth in SEQ ID NO: 91. In some embodiments, the 2-in-1 zinc finger nuclease variant is encoded by a nucleic acid comprising the nucleotide sequence set forth in SEQ ID NO: 92. In some embodiments, the 2-in-1 zinc finger nuclease variant is encoded by a nucleic acid comprising the nucleotide sequence set forth in SEQ ID NO: 93. In some embodiments, the 2-in-1 zinc finger nuclease variant is encoded by a nucleic acid comprising the nucleotide sequence set forth in SEQ ID NO: 94. In some embodiments, the 2-in-1 zinc finger nuclease variant is encoded by a nucleic acid comprising the nucleotide sequence set forth in SEQ ID NO: 95. In some embodiments, the 2-in-1 zinc finger nuclease variant is encoded by a nucleic acid comprising the nucleotide sequence set forth in SEQ ID NO: 96. In some embodiments, the 2-in-1 zinc finger nuclease variant is encoded by a nucleic acid comprising the nucleotide sequence set forth in SEQ ID NO: 97. In some embodiments, the 2-in-1 zinc finger nuclease variant is encoded by a nucleic acid comprising the nucleotide sequence set forth in SEQ ID NO: 98. In some embodiments, the 2-in-1 zinc finger nuclease variant is encoded by a nucleic acid comprising the nucleotide sequence set forth in SEQ ID NO: 99. In some embodiments, the 2-in-1 zinc finger nuclease variant is encoded by a nucleic acid comprising the nucleotide sequence set forth in SEQ ID NO: 100. In some embodiments, the 2-in-1 zinc finger nuclease variant is encoded by a nucleic acid comprising the nucleotide sequence set forth in SEQ ID NO: 101. In some embodiments, the 2-in-1 zinc finger nuclease variant is encoded by a nucleic acid comprising the nucleotide sequence set forth in SEQ ID NO: 102. In some embodiments, the 2-in-1 zinc finger nuclease variant is encoded by a nucleic acid comprising the nucleotide sequence set forth in SEQ ID NO: 103. In some embodiments, the 2-in-1 zinc finger nuclease variant is encoded by a nucleic acid comprising the nucleotide sequence set forth in SEQ ID NO: 104 In some embodiments, the 2-in-1 zinc finger nuclease variant is encoded by a nucleic acid comprising the nucleotide sequence set forth in SEQ ID NO: 105. In some embodiments, the 2-in-1 zinc finger nuclease variant is encoded by a nucleic acid comprising the nucleotide sequence set forth in SEQ ID NO: 106 In some embodiments, the 2-in-1 zinc finger nuclease variant is encoded by a nucleic acid comprising the nucleotide sequence set forth in SEQ ID NO: 107. In some embodiments, the 2-in-1 zinc finger nuclease variant is encoded by a nucleic acid comprising the nucleotide sequence set forth in SEQ ID NO: 108 In some embodiments, the 2-in-1 zinc finger nuclease variant is encoded by a nucleic acid comprising the nucleotide sequence set forth in SEQ ID NO: 109. In some embodiments, the 2-in-1 zinc finger nuclease variant is encoded by a nucleic acid comprising the nucleotide sequence set forth in SEQ ID NO: 110 In some embodiments, the 2-in-1 zinc finger nuclease variant is encoded by a nucleic acid comprising the nucleotide sequence set forth in SEQ ID NO: 111. In some embodiments, the 2-in-1 zinc finger nuclease variant is encoded by a nucleic acid comprising the nucleotide sequence set forth in SEQ ID NO: 112. In some embodiments, the 2-in-1 zinc finger nuclease variant is encoded by a nucleic acid comprising the nucleotide sequence set forth in SEQ ID NO: 113. In some embodiments, the 2-in-1 zinc finger nuclease variant is encoded by a nucleic acid comprising the nucleotide sequence set forth in SEQ ID NO: 114. In some embodiments, the 2-in-1 zinc finger nuclease variant is encoded by a nucleic acid comprising the nucleotide sequence set forth in SEQ ID NO: 115.

In some embodiments, the 2-in-1 zinc finger nuclease variant of the disclosure comprises at least about 60%, about 65%, about 70%, about 75%, about 80%, about 85%, about 90%, about 91%, about 92%, about 93%, about 94%, about 95%, about 96%, about 97%, about 98%, about 99% or more sequence identity to any of the sequences disclosed herein, as determined by sequence alignment programs known by skilled artisans.

In some embodiments, the 2-in-1 zinc finger nuclease variant comprising a first zinc finger nuclease and a second zinc finger nuclease separated by a 2A self-cleaving peptide positioned in between the first zinc finger nuclease and the second zinc finger nuclease is encoded by a polynucleotide comprising the nucleotide sequence as set forth in SEQ ID NOs: 100-115.

### Vectors and Delivery Systems

In one aspect, the present disclosure provides vectors comprising polynucleotide sequences encoding the 2-in-1 zinc finger nuclease variants as described herein. The 2-in-1 zinc finger nuclease variants described herein may be delivered in vivo or ex vivo by any suitable vector system, including, but not limited to, plasmid vectors, a mini-circle and a linear DNA form, non-viral vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Host cells containing said polynucleotide sequences or vectors are also provided. Any of the foregoing 2-in-1 zinc finger nuclease variants, polynucleotides encoding the 2-in-1 zinc finger nuclease variants, vectors or cells may be used in the methods disclosed herein.

Viral vector systems may also be used. Viral based systems for the delivery of ZFPs and ZFNs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been measured in many different cell types and target tissues.

In some embodiments, adeno-associated virus (“AAV”) vectors are also used to transduce cells with zinc finger nuclease constructs as described herein. AAV serotypes that may be employed, including by non-limiting example, AAV1, AAV2, AAV3, AAV4, AAV5, AAV6, AAV8, AAV 8.2, AAV9 and AAV rh10 and pseudotyped AAV such as AAV2/8, AAV2/5 and AAV2/6 can also be used in accordance with the present invention. In some embodiments, the AAV is AAV1. In some embodiments, the AAV is AAV2. In some embodiments, the AAV is AAV3. In some embodiments, the AAV is AAV4. In some embodiments, the AAV is AAV5. In some embodiments, the AAV is AAV6. In some embodiments, the AAV is AAV8. In some embodiments, the AAV is AAV8.2. In some embodiments, the AAV is AAV9. In some embodiments, the AAV is AAVrh10. In some embodiments, the AAV is AAV2/5. In some embodiments, the AAV is AAV2/6.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Packaging cells are used to form virus particles (e.g., AAV particles) that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Non-viral vector delivery systems include DNA plasmids, naked nucleic acid, mRNA, and nucleic acid complexed with a delivery vehicle such as a liposome or poloxamer. Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024.

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al. (2009) Nature Biotechnology 27(7):643).

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Vectors (e.g., retroviruses, adenoviruses, liposomes, etc.) containing the nuclease constructs disclosed herein can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

It will be apparent that the nuclease-encoding sequences and donor constructs can be delivered using the same or different systems. For example, a donor polynucleotide can be carried by a plasmid, while the one or more nucleases can be carried by an AAV vector. In certain embodiments, the nuclease and donors are both delivered using AAV vectors (e.g., both using AAV2, both using AAV6, both using AAV2/6, nuclease using AAV2, AAV6 or AAV2/6 and donor using AAV 2, AAV6 or AAV2/6). Furthermore, the different vectors can be administered by the same or different routes (intramuscular injection, intravenous injection, intraperitoneal administration and/or intramuscular injection. The vectors can be delivered simultaneously or in any sequential order.

### Pharmaceutical Composition

In one aspect, the disclosure relates to a pharmaceutical composition (also referred to as a “formulation” or an “article of manufacture” or a “drug product” or a “set of drug products”) comprising any of the nucleic acids, proteins or vectors described herein. In some embodiments, the pharmaceutical composition comprises a nucleic acid encoding the 2-in-1 zinc finger nuclease variant as disclosed herein. In some embodiments, the pharmaceutical composition comprises a polynucleotide encoding a zinc-finger nucleotide binding domain as disclosed herein. In some embodiments, the pharmaceutical composition comprises a zinc finger nuclease as disclosed herein. In some embodiments, the pharmaceutical composition comprises a zinc finger nucleotide binding domain as disclosed herein. In some embodiments, the pharmaceutical composition comprises a vector as described herein.

Pharmaceutical compositions for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

Pharmaceutically acceptable carriers are determined in part by the particular composition being administered, as well as by the particular method used to administer the composition. Accordingly, there is a wide variety of suitable formulations of pharmaceutical compositions available (see, e.g., Remington's Pharmaceutical Sciences, 17th ed., 1989).

The pharmaceutical composition comprises a combination of the same or different composition in any concentrations. For example, provided herein is an article of manufacture comprising a set of drug products, which include two separate pharmaceutical compositions as follows: a first pharmaceutical composition comprising a purified AAV vector carrying both a first ZFN and a second ZFN pair and a second pharmaceutical composition comprising a purified AAV vector carrying a donor sequence comprising a transgene encoding a therapeutic protein for the treatment of LSD. One or both of pharmaceutical compositions may be individually formulated in phosphate buffered saline (PBS) containing CaCl₂, MgCl₂, NaCl, sucrose and a Poloxamer (e.g., Poloxamer P188) or in a Normal Saline (NS) formulation. In some embodiments, the composition comprises phosphate buffered saline (PBS) comprising approximately 1.15 mg/ML of sodium phosphate, 0.2 mg/mL potassium phosphate, 8.0 mg/mL sodium chloride, 0.2 mg/mL potassium chloride, 0.13 mg/mL calcium chloride, and 0.1 mg/mL Magnesium chloride. The PBS is further modified with 2.05 mg/mL sodium chloride, 10 mg/mL to 12 mg/mL of sucrose and 0.5 to 1.0 mg/mL of Kolliphor® (poloxamer or P188). Further, the article of manufacture may include any ratio of the two pharmaceutical compositions can be used.

In another aspect, provided herein is the use of any of the nucleic acids encoding the 2-in-1 zinc finger nuclease variants disclosed herein, for the preparation of a medicament for treating or preventing a lysosomal storage disorder.

In another aspect, provided herein is the use of any of the 2-in-1 zinc finger nuclease variants disclosed herein, for the preparation of a medicament for treating or preventing a lysosomal storage disorder.

In another aspect, provided herein is the use of any of the vectors disclosed herein, for the preparation of a medicament for treating or preventing a lysosomal storage disorder.

In another aspect, provided herein is the use of any of the cells disclosed herein, for the preparation of a medicament for treating or preventing a lysosomal storage disorder.

In another aspect, provided herein is the use of any of the nucleic acids encoding the 2-in-1 zinc finger nuclease variants disclosed herein, for the preparation of a medicament for correcting a lysosomal storage disease-causing mutation in the genome of a cell.

In another aspect, provided herein is the use of any of the 2-in-1 zinc finger nuclease variants disclosed herein, for the preparation of a medicament for correcting a lysosomal storage disease-causing mutation in the genome of a cell.

In another aspect, provided herein is the use of any of the vectors disclosed herein, for the preparation of a medicament for correcting a lysosomal storage disease-causing mutation in the genome of a cell.

In another aspect, provided herein is the use of any of the cells disclosed herein, for the preparation of a medicament for correcting a lysosomal storage disease-causing mutation in the genome of a cell.

In another aspect, provided herein is the use of any of the nucleic acids encoding the 2-in-1 zinc finger nuclease variants disclosed herein, for the preparation of a medicament for integrating an exogenous nucleotide sequence into a target nucleotide sequence in a gene of a cell.

In another aspect, provided herein is the use of any of the 2-in-1 zinc finger nuclease variants disclosed herein, for the preparation of a medicament for integrating an exogenous nucleotide sequence into a target nucleotide sequence in a gene of a cell.

In another aspect, provided herein is the use of any of the vectors disclosed herein, for the preparation of a medicament for integrating an exogenous nucleotide sequence into a target nucleotide sequence in a gene of a cell.

In another aspect, provided herein is the use of any of the cells disclosed herein, for the preparation of a medicament for integrating an exogenous nucleotide sequence into a target nucleotide sequence in a gene of a cell.

In another aspect, provided herein is the use of any of the nucleic acids encoding the 2-in-1 zinc finger nuclease variants disclosed herein, for the preparation of a medicament for disrupting a target nucleotide sequence in a gene of a cell, wherein said gene comprises a mutation associated with a lysosomal storage disease.

In another aspect, provided herein is the use of any of the 2-in-1 zinc finger nuclease variants disclosed herein, for the preparation of a medicament for disrupting a target nucleotide sequence in a gene of a cell, wherein said gene comprises a mutation associated with a lysosomal storage disease.

In another aspect, provided herein is the use of any of the vectors disclosed herein, for the preparation of a medicament for disrupting a target nucleotide sequence in a gene of a cell, wherein said gene comprises a mutation associated with a lysosomal storage disease.

In another aspect, provided herein is the use of any of the cells disclosed herein, for the preparation of a medicament for disrupting a target nucleotide sequence in a gene of a cell, wherein said gene comprises a mutation associated with a lysosomal storage disease.

### Methods for Modifying the Genome of a Cell

In one aspect, the present disclosure provides methods for modifying the genome of a cell, the method comprising introducing into the cell the 2-in-1 zinc finger nuclease variant of the disclosure, zinc-finger nuclease protein of the disclosure or a nucleic acid encoding 2-in-1 zinc finger nuclease variant of the disclosure.

In another aspect, the present disclosure provides a method for integrating an exogenous nucleotide sequence into a target nucleotide sequence in a gene of a cell, the method comprising introducing into a cell the 2-in-1 zinc finger nuclease variant of the disclosure.

In another aspect, the present disclosure provides a method for integrating an exogenous nucleotide sequence into a target nucleotide sequence in a gene of a cell, the method comprising introducing into a cell a nucleic acid encoding 2-in-1 zinc finger nuclease variant of the disclosure.

The methods and compositions disclosed herein can be used in any type of cell including a eukaryotic or prokaryotic cell and/or cell line. Examples of cells include, but are not limited to, prokaryotic cells, fungal cells, Archaeal cells, plant cells, insect cells, animal cells, vertebrate cells, mammalian cells and human cells. In some embodiments, the cell is a eukaryotic cell. In some embodiments, the cell is a mammalian cell. In some embodiments, the mammalian cell is a stem cell. In some embodiments, the eukaryotic cell is a human cell. In some embodiments, the eukaryotic cell is a plant cell. Non-limiting examples of eukaryotic cells or cell lines generated from such cells include T-cells, COS, K562, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6, HepG2, and 348A cells, as well as, insect cells such as Spodoptera fugiperda (Sf), or fungal cells such as Saccharomyces, Pichia and Schizosaccharomyces. Examples of stem cells include, but are not limited to, embryonic stem cells, induced pluripotent stem cells (iPS cells), hematopoietic stem cells, neuronal stem cells and mesenchymal stem cells.

In some embodiments, in order to introduce the zinc finger nuclease protein into the cell, the nucleic acid encoding the zinc-finger nuclease variant is incorporated into a plasmid, a viral vector, a mini-circle, a linear DNA form or other delivery system. Such delivery systems are well known to those of skill in the art.

In some embodiments, the target nucleotide sequence is an endogenous locus. In some embodiments, the endogenous locus is selected from the group consisting of Iduronidase Alpha-L (IDUA) gene (associated with mucopolysaccharidosis type I (MPS I)), Iduronate 2-Sulfatase (IDS) gene (associated with mucopolysaccharidosis type II (MPS II)), alpha-Galactosidase (GLA) gene (associated with Fabry disease), alpha-Glucosidase (GAA) gene (associated with Pompe disease), Phenylalanine Hydroxylase (PAH) gene (associated with phenylketonuria (PKU)), and a safe-harbor locus.

In some embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In some embodiments, the donor sequence can contain sequences that are homologous, but not identical, to genomic sequences in the region of interest, thereby stimulating homologous recombination to insert a non-identical sequence in the region of interest. In some embodiments, portions of the donor sequence that are homologous to sequences in the region of interest exhibit between about 80 to 99% (or any integer therebetween) sequence identity to the genomic sequence that is replaced. In some embodiments, the homology between the donor and genomic sequence is higher than 99%, for example if only 1 nucleotide differs as between donor and genomic sequences of over 100 contiguous base pairs. In some embodiments, a non-homologous portion of the donor sequence contains sequences that are not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In some embodiments, the donor sequence is non-homologous to the first target sequence, and is inserted into the genome by non-homologous recombination mechanisms.

In some embodiments, the disclosure provides for the integration of an exogenous nucleic acid sequence into a safe harbor locus in the genome of a cell. A safe harbor locus is typically a genomic locus where transgenes can integrate and function in a predictable manner without perturbing endogenous gene activity. Exemplary safe harbor loci in the human genome include, without limitation the Rosa26 locus, the AAVS 1 locus, and the safe harbor loci listed in Sadelain et al. Nat Rev Cancer. 2012; 12(1):51-8. In some embodiments, the safe harbor locus is located in chromosome 1.

The zinc finger nuclease protein or the nucleic acid encoding the zinc finger nuclease protein may be delivered to isolated cells (which in turn may be administered to a living subject for ex vivo cell therapy) or to a living subject. Delivery of gene editing molecules to cells and subjects are known in the art. Methods of delivering zinc finger nuclease proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Suitable cells include, but are not limited to, eukaryotic and prokaryotic cells and/or cell lines. Non-limiting examples of eukaryotic cells or cell lines generated from such cells include T-cells, COS, K562, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6, HepG2 and 348A cells, as well as, insect cells such as Spodoptera fugiperda (Sf), or fungal cells such as Saccharomyces, Pichia and Schizosaccharomyces. In some embodiments, the cell is a mammalian cell. In some embodiments, the cell is a stem cell, such as, by way of example, embryonic stem cells, induced pluripotent stem cells (iPS cells), hematopoietic stem cells, neuronal stem cells and mesenchymal stem cells.

The nucleic acid encoding the 2-in-1 zinc finger nuclease variant protein, as described herein, may also be delivered using vectors containing sequences encoding one or more of the components of the zinc finger nuclease protein. In some embodiments, additional nucleic acids (e.g., donor sequences) also may be delivered via these vectors. Furthermore, it will be apparent that any of these vectors may comprise one or more DNA-binding protein-encoding sequences and/or additional nucleic acids as appropriate. Thus, when one or more zinc finger nuclease protein as described herein are introduced into the cell, and additional DNAs as appropriate, they may be carried on the same vector or on different vectors. When multiple vectors are used, each vector may comprise a sequence encoding one or multiple zinc finger nuclease proteins and additional nucleic acids as desired. Conventional viral and non-viral based gene transfer methods can be used to introduce nucleic acids encoding engineered DNA-binding proteins in cells (e.g., in mammalian cells) and target tissues and to co-introduce additional nucleotide sequences as desired. Such methods can also be used to administer nucleic acids to cells in vitro. In certain embodiments, nucleic acids are administered for in vivo or ex vivo gene therapy uses.

Gene therapy vectors comprising the nucleic acid encoding the 2-in-1 zinc finger nuclease variants of the disclosure can be delivered in vivo by administration to an individual patient (subject), typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Ex vivo cell transfection for diagnostics, research, transplant or for gene therapy (e.g., via re-infusion of the transfected cells into the host organism) is well known to those of skill in the art. In some embodiments, cells are isolated from the subject organism, transfected with a nucleic acid encoding the 2-in-1 zinc finger nuclease variant, and re-infused back into the subject organism (e.g., patient). Various cell types suitable for ex vivo transfection are well known to those of skill in the art (see, e.g., Freshney, et al., Culture of Animal Cells, A Manual of Basic Technique (3rd ed. 1994)) and the references cited therein for a discussion of how to isolate and culture cells from patients).

In some embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba, et al. (1992) J. Exp. Med. 176:1693-1702).

### Exemplary Constructs

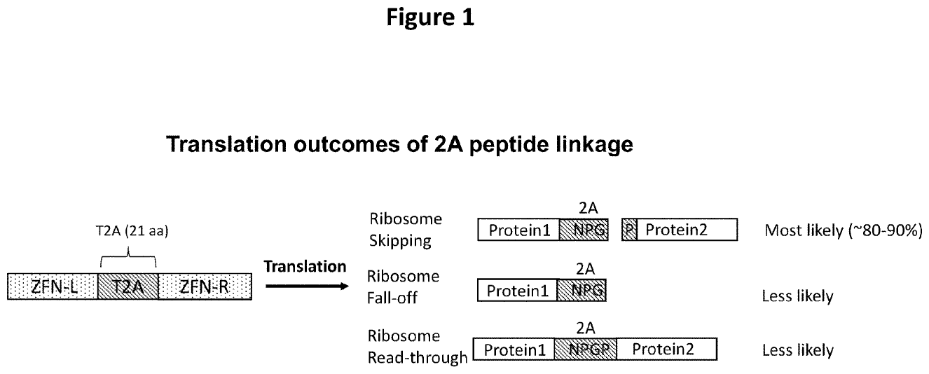

Non-limiting examples of 2-in-1 ZFN constructs include constructs as shown in FIG. 1; constructs comprising one or more of the sequences of Table 2 in any order or combination; and constructs as shown in Table 3.

TABLE 2

| SEQ | | |
|---|---|---|
| ID | Feature/ | |
| NO | Description | Amino Acid (aa) or Nucleic Acid (na) Sequence |
| 1 | FLAG tag (aa) | DYKDDDK |
| 2 | 3xFLAG (aa) | DYKDHDG-DYKDHDI-DYKDDDDK |
| 3 | NLS from the | PKKKRKV |
| | SV40 virus large T | |
| | gene protein (aa) | |
| 4 | NLS from c-myc | PAAKRVKLD |
| | protein (aa) | |
| 5 | NLS from hepatitis | EGAPPAKRAR |
| | delta virus (aa) | |
| 6 | NLS from polyoma | VSRKRPRP |
| | T protein (aa) | |
| 7 | NLS derived from | KRPAATKKAGQAKKKKLD |
| | the nucleoplasmin | |
| | carboxy tail (aa) | |
| 8 | NLS described by | NQSSNFGPMKGGNFGGRSSGPYGGGGQYFAKPRNQGGY |
| | Siomi and | |
| | Dreyfuss (aa) | |
| 9 | NLS from the Rex | PKTRRRPRRSQRKRPPT |
| | protein in HTLV-1 | |
| | (aa) | |
| 156 | NLS (aa) | PKKKRKVGIH |
| 130 | Left ZFN | AAMAERPFQCRICMQNFSQSGNLARHIRTHTGEKPFACDICGRKFAL |
| | (ZFN-L) (aa) | KQNLCMHTKIHTGEKPFQCRICMQKFAWQSNLQNHTKIHTGEKPFQC |
| | | RICMRNFSTSGNLTRHIRTHTGEKPFACDICGRKFARRSHLTSHTKI |
| | | HLRGSQLVKSELEEKKSELRHKLKYVPHEYIELIEIARNSTQDRILE |
| | | MKVMEFFMKVYGYRGKHLGGSRKPDGAIYTVGSPIDYGVIVDTKAYS |
| | | GGYNLPIGQADEMERYVEENQTRDKHLNPNEWWKVYPSSVTEFKFLF |
| | | VSGHFKGNYKAQLTRLNHITNCDGAVLSVEELLIGGEMIKAGTLTLE |
| | | EVRRKFNNGEINFRS |
| 131 | Right ZFN | AAMAERPFQCRICMRNFSQSSDLSRHIRTHTGEKPFACDICGRKFAL |
| | (ZFN-R) (aa) | KHNLLTHTKIHTGEKPFQCRICMQNFSDQSNLRAHIRTHTGEKPFAC |
| | | DICGRKFARNFSLTMHTKIHTGERGFQCRICMRNFSLRHDLERHIRT |
| | | HTGEKPFACDICGRKFAHRSNLNKHTKIHLRGSQLVKSELEEKKSEL |
| | | RHKLKYVPHEYIELIEIARNSTQDRILEMKVMEFFMKVYGYRGKHLG |
| | | GSRKPDGAIYTVGSPIDYGVIVDTKAYSGGYNLSIGQADEMQRYVKE |
| | | NQTRNKHINPNEWWKVYPSSVTEFKFLFVSGHFKGNYKAQLTRLNRK |
| | | TNCNGAVLSVEELLIGGEMIKAGTLTLEEVRRKFNNGEINF |
| 132 | Right ZFN-T2A- | DYKDHDGDYKDHDIDYKDDDDKMAPKKKRKVGIHGVPAAMAERPFQC |
| | Left ZFN | RICMRNFSQSSDLSRHIRTHTGEKPFACDICGRKFALKHNLLTHTKI |
| | with N-terminal | HTGEKPFQCRICMQNFSDQSNLRAHIRTHTGEKPFACDICGRKFARN |
| | modifications | FSLTMHTKIHTGERGFQCRICMRNFSLRHDLERHIRTHTGEKPFACD |
| | (comprising | ICGRKFAHRSNLNKHTKIHLRGSQLVKSELEEKKSELRHKLKYVPHE |
| | 3xFLAG, NLS, | YIELIEIARNSTQDRILEMKVMEFFMKVYGYRGKHLGGSRKPDGAIY |
| | ZFP-R, FokI, T2A, | TVGSPIDYGVIVDTKAYSGGYNLSIGQADEMQRYVKENQTRNKHINP |
| | 3xFLAG, NLS, | NEWWKVYPSSVTEFKFLFVSGHFKGNYKAQLTRLNRKTNCNGAVLSV |
| | ZFP-L, and FokI) | EELLIGGEMIKAGTLTLEEVRRKFNNGEINFGSGEGRGSLLTCGDVE |
| | (aa) | ENPGPTRAMDYKDHDGDYKDHDIDYKDDDDKMAPKKKRKVGIHGVPA |
| | | AMAERPFQCRICMQNFSQSGNLARHIRTHTGEKPFACDICGRKFALK |
| | | QNLCMHTKIHTGEKPFQCRICMQKFAWQSNLQNHTKIHTGEKPFQCR |
| | | ICMRNFSTSGNLTRHIRTHTGEKPFACDICGRKFARRSHLTSHTKIH |
| | | LRGSQLVKSELEEKKSELRHKLKYVPHEYIELIEIARNSTQDRILEM |
| | | KVMEFFMKVYGYRGKHLGGSRKPDGAIYTVGSPIDYGVIVDTKAYSG |
| | | GYNLPIGQADEMERYVEENQTRDKHLNPNEWWKVYPSSVTEFKFLFV |
| | | SGHFKGNYKAQLTRLNHITNCDGAVLSVEELLIGGEMIKAGTLTLEE |
| | | VRRKFNNGEINFRS- |
| 133 | Left ZFN-T2A- | DYKDHDGDYKDHDIDYKDDDDKMAPKKKRKVGIHGVPAAMAERPFQC |
| | Right ZFN | RICMQNFSQSGNLARHIRTHTGEKPFACDICGRKFALKQNLCMHTKI |
| | with N-terminal | HTGEKPFQCRICMQKFAWQSNLQNHTKIHTGEKPFQCRICMRNFSTS |
| | modifications | GNLTRHIRTHTGEKPFACDICGRKFARRSHLTSHTKIHLRGSQLVKS |
| | (comprising | ELEEKKSELRHKLKYVPHEYIELIEIARNSTQDRILEMKVMEFFMKV |
| | 3xFLAG, NLS, | YGYRGKHLGGSRKPDGAIYTVGSPIDYGVIVDTKAYSGGYNLPIGQA |
| | ZFP-L, FokI, T2A, | DEMERYVEENQTRDKHLNPNEWWKVYPSSVTEFKFLFVSGHFKGNYK |
| | 3xFLAG, NLS, | AQLTRLNHITNCDGAVLSVEELLIGGEMIKAGTLTLEEVRRKFNNGE |
| | ZFP-R, and FokI) | INFRSGSGEGRGSLLTCGDVEENPGPTRAMDYKDHDGDYKDHDIDYK |
| | (aa) | DDDDKMAPKKKRKVGIHGVPAAMAERPFQCRICMRNFSQSSDLSRHI |
| | | RTHTGEKPFACDICGRKFALKHNLLTHTKIHTGEKPFQCRICMQNFS |
| | | DQSNLRAHIRTHTGEKPFACDICGRKFARNFSLTMHTKIHTGERGFQ |
| | | CRICMRNFSLRHDLERHIRTHTGEKPFACDICGRKFAHRSNLNKHTK |
| | | IHLRGSQLVKSELEEKKSELRHKLKYVPHEYIELIEIARNSTQDRIL |
| | | EMKVMEFFMKVYGYRGKHLGGSRKPDGAIYTVGSPIDYGVIVDTKAY |
| | | SGGYNLSIGQADEMQRYVKENQTRNKHINPNEWWKVYPSSVTEFKFL |
| | | FVSGHFKGNYKAQLTRLNRKTNCNGAVLSVEELLIGGEMIKAGTLTL |
| | | EEVRRKFNNGEINF |
| 134 | Right ZFN-T2A- | AAMAERPFQCRICMRNFSQSSDLSRHIRTHTGEKPFACDICGRKFAL |
| | LeftZFN (aa) | KHNLLTHTKIHTGEKPFQCRICMQNFSDQSNLRAHIRTHTGEKPFAC |
| | | DICGRKFARNFSLTMHTKIHTGERGFQCRICMRNFSLRHDLERHIRT |
| | | HTGEKPFACDICGRKFAHRSNLNKHTKIHLRGSQLVKSELEEKKSEL |
| | | RHKLKYVPHEYIELIEIARNSTQDRILEMKVMEFFMKVYGYRGKHLG |
| | | GSRKPDGAIYTVGSPIDYGVIVDTKAYSGGYNLSIGQADEMQRYVKE |
| | | NQTRNKHINPNEWWKVYPSSVTEFKFLFVSGHFKGNYKAQLTRLNRK |
| | | TNCNGAVLSVEELLIGGEMIKAGTLTLEEVRRKFNNGEINFGSGEGR |
| | | GSLLTCGDVEENPGPAAMAERPFQCRICMQNFSQSGNLARHIRTHTG |
| | | EKPFACDICGRKFALKQNLCMHTKIHTGEKPFQCRICMQKFAWQSNL |
| | | QNHTKIHTGEKPFQCRICMRNFSTSGNLTRHIRTHTGEKPFACDICG |
| | | RKFARRSHLTSHTKIHLRGSQLVKSELEEKKSELRHKLKYVPHEYIE |
| | | LIEIARNSTQDRILEMKVMEFFMKVYGYRGKHLGGSRKPDGAIYTVG |
| | | SPIDYGVIVDTKAYSGGYNLPIGQADEMERYVEENQTRDKHLNPNEW |
| | | WKVYPSSVTEFKFLFVSGHFKGNYKAQLTRLNHITNCDGAVLSVEEL |
| | | LIGGEMIKAGTLTLEEVRRKFNNGEINFRS |
| 135 | Left ZFN-T2A- | AAMAERPFQCRICMQNFSQSGNLARHIRTHTGEKPFACDICGRKFAL |
| | Right ZFN (aa) | KQNLCMHTKIHTGEKPFQCRICMQKFAWQSNLQNHTKIHTGEKPFQC |
| | | RICMRNFSTSGNLTRHIRTHTGEKPFACDICGRKFARRSHLTSHTKI |
| | | HLRGSQLVKSELEEKKSELRHKLKYVPHEYIELIEIARNSTQDRILE |
| | | MKVMEFFMKVYGYRGKHLGGSRKPDGAIYTVGSPIDYGVIVDTKAYS |
| | | GGYNLPIGQADEMERYVEENQTRDKHLNPNEWWKVYPSSVTEFKFLF |
| | | VSGHFKGNYKAQLTRLNHITNCDGAVLSVEELLIGGEMIKAGTLTLE |
| | | EVRRKFNNGEINFRSGSGEGRGSLLTCGDVEENPGPVPAAMAERPFQ |
| | | CRICMRNFSQSSDLSRHIRTHTGEKPFACDICGRKFALKHNLLTHTK |
| | | IHTGEKPFQCRICMQNFSDQSNLRAHIRTHTGEKPFACDICGRKFAR |
| | | NFSLTMHTKIHTGERGFQCRICMRNFSLRHDLERHIRTHTGEKPFAC |
| | | DICGRKFAHRSNLNKHTKIHLRGSQLVKSELEEKKSELRHKLKYVPH |
| | | EYIELIETARNSTQDRILEMKVMEFFMKVYGYRGKHLGGSRKPDGAI |
| | | YTVGSPIDYGVIVDTKAYSGGYNLSIGQADEMQRYVKENQTRNKHIN |
| | | PNEWWKVYPSSVTEFKFLFVSGHFKGNYKAQLTRLNRKTNCNGAVLS |
| | | VEELLIGGEMIKAGTLTLEEVRRKFNNGEINF |
| 10 | 5′ ITR (na) | CTGCGCGCTCGCTCGCTCACTGAGGCCGCCCGGGCAAAGCCCGGGCG |
| | | TCGGGCGACCTTTGGTCGCCCGGCCTCAGTGAGCGAGCGAGCGCGCA |
| | | GAGAGGGAGTGGCCAACTCCATCACTAGGGGTTCCT |
| 11 | ApoE hepatic | AGGCTCAGAGGCACACAGGAGTTTCTGGGCTCACCCTGCCCCCTTCC |
| | control region | AACCCCTCAGTTCCCATCCTCCAGCAGCTGTTTGTGTGCTGCCTCTG |
| | (Enhancer) (na) | AAGTCCACACTGAACAAACTTCAGCCTACTCATGTCCCTAAAATGGG |
| | | CAAACATTGCAAGCAGCAAACAGCAAACACACAGCCCTCCCTGCCTG |
| | | CTGACCTTGGAGCTGGGGCAGAGGTCAGAGACCTCTCTGGGCCCATG |
| | | CCACCTCCAACATCCACTCGACCCCTTGGAATTTCGGTGGAGAGGAG |
| | | CAGAGGTTGTCCTGGCGTGGTTTAGGTAGTGTGAGAGGG |
| 12 | hAAT (Promoter) | GATCTTGCTACCAGTGGAACAGCCACTAAGGATTCTGCAGTGAGAGC |
| | (na) | AGAGGGCCAGCTAAGTGGTACTCTCCCAGAGACTGTCTGACTCACGC |
| | | CACCCCCTCCACCTTGGACACAGGACGCTGTGGTTTCTGAGCCAGGT |
| | | ACAATGACTCCTTTCGGTAAGTGCAGTGGAAGCTGTACACTGCCCAG |
| | | GCAAAGCGTCCGGGCAGCGTAGGCGGGCGACTCAGATCCCAGCCAGT |
| | | GGACTTAGCCCCTGTTTGCTCCTCCGATAACTGGGGTGACCTTGGTT |
| | | AATATTCACCAGCAGCCTCCCCCGTTGCCCCTCTGGATCCACTGCTT |
| | | AAATACGGACGAGGACAGGGCCCTGTCTCCTCAGCTTCAGGCACCAC |
| | | CACTGACCTGGGACAGT |
| 13 | 5′UTR (na) | CTTGTTCTTTTTGCAGAAGCTCAGAATAAACGCTCAACTTTGGCAGA |
| | | T |
| 14 | Human β-globin/ | GTAAGTATCAAGGTTACAAGACAGGTTTAAGGAGACCAATAGAAACT |
| | IgG chimeric | GGGCTTGTCGAGACAGAGAAGACTCTTGCGTTTCTGATAGGCACCTA |
| | intron (na) | TTGGTCTTACTGACATCCACTTTGCCTTTCTCTCCACAG |
| 15 | 3xFLAG (na) | GACTACAAAGACCATGACGGTGATTATAAAGATCATGACATCGATTA |
| | | CAAGGATGACGATGACAAG |
| 16 | 3xFLAG (na) | GATTATAAAGATCATGACGGGGACTATAAGGATCACGACATAGACTA |
| | | CAAAGACGATGATGACAAA |
| 153 | 3xFLAG (na) | GATTACAAAGATCACGACGGAGATTACAAAGATCACGACATTGACTA |
| | | TAAGGACGACGACGATAAA |
| 154 | 3XFLAG (na) | GATTACAAAGACCACGACGGAGACTACAAGGACCATGATATTGACTA |
| | | CAAAGACGATGATGATAAG |
| 17 | Left ZFN | GACTACAAAGACCATGACGGTGATTATAAAGATCATGACATCGATTA |
| | with N-terminal | CAAGGATGACGATGACAAGATGGCCCCCAAGAAGAAGAGGAAGGTCG |
| | modifications | GCATTCATGGGGTACCCGCCGCTATGGCTGAGAGGCCCTTCCAGTGT |
| | (comprising | CGAATCTGCATGCAGAACTTCAGTCAGTCCGGCAACCTGGCCCGCCA |
| | 3xFLAG, NLS, | CATCCGCACCCACACCGGCGAGAAGCCTTTTGCCTGTGACATTTGTG |
| | ZFP-L, and FokI) | GGAGGAAATTTGCCCTGAAGCAGAACCTGTGTATGCATACCAAGATA |
| | (na) | CACACGGGCGAGAAGCCCTTCCAGTGTCGAATCTGCATGCAGAAGTT |
| | Not diversified | TGCCTGGCAGTCCAACCTGCAGAACCATACCAAGATACACACGGGCG |
| | | AGAAGCCCTTCCAGTGTCGAATCTGCATGCGTAACTTCAGTACCTCC |
| | | GGCAACCTGACCCGCCACATCCGCACCCACACCGGCGAGAAGCCTTT |
| | | TGCCTGTGACATTTGTGGGAGGAAATTTGCCCGCCGCTCCCACCTGA |
| | | CCTCCCATACCAAGATACACCTGCGGGGATCCCAGCTGGTGAAGAGC |
| | | GAGCTGGAGGAGAAGAAGTCCGAGCTGCGGCACAAGCTGAAGTACGT |
| | | GCCCCACGAGTACATCGAGCTGATCGAGATCGCCAGGAACAGCACCC |
| | | AGGACCGCATCCTGGAGATGAAGGTGATGGAGTTCTTCATGAAGGTG |
| | | TACGGCTACAGGGGAAAGCACCTGGGCGGAAGCAGAAAGCCTGACGG |
| | | CGCCATCTATACAGTGGGCAGCCCCATCGATTACGGCGTGATCGTGG |
| | | ACACAAAGGCCTACAGCGGCGGCTACAATCTGCCTATCGGCCAGGCC |
| | | GACGAGATGGAGAGATACGTGGAGGAGAACCAGACCCGGGATAAGCA |
| | | CCTCAACCCCAACGAGTGGTGGAAGGTGTACCCTAGCAGCGTGACCG |
| | | AGTTCAAGTTCCTGTTCGTGAGCGGCCACTTCAAGGGCAACTACAAG |
| | | GCCCAGCTGACCAGGCTGAACCACATCACCAACTGCGACGGCGCCGT |
| | | GCTGAGCGTGGAGGAGCTGCTGATCGGCGGCGAGATGATCAAAGCCG |
| | | GCACCCTGACACTGGAGGAGGTGCGGCGCAAGTTCAACAACGGCGAG |
| | | ATCAACTTCAGATCT |
| 18 | Left ZFN | GATTACAAAGATCACGACGGAGATTACAAAGATCACGACATTGACTA |
| | with N-terminal | TAAGGACGACGACGATAAAATGGCTCCAAAGAAGAAAAGAAAAGTGG |
| | modifications | GGATCCATGGTGTACCCGCAGCAATGGCCGAACGACCCTTCCAATGC |
| | (comprising | AGAATATGTATGCAGAATTTTTCTCAGAGCGGGAACCTGGCGAGGCA |
| | 3xFLAG, NLS, | CATAAGAACCCATACAGGAGAGAAGCCATTCGCATGCGATATTTGCG |
| | ZFP-L, and FokI) | GTAGAAAATTTGCACTCAAACAAAATCTCTGTATGCACACTAAAATC |
| | (na) | CATACAGGTGAAAAGCCTTTTCAGTGCAGGATTTGTATGCAAAAATT |
| | Codon diversified | TGCTTGGCAAAGTAACTTGCAGAACCACACAAAGATACACACAGGAG |
| | Version 1 | AGAAACCCTTCCAATGCCGAATCTGTATGCGCAACTTCAGTACATCC |
| | | GGAAATTTGACTAGACATATTAGGACCCACACCGGCGAGAAGCCATT |
| | | TGCCTGCGATATTTGTGGACGGAAATTCGCACGACGCAGCCATCTGA |
| | | CCAGTCATACTAAGATTCATCTCCGCGGCAGCCAGCTTGTGAAGTCC |
| | | GAACTGGAGGAAAAGAAGAGCGAACTGCGCCACAAATTGAAATACGT |
| | | TCCGCATGAGTACATAGAGCTCATTGAAATCGCTAGAAACTCTACCC |
| | | AAGACAGGATACTGGAAATGAAAGTGATGGAATTTTTCATGAAAGTT |
| | | TATGGTTATAGGGGCAAACATCTGGGTGGCTCTCGCAAGCCCGATGG |
| | | GGCCATTTATACTGTCGGCTCACCTATCGACTATGGCGTCATTGTGG |
| | | ATACCAAGGCTTATTCTGGAGGATACAACCTGCCCATCGGACAAGCA |
| | | GACGAAATGGAAAGATACGTCGAGGAGAATCAAACCCGAGACAAGCA |
| | | TCTGAACCCAAACGAGTGGTGGAAAGTGTACCCGAGCAGCGTTACTG |
| | | AGTTCAAATTTCTCTTTGTAAGCGGACATTTTAAAGGGAATTACAAA |
| | | GCACAACTGACTAGGCTGAACCATATAACCAACTGTGACGGGGCCGT |
| | | ATTGAGTGTGGAAGAGCTTCTGATTGGAGGAGAGATGATTAAGGCTG |
| | | GCACACTGACTCTCGAAGAAGTGAGGCGCAAATTCAATAACGGTGAA |
| | | ATCAACTTCCGGTCT |
| 19 | Left ZFN | GACTACAAGGACCACGACGGTGACTACAAAGACCACGATATAGACTA |
| | with N-terminal | TAAAGATGACGATGATAAGATGGCACCTAAAAAAAAGCGGAAAGTGG |
| | modifications | GAATTCACGGCGTGCCCGCCGCCATGGCAGAGAGACCCTTTCAATGT |
| | (comprising | AGAATCTGTATGCAAAATTTCTCTCAGAGTGGTAACCTTGCAAGACA |
| | 3xFLAG, NLS, | CATCAGAACTCATACAGGTGAGAAGCCGTTTGCATGTGACATTTGCG |
| | ZFP-L, and FokI | GTAGGAAATTTGCCTTGAAACAGAATCTTTGTATGCACACAAAAATC |
| | (na) | CATACTGGTGAAAAGCCATTCCAATGCCGCATCTGTATGCAAAAATT |
| | Codon diversified | CGCGTGGCAGTCCAATTTGCAGAACCATACCAAGATTCACACGGGAG |
| | Version 2 | AAAAACCATTTCAGTGCCGCATCTGCATGCGCAACTTTTCTACATCA |
| | | GGAAACCTTACACGACATATTCGGACGCACACTGGAGAAAAACCATT |
| | | TGCTTGTGACATATGCGGCCGAAAATTTGCCAGACGCTCTCATCTCA |
| | | CCTCACATACTAAGATTCATTTGCGCGGAAGTCAGCTGGTGAAGAGT |
| | | GAATTGGAAGAAAAAAAGTCAGAGCTGAGACACAAACTGAAATATGT |
| | | TCCACACGAGTACATCGAGCTTATCGAGATAGCAAGAAACTCCACCC |
| | | AGGACAGAATTTTGGAAATGAAAGTTATGGAATTCTTTATGAAAGTG |
| | | TATGGCTACAGGGGTAAACATCTGGGGGGATCAAGAAAGCCTGATGG |
| | | TGCAATTTACACAGTGGGCTCTCCTATCGACTACGGTGTGATCGTGG |
| | | ATACAAAGGCCTACTCTGGAGGATATAATTTGCCTATTGGACAAGCC |
| | | GATGAAATGGAAAGATATGTGGAGGAAAACGAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGT |
| 20 | Left ZFN | GATTACAAAGACCACGACGGAGACTACAAGGACCATGATATTGACTA |
| | with N-terminal | CAAAGACGATGATGATAAGATGGCACCCAAAAAGAAGAGAAAAGTGG |
| | modifications | GAATCCACGGTGTACCGGCCGCGATGGCAGAGAGACCATTTCAGTGT |
| | (comprising | AGAATCTGTATGCAGAACTTTTCCCAATCAGGAAACCTGGCACGACA |
| | 3xFLAG, NLS, | CATTAGAACCCATACTGGAGAAAAGCCGTTCGCTTGCGACATTTGCG |
| | ZFP-L, and FokI) | GTAGAAAATTTGCTTTGAAACAGAACTTGTGTATGCATACCAAGATT |
| | (na) | CATACCGGCGAAAAACCATTTCAATGCAGGATTTGTATGCAGAAGTT |
| | Codon diversified | CGCCTGGCAATCCAATTTGCAGAATCATACTAAAATTCATACCGGAG |
| | Version 3 | AAAAACCATTCCAATGCCGCATTTGTATGAGAAACTTTTCTACCTCT |
| | | GGCAATCTCACCAGACATATCAGAACACACACAGGCGAGAAACCGTT |
| | | CGCATGCGATATCTGTGGGCGAAAGTTTGCCAGAAGATCCCATCTCA |
| | | CATCACATACTAAAATACATTTGCGAGGAAGTCAACTGGTCAAGTCC |
| | | GAACTGGAGGAAAAAAAAAGTGAGCTGCGAGACAAGTTGAAGTACGT |
| | | ACCACACGAATACATCGAGCTGATTGAGATAGCACGGAAGTCTACCC |
| | | AGGATAGAATACTGGAGATGAAAGTTATGGAATTCTTTATGAAGGTG |
| | | TACGGATACAGGGGGAAGCATCTTGGCGGGAGCCGGAAACCAGACGG |
| | | AGCAATCTATACCGTCGGGTCACCTATAGACTATGGAGTTATTGTCG |
| | | ATACAAAGGCCTATTCAGGAGGTTATAATCTGCCAATCGGCCAAGCC |
| | | GACGAGATGGAGAGGTACGTGGAGGAAAATCAGACCAGAGACAAGCA |
| | | CCTGAACCCTAATGAATGGTGGAAAGTGTACCCTAGCAGCGTCACTG |
| | | AGTTCAAATTCCTGTTCGTCAGCGGTCATTTTAAAGGAAATTATAAA |
| | | GCCCAGCTCACTAGACTCAACCATATTACAAACTGCGACGGAGCCGT |
| | | ACTTAGCGTTGAAGAGTTGCTTATCGGAGGAGAGATGATCAAAGCCG |
| | | GAACCCTCACACTTGAAGAAGTGCGAAGAAAATTCAATAACGGAGAG |
| | | ATAAATTTTAGGAGT |
| 21 | Left ZFN | GACTATAAAGACCACGATGGCGACTACAAAGACCACGACATCGATTA |
| | with N-terminal | CAAGGACGATGATGACAAAATGGCACCTAAGAAGAAGAGAAAAGTTG |
| | modifications | GAATACATGGAGTCCCCGCAGCAATGGCCGAGAGACCTTTTCAGTGC |
| | (comprising | AGGATTTGTATGCAAAACTTCTCTCAGTCCGGTAACCTGGCCCGGCA |
| | 3xFLAG, NLS, | CATACGAACACATACCGGCGAAAAACCCTTTGCTTGCGACATCTGCG |
| | ZFP-L, and FokI) | GAAGAAAGTTCGCTCTTAAACAGAACCTGTGCATGCATACAAAAATT |
| | (na) | CATACAGGTGAGAAGCCATTCCAATGCAGAATATGTATGCAGAAATT |
| | Codon diversified | CGCCTGGCAAAGCAACCTGCAAAACCACACTAAGATCCACACAGGGG |
| | Version 4 | AAAAGCCTTTTCAATGTAGAATCTGTATGAGAAACTTTAGTACATCC |
| | | GGAAATCTCACACGACATATCAGAACCCACACTGGAGAAAAACCTTT |
| | | TGCCTGCGACATCTGCGGAAGAAAATTCGCCCGAAGGTCCCACTTGA |
| | | CTAGTCATACCAAAATCCACTTGCGAGGCTCACAGCTGGTTAAATCC |
| | | GAACTTGAAGAAAAAAAAAGTGAACTGCGGCATAAACTGAAGTATGT |
| | | CCCCCATGAATATATCGAAGTGATAGAAATCGCCCGAAATAGCACCC |
| | | AAGATAGAATCCTCGAAATGAAGGTTATGGAATTTTTCATGAAGGTC |
| | | TATGGATATAGGGGCAAGCACCTTGGCGGATCCCGGAAACCTGATGG |
| | | AGCTATCTACACAGTGGGCTCACCAATAGACTATGGAGTTATCGTCG |
| | | ATACAAAAGCATACAGCGGAGGATACAATTTGCCAATAGGTCAAGCA |
| | | GATGAGATGGAAAGATACGTGGAGGAAAACCAAACAAGAGATAAGCA |
| | | TCTGAACCCCAACGAATGGTGGAAAGTGTACCCCAGTTCTGTAACCG |
| | | AATTTAAGTTCTTGTTCGTTTCAGGTCACTTCAAGGGTAATTACAAG |
| | | GCTCAACTGACTAGACTCAACCATATTACAAATTGCGATGGTGCTGT |
| | | GCTTTCCGTGGAAGAATTGCTGATTGGTGGAGAGATGATAAAAGCTG |
| | | GTACCCTCACCTTGGAAGAAGTGCGCAGAAAATTCAATAATGGCGAG |
| | | ATCAACTTCCGAAGT |
| 22 | Left ZFN | GATTATAAGGACCATGACGGAGACTATAAAGACCATGATATTGACTA |
| | with N-terminal | CAAAGACGACGATGATAAGATGGCCCCCAAGAAGAAACGAAAAGTAG |
| | modifications | GAATCCATGGCGTGCCTGCAGCAATGGCAGAGAGACCATTTCAGTGC |
| | (comprising | AGAATATGTATGCAAAACTTCTCCCAGAGCGGTAATCTGGCTAGGCA |
| | 3xFLAG, NLS, | TATTAGAACACACACCGGGGAAAAACCTTTCGCTTGCGATATATGTG |
| | ZFP-L, and FokI) | GTAGAAAGTTCGCCCTCAAACAGAATCTGTGCATGCACACTAAAATC |
| | (na) | CATACAGGAGAAAAGCCCTTTCAGTGTAGAATTTGTATGCAGAAATT |
| | Codon diversified | TGCTTGGCAGTCAAATTTGCAAAATCACACCAAAATACACACAGGAG |
| | Version 5 | AAAAACCATTTCAGTGTAGAATATGTATGAGAAATTTTTCCACTTCC |
| | | GGAAATCTGACCAGACATATACGGACACACACTGGGGAAAAGCCCTT |
| | | CGCTTGCGACATCTGCGGAAGAAAGTTCGCTAGACGGTCCCACTTGA |
| | | CATCCCACACTAAGATACATCTTCGCGGTAGCCAACTGGTGAAAAGT |
| | | GAACTGGAGGAAAAAAAATCTGAGCTGAGACATAAACTGAAATACGT |
| | | ACCACATGAATACATAGAACTTATAGAAATAGCTAGGAACTCCACCC |
| | | AGGACAGAATACTTGAAATGAAGGTCATGGAGTTTTTTATGAAAGTT |
| | | TACGGATACAGGGGCAAACACCTTGGAGGGTCTCGGAAGCCTGATGG |
| | | CGCAATTTATACCGTGGGTAGCCCTATAGATTATGGAGTGATTGTGG |
| | | ATACAAAGGCTTACAGTGGCGGCTATAATTTGCCTATCGGACAGGCC |
| | | GATGAGATGGAAAGATACGTTGAAGAAAACCAAACACGAGATAAGCA |
| | | TCTGAACCCCAATGAATGGTGGAAAGTGTATCCTTCAAGCGTTACCG |
| | | AGTTTAAGTTCCTCTTCGTTTCTGGGCATTTCAAGGGCAACTACAAA |
| | | GCTGAGCTTACAAGACTCAACCACATAACCAATTGTGATGGAGCAGT |
| | | CCTCAGCGTGGAAGAACTCCTTATTGGGGGTGAGATGATTAAAGCAG |
| | | GGAGCCTTACTCTTGAAGAGGTTAGAAGAAAATTCAATAACGGAGAG |
| | | ATTAATTTTAGAAGT |
| 23 | Left ZFN | GACTATAAGGACCATGATGGAGACTATAAAGATCACGATATTGACTA |
| | with N-terminal | TAAAGATGATGATGATAAGATGGCACCTAAGAAGAAAAGAAAGGTCG |
| | modifications | GCATTCATGGTGTGCCTGCAGCCATGGCCGAACGCCCATTTCAATGT |
| | (comprising | AGAATTTGTATGCAGAATTTTTCACAATCAGGAAACCTGGCTAGACA |
| | 3xFLAG, NLS, | TATCAGAACACATACTGGAGAAAAGCCCTTTGCTTGTGATATCTGTG |
| | ZFP-L, and FokI) | GAAGGAAATTCGCCCTGAAACAAAACCTCTGTATGCACACAAAGATC |
| | (na) | CACACCGGCGAAAAGCCTTTCCAGTGTAGGATATGCATGCAAAAATT |
| | Codon diversified | CGCCTGGCAGTCCAATCTGCAGAACCATACCAAAATTCATACTGGTG |
| | Version 6 | AAAAGCCATTTCAGTGCAGAATATGTATGAGAAACTTTAGCACTTCA |
| | | GGAAATCTCACAAGACATATAAGAACACATACAGGGGAAAAACCTTT |
| | | TGCTTGCGATATCTGCGGCAGGAAATTCGCTCGGAGAAGTCATCTCA |
| | | CAAGCCATACAAAAATCCACCTGCGAGGAAGCCAGCTGGTCAAGTCT |
| | | GAACTGGAAGAAAAAAAAAGCGAACTGCGGCATAAACTGAAATACGT |
| | | CCCACATGAATACATTGAGCTCATCGAAATTGCTAGAAACTCTACTC |
| | | AAGATAGGATATTGGAGATGAAGGTAATGGAATTCTTCATGAAGGTT |
| | | TATGGATATAGAGGAAAACATCTTGGAGGCAGTAGGAAACCCGATGG |
| | | CGCTATCTACACCGTAGGGAGTCCAATCGACTACGGCGTGATTGTTG |
| | | ACACCAAAGCCTATTCTGGAGGGTATAATCTCCCAATTGGACAGGCA |
| | | GATGAGATGGAAAGATATGTAGAAGAAAATCAGACAAGAGATAAGCA |
| | | CCTTAACCCTAACGAGTGGTGGAAAGTGTACCCAAGCAGTGTTACTG |
| | | AATTTAAATTTCTTTTTGTATCAGGACACTTTAAAGGCAATTACAAA |
| | | GCACAACTGACCAGACTCAATCACATTACCAATTGCGACGGAGCCGT |
| | | ACTGAGCGTGGAGGAGTTGCTGATCGGAGGCGAAATGATTAAAGCTG |
| | | GCACTCTGACCCTGGAAGAAGTAAGAAGAAAGTTCAATAATGGAGAA |
| | | ATAAACTTTCGCTCC |
| 24 | 2A peptide (T2A) | GGCAGCGGAGAGGGCAGAGGAAGCCTGCTCACCTGCGGTGACGTGGA |
| | (na) | GGAAAACCCTGGCCCT |
| 25 | Right ZFN | GACTACAAAGACCATGACGGTGATTATAAAGATCATGACATCGATTA |
| | with N-terminal | CAAGGATGACGATGACAAGATGGCCCCCAAGAAGAAGAGGAAGGTCG |
| | modifications | GCATTCATGGGGTACCCGCCGCTATGGCTGAGAGGCCCTTCCAGTGT |
| | (comprising | CGAATCTGCATGCGTAACTTCAGTCAGTCCTCCGACCTGTCCCGCCA |
| | 3xFLAG, NLS, | CATCCGCACCCACACCGGCGAGAAGCCTTTTGCCTGTGACATTTGTG |
| | ZFP-R, and FokI) | GGAGGAAATTTGCCCTGAAGCACAACCTGCTGACCCATACCAAGATA |
| | (na) | CACACGGGCGAGAAGCCCTTCCAGTGTCGAATCTGCATGCAGAACTT |
| | Not diversified | CAGTGACCAGTCCAACCTGCGCGCCCACATCCGCACCCACACCGGCG |
| | | AGAAGCCTTTTGCCTGTGACATTTGTGGGAGGAAATTTGCCCGCAAC |
| | | TTCTCCCTGACCATGCATACCAAGATACACACCGGAGAGCGCGGCTT |
| | | CCAGTGTCGAATCTGCATGCGTAACTTCAGTCTGCGCCACGACCTGG |
| | | AGCGCCACATCCGCACCCACACCGGCGAGAAGCCTTTTGCCTGTGAC |
| | | ATTTGTGGGAGGAAATTTGCCCACCGCTCCAACCTGAACAAGCATAC |
| | | CAAGATACACCTGCGGGGATCCCAGCTGGTGAAGAGCGAGCTGGAGG |
| | | AGAAGAAGTCCGAGCTGCGGCACAAGCTGAAGTACGTGCCCCACGAG |
| | | TACATCGAGCTGATCGAGATCGCCAGGAACAGCACCCAGGACCGCAT |
| | | CCTGGAGATGAAGGTGATGGAGTTCTTCATGAAGGTGTACGGCTACA |
| | | GGGGAAAGCACCTGGGCGGAAGCAGAAAGCCTGACGGCGCCATCTAT |
| | | ACAGTGGGCAGCCCCATCGATTACGGCGTGATCGTGGACACAAAGGC |
| | | CTACAGCGGCGGCTACAATCTGAGCATCGGCCAGGCCGACGAGATGC |
| | | AGAGATACGTGAAGGAGAACCAGACCCGGAATAAGGAGATCAACCCC |
| | | AACGAGTGGTGGAAGGTGTACCCTAGCAGCGTGACCGAGTTCAAGTT |
| | | CCTGTTCGTGAGCGGCCACTTCAAGGGCAACTACAAGGCCCAGCTGA |
| | | CCAGGCTGAACCGCAAAACCAACTGCAATGGCGCCGTGCTGAGCGTG |
| | | GAGGAGCTGCTGATCGGCGGCGAGATGATCAAAGCCGGCACCCTGAC |
| | | ACTGGAGGAGGTGCGGCGCAAGTTCAACAACGGCGAGATCAACTTC |
| 26 | Right ZFN | GATTATAAAGATCATGACGGGGACTATAAGGATGAGGACATAGACTA |
| | with N-terminal | CAAAGACGATGATGACAAAATGGCGCCTAAAAAGAAACGAAAAGTGG |
| | modifications | GCATTCACGGCGTACCTGCTGCTATGGCTGAAAGACCTTTTCAATGT |
| | (comprising | CGAATCTGCATGAGGAATTTTAGTGAGTCATCCGACCTGAGCAGACA |
| | 3xFLAG, NLS, | CATTCGAACCCATACTGGTGAAAAGCCATTTGCTTGCGATATATGTG |
| | ZFP-R, and FokI) | GGAGAAAATTTGCGTTGAAACACAATCTGCTGACCCATACCAAGATT |
| | (na) | CATACCGGAGAAAAACCATTCGAATGCCGCATTTGTATGGAGAACTT |
| | Codon diversified | TAGTGACCAGTCAAATCTCCGCGCTCACATTCGAACCCACACTGGCG |
| | Version 1 | AAAAACCCTTTGCTTGTGACATTTGCGGTCGGAAGTTTGCCCGAAAT |
| | | TTTTCTCTGACAATGCACACAAAAATCCACACCGGGGAACGCGGCTT |
| | | TGAATGTAGGATCTGTATGAGAAATTTTAGCCTTAGACATGATTTGG |
| | | AACGACATATCAGGACCCATACAGGCGAGAAACCATTTGCGTGCGAT |
| | | ATTTGTGGCAGGAAATTCGCACATAGAAGTAATCTGAACAAGCATAC |
| | | AAAAATTCATCTCAGAGGAAGTCAGCTGGTGAAAAGTGAACTGGAGG |
| | | AAAAAAAGAGCGAACTGAGACACAAACTGAAGTACGTGCGAGACGAA |
| | | TATATTGAGCTGATTGAGATCGCGAGGAACTCAACACAGGACCGCAT |
| | | TCTGGAGATGAAAGTGATGGAGTTTTTCATGAAAGTATATGGATATA |
| | | GAGGAAAACACCTTGGGGGTAGCCGAAAGCCGGACGGGGCGATCTAC |
| | | ACTGTGGGGTCACCAATTGATTATGGCGTAATTGTCGATACCAAAGC |
| | | CTACAGTGGGGGGTACAATCTGAGTATAGGACAGGCTGATGAAATGC |
| | | AACGATACGTTAAGGAGAATGAGACTAGGAATAAACATATGAATCCA |
| | | AATGAATGGTGGAAAGTCTATCCGAGGAGCGTGACAGAATTTAAATT |
| | | TTTGTTTGTCAGTGGACACTTCAAGGGAAATTATAAGGCCCAGCTGA |
| | | CTAGACTGAATAGGAAAACCAATTGTAATGGCGCAGTGCTTTCAGTG |
| | | GAGGAACTGCTCATTGGAGGTGAGATGATCAAGGCTGGAACCCTGAC |
| | | GCTGGAGGAGGTGCGGAGGAAGTTTAACAATGGAGAAATTAACTTT |
| 27 | Right ZFN | GATTATAAAGACCATGATGGTGATTACAAGGACCATGACATCGATTA |
| | with N-terminal | TAAAGACGACGACGACAAAATGGCCCCTAAGAAAAAGAGAAAAGTCG |
| | modifications | GAATCCACGGTGTCCCAGCTGCCATGGCCGAGAGACCATTTCAATGT |
| | (comprising | CGGATTTGCATGCGCAATTTTTCCCAGTCCTCTGACCTTAGCCGGCA |
| | 3xFLAG, NLS, | TATTCGGACACACACAGGTGAAAAACCCTTCGCATGCGACATTTGCG |
| | ZFP-R, and FokI) | GAAGAAAATTCGCTCTGAAACACAACCTGCTTACCCATACAAAGATC |
| | (na) | CACACCGGCGAGAAACCGTTTCAATGCCGAATCTGTATGCAAAATTT |
| | Codon diversified | TAGTGATCAAAGTAATCTGAGAGGACATATTAGGACTCACACGGGCG |
| | Version 2 | AGAAGCCATTTGCGTGTGATATCTGCGGCCGAAAATTCGCCCGGAAT |
| | | TTCTCTCTGACAATGCACACCAAAATCCACACTGGGGAACGAGGCTT |
| | | TCAATGTAGAATATGTATGCGGAATTTCAGTCTGAGGCACGACCTGG |
| | | AGCGGGACATCAGAACTCACACCGGAGAAAAACCATTCGCTTGTGAT |
| | | ATTTGCGGGAGGAAGTTCGCCCATAGGAGCAATCTCAATAAACACAC |
| | | CAAAATACATCTTCGGGGTTCTCAACTGGTGAAATCCGAACTGGAAG |
| | | AAAAGAAATCAGAATTGCGGCATAAACTGAAGTATGTGCCCCATGAG |
| | | TACATAGAACTGATCGAGATCGCAAGGAAGTCTACCCAGGACAGAAT |
| | | ACTTGAAATGAAGGTCATGGAATTTTTTATGAAAGTGTACGGCTACA |
| | | GAGGAAAACATTTGGGAGGCAGTCGAAAACCAGATGGCGCAATCTAT |
| | | ACAGTCGGGTCCCCCATAGATTACGGAGTGATTGTCGAGACAAAAGC |
| | | CTATTCCGGAGGATATAACCTTAGTATCGGCCAGGCCGACGAGATGC |
| | | AACGCTATGTGAAAGAAAACCAAACAAGAAATAAACATATCAATCCA |
| | | AACGAGTGGTGGAAGGTATATCCAAGCAGTGTCACAGAATTCAAATT |
| | | CCTCTTCGTGAGTGGGCACTTTAAAGGCAACTACAAAGCTCAATTGA |
| | | CCAGGCTCAATCGGAAAACTAATTGCAATGGCGCAGTCCTTAGCGTC |
| | | GAAGAATTGCTGATTGGCGGGGAAATGATTAAAGCAGGAACTTTGAC |
| | | CTTGGAGGAAGTACGGAGAAAGTTTAACAACGGCGAGATTAATTTT |
| 28 | Right ZFN | GATTATAAGGATCATGATGGAGACTATAAGGATCATGACATAGATTA |
| | with N-terminal | CAAAGATGACGATGACAAGATGGCACCCAAGAAGAAAAGAAAAGTAG |
| | modifications | GAATTCACGGAGTCCCTGCCGCCATGGCCGAGCGCCCCTTCCAATGC |
| | (comprising | CGCATATGCATGAGAAATTTCAGCCAAAGTAGCGACCTGTCACGACA |
| | 3xFLAG, NLS, | CATTAGAACTCATACGGGGGAGAAGCCATTTGCTTGCGATATTTGTG |
| | ZFP-R, and FokI) | GCAGAAAATTCGCACTCAAACACAACCTGCTCACACACACCAAGATA |
| | (na) | CACACGGGAGAGAAGCCCTTCCAATGTAGAATATGTATGCAAAATTT |
| | Codon diversified | CAGCGACCAAAGTAATTTGAGAGCGCATATTCGAACTCACACCGGCG |
| | Version 3 | AAAAACCATTTGCCTGCGATATTTGTGGGAGGAAATTTGCCAGGAAT |
| | | TTTTCACTCACCATGCACACTAAGATCCACACTGGCGAGCGCGGCTT |
| | | CCAATGCAGAATCTGTATGCGAAACTTCAGTCTGCGGCATGACCTGG |
| | | AAAGACATATAAGAACCCACACCGGAGAAAAACCCTTTGCCTGCGAC |
| | | ATATGTGGTAGAAAATTCGCACATCGGAGTAACCTTAACAAACATAC |
| | | AAAGATCCACTTGAGAGGCAGTCAGCTGGTGAAATCTGAGCTGGAAG |
| | | AGAAGAAATCTGAACTGCGACATAAATTGAAGTACGTCCCACACGAG |
| | | TACATCGAGTTGATCGAAATTGCCCGGAATAGCACCCAGGATAGAAT |
| | | ATTGGAAATGAAAGTAATGGAGTTTTTTATGAAGGTTTATGGTTACA |
| | | GAGGCAAGCACCTTGGAGGAAGCAGGAAACCAGATGGGGCGATTTAC |
| | | ACCGTTGGGAGTCCCATCGATTACGGAGTCATCGTGGACACAAAGGC |
| | | CTATTCCGGAGGCTACAACCTCAGTATCGGGCAAGCCGATGAGATGC |
| | | AGAGATATGTTAAAGAAAATCAGACGCGAAACAAGCACATTAACCCA |
| | | AACGAATGGTGGAAAGTTTACCCTAGCTCAGTGACAGAATTTAAGTT |
| | | TCTGTTTGTCAGCGGCCACTTCAAGGGGAATTATAAAGCACAACTGA |
| | | CCCGCCTGAACCGAAAAACCAACTGTAACGGTGCTGTGCTGAGTGTC |
| | | GAAGAGTTGCTTATCGGAGGAGAGATGATAAAGGCCGGCACACTGAC |
| | | GCTTGAAGAGGTACGGCGAAAATTCAATAACGGAGAGATTAATTTT |
| 29 | Right ZFN | GACTACAAAGATCATGATGGCGACTACAAAGATCATGATATAGATTA |
| | with N-terminal | CAAAGACGATGACGACAAAATGGCTCCAAAAAAAAAACGCAAGGTTG |
| | modifications | GAATACACGGTGTACCTGCCGCTATGGCTGAAAGACCTTTCCAGTGT |
| | (comprising | AGGATTTGCATGAGAAATTTTTCCCAATCATCCGACCTTTCAAGGCA |
| | 3xFLAG, NLS, | TATTAGGACACACACCGGGGAAAAGCCATTTGCTTGTGATATCTGCG |
| | ZFP-R, and FokI) | GGCGCAAATTTGCTCTTAAGCACAATCTTCTTACCCACACCAAAATT |
| | (na) | CATACAGGAGAAAAACCTTTTCAATGTAGAATCTGCATGCAAAACTT |
| | Codon diversified | TTCCGATCAGTCAAATCTTAGAGCTCATATCAGAACCCATACCGGGG |
| | Version 4 | AGAAACCCTTTGCCTGCGACATATGCGGAAGAAAATTTGCTAGGAAC |
| | | TTTAGTCTGACCATGCATACCAAAATTCATACCGGCGAACGCGGTTT |
| | | CCAGTGCAGGATTTGTATGAGAAATTTCTCACTGCGGCATGATCTTG |
| | | AAAGACACATACGAACTCATACCGGAGAAAAGCCATTCGCTTGCGAT |
| | | ATTTGTGGTAGAAAATTTGCCCACAGGTCTAACCTTAATAAGCACAC |
| | | CAAGATTCATCTCAGAGGATCTCAGCTGGTCAAATCAGAACTTGAAG |
| | | AGAAAAAAAGCGAACTGAGACATAAACTGAAGTACGTGCCTCATGAA |
| | | TACATAGAGCTCATTGAAATAGCTAGGAATAGTACACAGGACAGGAT |
| | | ACTTGAAATGAAGGTAATGGAATTTTTCATGAAGGTTTATGGATACC |
| | | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGACTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAACGGCGAAATCAACTTT |
| 30 | Right ZFN | GATTACAAAGACCATGATGGCGACTATAAAGACCATGACATCGACTA |
| | with N-terminal | CAAGGATGATGATGATAAAATGGCTCCAAAGAAAAAGAGGAAGGTGG |
| | modifications | GAATACATGGAGTACCAGCAGCTATGGCCGAACGCCCTTTTCAATGC |
| | (comprising | AGAATATGTATGCGAAACTTCTCCCAAAGCTCTGATCTGTCAAGGCA |
| | 3xFLAG, NLS, | CATACGGAGACACACCGGCGAAAAACCCTTTGCATGTGACATTTGTG |
| | ZFP-R, and FokI) | GAAGAAAATTCGCACTTAAACACAATCTCCTGACTCATACAAAAATA |
| | (na) | CATACAGGCGAAAAACCTTTCCAGTGCAGAATCTGTATGCAGAACTT |
| | Codon diversified | TTCCGACCAATCCAATCTTCGCGCCCACATTAGAACTCACACAGGGG |
| | Version 5 | AGAAACCTTTCGCTTGCGACATATGCGGAAGAAAATTTGCCAGAAAT |
| | | TTTTCACTTACAATGCACACAAAAATACATACTGGGGAAAGAGGGTT |
| | | TCAATGTCGAATCTGTATGAGAAATTTCAGTCTGCGCCATGATCTGG |
| | | AGAGACATATAAGAACACACACAGGAGAGAAACCTTTTGCTTGTGAC |
| | | ATATGCGGCCGAAAGTTTGCTCATAGATCTAATCTTAACAAACATAC |
| | | AAAGATCCATCTTCGGGGTTCACAACTGGTCAAGTCAGAATTGGAAG |
| | | AGAAAAAATCTGAGCTGAGGCACAAATTGAAATACGTTCCTCACGAG |
| | | TATATTGAACTTATCGAGATAGCCCGCAATAGTACACAAGATAGAAT |
| | | CTTGGAGATGAAAGTTATGGAATTCTTTATGAAAGTCTATGGCTATA |
| | | GGGGAAAACACCTGGGGGGTAGCAGGAAACCTGATGGAGCTATCTAT |
| | | ACCGTAGGATCACCTATTGATTATGGAGTAATTGTGGACACTAAGGC |
| | | ATATTCCGGAGGATATAATTTGAGTATTGGTCAGGCCGACGAAATGC |
| | | AACGATACGTGAAGGAAAATCAGACCCGCAACAAACACATTAATCCC |
| | | AATGAATGGTGGAAGGTATACCCTAGTAGCGTTACAGAGTTTAAATT |
| | | CCTTTTCGTCAGCGGCCACTTTAAAGGAAATTATAAAGCACAACTCA |
| | | CCAGACTTAATCGAAAAACTAACTGTAACGGCGCCGTACTGTCAGTG |
| | | GAGGAGCTGCTCATTGGAGGCGAGATGATCAAGGCCGGTACTCTCAC |
| | | ACTGGAAGAAGTTAGAAGAAAGTTCAACAACGGGGAAATTAATTTC |
| 31 | Right ZFN | GACTACAAGGACCACGACGGAGACTATAAAGACCATGATATAGATTA |
| | with N-terminal | CAAGGACGATGACGATAAAATGGCACCCAAAAAGAAAAGAAAGGTGG |
| | modifications | GTATTCACGGAGTTCCCGCTGCTATGGCTGAGAGACCTTTCCAATGT |
| | (comprising | AGGATCTGTATGCGAAACTTCTCCCAGAGCTCCGACCTGAGTCGCCA |
| | 3xFLAG, NLS, | TATAAGAACCCATACCGGAGAAAAACCATTTGCTTGTGACATTTGTG |
| | ZFP-R, and FokI) | GCAGAAAGTTCGCTCTTAAACACAACCTGCTTACACATACTAAAATA |
| | (na) | CACACAGGGGAGAAACCCTTTCAATGCCGGATCTGTATGCAAAACTT |
| | Codon diversified | TAGCGATCAATCAAACTTGCGAGCCCATATCCGCACTCACACCGGCG |
| | Version 6 | AGAAGCCTTTTGCATGCGATATATGTGGACGGAAATTTGCTAGAAAC |
| | | TTCTCATTGACCATGCATACAAAAATACACACCGGGGAACGAGGATT |
| | | TCAATGTCGAATTTGTATGAGAAATTTTAGCCTTAGGCACGACTTGG |
| | | AACGGCACATAAGAACCCACACCGGAGAGAAGCCTTTTGCTTGTGAT |
| | | ATTTGCGGCAGAAAGTTCGCCCATCGCAGCAATCTTAACAAGCACAC |
| | | CAAGATTCATTTGAGAGGTTCCGAGCTGGTCAAAAGCGAAGTTGAAG |
| | | AAAAGAAATCCGAGCTTAGACACAAACTGAAATACGTGCCTCACGAG |
| | | TATATTGAGCTGATTGAAATAGCAAGGAATTCAACACAAGACAGGAT |
| | | CCTCGAAATGAAGGTTATGGAGTTTTTCATGAAAGTTTACGGCTACA |
| | | GAGGGAAGCATCTGGGCGGATCAAGAAAACCAGACGGCGCAATCTAC |
| | | ACAGTTGGATCCCGAATAGATTACGGAGTGATTGTTGAGACCAAGGC |
| | | TTATTCAGGAGGTTACAATCTGTCCATTGGTCAGGCCGATGAAATGC |
| | | AAAGATATGTTAAGGAAAATCAAACTCGAAAGAAACACATTAATCCA |
| | | AACGAATGGTGGAAAGTATATCCAAGCTCCGTCACTGAATTTAAATT |
| | | TTTGTTTGTATCCGGACATTTTAAGGGCAACTATAAGGCTCAACTGA |
| | | CCAGACTGAATAGGAAGACCAATTGTAACGGAGCTGTACTCAGCGTG |
| | | GAAGAACTGCTTATTGGAGGCGAAATGATTAAGGCTGGCACACTTAC |
| | | ACTCGAAGAAGTTAGAAGAAAATTCAACAATGGTGAGATAAACTTC |
| 32 | WPREmut6 3′UTR | AATCAACCTCTGGATTACAAAATTTGTGAAAGATTGACTGATATTCT |
| | (na) | TAACTATGTTGCTCCTTTTACGCTGTGTGGATATGCTGCTTTAATGC |
| | | CTCTGTATCATGCTATTGCTTCCCGTACGGCTTTCGTTTTCTCCTCC |
| | | TTGTATAAATCCTGGTTGCTGTCTCTTTATGAGGAGTTGTGGCCCGT |
| | | TGTCCGTCAACGTGGCGTGGTGTGCTCTGTGTTTGCTGACGCAACCC |
| | | CCACTGGCTGGGGCATTGCCACCACCTGTCAACTCCTTTCTGGGACT |
| | | TTCGCTTTCCCCCTCCCGATCGCCACGGCAGAACTCATCGCCGCCTG |
| | | CCTTGCCCGCTGCTGGACAGGGGCTAGGTTGCTGGGCACTGATAATT |
| | | CCGTGGTGTTGTCGGGGAAATCATCGTCCTTTCCTTGGCTGCTCGCC |
| | | TGTGTTGCCAACTGGATCCTGCGCGGGACGTCCTTCTGCTACGTCCC |
| | | TTCGGCTCTCAATCCAGCGGACCTCCCTTCCCGAGGCCTTCTGCCGG |
| | | TTCTGCGGCCTCTCCCGCGTCTTCGCTTTCGGCCTCCGACGAGTCGG |
| | | ATCTCCCTTTGGGCCGCCTCCCCGCCTG |
| 33 | Polyadenylation | CTGTGCCTTCTAGTTGCCAGCCATCTGTTGTTTGCCCCTCCCCCGTG |
| | signal (na) | CCTTCCTTGACCCTGGAAGGTGCCACTCCCACTGTCCTTTCCTAATA |
| | | AAATGAGGAAATTGCATCGCATTGTCTGAGTAGGTGTCATTCTATTC |
| | | TGGGGGGTGGGGTGGGGCAGGACAGCAAGGGGGAGGATTGGGAAGAC |
| | | AATAGCAGGCATGCTGGGGATGCGGTGGGCTCTAT |
| 34 | 3′ ITR (na) | AGGAACCCCTAGTGATGGAGTTGGCCACTCCCTCTCTGCGCGCTCGC |
| | | TCGCTCACTGAGGCCGCCCGGGCTTTGCCCGGGCGGCCTCAGTGAGC |
| | | GAGCGAGCGCGCAG |
| 50 | 3xFLAG (na) | GATTATAAAGACCATGATGGTGATTACAAGGACCATGACATCGATTA |
| | | TAAAGACGACGACGACAAA |
| 51 | 3xFLAG (na) | GATTATAAGGATCATGATGGAGACTATAAGGATCATGACATAGATTA |
| | | CAAAGATGACGATGACAAG |
| 52 | 3xFLAG (na) | GACTACAAAGATCATGATGGCGACTACAAAGATCATGATATAGATTA |
| | | CAAAGACGATGACGACAAA |
| 53 | 3xFLAG (na) | GATTAGAAAGACCATGATGGCGACTATAAAGACCATGACATCGACTA |
| | | CAAGGATGATGATGATAAA |
| 54 | 3xFLAG (na) | GACTAGAAGGACCACGACGGAGACTATAAAGACCATGATATAGATTA |
| | | CAAGGACGATGACGATAAA |
| 55 | 3xFLAG (na) | GACTAGAAGGACCACGACGGTGACTAGAAAGACCACGATATAGACTA |
| | | TAAAGATGACGATGATAAG |
| 56 | 3xFLAG (na) | GACTATAAAGACCACGATGGCGACTAGAAAGACCACGACATCGATTA |
| | | CAAGGACGATGATGACAAA |
| 57 | 3xFLAG (na) | GATTATAAGGACCATGACGGAGACTATAAAGACCATGATATTGACTA |
| | | CAAAGACGACGATGATAAG |
| 58 | 3xFLAG (na) | GACTATAAGGACCATGATGGAGACTATAAAGATCACGATATTGACTA |
| | | TAAAGATGATGATGATAAG |
| 59 | Nuclear | CCTAAAAAGAAACGAAAAGTGGGCATTCAC |
| | Localization | |
| | Sequence (NLS) | |
| | (na) | |
| 60 | Nuclear | CCCAAGAAGAAGAGGAAGGTCGGCATTCAT |
| | Localization | |
| | Sequence (NLS) | |
| | (na) | |
| 61 | Nuclear | CCTAAGAAAAAGAGAAAAGTCGGAATCCAC |
| | Localization | |
| | Sequence (NLS) | |
| | (na) | |
| 62 | Nuclear | CCCAAGAAGAAAAGAAAAGTAGGAATTCAC |
| | Localization | |
| | Sequence (NLS) | |
| | (na) | |
| 63 | Nuclear | CCAAAAAAAAAACGCAAGGTTGGAATACAC |
| | Localization | |
| | Sequence (NLS) | |
| | (na) | |
| 64 | Nuclear | CCAAAGAAAAAGAGGAAGGTGGGAATACAT |
| | Localization | |
| | Sequence (NLS) | |
| | (na) | |
| 65 | Nuclear | CCCAAAAAGAAAAGAAAGGTGGGTATTCAC |
| | Localization | |
| | Sequence (NLS) | |
| | (na) | |
| 66 | Nuclear | CCAAAGAAGAAAAGAAAAGTGGGGATCCAT |
| | Localization | |
| | Sequence (NLS) | |
| | (na) | |
| 67 | Nuclear | CCTAAAAAAAAGCGGAAAGTGGGAATTCAC |
| | Localization | |
| | Sequence (NLS) | |
| | (na) | |
| 68 | Nuclear | CCTAAGAAGAAGAGAAAAGTTGGAATACAT |
| | Localization | |
| | Sequence (NLS) | |
| | (na) | |
| 69 | Nuclear | CCCAAGAAGAAACGAAAAGTAGGAATCCAT |
| | Localization | |
| | Sequence (NLS) | |
| | (na) | |
| 70 | Nuclear | CCTAAGAAGAAAAGAAAGGTCGGCATTCAT |
| | Localization | |
| | Sequence (NLS) | |
| | (na) | |
| 155 | Nuclear | CCCAAAAAGAAGAGAAAAGTGGGAATCCAC |
| | Localization | |
| | Sequence (NLS) | |
| | (na) | |
| 71 | Left ZFN | GCCGCTATGGCTGAGAGGCCCTTCCAGTGTCGAATCTGCATGCAGAA |
| | (ZFN-L comprises | CTTCAGTCAGTCCGGCAACCTGGCCCGCCACATCCGCACCCACACCG |
| | ZFP-L and FokI) | GCGAGAAGCCTTTTGCCTGTGACATTTGTGGGAGGAAATTTGCCCTG |
| | (na) | AAGCAGAACCTGTGTATGCATACCAAGATACACACGGGCGAGAAGCC |
| | Not diversified | CTTCCAGTGTCGAATCTGCATGCAGAAGTTTGCCTGGCAGTCCAACC |
| | | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCT |
| 72 | Left ZFN | GCAGCAATGGCCGAACGACCCTTCCAATGCAGAATATGTATGCAGAA |
| | (ZFN-L comprises | TTTTTCTCAGAGCGGGAACCTGGCGAGGCACATAAGAACCCATACAG |
| | ZFP-L and FokI) | GAGAGAAGCCATTCGCATGCGATATTTGCGGTAGAAAATTTGCACTC |
| | (na) | AAACAAAATCTCTGTATGCACACTAAAATCCATACAGGTGAAAAGCC |
| | Codon diversified | TTTTCAGTGCAGGATTTGTATGCAAAAATTTGCTTGGCAAAGTAACT |
| | Version 1 | TGCAGAACCACACAAAGATACACACAGGAGAGAAACCCTTCCAATGC |
| | | CGAATCTGTATGCGCAACTTCAGTACATCCGGAAATTTGACTAGACA |
| | | TATTAGGACCCACACCGGCGAGAAGCCATTTGCCTGCGATATTTGTG |
| | | GACGGAAATTCGCACGACGCAGCCATCTGACCAGTCATACTAAGATT |
| | | CATCTCCGCGGCAGCCAGCTTGTGAAGTCCGAACTGGAGGAAAAGAA |
| | | GAGCGAACTGCGCCACAAATTGAAATACGTTCCGCATGAGTACATAG |
| | | AGCTCATTGAAATCGCTAGAAACTCTACCCAAGACAGGATACTGGAA |
| | | ATGAAAGTGATGGAATTTTTCATGAAAGTTTATGGTTATAGGGGCAA |
| | | ACATCTGGGTGGCTCTCGCAAGCCCGATGGGGCCATTTATACTGTCG |
| | | GCTCACCTATCGACTATGGCGTCATTGTGGATACCAAGGCTTATTCT |
| | | GGAGGATACAACCTGCCCATCGGACAAGCAGACGAAATGGAAAGATA |
| | | CGTCGAGGAGAATCAAACCCGAGACAAGCATCTGAACCCAAACGAGT |
| | | GGTGGAAAGTGTACCCGAGCAGCGTTACTGAGTTCAAATTTCTCTTT |
| | | GTAAGCGGACATTTTAAAGGGAATTACAAAGCACAACTGACTAGGCT |
| | | GAACCATATAACCAACTGTGACGGGGCCGTATTGAGTGTGGAAGAGC |
| | | TTCTGATTGGAGGAGAGATGATTAAGGCTGGCACACTGAGTCTCGAA |
| | | GAAGTGAGGCGCAAATTCAATAACGGTGAAATCAACTTCCGGTCT |
| 73 | Left ZFN | GCCGCCATGGCAGAGAGACCCTTTCAATGTAGAATCTGTATGCAAAA |
| | (ZFN-L comprises | TTTCTCTCAGAGTGGTAACCTTGCAAGACACATCAGAACTCATACAG |
| | ZFP-L and FokI) | GTGAGAAGCCGTTTGCATGTGACATTTGCGGTAGGAAATTTGCCTTG |
| | (na) | AAACAGAATCTTTGTATGCACACAAAAATCCATACTGGTGAAAAGCC |
| | Codon diversified | ATTCCAATGCCGCATCTGTATGCAAAAATTCGCGTGGCAGTCCAATT |
| | Version 2 | TGCAGAACCATACCAAGATTCACACGGGAGAAAAACCATTTCAGTGC |
| | | CGCATCTGCATGCGCAACTTTTCTACATCAGGAAACCTTACACGACA |
| | | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCCAGGACAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAACCAGACTCGCGATAAGCACCTGAACCCAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGT |
| 74 | Left ZFN | GCCGCGATGGCAGAGAGACCATTTGAGTGTAGAATCTGTATGCAGAA |
| | (ZFN-L comprises | CTTTTCCCAATGAGGAAACCTGGCACGAGACATTAGAACCCATACTG |
| | ZFP-L and FokI) | GAGAAAAGCCGTTCGCTTGCGACATTTGCGGTAGAAAATTTGCTTTG |
| | (na) | AAACAGAACTTGTGTATGCATACCAAGATTCATACCGGCGAAAAACC |
| | Codon diversified | ATTTCAATGCAGGATTTGTATGCAGAAGTTCGCCTGGCAATCCAATT |
| | Version 3 | TGCAGAATCATACTAAAATTCATACCGGAGAAAAACCATTCCAATGC |
| | | CGCATTTGTATGAGAAACTTTTCTACCTCTGGCAATCTCACCAGACA |
| | | TATCAGAACACACACAGGCGAGAAACCGTTCGCATGCGATATCTGTG |
| | | GGCGAAAGTTTGCCAGAAGATCCCATCTGAGATCACATACTAAAATA |
| | | CATTTGCGAGGAAGTCAACTGGTCAAGTCCGAACTGGAGGAAAAAAA |
| | | AAGTGAGCTGCGAGACAAGTTGAAGTACGTACCACACGAATACATCG |
| | | AGCTGATTGAGATAGCACGGAACTCTAGCCAGGATAGAATACTGGAG |
| | | ATGAAAGTTATGGAATTCTTTATGAAGGTGTACGGATACAGGGGGAA |
| | | GCATCTTGGCGGGAGCCGGAAACCAGACGGAGCAATCTATACCGTCG |
| | | GGTCACCTATAGACTATGGAGTTATTGTCGATACAAAGGCCTATTCA |
| | | GGAGGTTATAATCTGCCAATCGGCCAAGCCGACGAGATGGAGAGGTA |
| | | CGTGGAGGAAAATCAGACCAGAGACAAGCACCTGAACCCTAATGAAT |
| | | GGTGGAAAGTGTACCCTAGCAGCGTCACTGAGTTCAAATTCCTGTTC |
| | | GTCAGCGGTCATTTTAAAGGAAATTATAAAGCCCAGCTCACTAGACT |
| | | CAACCATATTACAAACTGCGACGGAGCCGTACTTAGCGTTGAAGAGT |
| | | TGCTTATCGGAGGAGAGATGATGAAAGCCGGAACCCTCACACTTGAA |
| | | GAAGTGCGAAGAAAATTCAATAACGGAGAGATAAATTTTAGGAGT |
| 75 | Left ZFN | GCAGCAATGGCCGAGAGACCTTTTCAGTGCAGGATTTGTATGCAAAA |
| | (ZFN-L comprises | CTTCTCTCAGTCCGGTAACCTGGCCCGGCACATACGAACACATACCG |
| | ZFP-L and FokI) | GCGAAAAACCCTTTGCTTGCGACATCTGCGGAAGAAAGTTCGCTCTT |
| | (na) | AAACAGAACCTGTGCATGCATACAAAAATTCATACAGGTGAGAAGCC |
| | Codon diversified | ATTCCAATGCAGAATATGTATGCAGAAATTCGCCTGGCAAAGCAACC |
| | Version 4 | TGCAAAACCACACTAAGATCCACACAGGGGAAAAGCCTTTTCAATGT |
| | | AGAATCTGTATGAGAAACTTTAGTAGATCCGGAAATCTCACACGAGA |
| | | TATCAGAACCCACACTGGAGAAAAACCTTTTGCCTGCGAGATCTGCG |
| | | GAAGAAAATTCGCCCGAAGGTCCCACTTGAGTAGTCATACGAAAATC |
| | | CACTTGCGAGGCTCACAGCTGGTTAAATCCGAACTTGAAGAAAAAAA |
| | | AAGTGAACTGCGGCATAAACTGAAGTATGTCCCCCATGAATATATCG |
| | | AACTGATAGAAATCGCCCGAAATAGCACGCAAGATAGAATCCTCGAA |
| | | ATGAAGGTTATGGAATTTTTCATGAAGGTCTATGGATATAGGGGCAA |
| | | GCACCTTGGCGGATCCCGGAAACCTGATGGAGCTATCTACACAGTGG |
| | | GCTCACGAATAGACTATGGAGTTATCGTCGATACAAAAGCATACAGC |
| | | GGAGGATAGAATTTGCCAATAGGTCAAGCAGATGAGATGGAAAGATA |
| | | CGTGGAGGAAAAGGAAAGAAGAGATAAGCATCTGAACCCGAACGAAT |
| | | GGTGGAAAGTGTACCCCAGTTCTGTAACCGAATTTAAGTTCTTGTTC |
| | | GTTTCAGGTCACTTCAAGGGTAATTACAAGGCTGAACTGAGTAGACT |
| | | CAACCATATTACAAATTGCGATGGTGCTGTGCTTTCCGTGGAAGAAT |
| | | TGCTGATTGGTGGAGAGATGATAAAAGCTGGTACCCTCACCTTGGAA |
| | | GAAGTGCGCAGAAAATTGAATAATGGCGAGATGAACTTCCGAAGT |
| 76 | Left ZFN | GCAGCAATGGCAGAGAGACCATTTCAGTGCAGAATATGTATGCAAAA |
| | (ZFN-L comprises | CTTCTCCCAGAGCGGTAATCTGGCTAGGCATATTAGAACACACACCG |
| | ZFP-L and FokI) | GGGAAAAACCTTTCGCTTGCGATATATGTGGTAGAAAGTTCGCCCTC |
| | (na) | AAACAGAATCTGTGCATGCACACTAAAATCCATACAGGAGAAAAGCC |
| | Codon diversified | CTTTCAGTGTAGAATTTGTATGCAGAAATTTGCTTGGCAGTCAAATT |
| | Version 5 | TGCAAAATCACACCAAAATACACACAGGAGAAAAACCATTTCAGTGT |
| | | AGAATATGTATGAGAAATTTTTCCACTTCCGGAAATCTGAGCAGACA |
| | | TATACGGACACACACTGGGGAAAAGCCCTTCGCTTGCGACATCTGCG |
| | | GAAGAAAGTTCGCTAGACGGTCCCACTTGAGATCCCACACTAAGATA |
| | | CATCTTCGCGGTAGCCAACTGGTGAAAAGTGAACTGGAGGAAAAAAA |
| | | ATCTGAGCTGAGACATAAACTGAAATACGTACCACATGAATACATAG |
| | | AACTTATAGAAATAGCTAGGAACTCCACCCAGGACAGAATACTTGAA |
| | | ATGAAGGTCATGGAGTTTTTTATGAAAGTTTACGGATACAGGGGCAA |
| | | ACACCTTGGAGGGTCTCGGAAGCCTGATGGCGCAATTTATACCGTGG |
| | | GTAGCCCTATAGATTATGGAGTGATTGTGGATACAAAGGCTTACAGT |
| | | GGCGGCTATAATTTGCCTATCGGACAGGCCGATGAGATGGAAAGATA |
| | | CGTTGAAGAAAACCAAACACGAGATAAGCATCTGAACCCCAATGAAT |
| | | GGTGGAAAGTGTATCCTTCAAGCGTTACCGAGTTTAAGTTCCTCTTC |
| | | GTTTCTGGGCATTTCAAGGGCAACTACAAAGCTCAGCTTACAAGACT |
| | | CAACCACATAACCAATTGTGATGGAGCAGTCCTCAGCGTGGAAGAAC |
| | | TCCTTATTGGGGGTGAGATGATTAAAGCAGGGACCCTTACTCTTGAA |
| | | GAGGTTAGAAGAAAATTCAATAACGGAGAGATTAATTTTAGAAGT |
| 77 | Left ZFN | GCAGCCATGGCCGAACGCCCATTTCAATGTAGAATTTGTATGCAGAA |
| | (ZFN-L comprises | TTTTTCACAATCAGGAAACCTGGCTAGACATATCAGAACACATACTG |
| | ZFP-L and FokI) | GAGAAAAGCCCTTTGCTTGTGATATCTGTGGAAGGAAATTCGCCCTG |
| | (na) | AAACAAAACCTCTGTATGCACACAAAGATCCACACCGGCGAAAAGCC |
| | Codon diversified | TTTCCAGTGTAGGATATGCATGCAAAAATTCGCCTGGCAGTCCAATC |
| | Version 6 | TGCAGAACCATACCAAAATTCATACTGGTGAAAAGCCATTTCAGTGC |
| | | AGAATATGTATGAGAAACTTTAGCACTTCAGGAAATCTCACAAGACA |
| | | TATAAGAACACATACAGGGGAAAAACCTTTTGCTTGCGATATCTGCG |
| | | GCAGGAAATTGGCTCGGAGAAGTCATCTCACAAGCCATACAAAAATC |
| | | CACCTGCGAGGAAGCCAGCTGGTCAAGTCTGAACTGGAAGAAAAAAA |
| | | AAGCGAACTGCGGCATAAACTGAAATACGTCCCACATGAATACATTG |
| | | AGCTCATCGAAATTGCTAGAAACTCTAGTCAAGATAGGATATTGGAG |
| | | ATGAAGGTAATGGAATTCTTCATGAAGGTTTATGGATATAGAGGAAA |
| | | ACATCTTGGAGGCAGTAGGAAACCCGATGGCGCTATCTACACCGTAG |
| | | GGAGTCCAATCGACTACGGCGTGATTGTTGACACCAAAGCCTATTCT |
| | | GGAGGGTATAATCTCCCAATTGGACAGGCAGATGAGATGGAAAGATA |
| | | TGTAGAAGAAAATCAGACAAGAGATAAGCACCTTAACCCTAACGAGT |
| | | GGTGGAAAGTGTACCCAAGCAGTGTTACTGAATTTAAATTTCTTTTT |
| | | GTATCAGGACACTTTAAAGGCAATTACAAAGCACAACTGACCAGACT |
| | | CAATCACATTACCAATTGCGACGGAGCCGTACTGAGCGTGGAGGAGT |
| | | TGCTGATCGGAGGCGAAATGATTAAAGCTGGCACTCTGACCCTGGAA |
| | | GAAGTAAGAAGAAAGTTCAATAATGGAGAAATAAACTTTCGCTCC |
| 78 | Right ZFN | GCCGCTATGGCTGAGAGGCCCTTCCAGTGTCGAATCTGCATGCGTAA |
| | (ZFN-R comprises | CTTCAGTCAGTCCTCCGACCTGTCCCGCCACATCCGCACCCACACCG |
| | ZFP-R and FokI) | GCGAGAAGCCTTTTGCCTGTGACATTTGTGGGAGGAAATTTGCCCTG |
| | (na) | AAGCACAACCTGCTGACCCATACCAAGATACACACGGGCGAGAAGCC |
| | Not diversified | CTTCCAGTGTCGAATCTGCATGCAGAACTTCAGTGACCAGTCCAACC |
| | | TGCGCGCCCACATCCGCACCCACACCGGCGAGAAGCCTTTTGCCTGT |
| | | GACATTTGTGGGAGGAAATTTGCCCGCAACTTCTCCCTGACCATGCA |
| | | TACCAAGATACACACCGGAGAGCGCGGCTTCCAGTGTCGAATCTGCA |
| | | TGCGTAACTTCAGTCTGCGCCACGACCTGGAGCGCCACATCCGCACC |
| | | CACACCGGCGAGAAGCCTTTTGCCTGTGACATTTGTGGGAGGAAATT |
| | | TGCCCACCGCTCCAACCTGAACAAGCATACCAAGATACACCTGCGGG |
| | | GATCCCAGCTGGTGAAGAGCGAGCTGGAGGAGAAGAAGTCCGAGCTG |
| | | CGGCACAAGCTGAAGTACGTGCCCCACGAGTACATCGAGCTGATCGA |
| | | GATCGCCAGGAACAGCACCCAGGACCGCATCCTGGAGATGAAGGTGA |
| | | TGGAGTTCTTCATGAAGGTGTACGGCTACAGGGGAAAGCACCTGGGC |
| | | GGAAGCAGAAAGCCTGACGGCGCCATCTATACAGTGGGCAGCCCCAT |
| | | CGATTACGGCGTGATCGTGGACACAAAGGCCTACAGCGGCGGCTACA |
| | | ATCTGAGCATCGGCCAGGCCGACGAGATGCAGAGATACGTGAAGGAG |
| | | AACCAGACCCGGAATAAGCACATCAACCCCAACGAGTGGTGGAAGGT |
| | | GTACCCTAGCAGCGTGACCGAGTTCAAGTTCCTGTTCGTGAGCGGCC |
| | | ACTTCAAGGGCAACTACAAGGCCCAGCTGACCAGGCTGAACCGCAAA |
| | | ACCAACTGCAATGGCGCCGTGCTGAGCGTGGAGGAGCTGCTGATCGG |
| | | CGGCGAGATGATCAAAGCCGGCACCCTGACACTGGAGGAGGTGCGGC |
| | | GCAAGTTCAACAACGGCGAGATCAACTTC |
| 79 | Right ZFN | GCTGCTATGGCTGAAAGACCTTTTCAATGTCGAATCTGCATGAGGAA |
| | (ZFN-R comprises | TTTTAGTGAGTCATCCGACCTGAGCAGACACATTCGAACCCATACTG |
| | ZFP-R and FokI) | GTGAAAAGCCATTTGCTTGCGATATATGTGGGAGAAAATTTGCGTTG |
| | (na) | AAACACAATCTGCTGACCCATACCAAGATTCATACCGGAGAAAAACC |
| | Codon diversified | ATTCCAATGCCGCATTTGTATGCAGAACTTTAGTGACGAGTCAAATC |
| | Version 1 | TCCGCGCTCACATTCGAACCCACACTGGCGAAAAACCCTTTGCTTGT |
| | | GACATTTGCGGTCGGAAGTTTGCCCGAAATTTTTCTCTGACAATGCA |
| | | CACAAAAATCCACACCGGGGAACGCGGCTTTCAATGTAGGATCTGTA |
| | | TGAGAAATTTTAGCCTTAGACATGATTTGGAACGACATATCAGGACC |
| | | CATACAGGCGAGAAACCATTTGCGTGCGATATTTGTGGCAGGAAATT |
| | | CGCACATAGAAGTAATCTGAACAAGCATACAAAAATTCATCTCAGAG |
| | | GAAGTCAGCTGGTCAAAAGTGAACTGGAGGAAAAAAAGAGGGAACTG |
| | | AGACACAAACTGAAGTACGTGCCAGACGAATATATTGAGCTGATTGA |
| | | GATCGCGAGGAACTCAACACAGGACCGCATTCTGGAGATGAAAGTGA |
| | | TGGAGTTTTTCATGAAAGTATATGGATATAGAGGAAAACACCTTGGG |
| | | GGTAGCCGAAAGCCGGACGGGGCGATCTACACTGTGGGGTCACCAAT |
| | | TGATTATGGCGTAATTGTCGATACCAAAGCCTACAGTGGGGGGTACA |
| | | ATCTGAGTATAGGACAGGCTGATGAAATGCAAGGATACGTTAAGGAG |
| | | AATCAGACTAGGAATAAACATATCAATCGAAATGAATGGTGGAAAGT |
| | | CTATCCCAGCAGCGTGACAGAATTTAAATTTTTGTTTGTCAGTGGAC |
| | | ACTTCAAGGGAAATTATAAGGCCCAGCTGACTAGACTGAATAGGAAA |
| | | ACCAATTGTAATGGCGCAGTGCTTTCAGTGGAGGAACTGCTCATTGG |
| | | AGGTGAGATGATCAAGGCTGGAACCCTGACGCTGGAGGAGGTGCGGA |
| | | GGAAGTTTAACAATGGAGAAATTAACTTT |
| 80 | Right ZFN | GCTGCCATGGCCGAGAGACCATTTCAATGTCGGATTTGCATGCGCAA |
| | (ZFN-R comprises | TTTTTCCCAGTCCTCTGACCTTAGCCGGCATATTCGGACACACACAG |
| | ZFP-R and FokI) | GTGAAAAACCCTTCGCATGCGACATTTGCGGAAGAAAATTCGCTCTG |
| | (na) | AAACACAACCTGCTTACCCATACAAAGATCCAGACCGGCGAGAAACC |
| | Codon diversified | GTTTCAATGCCGAATCTGTATGCAAAATTTTAGTGATCAAAGTAATC |
| | Version 2 | TGAGAGCACATATTAGGACTCACACGGGCGAGAAGCCATTTGCGTGT |
| | | GATATCTGCGGCCGAAAATTCGCCCGGAATTTCTCTCTGACAATGCA |
| | | CACCAAAATCCACACTGGGGAACGAGGCTTTCAATGTAGAATATGTA |
| | | TGCGGAATTTCAGTCTGAGGCACGACCTGGAGCGGCACATCAGAACT |
| | | CACACCGGAGAAAAACCATTCGCTTGTGATATTTGCGGGAGGAAGTT |
| | | CGCCCATAGGAGCAATCTCAATAAAGACACCAAAATACATCTTCGGG |
| | | GTTCTCAACTGGTGAAATCCGAACTGGAAGAAAAGAAATCAGAATTG |
| | | CGGCATAAAGTGAAGTATGTGCCCCATGAGTACATAGAACTGATCGA |
| | | GATCGCAAGGAACTCTACCCAGGACAGAATACTTGAAATGAAGGTCA |
| | | TGGAATTTTTTATGAAAGTGTACGGCTACAGAGGAAAACATTTGGGA |
| | | GGCAGTCGAAAACCAGATGGCGCAATCTATACAGTCGGGTCCCCCAT |
| | | AGATTACGGAGTGATTGTCGACACAAAAGCCTATTCCGGAGGATATA |
| | | ACCTTAGTATCGGCCAGGCCGACGAGATGCAACGCTATGTGAAAGAA |
| | | AACCAAACAAGAAATAAACATATCAATCCAAACGAGTGGTGGAAGGT |
| | | ATATCCAAGCAGTGTCACAGAATTCAAATTCCTCTTCGTGAGTGGGC |
| | | ACTTTAAAGGCAACTACAAAGCTCAATTGACCAGGCTCAATCGGAAA |
| | | ACTAATTGCAATGGCGCAGTCCTTAGCGTCGAAGAATTGCTGATTGG |
| | | CGGGGAAATGATTAAAGCAGGAACTTTGACCTTGGAGGAAGTACGGA |
| | | GAAAGTTTAACAACGGCGAGATTAATTTT |
| 81 | Right ZFN | GCCGCCATGGCCGAGCGCCCCTTCCAATGCCGCATATGCATGAGAAA |
| | (ZFN-R comprises | TTTCAGCCAAAGTAGCGACCTGTCACGACACATTAGAACTCATACGG |
| | ZFP-R and FokI) | GGGAGAAGCCATTTGCTTGCGATATTTGTGGCAGAAAATTCGCACTC |
| | (na) | AAACACAACCTGCTCACACACACCAAGATACACACGGGAGAGAAGCC |
| | Codon diversified | CTTCCAATGTAGAATATGTATGCAAAATTTCAGCGACCAAAGTAATT |
| | Version 3 | TGAGAGCGCATATTCGAACTCACACCGGCGAAAAACCATTTGCCTGC |
| | | GATATTTGTGGGAGGAAATTTGCCAGGAATTTTTCACTCACCATGCA |
| | | CACTAAGATCCACACTGGCGAGCGCGGCTTCCAATGCAGAATCTGTA |
| | | TGCGAAACTTCAGTCTGCGGCATGACCTGGAAAGACATATAAGAACC |
| | | CACACCGGAGAAAAACCCTTTGCCTGCGAGATATGTGGTAGAAAATT |
| | | CGCACATCGGAGTAACCTTAACAAACATACAAAGATCCACTTGAGAG |
| | | GCAGTCAGCTGGTGAAATCTGAGCTGGAAGAGAAGAAATCTGAACTG |
| | | CGACATAAATTGAAGTACGTCCCAGACGAGTACATCGAGTTGATCGA |
| | | AATTGCCCGGAATAGCACCCAGGATAGAATATTGGAAATGAAAGTAA |
| | | TGGAGTTTTTTATGAAGGTTTATGGTTACAGAGGCAAGCACCTTGGA |
| | | GGAAGCAGGAAACCAGATGGGGCGATTTACACCGTTGGGAGTCCCAT |
| | | CGATTACGGAGTCATCGTGGACACAAAGGCCTATTCCGGAGGCTACA |
| | | ACCTCAGTATCGGGCAAGCCGATGAGATGCAGAGATATGTTAAAGAA |
| | | AATCAGACGCGAAACAAGCACATTAACCGAAACGAATGGTGGAAAGT |
| | | TTACCCTAGCTCAGTGACAGAATTTAAGTTTCTGTTTGTCAGCGGCC |
| | | ACTTCAAGGGGAATTATAAAGCACAACTGACCCGCCTGAACCGAAAA |
| | | ACCAACTGTAACGGTGCTGTGCTGAGTGTCGAAGAGTTGCTTATCGG |
| | | AGGAGAGATGATAAAGGCCGGCACACTGACGCTTGAAGAGGTACGGC |
| | | GAAAATTCAATAACGGAGAGATTAATTTT |
| 82 | Right ZFN | GCCGCTATGGCTGAAAGACCTTTCCAGTGTAGGATTTGCATGAGAAA |
| | (ZFN-R comprises | TTTTTCCCAATCATCCGACCTTTGAAGGCATATTAGGACACACACCG |
| | ZFP-R and FokI) | GGGAAAAGCCATTTGCTTGTGATATCTGCGGGCGCAAATTTGCTCTT |
| | (na) | AAGGACAATCTTCTTACCCACACCAAAATTCATACAGGAGAAAAACC |
| | Codon diversified | TTTTCAATGTAGAATCTGCATGCAAAACTTTTCCGATCAGTCAAATC |
| | Version 4 | TTAGAGCTCATATCAGAACCCATACCGGGGAGAAACCCTTTGCCTGC |
| | | GACATATGCGGAAGAAAATTTGCTAGGAACTTTAGTCTGACCATGCA |
| | | TACCAAAATTCATACCGGCGAACGCGGTTTCCAGTGCAGGATTTGTA |
| | | TGAGAAATTTCTCACTGCGGCATGATCTTGAAAGACACATACGAACT |
| | | CATACCGGAGAAAAGCCATTCGCTTGCGATATTTGTGGTAGAAAATT |
| | | TGCCCACAGGTCTAACCTTAATAAGCACACCAAGATTCATCTCAGAG |
| | | GATCTCAGCTGGTCAAATCAGAACTTGAAGAGAAAAAAAGCGAACTG |
| | | AGACATAAACTGAAGTACGTGCCTCATGAATACATAGAGCTCATTGA |
| | | AATAGCTAGGAATAGTACACAGGACAGGATACTTGAAATGAAGGTAA |
| | | TGGAATTTTTCATGAAGGTTTATGGATACCGGGGGAAACATCTCGGG |
| | | GGCAGCAGAAAACCAGACGGAGCAATTTATACTGTCGGGAGTCCTAT |
| | | AGATTATGGCGTTATCGTCGATACAAAGGCCTATTCCGGTGGGTACA |
| | | ACCTCTCAATTGGTCAGGCTGATGAGATGCAAAGATACGTCAAAGAA |
| | | AACCAAACGAGAAATAAACATATAAATCCCAATGAATGGTGGAAAGT |
| | | ATACCCAAGTTCCGTGACTGAATTCAAGTTCCTTTTCGTGTCTGGCC |
| | | ACTTTAAAGGAAATTATAAAGCACAATTGAGTAGACTGAATAGAAAA |
| | | ACAAACTGTAACGGCGCAGTGCTGTCAGTGGAAGAACTGCTCATAGG |
| | | TGGAGAGATGATCAAGGCCGGGACACTTACTCTTGAGGAAGTTAGAA |
| | | GGAAGTTCAACAACGGCGAAATCAACTTT |
| 83 | Right ZFN | GCAGCTATGGCCGAACGCCCTTTTCAATGCAGAATATGTATGCGAAA |
| | (ZFN-R comprises | CTTCTCCCAAAGCTCTGATCTGTGAAGGCACATACGGAGACACACCG |
| | ZFP-R and FokI) | GCGAAAAACCCTTTGCATGTGACATTTGTGGAAGAAAATTCGCACTT |
| | Codon diversified | AAACACAATCTCCTGAGTCATACAAAAATACATACAGGCGAAAAACC |
| | Version 5 | TTTCCAGTGCAGAATCTGTATGCAGAACTTTTCCGACCAATCCAATC |
| | | TTCGCGCCCACATTAGAACTCACACAGGGGAGAAACCTTTCGCTTGC |
| | | GACATATGCGGAAGAAAATTTGCCAGAAATTTTTCACTTACAATGCA |
| | | CACAAAAATACATACTGGGGAAAGAGGGTTTCAATGTCGAATCTGTA |
| | | TGAGAAATTTCAGTCTGCGCCATGATCTGGAGAGACATATAAGAACA |
| | | CACACAGGAGAGAAACCTTTTGCTTGTGACATATGCGGCCGAAAGTT |
| | | TGCTCATAGATCTAATCTTAACAAACATACAAAGATCCATCTTCGGG |
| | | GTTCACAACTGGTCAAGTCAGAATTGGAAGAGAAAAAATCTGAGCTG |
| | | AGGCACAAATTGAAATACGTTCCTCACGAGTATATTGAACTTATCGA |
| | | GATAGCCCGCAATAGTACACAAGATAGAATCTTGGAGATGAAAGTTA |
| | | TGGAATTCTTTATGAAAGTCTATGGCTATAGGGGAAAACACCTGGGG |
| | | GGTAGCAGGAAACCTGATGGAGCTATCTATACCGTAGGATCACCTAT |
| | | TGATTATGGAGTAATTGTGGACACTAAGGCATATTCCGGAGGATATA |
| | | ATTTGAGTATTGGTCAGGCCGACGAAATGCAACGATACGTGAAGGAA |
| | | AATCAGAGCCGCAACAAACACATTAATCCCAATGAATGGTGGAAGGT |
| | | ATACCCTAGTAGCGTTACAGAGTTTAAATTCCTTTTCGTCAGCGGCC |
| | | ACTTTAAAGGAAATTATAAAGCACAACTCACCAGACTTAATCGAAAA |
| | | ACTAACTGTAACGGCGCCGTACTGTCAGTGGAGGAGCTGCTCATTGG |
| | | AGGCGAGATGATCAAGGCCGGTACTCTCACACTGGAAGAAGTTAGAA |
| | | GAAAGTTCAACAACGGGGAAATTAATTTC |
| 84 | Right ZFN | GCTGCTATGGCTGAGAGACCTTTCCAATGTAGGATCTGTATGCGAAA |
| | (ZFN-R comprises | CTTCTCCCAGAGCTCCGACCTGAGTCGCCATATAAGAACCCATACCG |
| | ZFP-R and FokI) | GAGAAAAACCATTTGCTTGTGACATTTGTGGCAGAAAGTTCGCTCTT |
| | (na) | AAACACAACCTGCTTACACATACTAAAATACACACAGGGGAGAAACC |
| | Codon diversified | CTTTCAATGCCGGATCTGTATGCAAAACTTTAGCGATCAATCAAACT |
| | Version 6 | TGCGAGCCCATATCCGCACTCACACCGGCGAGAAGCCTTTTGCATGC |
| | | GATATATGTGGACGGAAATTTGCTAGAAACTTCTCATTGAGCATGCA |
| | | TACAAAAATACACACCGGGGAACGAGGATTTCAATGTCGAATTTGTA |
| | | TGAGAAATTTTAGCCTTAGGCAGGACTTGGAACGGCACATAAGAACC |
| | | CACACCGGAGAGAAGCCTTTTGCTTGTGATATTTGCGGCAGAAAGTT |
| | | CGCCCATCGGAGCAATCTTAACAAGCAGACCAAGATTCATTTGAGAG |
| | | GTTCCCAGCTGGTCAAAAGCGAACTTGAAGAAAAGAAATCCGAGCTT |
| | | AGACACAAACTGAAATACGTGCCTCACGAGTATATTGAGCTGATTGA |
| | | AATAGCAAGGAATTCAACACAAGACAGGATCCTGGAAATGAAGGTTA |
| | | TGGAGTTTTTCATGAAAGTTTACGGCTACAGAGGGAAGCATCTGGGC |
| | | GGATCAAGAAAACCAGACGGCGCAATCTACACAGTTGGATCCCCAAT |
| | | AGATTACGGAGTGATTGTTGACACCAAGGCTTATTCAGGAGGTTACA |
| | | ATCTGTCCATTGGTCAGGCCGATGAAATGCAAAGATATGTTAAGGAA |
| | | AATCAAACTCGAAACAAACACATTAATCCAAACGAATGGTGGAAAGT |
| | | ATATCCAAGCTCCGTCACTGAATTTAAATTTTTGTTTGTATCCGGAC |
| | | ATTTTAAGGGCAACTATAAGGCTGAACTGACCAGACTGAATAGGAAG |
| | | ACCAATTGTAACGGAGCTGTACTCAGCGTGGAAGAACTGCTTATTGG |
| | | AGGCGAAATGATTAAGGCTGGCACACTTACACTCGAAGAAGTTAGAA |
| | | GAAAATTCAACAATGGTGAGATAAACTTC |
| 85 | Right ZFN-T2A- | GATTATAAAGATCATGACGGGGACTATAAGGATCACGAGATAGACTA |
| | Left ZFN | CAAAGACGATGATGACAAAATGGCGCCTAAAAAGAAACGAAAAGTGG |
| | with N-terminal | GCATTCACGGCGTACCTGCTGCTATGGCTGAAAGACCTTTTCAATGT |
| | modifications | CGAATCTGCATGAGGAATTTTAGTGAGTCATCCGACCTGAGCAGACA |
| | (comprising | CATTCGAACCCATACTGGTGAAAAGCCATTTGCTTGCGATATATGTG |
| | 3xFLAG, NLS, | GGAGAAAATTTGCGTTGAAACACAATCTGCTGACCCATACCAAGATT |
| | ZFP-R, FokI, T2A, | CATACCGGAGAAAAACCATTCCAATGCCGCATTTGTATGGAGAACTT |
| | 3xFLAG, NLS, | TAGTGACCAGTCAAATCTCCGCGCTCACATTCGAACCCACACTGGCG |
| | ZFP-L, and FokI) | AAAAACCCTTTGCTTGTGACATTTGCGGTCGGAAGTTTGCCCGAAAT |
| | (na) | TTTTCTCTGACAATGCACACAAAAATCCACACCGGGGAACGCGGCTT |
| | ZFN-R | TCAATGTAGGATCTGTATGAGAAATTTTAGCCTTAGACATGATTTGG |
| | Codon diversified | AACGACATATCAGGACCCATACAGGCGAGAAACCATTTGCGTGCGAT |
| | Version 1 | ATTTGTGGCAGGAAATTCGCACATAGAAGTAATCTGAACAAGCATAC |
| | ZFN-L | AAAAATTCATCTCAGAGGAAGTCAGCTGGTCAAAAGTGAACTGGAGG |
| | Not diversified | AAAAAAAGAGCGAACTGAGACACAAACTGAAGTACGTGCCAGACGAA |
| | | TATATTGAGCTGATTGAGATCGCGAGGAACTCAACACAGGACCGCAT |
| | | TCTGGAGATGAAAGTGATGGAGTTTTTCATGAAAGTATATGGATATA |
| | | GAGGAAAACACCTTGGGGGTAGCCGAAAGCCGGACGGGGCGATCTAC |
| | | ACTGTGGGGTCACCAATTGATTATGGCGTAATTGTCGATACCAAAGC |
| | | CTACAGTGGGGGGTACAATCTGAGTATAGGACAGGCTGATGAAATGC |
| | | AACGATACGTTAAGGAGAATCAGACTAGGAATAAACATATCAATCGA |
| | | AATGAATGGTGGAAAGTCTATCCGAGGAGCGTGACAGAATTTAAATT |
| | | TTTGTTTGTCAGTGGACACTTCAAGGGAAATTATAAGGCCCAGCTGA |
| | | CTAGACTGAATAGGAAAACCAATTGTAATGGCGCAGTGCTTTCAGTG |
| | | GAGGAACTGCTCATTGGAGGTGAGATGATCAAGGCTGGAACCCTGAC |
| | | GCTGGAGGAGGTGCGGAGGAAGTTTAACAATGGAGAAATTAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGAGGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCGAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 86 | Right ZFN-T2A- | GATTATAAAGACCATGATGGTGATTAGAAGGACCATGAGATCGATTA |
| | Left ZFN | TAAAGACGACGACGACAAAATGGCCCCTAAGAAAAAGAGAAAAGTCG |
| | with N-terminal | GAATCCACGGTGTCCCAGCTGCCATGGCCGAGAGACCATTTCAATGT |
| | modifications | CGGATTTGCATGCGCAATTTTTCCCAGTCCTCTGACCTTAGCCGGCA |
| | (comprising | TATTCGGACACACACAGGTGAAAAACCCTTCGCATGCGACATTTGCG |
| | 3xFLAG, NLS, | GAAGAAAATTCGCTCTGAAACACAAGCTGCTTACCCATACAAAGATC |
| | ZFP-R, FokI, T2A, | GACACCGGCGAGAAACCGTTTCAATGCCGAATCTGTATGCAAAATTT |
| | 3xFLAG, NLS, | TAGTGATCAAAGTAATCTGAGAGGACATATTAGGACTCACACGGGCG |
| | ZFP-L, and FokI) | AGAAGCCATTTGCGTGTGATATCTGCGGCCGAAAATTCGCCCGGAAT |
| | (na) | TTCTCTCTGACAATGCACACCAAAATCCACACTGGGGAACGAGGCTT |
| | ZFN-R | TCAATGTAGAATATGTATGCGGAATTTCAGTCTGAGGCACGACCTGG |
| | Codon diversified | AGCGGGAGATGAGAAGTCACACCGGAGAAAAACCATTCGCTTGTGAT |
| | Version 2 | ATTTGCGGGAGGAAGTTCGCCCATAGGAGCAATCTCAATAAACACAC |
| | ZFN-L | CAAAATACATCTTCGGGGTTCTCAACTGGTGAAATCCGAACTGGAAG |
| | Not diversified | AAAAGAAATCAGAATTGCGGCATAAACTGAAGTATGTGCCCCATGAG |
| | | TACATAGAACTGATCGAGATCGCAAGGAAGTCTACCGAGGACAGAAT |
| | | ACTTGAAATGAAGGTCATGGAATTTTTTATGAAAGTGTACGGCTACA |
| | | GAGGAAAACATTTGGGAGGCAGTCGAAAACCAGATGGCGCAATCTAT |
| | | ACAGTCGGGTCCCCCATAGATTACGGAGTGATTGTCGACACAAAAGC |
| | | CTATTCCGGAGGATATAACCTTAGTATCGGCCAGGCCGACGAGATGC |
| | | AACGCTATGTGAAAGAAAACGAAACAAGAAATAAACATATCAATCCA |
| | | AACGAGTGGTGGAAGGTATATCCAAGGAGTGTGACAGAATTGAAATT |
| | | CCTCTTCGTGAGTGGGCACTTTAAAGGCAACTACAAAGCTCAATTGA |
| | | CCAGGCTCAATCGGAAAACTAATTGCAATGGCGCAGTCCTTAGCGTC |
| | | GAAGAATTGCTGATTGGCGGGGAAATGATTAAAGCAGGAACTTTGAC |
| | | CTTGGAGGAAGTACGGAGAAAGTTTAACAACGGCGAGATTAATTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGAGATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCGAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 87 | Right ZFN-T2A- | GATTATAAGGATCATGATGGAGACTATAAGGATCATGACATAGATTA |
| | Left ZFN | CAAAGATGACGATGACAAGATGGGAGCCAAGAAGAAAAGAAAAGTAG |
| | with N-terminal | GAATTCACGGAGTCCCTGCCGCCATGGCCGAGCGCCCCTTCCAATGC |
| | modifications | CGCATATGCATGAGAAATTTGAGCCAAAGTAGCGACCTGTCACGAGA |
| | (comprising | CATTAGAACTCATACGGGGGAGAAGCCATTTGCTTGCGATATTTGTG |
| | 3xFLAG, NLS, | GCAGAAAATTCGCACTCAAACACAACCTGCTCACACACACCAAGATA |
| | ZFP-R, FokI, T2A, | CACACGGGAGAGAAGCCCTTCCAATGTAGAATATGTATGCAAAATTT |
| | 3xFLAG, NLS, | CAGCGACCAAAGTAATTTGAGAGCGCATATTCGAACTCACACCGGCG |
| | ZFP-L, and FokI) | AAAAACCATTTGCCTGCGATATTTGTGGGAGGAAATTTGCCAGGAAT |
| | (na) | TTTTCACTCACCATGCACACTAAGATCCACACTGGCGAGCGCGGCTT |
| | ZFN-R | CCAATGCAGAATCTGTATGCGAAACTTCAGTCTGCGGCATGACCTGG |
| | Codon diversified | AAAGACATATAAGAACCGAGACCGGAGAAAAACCCTTTGCCTGCGAC |
| | Version 3 | ATATGTGGTAGAAAATTCGGAGATCGGAGTAACCTTAAGAAAGATAC |
| | ZFN-L | AAAGATCCACTTGAGAGGCAGTCAGCTGGTGAAATCTGAGCTGGAAG |
| | Not diversified | AGAAGAAATCTGAACTGCGAGATAAATTGAAGTACGTCCGAGACGAG |
| | | TACATCGAGTTGATCGAAATTGCCCGGAATAGCACCCAGGATAGAAT |
| | | ATTGGAAATGAAAGTAATGGAGTTTTTTATGAAGGTTTATGGTTACA |
| | | GAGGCAAGCACCTTGGAGGAAGCAGGAAACCAGATGGGGCGATTTAC |
| | | ACCGTTGGGAGTCCCATCGATTACGGAGTCATCGTGGACACAAAGGC |
| | | CTATTCCGGAGGCTACAACCTCAGTATCGGGCAAGCCGATGAGATGC |
| | | AGAGATATGTTAAAGAAAATCAGACGCGAAACAAGCACATTAACCCA |
| | | AACGAATGGTGGAAAGTTTACCCTAGCTCAGTGACAGAATTTAAGTT |
| | | TCTGTTTGTCAGCGGCCACTTCAAGGGGAATTATAAAGCACAACTGA |
| | | CCCGCCTGAACCGAAAAACCAACTGTAACGGTGCTGTGCTGAGTGTC |
| | | GAAGAGTTGCTTATCGGAGGAGAGATGATAAAGGCCGGCACACTGAC |
| | | GCTTGAAGAGGTACGGCGAAAATTCAATAACGGAGAGATTAATTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 88 | Right ZFN-T2A- | GACTACAAAGATCATGATGGCGACTACAAAGATCATGATATAGATTA |
| | Left ZFN | CAAAGACGATGACGACAAAATGGCTCCAAAAAAAAAACGCAAGGTTG |
| | with N-terminal | GAATACACGGTGTACCTGCCGCTATGGCTGAAAGACCTTTCCAGTGT |
| | modifications | AGGATTTGCATGAGAAATTTTTCCCAATCATCCGACCTTTCAAGGCA |
| | (comprising | TATTAGGACACACACCGGGGAAAAGCCATTTGCTTGTGATATCTGCG |
| | 3xFLAG, NLS, | GGCGCAAATTTGCTCTTAAGCACAATCTTCTTACCCACACCAAAATT |
| | ZFP-R, FokI, T2A, | CATACAGGAGAAAAACCTTTTCAATGTAGAATCTGCATGCAAAACTT |
| | 3xFLAG, NLS, | TTCCGATCAGTCAAATCTTAGAGCTCATATCAGAACCCATACCGGGG |
| | ZFP-L, and FokI) | AGAAACCCTTTGCCTGCGACATATGCGGAAGAAAATTTGCTAGGAAC |
| | (na) | TTTAGTCTGACCATGCATACCAAAATTCATACCGGCGAACGCGGTTT |
| | ZFN-R | CCAGTGCAGGATTTGTATGAGAAATTTCTCACTGCGGCATGATCTTG |
| | Codon diversified | AAAGACACATACGAACTCATACCGGAGAAAAGCCATTCGCTTGCGAT |
| | Version 4 | ATTTGTGGTAGAAAATTTGCCCACAGGTCTAACCTTAATAAGCACAC |
| | ZFN-L | CAAGATTCATCTCAGAGGATCTCAGCTGGTCAAATCAGAACTTGAAG |
| | Not diversified | AGAAAAAAAGCGAACTGAGACATAAACTGAAGTACGTGCCTCATGAA |
| | | TACATAGAGCTCATTGAAATAGCTAGGAATAGTACACAGGAGAGGAT |
| | | ACTTGAAATGAAGGTAATGGAATTTTTCATGAAGGTTTATGGATACC |
| | | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGACTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAACGGCGAAATCAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 89 | Right ZFN-T2A- | GATTACAAAGACCATGATGGCGACTATAAAGACCATGACATCGACTA |
| | Left ZFN | CAAGGATGATGATGATAAAATGGCTCCAAAGAAAAAGAGGAAGGTGG |
| | with N-terminal | GAATACATGGAGTACCAGCAGCTATGGCCGAACGCCCTTTTCAATGC |
| | modifications | AGAATATGTATGCGAAACTTCTCCCAAAGCTCTGATCTGTCAAGGCA |
| | (comprising | CATACGGACACACACCGGCGAAAAACCCTTTGCATGTGACATTTGTG |
| | 3xFLAG, NLS, | GAAGAAAATTCGCACTTAAACACAATCTCCTGACTCATACAAAAATA |
| | ZFP-R, FokI, T2A, | CATACAGGCGAAAAACCTTTCCAGTGCAGAATCTGTATGCAGAACTT |
| | 3xFLAG, NLS, | TTCCGACCAATCCAATCTTCGCGCCCACATTAGAACTCACACAGGGG |
| | ZFP-L, and FokI) | AGAAACCTTTCGCTTGCGACATATGCGGAAGAAAATTTGCCAGAAAT |
| | (na) | TTTTCACTTACAATGCACACAAAAATACATACTGGGGAAAGAGGGTT |
| | ZFN-R | TCAATGTCGAATCTGTATGAGAAATTTCAGTCTGCGCCATGATCTGG |
| | Codon diversified | AGAGACATATAAGAACACACACAGGAGAGAAACCTTTTGCTTGTGAC |
| | Version 5 | ATATGCGGCCGAAAGTTTGCTCATAGATCTAATCTTAACAAACATAC |
| | ZFN-L | AAAGATCCATCTTCGGGGTTCACAACTGGTCAAGTCAGAATTGGAAG |
| | Not diversified | AGAAAAAATCTGAGCTGAGGCACAAATTGAAATACGTTCCTCACGAG |
| | | TATATTGAACTTATCGAGATAGCCCGCAATAGTACACAAGATAGAAT |
| | | CTTGGAGATGAAAGTTATGGAATTCTTTATGAAAGTCTATGGCTATA |
| | | GGGGAAAACACCTGGGGGGTAGCAGGAAACCTGATGGAGCTATCTAT |
| | | ACCGTAGGATCACCTATTGATTATGGAGTAATTGTGGACACTAAGGC |
| | | ATATTCCGGAGGATATAATTTGAGTATTGGTCAGGCCGACGAAATGC |
| | | AACGATACGTGAAGGAAAATCAGACCCGCAACAAACACATTAATCCC |
| | | AATGAATGGTGGAAGGTATACCCTAGTAGCGTTACAGAGTTTAAATT |
| | | CCTTTTCGTCAGCGGCCACTTTAAAGGAAATTATAAAGCACAACTCA |
| | | CCAGACTTAATCGAAAAACTAACTGTAACGGCGCCGTAGTGTGAGTG |
| | | GAGGAGCTGCTCATTGGAGGCGAGATGATCAAGGCCGGTACTCTCAC |
| | | ACTGGAAGAAGTTAGAAGAAAGTTCAACAACGGGGAAATTAATTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGAGATCGATTACAAGGATGAGGATGAGAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCGAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 90 | Right ZFN-T2A- | GCGTACAAGGACCACGACGGAGACTATAAAGACCATGATATAGATTA |
| | Left ZFN | CAAGGACGATGACGATAAAATGGCACCCAAAAAGAAAAGAAAGGTGG |
| | with N-terminal | GTATTCACGGAGTTCCCGCTGCTATGGCTGAGAGACCTTTCCAATGT |
| | modifications | AGGATCTGTATGCGAAACTTCTCCCAGAGCTCCGACCTGAGTCGCCA |
| | (comprising | TATAAGAACCCATACCGGAGAAAAACCATTTGCTTGTGACATTTGTG |
| | 3xFLAG, NLS, | GCAGAAAGTTCGCTCTTAAACACAACCTGCTTACACATACTAAAATA |
| | ZFP-R, FokI, T2A, | CACACAGGGGAGAAACCCTTTCAATGCCGGATCTGTATGCAAAACTT |
| | 3xFLAG, NLS, | TAGCGATCAATCAAACTTGCGAGCCCATATCCGCACTCACACCGGCG |
| | ZFP-L, and FokI) | AGAAGCCTTTTGCATGCGATATATGTGGACGGAAATTTGCTAGAAAC |
| | (na) | TTCTCATTGACCATGCATACAAAAATACACACCGGGGAACGAGGATT |
| | ZFN-R | TCAATGTCGAATTTGTATGAGAAATTTTAGCCTTAGGCACGACTTGG |
| | Codon diversified | AACGGCACATAAGAACCCACACCGGAGAGAAGCCTTTTGCTTGTGAT |
| | Version 6 | ATTTGCGGCAGAAAGTTCGCCCATCGCAGCAATCTTAACAAGCACAC |
| | ZFN-L | CAAGATTCATTTGAGAGGTTCCGAGCTGGTCAAAAGCGAACTTGAAG |
| | Not diversified | AAAAGAAATCCGAGCTTAGACACAAACTGAAATACGTGCCTCACGAG |
| | | TATATTGAGCTGATTGAAATAGCAAGGAATTCAACACAAGACAGGAT |
| | | CCTCGAAATGAAGGTTATGGAGTTTTTCATGAAAGTTTACGGCTACA |
| | | GAGGGAAGCATCTGGGCGGATCAAGAAAACCAGACGGCGCAATCTAC |
| | | ACAGTTGGATCCCCAATAGATTACGGAGTGATTGTTGACACCAAGGC |
| | | TTATTCAGGAGGTTACAATCTGTCCATTGGTCAGGCCGATGAAATGC |
| | | AAAGATATGTTAAGGAAAATCAAACTCGAAACAAACACATTAATCCA |
| | | AACGAATGGTGGAAAGTATATCCAAGCTCCGTCACTGAATTTAAATT |
| | | TTTGTTTGTATCCGGACATTTTAAGGGCAACTATAAGGCTCAACTGA |
| | | CCAGACTGAATAGGAAGACGAATTGTAACGGAGCTGTACTCAGCGTG |
| | | GAAGAACTGCTTATTGGAGGCGAAATGATTAAGGCTGGCACACTTAC |
| | | ACTCGAAGAAGTTAGAAGAAAATTCAACAATGGTGAGATAAACTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGAGATCGATTAGAAGGATGAGGATGAGAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCGAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 91 | Right ZFN-T2A- | GACTACAAAGACCATGACGGTGATTATAAAGATCATGACATCGATTA |
| | Left ZFN | CAAGGATGACGATGACAAGATGGCCCCCAAGAAGAAGAGGAAGGTCG |
| | with N-terminal | GCATTCATGGGGTACCCGCCGCTATGGCTGAGAGGCCCTTCCAGTGT |
| | modifications | CGAATCTGCATGCGTAACTTCAGTCAGTCCTCCGACCTGTCCCGCCA |
| | (comprising | CATCCGCACCCACACCGGCGAGAAGCCTTTTGCCTGTGACATTTGTG |
| | 3xFLAG, NLS, | GGAGGAAATTTGCCCTGAAGCACAACCTGCTGACCCATACCAAGATA |
| | ZFP-R, FokI, T2A, | CACACGGGCGAGAAGCCCTTCCAGTGTCGAATCTGCATGCAGAACTT |
| | 3xFLAG, NLS, | CAGTGACCAGTCCAACCTGCGCGCCCACATCCGCACCCACACCGGCG |
| | ZFP-L, and FokI) | AGAAGCCTTTTGCCTGTGACATTTGTGGGAGGAAATTTGCCCGCAAC |
| | (na) | TTCTCCCTGACCATGCATACCAAGATACACACCGGAGAGCGCGGCTT |
| | ZFN-R | CCAGTGTCGAATCTGCATGCGTAACTTCAGTCTGCGCCACGACCTGG |
| | Not diversified | AGCGCCACATCCGCACCCACACCGGCGAGAAGCCTTTTGCCTGTGAC |
| | ZFN-L | ATTTGTGGGAGGAAATTTGCCCACCGCTCCAACCTGAACAAGCATAC |
| | Not diversified | CAAGATACACCTGCGGGGATCCCAGCTGGTGAAGAGCGAGCTGGAGG |
| | | AGAAGAAGTCCGAGCTGCGGCACAAGCTGAAGTACGTGCCCCACGAG |
| | | TACATCGAGCTGATCGAGATCGCCAGGAACAGCACCCAGGACCGCAT |
| | | CCTGGAGATGAAGGTGATGGAGTTCTTCATGAAGGTGTACGGCTACA |
| | | GGGGAAAGCACCTGGGCGGAAGCAGAAAGCCTGACGGCGCCATCTAT |
| | | ACAGTGGGCAGCCCCATCGATTACGGCGTGATCGTGGACACAAAGGC |
| | | CTACAGCGGCGGCTACAATCTGAGCATCGGCCAGGCCGACGAGATGC |
| | | AGAGATACGTGAAGGAGAACCAGACCCGGAATAAGCACATCAACCCC |
| | | AACGAGTGGTGGAAGGTGTACCCTAGCAGCGTGACCGAGTTCAAGTT |
| | | CCTGTTCGTGAGCGGCCACTTCAAGGGCAACTACAAGGCCCAGCTGA |
| | | CCAGGCTGAACCGCAAAACCAACTGCAATGGCGCCGTGCTGAGCGTG |
| | | GAGGAGCTGCTGATCGGCGGCGAGATGATCAAAGCCGGCACCCTGAC |
| | | ACTGGAGGAGGTGCGGCGCAAGTTCAACAACGGCGAGATCAACTTCG |
| | | GCAGCGGAGAGGGCAGAGGAAGCCTGCTCACCTGCGGTGACGTGGAG |
| | | GAAAACCCTGGCCCTACGCGTGCCATGGACTACAAAGACCATGACGG |
| | | TGATTATAAAGATCATGACATCGATTACAAGGATGACGATGACAAGA |
| | | TGGCCCCCAAGAAGAAGAGGAAGGTCGGCATTCATGGGGTACCCGCC |
| | | GCTATGGCTGAGAGGCCCTTCCAGTGTCGAATCTGCATGCAGAACTT |
| | | CAGTCAGTCCGGCAACCTGGCCCGCCACATCCGCACCCACACCGGCG |
| | | AGAAGCCTTTTGCCTGTGACATTTGTGGGAGGAAATTTGCCCTGAAG |
| | | CAGAACCTGTGTATGCATACCAAGATACACACGGGCGAGAAGCCCTT |
| | | CCAGTGTCGAATCTGCATGCAGAAGTTTGCCTGGCAGTCCAACCTGC |
| | | AGAACCATACCAAGATACACACGGGCGAGAAGCCCTTCCAGTGTCGA |
| | | ATCTGCATGCGTAACTTCAGTACCTCCGGCAACCTGACCCGCCACAT |
| | | CCGCACCCACACCGGCGAGAAGCCTTTTGCCTGTGACATTTGTGGGA |
| | | GGAAATTTGCCCGCCGCTCCCACCTGACCTCCCATACCAAGATACAC |
| | | CTGCGGGGATCCCAGCTGGTGAAGAGCGAGCTGGAGGAGAAGAAGTC |
| | | CGAGCTGCGGCACAAGCTGAAGTACGTGCCCCACGAGTACATCGAGC |
| | | TGATCGAGATCGCCAGGAACAGCACCCAGGACCGCATCCTGGAGATG |
| | | AAGGTGATGGAGTTCTTCATGAAGGTGTACGGCTACAGGGGAAAGCA |
| | | CCTGGGCGGAAGCAGAAAGCCTGACGGCGCCATCTATACAGTGGGCA |
| | | GCCCCATCGATTACGGCGTGATCGTGGACACAAAGGCCTACAGCGGC |
| | | GGCTACAATCTGCCTATCGGCCAGGCCGACGAGATGGAGAGATACGT |
| | | GGAGGAGAACCAGACCCGGGATAAGCACCTCAACCCCAACGAGTGGT |
| | | GGAAGGTGTACCCTAGCAGCGTGACCGAGTTCAAGTTCCTGTTCGTG |
| | | AGCGGCCACTTCAAGGGCAACTACAAGGCCCAGCTGACCAGGCTGAA |
| | | CCACATCACCAACTGCGACGGCGCCGTGCTGAGCGTGGAGGAGCTGC |
| | | TGATCGGCGGCGAGATGATCAAAGCCGGCACCCTGACACTGGAGGAG |
| | | GTGCGGCGCAAGTTCAACAACGGCGAGATCAACTTCAGATCT |
| 92 | Left ZFN-T2A- | GATTACAAAGATCACGACGGAGATTACAAAGATCACGACATTGACTA |
| | Right ZFN | TAAGGACGACGACGATAAAATGGCTCCAAAGAAGAAAAGAAAAGTGG |
| | with N-terminal | GGATCCATGGTGTACCCGCAGCAATGGCCGAACGACCCTTCCAATGC |
| | modifications | AGAATATGTATGCAGAATTTTTCTCAGAGCGGGAACCTGGCGAGGCA |
| | (comprising | CATAAGAACCCATACAGGAGAGAAGCCATTCGCATGCGATATTTGCG |
| | 3xFLAG, NLS, | GTAGAAAATTTGCACTCAAACAAAATCTCTGTATGCACACTAAAATC |
| | ZFP-L, FokI, T2A, | CATACAGGTGAAAAGCCTTTTCAGTGCAGGATTTGTATGCAAAAATT |
| | 3xFLAG, NLS, | TGCTTGGCAAAGTAACTTGCAGAACCACACAAAGATACACACAGGAG |
| | ZFP-R, and FokI) | AGAAACCCTTCCAATGCCGAATCTGTATGCGCAACTTCAGTACATCC |
| | (na) | GGAAATTTGACTAGACATATTAGGACCGAGACCGGCGAGAAGCCATT |
| | ZFN-L | TGCCTGCGATATTTGTGGACGGAAATTCGCACGACGCAGCCATCTGA |
| | Codon diversified | CCAGTCATACTAAGATTCATCTCCGCGGCAGCCAGCTTGTGAAGTCC |
| | Version 1 | GAACTGGAGGAAAAGAAGAGCGAACTGCGCCACAAATTGAAATACGT |
| | ZFN-R | TCCGCATGAGTACATAGAGCTCATTGAAATCGCTAGAAACTCTACCC |
| | Not diversified | AAGACAGGATACTGGAAATGAAAGTGATGGAATTTTTCATGAAAGTT |
| | | TATGGTTATAGGGGCAAACATCTGGGTGGCTCTCGCAAGCCCGATGG |
| | | GGCCATTTATACTGTCGGCTCACCTATCGACTATGGCGTCATTGTGG |
| | | ATACCAAGGCTTATTCTGGAGGATACAACCTGCCCATCGGACAAGCA |
| | | GACGAAATGGAAAGATACGTCGAGGAGAATCAAACCCGAGACAAGCA |
| | | TCTGAACCCAAACGAGTGGTGGAAAGTGTACCCGAGCAGCGTTACTG |
| | | AGTTCAAATTTCTCTTTGTAAGCGGACATTTTAAAGGGAATTACAAA |
| | | GCACAACTGACTAGGCTGAACCATATAACCAACTGTGACGGGGCCGT |
| | | ATTGAGTGTGGAAGAGCTTCTGATTGGAGGAGAGATGATTAAGGCTG |
| | | GCACACTGACTCTCGAAGAAGTGAGGCGGAAATTCAATAAGGGTGAA |
| | | ATCAACTTCCGGTCTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGAGATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAAGGAGACCCGGAATAAGGAGATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 93 | Left ZFN-T2A- | GACTACAAGGACCACGACGGTGACTACAAAGACCACGATATAGACTA |
| | Right ZFN | TAAAGATGACGATGATAAGATGGCACCTAAAAAAAAGCGGAAAGTGG |
| | with N-terminal | GAATTCACGGCGTGCCCGCCGCCATGGCAGAGAGACCCTTTCAATGT |
| | modifications | AGAATCTGTATGCAAAATTTCTCTCAGAGTGGTAAGCTTGCAAGACA |
| | (comprising | CATCAGAACTCATACAGGTGAGAAGCCGTTTGCATGTGACATTTGCG |
| | 3xFLAG, NLS, | GTAGGAAATTTGCCTTGAAACAGAATCTTTGTATGCACACAAAAATC |
| | ZFP-L, FokI, T2A, | CATACTGGTGAAAAGCCATTCCAATGCCGCATCTGTATGCAAAAATT |
| | 3xFLAG, NLS, | CGCGTGGCAGTCCAATTTGCAGAACCATACCAAGATTCACACGGGAG |
| | ZFP-R, and FokI) | AAAAACCATTTCAGTGCCGCATCTGCATGCGCAACTTTTCTACATCA |
| | (na) | GGAAACCTTACACGACATATTCGGACGCACACTGGAGAAAAACCATT |
| | ZFN-L | TGCTTGTGACATATGCGGCCGAAAATTTGCCAGACGCTCTCATCTCA |
| | Codon diversified | CCTCACATACTAAGATTCATTTGCGCGGAAGTCAGCTGGTGAAGAGT |
| | Version 2 | GAATTGGAAGAAAAAAAGTCAGAGCTGAGACACAAACTGAAATATGT |
| | ZFN-R | TCCACACGAGTACATCGAGCTTATCGAGATAGCAAGAAACTCCACCC |
| | Not diversified | AGGACAGAATTTTGGAAATGAAAGTTATGGAATTCTTTATGAAAGTG |
| | | TATGGCTACAGGGGTAAACATCTGGGGGGATCAAGAAAGCCTGATGG |
| | | TGCAATTTACACAGTGGGCTCTCCTATCGACTACGGTGTGATCGTGG |
| | | ATACAAAGGCCTACTCTGGAGGATATAATTTGCCTATTGGACAAGCC |
| | | GATGAAATGGAAAGATATGTGGAGGAAAACCAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGAGGGTGATTATAAAGATCATGAGATCGATTAGAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGAGCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCGCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 94 | Left ZFN-T2A- | GACTATAAAGACCACGATGGCGACTACAAAGACCACGACATCGATTA |
| | Right ZFN | CAAGGACGATGATGACAAAATGGCACCTAAGAAGAAGAGAAAAGTTG |
| | with N-terminal | GAATACATGGAGTCCCCGCAGCAATGGCCGAGAGACCTTTTCAGTGC |
| | modifications | AGGATTTGTATGCAAAACTTCTCTCAGTCCGGTAACCTGGCCCGGCA |
| | (comprising | CATACGAACACATACCGGCGAAAAACCCTTTGCTTGCGACATCTGCG |
| | 3xFLAG, NLS, | GAAGAAAGTTCGCTCTTAAACAGAACCTGTGCATGCATACAAAAATT |
| | ZFP-L, FokI, T2A, | CATACAGGTGAGAAGCCATTCCAATGCAGAATATGTATGCAGAAATT |
| | 3xFLAG, NLS, | CGCCTGGCAAAGCAACCTGCAAAACCACACTAAGATCCACACAGGGG |
| | ZFP-R, and FokI) | AAAAGCCTTTTCAATGTAGAATCTGTATGAGAAACTTTAGTACATCC |
| | (na) | GGAAATCTCACACGAGATATGAGAACCCACACTGGAGAAAAACCTTT |
| | ZFN-L | TGCCTGCGACATCTGCGGAAGAAAATTCGCCCGAAGGTCCCACTTGA |
| | Codon diversified | CTAGTCATACCAAAATCCACTTGCGAGGCTCACAGCTGGTTAAATCC |
| | Version 4 | GAACTTGAAGAAAAAAAAAGTGAACTGCGGCATAAACTGAAGTATGT |
| | ZFN-R | CCCCCATGAATATATCGAACTGATAGAAATCGCCCGAAATAGCACCC |
| | Not diversified | AAGATAGAATCCTCGAAATGAAGGTTATGGAATTTTTCATGAAGGTC |
| | | TATGGATATAGGGGCAAGCACCTTGGCGGATCCCGGAAACCTGATGG |
| | | AGCTATCTACACAGTGGGCTCACCAATAGACTATGGAGTTATCGTCG |
| | | ATACAAAAGCATACAGCGGAGGATACAATTTGCCAATAGGTCAAGCA |
| | | GATGAGATGGAAAGATACGTGGAGGAAAACCAAACAAGAGATAAGCA |
| | | TCTGAACCCCAACGAATGGTGGAAAGTGTACCCCAGTTCTGTAACCG |
| | | AATTTAAGTTCTTGTTCGTTTCAGGTCACTTCAAGGGTAATTACAAG |
| | | GCTCAACTGAGTAGACTGAACCATATTAGAAATTGCGATGGTGCTGT |
| | | GCTTTCCGTGGAAGAATTGCTGATTGGTGGAGAGATGATAAAAGCTG |
| | | GTACCCTCACCTTGGAAGAAGTGCGCAGAAAATTCAATAATGGCGAG |
| | | ATCAACTTCCGAAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGAGGGTGATTATAAAGATCATGAGATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGGAGAACCTGCTGAGCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCGCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 95 | Left ZFN-T2A- | GATTATAAGGACCATGACGGAGACTATAAAGACCATGATATTGACTA |
| | Right ZFN | CAAAGACGAGGATGATAAGATGGCCCCCAAGAAGAAACGAAAAGTAG |
| | with N-terminal | GAATCCATGGCGTGCCTGCAGCAATGGCAGAGAGACCATTTCAGTGC |
| | modifications | AGAATATGTATGCAAAACTTCTCCCAGAGCGGTAATCTGGCTAGGCA |
| | (comprising | TATTAGAACACACACCGGGGAAAAACCTTTCGCTTGCGATATATGTG |
| | 3xFLAG, NLS, | GTAGAAAGTTCGCCCTCAAACAGAATCTGTGCATGCACACTAAAATC |
| | ZFP-L, FokI, T2A, | CATACAGGAGAAAAGCCCTTTCAGTGTAGAATTTGTATGCAGAAATT |
| | 3xFLAG, NLS, | TGCTTGGCAGTGAAATTTGCAAAATCACACCAAAATACACACAGGAG |
| | ZFP-R, and FokI) | AAAAACCATTTGAGTGTAGAATATGTATGAGAAATTTTTCCACTTCC |
| | (na) | GGAAATCTGACCAGACATATACGGACACACACTGGGGAAAAGCCCTT |
| | ZFN-L | CGCTTGCGACATCTGCGGAAGAAAGTTCGCTAGACGGTCCCACTTGA |
| | Codon diversified | CATCCCACACTAAGATACATCTTCGCGGTAGCCAACTGGTGAAAAGT |
| | Version 5 | GAACTGGAGGAAAAAAAATCTGAGCTGAGACATAAACTGAAATACGT |
| | ZFN-R | ACCACATGAATACATAGAACTTATAGAAATAGCTAGGAACTCCACCC |
| | Not diversified | AGGACAGAATACTTGAAATGAAGGTCATGGAGTTTTTTATGAAAGTT |
| | | TACGGATACAGGGGCAAACACCTTGGAGGGTCTCGGAAGCCTGATGG |
| | | CGCAATTTATACCGTGGGTAGCCCTATAGATTATGGAGTGATTGTGG |
| | | ATACAAAGGCTTACAGTGGCGGCTATAATTTGCCTATCGGACAGGCC |
| | | GATGAGATGGAAAGATACGTTGAAGAAAACCAAACACGAGATAAGCA |
| | | TCTGAACCCCAATGAATGGTGGAAAGTGTATCCTTCAAGCGTTACCG |
| | | AGTTTAAGTTCCTCTTCGTTTCTGGGCATTTCAAGGGCAACTACAAA |
| | | GCTCAGCTTACAAGACTGAAGCACATAACCAATTGTGATGGAGCAGT |
| | | CCTCAGCGTGGAAGAACTCCTTATTGGGGGTGAGATGATTAAAGCAG |
| | | GGAGCCTTACTCTTGAAGAGGTTAGAAGAAAATTCAATAACGGAGAG |
| | | ATTAATTTTAGAAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGAGGGTGATTATAAAGATCATGAGATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGGAGAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 96 | Left ZFN-T2A- | GACTATAAGGACCATGATGGAGACTATAAAGATCACGATATTGACTA |
| | Right ZFN | TAAAGATGATGATGATAAGATGGCACCTAAGAAGAAAAGAAAGGTCG |
| | with N-terminal | GCATTCATGGTGTGCCTGCAGCCATGGCCGAACGCCCATTTCAATGT |
| | modifications | AGAATTTGTATGCAGAATTTTTCACAATCAGGAAACCTGGCTAGACA |
| | (comprising | TATCAGAACACATACTGGAGAAAAGCCCTTTGCTTGTGATATCTGTG |
| | 3xFLAG, NLS, | GAAGGAAATTCGCCCTGAAACAAAACCTCTGTATGCACACAAAGATC |
| | ZFP-L, FokI, T2A, | CACACCGGCGAAAAGCCTTTCCAGTGTAGGATATGCATGCAAAAATT |
| | 3xFLAG, NLS, | CGCCTGGCAGTCCAATCTGCAGAACCATACCAAAATTCATACTGGTG |
| | ZFP-R, and FokI) | AAAAGCCATTTCAGTGCAGAATATGTATGAGAAACTTTAGCACTTCA |
| | (na) | GGAAATCTCACAAGACATATAAGAACACATACAGGGGAAAAACCTTT |
| | ZFN-L | TGCTTGCGATATCTGCGGCAGGAAATTCGCTCGGAGAAGTCATCTCA |
| | Codon diversified | CAAGCCATACAAAAATCCACCTGCGAGGAAGCCAGCTGGTCAAGTCT |
| | Version 6 | GAACTGGAAGAAAAAAAAAGCGAACTGCGGCATAAACTCAAATACGT |
| | ZFN-R | CCCACATGAATACATTGAGCTCATCGAAATTGCTAGAAACTCTACTC |
| | Not diversified | AAGATAGGATATTGGAGATGAAGGTAATGGAATTCTTCATGAAGGTT |
| | | TATGGATATAGAGGAAAACATCTTGGAGGCAGTAGGAAACCCGATGG |
| | | CGCTATCTACACCGTAGGGAGTCCAATCGACTACGGCGTGATTGTTG |
| | | ACACCAAAGCCTATTCTGGAGGGTATAATCTCCCAATTGGACAGGCA |
| | | GATGAGATGGAAAGATATGTAGAAGAAAATCAGACAAGAGATAAGCA |
| | | CCTTAACCCTAACGAGTGGTGGAAAGTGTACCCAAGCAGTGTTACTG |
| | | AATTTAAATTTCTTTTTGTATCAGGACACTTTAAAGGCAATTACAAA |
| | | GCACAACTGACCAGACTCAATCACATTACCAATTGCGACGGAGCCGT |
| | | ACTGAGCGTGGAGGAGTTGCTGATCGGAGGCGAAATGATTAAAGCTG |
| | | GCACTCTGAGCCTGGAAGAAGTAAGAAGAAAGTTCAATAATGGAGAA |
| | | ATAAACTTTCGCTCCGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGACCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGCACATCAACCGCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 97 | Left ZFN-T2A- | GACTACAAAGACCATGACGGTGATTATAAAGATCATGACATCGATTA |
| | Right ZFN | CAAGGATGACGATGACAAGATGGCCCCCAAGAAGAAGAGGAAGGTCG |
| | with N-terminal | GCATTCATGGGGTACCCGCCGCTATGGCTGAGAGGCCCTTCCAGTGT |
| | modifications | CGAATCTGCATGCAGAACTTCAGTCAGTCCGGCAACCTGGCCCGCCA |
| | (comprising | CATCCGCACCCACACCGGCGAGAAGCCTTTTGCCTGTGACATTTGTG |
| | 3xFLAG, NLS, | GGAGGAAATTTGCCCTGAAGCAGAACCTGTGTATGCATACCAAGATA |
| | ZFP-L, FokI, T2A, | CACACGGGCGAGAAGCCCTTCCAGTGTCGAATCTGCATGCAGAAGTT |
| | 3xFLAG, NLS, | TGCCTGGCAGTCCAACCTGCAGAACCATACCAAGATACACACGGGCG |
| | ZFP-R, and FokI) | AGAAGCCCTTCCAGTGTCGAATCTGCATGCGTAACTTCAGTACCTCC |
| | (na) | GGCAACCTGACCCGCCACATCCGCACCCACACCGGCGAGAAGCCTTT |
| | ZFN-L | TGCCTGTGACATTTGTGGGAGGAAATTTGCCCGCCGCTCCCACCTGA |
| | Not diversified | CCTCCCATACCAAGATACACCTGCGGGGATCCCAGCTGGTGAAGAGC |
| | ZFN-R | GAGCTGGAGGAGAAGAAGTCCGAGCTGCGGCACAAGCTGAAGTACGT |
| | Not diversified | GCCCCACGAGTAGATCGAGCTGATCGAGATCGCCAGGAACAGCACCC |
| | | AGGACCGCATCCTGGAGATGAAGGTGATGGAGTTCTTCATGAAGGTG |
| | | TACGGCTACAGGGGAAAGCACCTGGGCGGAAGCAGAAAGCCTGACGG |
| | | CGCCATCTATACAGTGGGCAGCCCCATCGATTACGGCGTGATCGTGG |
| | | ACACAAAGGCCTACAGCGGCGGCTACAATCTGCCTATCGGCCAGGCC |
| | | GACGAGATGGAGAGATACGTGGAGGAGAACCAGACCCGGGATAAGCA |
| | | CCTCAACCCCAACGAGTGGTGGAAGGTGTACCCTAGCAGCGTGACCG |
| | | AGTTCAAGTTCCTGTTCGTGAGCGGCCACTTCAAGGGCAACTACAAG |
| | | GCCCAGCTGACCAGGCTGAACCACATCACCAACTGCGACGGCGCCGT |
| | | GCTGAGCGTGGAGGAGCTGCTGATCGGCGGCGAGATGATCAAAGCCG |
| | | GCACCCTGACACTGGAGGAGGTGCGGCGCAAGTTCAACAACGGCGAG |
| | | ATCAACTTCAGATCTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGACCATGACGGTGATTATAAAGATCATGACATCGATTACAAG |
| | | GATGACGATGACAAGATGGCCCCCAAGAAGAAGAGGAAGGTCGGCAT |
| | | TCATGGGGTACCCGCCGCTATGGCTGAGAGGCCCTTCCAGTGTCGAA |
| | | TCTGCATGCGTAACTTCAGTCAGTCCTCCGACCTGTCCCGCCACATC |
| | | CGCACCCACACCGGCGAGAAGCCTTTTGCCTGTGACATTTGTGGGAG |
| | | GAAATTTGCCCTGAAGCACAACCTGCTGAGCCATACCAAGATACACA |
| | | CGGGCGAGAAGCCCTTCCAGTGTCGAATCTGCATGCAGAACTTCAGT |
| | | GACCAGTCCAACCTGCGCGCCCACATCCGCACCCACACCGGCGAGAA |
| | | GCCTTTTGCCTGTGACATTTGTGGGAGGAAATTTGCCCGCAACTTCT |
| | | CCCTGACCATGCATACCAAGATACACACCGGAGAGCGCGGCTTCCAG |
| | | TGTCGAATCTGCATGCGTAACTTCAGTCTGCGCCACGACCTGGAGCG |
| | | CCACATCCGCACCCACACCGGCGAGAAGCCTTTTGCCTGTGACATTT |
| | | GTGGGAGGAAATTTGCCCACCGCTCCAACCTGAACAAGCATACCAAG |
| | | ATACACCTGCGGGGATCCCAGCTGGTGAAGAGCGAGCTGGAGGAGAA |
| | | GAAGTCCGAGCTGCGGCACAAGCTGAAGTACGTGCCCCACGAGTACA |
| | | TCGAGCTGATCGAGATCGCCAGGAACAGCACCCAGGACCGCATCCTG |
| | | GAGATGAAGGTGATGGAGTTCTTCATGAAGGTGTACGGCTACAGGGG |
| | | AAAGCACCTGGGCGGAAGCAGAAAGCCTGACGGCGCCATCTATACAG |
| | | TGGGCAGCCCCATCGATTACGGCGTGATCGTGGACACAAAGGCCTAC |
| | | AGCGGCGGCTACAATCTGAGCATCGGCCAGGCCGACGAGATGCAGAG |
| | | ATACGTGAAGGAGAACCAGACCCGGAATAAGGACATCAACCCCAACG |
| | | AGTGGTGGAAGGTGTACCCTAGCAGCGTGACCGAGTTCAAGTTCCTG |
| | | TTCGTGAGCGGCCACTTCAAGGGCAACTACAAGGCCCAGCTGACCAG |
| | | GCTGAACCGCAAAACCAACTGCAATGGCGCCGTGCTGAGCGTGGAGG |
| | | AGCTGCTGATCGGCGGCGAGATGATCAAAGCCGGCACCCTGACACTG |
| | | GAGGAGGTGCGGCGCAAGTTCAACAACGGCGAGATCAACTTC |
| 98 | Left ZFN-T2A- | GACTACAAGGACCACGACGGTGACTACAAAGACCACGATATAGACTA |
| | Right ZFN | TAAAGATGACGATGATAAGATGGCACCTAAAAAAAAGCGGAAAGTGG |
| | with N-terminal | GAATTCACGGCGTGCCCGCCGCCATGGCAGAGAGACCCTTTCAATGT |
| | modifications | AGAATCTGTATGCAAAATTTCTCTCAGAGTGGTAACCTTGCAAGACA |
| | (comprising | CATCAGAACTCATACAGGTGAGAAGCCGTTTGCATGTGAGATTTGCG |
| | 3xFLAG, NLS, | CTACGAAATTTGCCTTGAAACAGAATCTTTGTATGCACACAAAAATC |
| | ZFP-L, FokI, T2A, | CATACTGGTGAAAAGCCATTCCAATGCCGCATCTGTATGCAAAAATT |
| | 3xFLAG, NLS, | CGCGTGGCAGTCCAATTTGCAGAACCATACCAAGATTCACACGGGAG |
| | ZFP-R, and FokI) | AAAAACCATTTCAGTGCCGCATCTGCATGCGCAACTTTTCTACATCA |
| | (na) | GGAAACCTTAGACGAGATATTCGGAGGGAGACTGGAGAAAAACCATT |
| | ZFN-L | TGCTTGTGACATATGCGGCCGAAAATTTGCCAGACGCTCTCATCTCA |
| | Codon diversified | CCTCACATACTAAGATTCATTTGCGCGGAAGTCAGCTGGTGAAGAGT |
| | Version 2 | GAATTGGAAGAAAAAAAGTCAGAGCTGAGACACAAACTGAAATATGT |
| | ZFN-R | TCCACACGAGTAGATCGAGCTTATCGAGATAGCAAGAAACTCCACCC |
| | Codon diversified | AGGACAGAATTTTGGAAATGAAAGTTATGGAATTCTTTATGAAAGTG |
| | Version 4 | TATGGCTACAGGGGTAAACATCTGGGGGGATCAAGAAAGCCTGATGG |
| | | TGCAATTTACACAGTGGGCTCTCCTATCGACTACGGTGTGATCGTGG |
| | | ATACAAAGGCCTACTCTGGAGGATATAATTTGCCTATTGGACAAGCC |
| | | GATGAAATGGAAAGATATGTGGAGGAAAACCAGACTCGCGATAAGCA |
| | | CCTGAACCCAAATGAATGGTGGAAAGTGTACCCTTCATCTGTTACCG |
| | | AATTTAAATTTTTGTTCGTTTCCGGGCATTTCAAGGGGAACTACAAG |
| | | GCACAGCTGACGAGACTGAATCACATCACGAACTGCGACGGCGCTGT |
| | | ACTGTCCGTGGAAGAGCTTTTGATCGGGGGCGAAATGATTAAGGCCG |
| | | GCACACTGACGCTGGAGGAGGTGCGGCGAAAATTTAATAATGGCGAG |
| | | ATCAATTTTAGGAGTGGCAGCGGAGAGGGCAGAGGAAGCCTGCTCAC |
| | | CTGCGGTGACGTGGAGGAAAACCCTGGCCCTACGCGTGCCATGGACT |
| | | ACAAAGATCATGATGGCGACTACAAAGATCATGATATAGATTACAAA |
| | | GACGATGACGACAAAATGGCTCCAAAAAAAAAACGCAAGGTTGGAAT |
| | | ACACGGTGTACCTGCCGCTATGGCTGAAAGACCTTTCCAGTGTAGGA |
| | | TTTGCATGAGAAATTTTTCCCAATCATCCGACCTTTCAAGGCATATT |
| | | AGGACACACACCGGGGAAAAGCCATTTGCTTGTGATATCTGCGGGCG |
| | | CAAATTTGCTCTTAAGCACAATCTTCTTACCCACACCAAAATTCATA |
| | | CAGGAGAAAAACCTTTTCAATGTAGAATCTGCATGCAAAACTTTTCC |
| | | GATCAGTCAAATCTTAGAGCTCATATCAGAACCCATACCGGGGAGAA |
| | | ACCCTTTGCCTGCGACATATGCGGAAGAAAATTTGCTAGGAACTTTA |
| | | GTCTGACCATGCATACCAAAATTCATACCGGCGAACGCGGTTTCCAG |
| | | TGCAGGATTTGTATGAGAAATTTCTCACTGCGGCATGATCTTGAAAG |
| | | ACACATACGAACTCATACCGGAGAAAAGCCATTCGCTTGCGATATTT |
| | | GTGGTAGAAAATTTGCCCACAGGTCTAACCTTAATAAGCACACCAAG |
| | | ATTCATCTCAGAGGATCTCAGCTGGTCAAATCAGAACTTGAAGAGAA |
| | | AAAAAGCGAACTGAGACATAAACTGAAGTACGTGCCTCATGAATACA |
| | | TAGAGCTCATTGAAATAGCTAGGAATAGTACACAGGACAGGATACTT |
| | | GAAATGAAGGTAATGGAATTTTTCATGAAGGTTTATGGATACCGGGG |
| | | GAAACATCTCGGGGGCAGCAGAAAACCAGACGGAGCAATTTATACTG |
| | | TCGGGAGTCCTATAGATTATGGCGTTATCGTCGATACAAAGGCCTAT |
| | | TCCGGTGGGTACAACCTCTCAATTGGTCAGGCTGATGAGATGCAAAG |
| | | ATACGTCAAAGAAAACGAAAGGAGAAATAAACATATAAATCCCAATG |
| | | AATGGTGGAAAGTATACCCAAGTTCCGTGACTGAATTCAAGTTCCTT |
| | | TTCGTGTCTGGCCACTTTAAAGGAAATTATAAAGCACAATTGACTAG |
| | | ACTGAATAGAAAAACAAACTGTAACGGCGCAGTGCTGTCAGTGGAAG |
| | | AACTGCTCATAGGTGGAGAGATGATCAAGGCCGGGACACTTACTCTT |
| | | GAGGAAGTTAGAAGGAAGTTCAACAACGGCGAAATCAACTTT |
| 99 | Right ZFN-T2A- | GACTACAAAGATCATGATGGCGACTACAAAGATCATGATATAGATTA |
| | Left ZFN | CAAAGACGATGACGAGAAAATGGCTCCAAAAAAAAAACGCAAGGTTG |
| | with N-terminal | GAATACACGGTGTACCTGCCGCTATGGCTGAAAGACCTTTCCAGTGT |
| | modifications | AGGATTTGCATGAGAAATTTTTCCCAATCATCCGACCTTTCAAGGCA |
| | (comprising | TATTAGGACACACACCGGGGAAAAGCCATTTGCTTGTGATATCTGCG |
| | 3xFLAG, NLS, | GGCGCAAATTTGCTCTTAAGCACAATCTTCTTACCCACACCAAAATT |
| | ZFP-R, FokI, T2A, | CATACAGGAGAAAAACCTTTTCAATGTAGAATCTGCATGCAAAACTT |
| | 3xFLAG, NLS, | TTCCGATGAGTCAAATCTTAGAGCTCATATCAGAACCCATACCGGGG |
| | ZFP-L, and FokI) | AGAAACCCTTTGCCTGCGACATATGCGGAAGAAAATTTGCTAGGAAC |
| | (na) | TTTAGTCTGACCATGCATACCAAAATTCATACCGGCGAACGCGGTTT |
| | ZFN-R | CCAGTGCAGGATTTGTATGAGAAATTTCTCACTGCGGCATGATCTTG |
| | Codon diversified | AAAGACACATACGAACTCATACCGGAGAAAAGCCATTCGCTTGCGAT |
| | Version 4 | ATTTGTGGTAGAAAATTTGCCCACAGGTCTAACCTTAATAAGCACAC |
| | ZFN-L | CAAGATTCATCTCAGAGGATCTCAGCTGGTCAAATCAGAACTTGAAG |
| | Codon diversified | AGAAAAAAAGCGAACTGAGACATAAACTGAAGTACGTGCCTCATGAA |
| | Version 2 | TACATAGAGCTCATTGAAATAGCTAGGAATAGTAGACAGGAGAGGAT |
| | | ACTTGAAATGAAGGTAATGGAATTTTTCATGAAGGTTTATGGATACC |
| | | GGGGGAAACATCTCGGGGGCAGCAGAAAACCAGACGGAGCAATTTAT |
| | | ACTGTCGGGAGTCCTATAGATTATGGCGTTATCGTCGATACAAAGGC |
| | | CTATTCCGGTGGGTACAACCTCTCAATTGGTCAGGCTGATGAGATGC |
| | | AAAGATACGTCAAAGAAAACCAAACCAGAAATAAACATATAAATCCC |
| | | AATGAATGGTGGAAAGTATACCCAAGTTCCGTGAGTGAATTCAAGTT |
| | | CCTTTTCGTGTCTGGCCACTTTAAAGGAAATTATAAAGCACAATTGA |
| | | CTAGACTGAATAGAAAAACAAACTGTAACGGCGCAGTGCTGTCAGTG |
| | | GAAGAACTGCTCATAGGTGGAGAGATGATCAAGGCCGGGACACTTAC |
| | | TCTTGAGGAAGTTAGAAGGAAGTTCAACAAGGGCGAAATCAACTTTG |
| | | GCAGCGGAGAGGGCAGAGGAAGCCTGCTCACCTGCGGTGACGTGGAG |
| | | GAAAACCCTGGCCCTACGCGTGCCATGGACTACAAGGACCACGACGG |
| | | TGACTACAAAGACCACGATATAGACTATAAAGATGACGATGATAAGA |
| | | TGGCACCTAAAAAAAAGCGGAAAGTGGGAATTCACGGCGTGCCCGCC |
| | | GCCATGGCAGAGAGACCCTTTCAATGTAGAATCTGTATGCAAAATTT |
| | | CTCTCAGAGTGGTAACCTTGCAAGACACATCAGAACTCATACAGGTG |
| | | AGAAGCCGTTTGCATGTGACATTTGCGGTAGGAAATTTGCCTTGAAA |
| | | CAGAATCTTTGTATGCACACAAAAATCCATACTGGTGAAAAGCCATT |
| | | CCAATGCCGCATCTGTATGCAAAAATTCGCGTGGCAGTCCAATTTGC |
| | | AGAACCATACCAAGATTCACACGGGAGAAAAACCATTTCAGTGCCGC |
| | | ATCTGCATGCGCAACTTTTCTACATCAGGAAACCTTACACGACATAT |
| | | TCGGACGCACACTGGAGAAAAACCATTTGCTTGTGACATATGCGGCC |
| | | GAAAATTTGCCAGACGCTCTCATCTCACCTCACATACTAAGATTCAT |
| | | TTGCGCGGAAGTCAGCTGGTGAAGAGTGAATTGGAAGAAAAAAAGTC |
| | | AGAGCTGAGACACAAACTGAAATATGTTCCACACGAGTACATCGAGC |
| | | TTATCGAGATAGCAAGAAACTCCACCGAGGACAGAATTTTGGAAATG |
| | | AAAGTTATGGAATTCTTTATGAAAGTGTATGGCTACAGGGGTAAACA |
| | | TCTGGGGGGATCAAGAAAGCCTGATGGTGCAATTTACACAGTGGGCT |
| | | CTCCTATCGACTACGGTGTGATCGTGGATACAAAGGCCTACTCTGGA |
| | | GGATATAATTTGGCTATTGGACAAGCCGATGAAATGGAAAGATATGT |
| | | GGAGGAAAACCAGACTCGCGATAAGCACCTGAACCCAAATGAATGGT |
| | | GGAAAGTGTACCCTTCATCTGTTACCGAATTTAAATTTTTGTTCGTT |
| | | TCCGGGCATTTCAAGGGGAACTACAAGGCACAGCTGACGAGACTGAA |
| | | TCACATCACGAACTGCGACGGCGCTGTACTGTCCGTGGAAGAGCTTT |
| | | TGATCGGGGGCGAAATGATTAAGGCCGGCACACTGACGCTGGAGGAG |
| | | GTGCGGCGAAAATTTAATAATGGCGAGATCAATTTTAGGAGT |
| 100 | Right ZFN-T2A- | GTACCTGCTGCTATGGCTGAAAGACCTTTTCAATGTCGAATCTGCAT |
| | LeftZFN (na) | GAGGAATTTTAGTCAGTCATCCGACCTGAGCAGACACATTCGAAGCC |
| | ZFN-R | ATACTGGTGAAAAGCCATTTGCTTGCGATATATGTGGGAGAAAATTT |
| | Codon diversified | GCGTTGAAACACAATCTGCTGACCCATACCAAGATTCATACCGGAGA |
| | Version 1 | AAAACCATTCCAATGCCGCATTTGTATGCAGAACTTTAGTGACCAGT |
| | ZFN-L | CAAATCTCCGCGCTCACATTCGAACCCACACTGGCGAAAAACCCTTT |
| | Not diversified | GCTTGTGACATTTGCGGTCGGAAGTTTGCCCGAAATTTTTCTCTGAC |
| | | AATGCACACAAAAATCCACACCGGGGAACGCGGCTTTCAATGTAGGA |
| | | TCTGTATGAGAAATTTTAGCCTTAGACATGATTTGGAACGACATATC |
| | | AGGACCCATACAGGCGAGAAACCATTTGCGTGCGATATTTGTGGCAG |
| | | GAAATTCGCACATAGAAGTAATCTGAACAAGCATACAAAAATTCATC |
| | | TCAGAGGAAGTCAGCTGGTCAAAAGTGAACTGGAGGAAAAAAAGAGC |
| | | GAACTGAGACACAAACTGAAGTACGTGCCACACGAATATATTGAGCT |
| | | GATTGAGATCGCGAGGAACTCAACACAGGACCGCATTCTGGAGATGA |
| | | AAGTGATGGAGTTTTTCATGAAAGTATATGGATATAGAGGAAAACAC |
| | | CTTGGGGGTAGCCGAAAGCCGGACGGGGCGATCTACACTGTGGGGTC |
| | | ACCAATTGATTATGGCGTAATTGTCGATACCAAAGCCTACAGTGGGG |
| | | GGTACAATCTGAGTATAGGACAGGCTGATGAAATGCAACGATACGTT |
| | | AAGGAGAATCAGACTAGGAATAAACATATCAATCCAAATGAATGGTG |
| | | GAAAGTCTATCCCAGCAGCGTGACAGAATTTAAATTTTTGTTTGTCA |
| | | GTGGACACTTCAAGGGAAATTATAAGGCCCAGCTGAGTAGACTGAAT |
| | | AGGAAAACCAATTGTAATGGCGCAGTGCTTTCAGTGGAGGAACTGCT |
| | | CATTGGAGGTGAGATGATCAAGGCTGGAACCCTGACGCTGGAGGAGG |
| | | TGCGGAGGAAGTTTAACAATGGAGAAATTAACTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 101 | Right ZFN-T2A- | GTCCCAGCTGCCATGGCCGAGAGACCATTTCAATGTCGGATTTGCAT |
| | LeftZFN (na) | GCGCAATTTTTCCCAGTCCTCTGACCTTAGCCGGCATATTCGGACAC |
| | ZFN-R | ACACAGGTGAAAAACCCTTCGCATGCGACATTTGCGGAAGAAAATTC |
| | Codon diversified | GCTCTGAAACACAACCTGCTTACCCATACAAAGATCCAGACCGGCGA |
| | Version 2 | GAAACCGTTTCAATGCCGAATCTGTATGCAAAATTTTAGTGATCAAA |
| | ZFN-L | GTAATCTGAGAGCACATATTAGGACTCACACGGGCGAGAAGCCATTT |
| | Not diversified | GCGTGTGATATCTGCGGCCGAAAATTCGCCCGGAATTTCTCTCTGAC |
| | | AATGGACACCAAAATCCACACTGGGGAACGAGGCTTTCAATGTAGAA |
| | | TATGTATGCGGAATTTCAGTCTGAGGCACGACCTGGAGCGGCACATC |
| | | AGAACTCACACCGGAGAAAAACCATTCGCTTGTGATATTTGCGGGAG |
| | | GAAGTTCGCCCATAGGAGCAATCTCAATAAACACACCAAAATACATC |
| | | TTCGGGGTTCTCAACTGGTGAAATCCGAACTGGAAGAAAAGAAATCA |
| | | GAATTGCGGCATAAACTGAAGTATGTGCCCCATGAGTACATAGAACT |
| | | GATCGAGATCGCAAGGAACTCTACCCAGGACAGAATACTTGAAATGA |
| | | AGGTCATGGAATTTTTTATGAAAGTGTACGGCTACAGAGGAAAACAT |
| | | TTGGGAGGCAGTCGAAAACCAGATGGCGCAATCTATACAGTCGGGTC |
| | | CCCCATAGATTACGGAGTGATTGTCGACACAAAAGCCTATTCCGGAG |
| | | GATATAACCTTAGTATCGGCCAGGCCGACGAGATGCAACGCTATGTG |
| | | AAAGAAAACCAAACAAGAAATAAACATATCAATCCAAACGAGTGGTG |
| | | GAAGGTATATCCAAGCAGTGTCACAGAATTCAAATTCCTCTTCGTGA |
| | | GTGGGCACTTTAAAGGCAACTACAAAGCTCAATTGACCAGGCTCAAT |
| | | CGGAAAACTAATTGCAATGGCGCAGTCCTTAGCGTCGAAGAATTGCT |
| | | GATTGGCGGGGAAATGATTAAAGCAGGAACTTTGACCTTGGAGGAAG |
| | | TACGGAGAAAGTTTAACAACGGCGAGATTAATTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 102 | Right ZFN-T2A- | GTCCCTGCCGCCATGGCCGAGCGCCCCTTCCAATGCCGCATATGCAT |
| | LeftZFN (na) | GAGAAATTTCAGCCAAAGTAGCGACCTGTCACGACACATTAGAACTC |
| | ZFN-R | ATACGGGGGAGAAGCCATTTGCTTGCGATATTTGTGGCAGAAAATTC |
| | Codon diversified | GCACTCAAACACAACCTGCTCACACACACCAAGATACACACGGGAGA |
| | Version 3 | GAAGCCCTTCCAATGTAGAATATGTATGCAAAATTTCAGCGACCAAA |
| | ZFN-L | GTAATTTGAGAGCGCATATTCGAACTCACACCGGCGAAAAACCATTT |
| | Not diversified | GCCTGCGATATTTGTGGGAGGAAATTTGCCAGGAATTTTTCACTCAC |
| | | CATGCACACTAAGATCCACACTGGCGAGCGCGGCTTCCAATGCAGAA |
| | | TCTGTATGCGAAACTTCAGTCTGCGGCATGACCTGGAAAGACATATA |
| | | AGAACCCACACCGGAGAAAAACCCTTTGCCTGCGACATATGTGGTAG |
| | | AAAATTCGCACATCGGAGTAACCTTAACAAACATACAAAGATCCACT |
| | | TGAGAGGCAGTCAGCTGGTGAAATCTGAGCTGGAAGAGAAGAAATCT |
| | | GAACTGCGACATAAATTGAAGTACGTCCGAGACGAGTACATCGAGTT |
| | | GATCGAAATTGCCCGGAATAGCACCCAGGATAGAATATTGGAAATGA |
| | | AAGTAATGGAGTTTTTTATGAAGGTTTATGGTTACAGAGGCAAGCAC |
| | | CTTGGAGGAAGCAGGAAACCAGATGGGGCGATTTACACCGTTGGGAG |
| | | TCCCATCGATTACGGAGTCATCGTGGACACAAAGGCCTATTCCGGAG |
| | | GCTACAACCTCAGTATCGGGCAAGCCGATGAGATGCAGAGATATGTT |
| | | AAAGAAAATCAGACGCGAAACAAGCACATTAACCCAAACGAATGGTG |
| | | GAAAGTTTACCCTAGCTCAGTGACAGAATTTAAGTTTCTGTTTGTCA |
| | | GCGGCCACTTCAAGGGGAATTATAAAGCACAACTGACCCGCCTGAAC |
| | | CGAAAAACCAACTGTAACGGTGCTGTGCTGAGTGTCGAAGAGTTGCT |
| | | TATCGGAGGAGAGATGATAAAGGCCGGCACACTGACGCTTGAAGAGG |
| | | TACGGCGAAAATTCAATAACGGAGAGATTAATTTTGGCAGCGGAGAG |
| | | GGCAGAGGAAGCCTGCTCACCTGCGGTGACGTGGAGGAAAACCCTGG |
| | | CCCT |
| | | GCCGCTATGGCTGAGAGGCCCTTCCAGTGTCGAATCTGCATGCAGAA |
| | | CTTCAGTCAGTCCGGCAACCTGGCCCGCCACATCCGCACCCACACCG |
| | | GCGAGAAGCCTTTTGCCTGTGACATTTGTGGGAGGAAATTTGCCCTG |
| | | AAGCAGAACCTGTGTATGCATACCAAGATACACACGGGCGAGAAGCC |
| | | CTTCCAGTGTCGAATCTGCATGCAGAAGTTTGCCTGGCAGTCCAACC |
| | | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCT |
| 103 | Right ZFN-T2A- | GTACCTGCCGCTATGGCTGAAAGACCTTTCCAGTGTAGGATTTGCAT |
| | LeftZFN (na) | GAGAAATTTTTCCCAATCATCCGACCTTTCAAGGCATATTAGGACAC |
| | ZFN-R | ACACCGGGGAAAAGCCATTTGCTTGTGATATCTGCGGGCGCAAATTT |
| | Codon diversified | GCTCTTAAGCACAATCTTCTTACCCACACCAAAATTCATACAGGAGA |
| | Version 4 | AAAACCTTTTCAATGTAGAATCTGCATGCAAAACTTTTCCGATCAGT |
| | ZFN-L | CAAATCTTAGAGCTCATATCAGAACCCATACCGGGGAGAAACCCTTT |
| | Not diversified | GCCTGCGACATATGCGGAAGAAAATTTGCTAGGAACTTTAGTCTGAC |
| | | CATGCATACCAAAATTCATACCGGCGAACGCGGTTTCCAGTGCAGGA |
| | | TTTGTATGAGAAATTTCTCACTGCGGCATGATCTTGAAAGACACATA |
| | | CGAACTCATACCGGAGAAAAGCCATTCGCTTGCGATATTTGTGGTAG |
| | | AAAATTTGCCCACAGGTCTAACCTTAATAAGCACACCAAGATTCATC |
| | | TCAGAGGATCTCAGCTGGTCAAATCAGAACTTGAAGAGAAAAAAAGC |
| | | GAACTGAGACATAAACTGAAGTACGTGCCTCATGAATACATAGAGCT |
| | | CATTGAAATAGCTAGGAATAGTACACAGGACAGGATACTTGAAATGA |
| | | AGGTAATGGAATTTTTCATGAAGGTTTATGGATACCGGGGGAAACAT |
| | | CTCGGGGGCAGCAGAAAACCAGACGGAGCAATTTATACTGTCGGGAG |
| | | TCCTATAGATTATGGCGTTATCGTCGATACAAAGGCCTATTCCGGTG |
| | | GGTACAACCTCTCAATTGGTCAGGCTGATGAGATGCAAAGATACGTC |
| | | AAAGAAAACCAAACCAGAAATAAACATATAAATCCCAATGAATGGTG |
| | | GAAAGTATACCCAAGTTCCGTGACTGAATTCAAGTTCCTTTTCGTGT |
| | | CTGGCCACTTTAAAGGAAATTATAAAGCACAATTGAGTAGACTGAAT |
| | | AGAAAAACAAACTGTAACGGCGCAGTGCTGTCAGTGGAAGAACTGCT |
| | | CATAGGTGGAGAGATGATCAAGGCCGGGACACTTACTCTTGAGGAAG |
| | | TTAGAAGGAAGTTCAACAACGGCGAAATCAACTTTGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 104 | Right ZFN-T2A- | GTACCAGCAGCTATGGCCGAACGCCCTTTTCAATGCAGAATATGTAT |
| | LeftZFN (na) | GCGAAACTTCTCCCAAAGCTCTGATCTGTCAAGGCACATACGGACAC |
| | ZFN-R | ACACCGGCGAAAAACCCTTTGCATGTGAGATTTGTGGAAGAAAATTC |
| | Codon diversified | GCACTTAAACACAATCTCCTGAGTCATACAAAAATACATACAGGCGA |
| | Version 5 | AAAACCTTTCGAGTGCAGAATCTGTATGCAGAACTTTTCCGAGGAAT |
| | ZFN-L | CCAATCTTCGCGCCCACATTAGAACTCACACAGGGGAGAAACCTTTC |
| | Not diversified | GCTTGCGACATATGCGGAAGAAAATTTGCCAGAAATTTTTGAGTTAG |
| | | AATGCACACAAAAATACATACTGGGGAAAGAGGGTTTGAATGTCGAA |
| | | TCTGTATGAGAAATTTGAGTCTGCGCCATGATCTGGAGAGACATATA |
| | | AGAACACACACAGGAGAGAAACCTTTTGCTTGTGACATATGCGGCCG |
| | | AAAGTTTGCTCATAGATCTAATCTTAACAAACATACAAAGATCCATC |
| | | TTCGGGGTTCACAACTGGTCAAGTCAGAATTGGAAGAGAAAAAATCT |
| | | GAGCTGAGGGAGAAATTGAAATACGTTCCTGAGGAGTATATTGAACT |
| | | TATCGAGATAGCCCGGAATAGTAGACAAGATAGAATCTTGGAGATGA |
| | | AAGTTATGGAATTCTTTATGAAAGTCTATGGCTATAGGGGAAAACAC |
| | | CTGGGGGGTAGCAGGAAACCTGATGGAGCTATCTATACCGTAGGATC |
| | | ACCTATTGATTATGGAGTAATTGTGGACACTAAGGCATATTCCGGAG |
| | | GATATAATTTGAGTATTGGTGAGGCCGAGGAAATGCAACGATACGTG |
| | | AAGGAAAATCAGACCCGCAACAAACACATTAATCCCAATGAATGGTG |
| | | GAAGGTATACCCTAGTAGCGTTACAGAGTTTAAATTCCTTTTCGTCA |
| | | GCGGCCACTTTAAAGGAAATTATAAAGCACAACTCACCAGACTTAAT |
| | | CGAAAAACTAACTGTAACGGCGCCGTACTGTCAGTGGAGGAGCTGCT |
| | | CATTGGAGGCGAGATGATCAAGGCCGGTACTCTCACACTGGAAGAAG |
| | | TTAGAAGAAAGTTCAACAACGGGGAAATTAATTTCGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 105 | Right ZFN-T2A- | GTTCCCGCTGCTATGGCTGAGAGACCTTTCCAATGTAGGATCTGTAT |
| | LeftZFN (na) | GCGAAACTTCTCCCAGAGCTCCGACCTGAGTCGCCATATAAGAACCC |
| | ZFN-R | ATACCGGAGAAAAACCATTTGCTTGTGAGATTTGTGGCAGAAAGTTC |
| | Codon diversified | GCTCTTAAACACAACCTGCTTACACATACTAAAATACACACAGGGGA |
| | Version 6 | GAAACCCTTTCAATGCCGGATCTGTATGCAAAACTTTAGCGATCAAT |
| | ZFN-L | CAAACTTGCGAGCCCATATCCGCACTCACACCGGCGAGAAGCCTTTT |
| | Not diversified | GCATGCGATATATGTGGACGGAAATTTGCTAGAAACTTCTCATTGAC |
| | | CATGCATACAAAAATACACACCGGGGAACGAGGATTTCAATGTCGAA |
| | | TTTGTATGAGAAATTTTAGCCTTAGGCACGACTTGGAACGGCACATA |
| | | AGAACCCACACCGGAGAGAAGCCTTTTGCTTGTGATATTTGCGGCAG |
| | | AAAGTTCGCCCATCGGAGCAATCTTAACAAGCACACCAAGATTCATT |
| | | TGAGAGGTTCCGAGCTGGTCAAAAGCGAACTTGAAGAAAAGAAATCC |
| | | GAGCTTAGACACAAACTGAAATACGTGCCTCACGAGTATATTGAGCT |
| | | GATTGAAATAGCAAGGAATTCAACACAAGACAGGATCCTCGAAATGA |
| | | AGGTTATGGAGTTTTTCATGAAAGTTTACGGCTACAGAGGGAAGCAT |
| | | CTGGGCGGATCAAGAAAACCAGACGGCGCAATCTACACAGTTGGATC |
| | | CCCAATAGATTACGGAGTGATTGTTGAGACCAAGGCTTATTCAGGAG |
| | | GTTACAATCTGTCCATTGGTCAGGCCGATGAAATGCAAAGATATGTT |
| | | AAGGAAAATCAAACTCGAAACAAACACATTAATCCAAACGAATGGTG |
| | | GAAAGTATATCCAAGCTCCGTCACTGAATTTAAATTTTTGTTTGTAT |
| | | CCGGACATTTTAAGGGCAACTATAAGGCTCAACTGACCAGACTGAAT |
| | | AGGAAGACCAATTGTAACGGAGCTGTACTCAGCGTGGAAGAACTGCT |
| | | TATTGGAGGCGAAATGATTAAGGCTGGCACACTTACACTCGAAGAAG |
| | | TTAGAAGAAAATTCAACAATGGTGAGATAAACTTCGGCAGCGGAGAG |
| | | GGCAGAGGAAGCCTGCTCACCTGCGGTGACGTGGAGGAAAACCCTGG |
| | | CCCTGCCGCTATGGCTGAGAGGCCCTTCCAGTGTCGAATCTGCATGC |
| | | AGAACTTCAGTCAGTCCGGCAACCTGGCCCGCCACATCCGCACCCAC |
| | | ACCGGCGAGAAGCCTTTTGCCTGTGACATTTGTGGGAGGAAATTTGC |
| | | CCTGAAGCAGAACCTGTGTATGCATACCAAGATACACACGGGCGAGA |
| | | AGCCCTTCCAGTGTCGAATCTGCATGCAGAAGTTTGCCTGGCAGTCC |
| | | AACCTGCAGAACCATACCAAGATACACACGGGCGAGAAGCCCTTCCA |
| | | GTGTCGAATCTGCATGCGTAACTTCAGTACCTCCGGCAACCTGACCC |
| | | GCCACATCCGCACCCACACCGGCGAGAAGCCTTTTGCCTGTGACATT |
| | | TGTGGGAGGAAATTTGCCCGCCGCTCCCACCTGACCTCCCATACCAA |
| | | GATACACCTGCGGGGATCCCAGCTGGTGAAGAGCGAGCTGGAGGAGA |
| | | AGAAGTCCGAGCTGCGGCACAAGCTGAAGTACGTGCCCCACGAGTAC |
| | | ATCGAGCTGATCGAGATCGCCAGGAACAGCACCCAGGACCGCATCCT |
| | | GGAGATGAAGGTGATGGAGTTCTTCATGAAGGTGTACGGCTACAGGG |
| | | GAAAGCACCTGGGCGGAAGCAGAAAGCCTGACGGCGCCATCTATACA |
| | | GTGGGCAGCCCCATCGATTACGGCGTGATCGTGGACACAAAGGCCTA |
| | | CAGCGGCGGCTACAATCTGCCTATCGGCCAGGCCGACGAGATGGAGA |
| | | GATACGTGGAGGAGAACCAGACCCGGGATAAGCACCTCAACCCCAAC |
| | | GAGTGGTGGAAGGTGTACCCTAGCAGCGTGACCGAGTTCAAGTTCCT |
| | | GTTCGTGAGCGGCCACTTCAAGGGCAACTACAAGGCCCAGCTGACCA |
| | | GGCTGAACCACATCACCAACTGCGACGGCGCCGTGCTGAGCGTGGAG |
| | | GAGCTGCTGATCGGCGGCGAGATGATCAAAGCCGGCACCCTGACACT |
| | | GGAGGAGGTGCGGCGCAAGTTCAACAACGGCGAGATCAACTTCAGAT |
| | | CT |
| 106 | Right ZFN-T2A- | GTACCCGCCGCTATGGCTGAGAGGCCCTTCCAGTGTCGAATCTGCATGCGTAACTTC |
| | LeftZFN (na) | AGTCAGTCCTCCGACCTGTCCCGCCACATCCGCACCCACACCGGCGAGAAGCCTTTT |
| | ZFN-R | GCCTGTGACATTTGTGGGAGGAAATTTGCCCTGAAGCACAACCTGCTGACCCATACC |
| | Not diversified | AAGATACACACGGGCGAGAAGCCCTTCCAGTGTCGAATCTGCATGCAGAACTTCAGT |
| | ZFN-L | GACCAGTCCAACCTGCGCGCCCACATCCGCACCCACACCGGCGAGAAGCCTTTTGCC |
| | Not diversified | TGTGACATTTGTGGGAGGAAATTTGCCCGCAACTTCTCCCTGACCATGCATACCAAG |
| | | ATACACACCGGAGAGCGCGGCTTCCAGTGTCGAATCTGCATGCGTAACTTCAGTCTG |
| | | CGCCACGACCTGGAGCGCCACATCCGCACCCACACCGGCGAGAAGCCTTTTGCCTGT |
| | | GACATTTGTGGGAGGAAATTTGCCCACCGCTCCAACCTGAACAAGCATACCAAGATA |
| | | CACCTGCGGGGATCCCAGCTGGTGAAGAGCGAGCTGGAGGAGAAGAAGTCCGAGCTG |
| | | CGGCACAAGCTGAAGTACGTGCCCCACGAGTACATCGAGCTGATCGAGATCGCCAGG |
| | | AACAGCACCCAGGACCGCATCCTGGAGATGAAGGTGATGGAGTTCTTCATGAAGGTG |
| | | TACGGCTACAGGGGAAAGCACCTGGGCGGAAGCAGAAAGCCTGACGGCGCCATCTAT |
| | | ACAGTGGGCAGCCCCATCGATTACGGCGTGATCGTGGACACAAAGGCCTACAGCGGC |
| | | GGCTACAATCTGAGCATCGGCCAGGCCGACGAGATGCAGAGATACGTGAAGGAGAAC |
| | | CAGACCCGGAATAAGCACATCAACCCCAACGAGTGGTGGAAGGTGTACCCTAGCAGC |
| | | GTGACCGAGTTCAAGTTCCTGTTCGTGAGCGGCCACTTCAAGGGCAACTACAAGGCC |
| | | CAGCTGACCAGGCTGAACCGCAAAACCAACTGCAATGGCGCCGTGCTGAGCGTGGAG |
| | | GAGCTGCTGATCGGCGGCGAGATGATCAAAGCCGGCACCCTGACACTGGAGGAGGTG |
| | | CGGCGCAAGTTCAACAACGGCGAGATCAACTTCGGCAGCGGAGAGGGCAGAGGAAGC |
| | | CTGCTCACCTGCGGTGACGTGGAGGAAAACCCTGGCCCTGCCGCTATGGCTGAGAGG |
| | | CCCTTCCAGTGTCGAATCTGCATGCAGAACTTCAGTCAGTCCGGCAACCTGGCCCGC |
| | | CACATCCGCACCCACACCGGCGAGAAGCCTTTTGCCTGTGACATTTGTGGGAGGAAA |
| | | TTTGCCCTGAAGCAGAACCTGTGTATGCATACCAAGATACACACGGGCGAGAAGCCC |
| | | TTCCAGTGTCGAATCTGCATGCAGAAGTTTGCCTGGCAGTCCAACCTGCAGAACCAT |
| | | ACCAAGATACACACGGGCGAGAAGCCCTTCCAGTGTCGAATCTGCATGCGTAACTTC |
| | | AGTACCTCCGGCAACCTGACCCGCCACATCCGCACCCACACCGGCGAGAAGCCTTTT |
| | | GCCTGTGACATTTGTGGGAGGAAATTTGCCCGCCGCTCCCACCTGACCTCCCATACC |
| | | AAGATACACCTGCGGGGATCCCAGCTGGTGAAGAGCGAGCTGGAGGAGAAGAAGTCC |
| | | GAGCTGCGGCACAAGCTGAAGTACGTGCCCCACGAGTACATCGAGCTGATCGAGATC |
| | | GCCAGGAACAGCACCCAGGACCGCATCCTGGAGATGAAGGTGATGGAGTTCTTCATG |
| | | AAGGTGTACGGCTACAGGGGAAAGCACCTGGGCGGAAGCAGAAAGCCTGACGGCGCC |
| | | ATCTATACAGTGGGCAGCCCCATCGATTACGGCGTGATCGTGGACACAAAGGCCTAC |
| | | AGCGGCGGCTACAATCTGCCTATCGGCCAGGCCGACGAGATGGAGAGATACGTGGAG |
| | | GAGAACCAGACCCGGGATAAGCACCTCAACCCCAACGAGTGGTGGAAGGTGTACCCT |
| | | AGCAGCGTGACCGAGTTCAAGTTCCTGTTCGTGAGCGGCCACTTCAAGGGCAACTAC |
| | | AAGGCCCAGCTGACCAGGCTGAACCACATCACCAACTGCGACGGCGCCGTGCTGAGC |
| | | GTGGAGGAGCTGCTGATCGGCGGCGAGATGATCAAAGCCGGCACCCTGACACTGGAG |
| | | GAGGTGCGGCGCAAGTTCAACAACGGCGAGATCAACTTCAGATCT |
| 107 | Left ZFN-T2A- | GCAGCAATGGCCGAACGACCCTTCCAATGCAGAATATGTATGCAGAA |
| | Right ZFN (na) | TTTTTCTCAGAGCGGGAACCTGGCGAGGCACATAAGAACCCATACAG |
| | ZFN-L | GAGAGAAGCCATTCGCATGCGATATTTGCGGTAGAAAATTTGCACTC |
| | Codon diversified | AAACAAAATCTCTGTATGGAGACTAAAATCCATACAGGTGAAAAGCC |
| | Version 1 | TTTTCAGTGCAGGATTTGTATGCAAAAATTTGCTTGGCAAAGTAACT |
| | ZFN-R | TGCAGAACCACACAAAGATACACACAGGAGAGAAACCCTTCCAATGC |
| | Not diversified | CGAATCTGTATGCGCAACTTGAGTAGATCCGGAAATTTGAGTAGACA |
| | | TATTAGGACCCACACCGGCGAGAAGCCATTTGCCTGCGATATTTGTG |
| | | GAGGGAAATTCGGAGGAGGGAGCCATCTGAGGAGTCATACTAAGATT |
| | | CATCTCCGCGGCAGCCAGCTTGTGAAGTCCGAACTGGAGGAAAAGAA |
| | | GAGCGAACTGCGCCACAAATTGAAATACGTTCCGCATGAGTAGATAG |
| | | AGCTCATTGAAATCGCTAGAAACTCTACCCAAGACAGGATACTGGAA |
| | | ATGAAAGTGATGGAATTTTTCATGAAAGTTTATGGTTATAGGGGCAA |
| | | ACATCTGGGTGGCTCTCGCAAGCCCGATGGGGCCATTTATACTGTCG |
| | | GCTCACCTATCGACTATGGCGTCATTGTGGATACCAAGGCTTATTCT |
| | | GGAGGATACAACCTGCCCATCGGACAAGCAGACGAAATGGAAAGATA |
| | | CGTCGAGGAGAATCAAACCCGAGACAAGCATCTGAACCCAAACGAGT |
| | | GGTGGAAAGTGTACCCGAGCAGCGTTACTGAGTTCAAATTTCTCTTT |
| | | GTAAGCGGACATTTTAAAGGGAATTACAAAGCACAACTGAGTAGGCT |
| | | GAACCATATAACCAACTGTGACGGGGCCGTATTGAGTGTGGAAGAGC |
| | | TTCTGATTGGAGGAGAGATGATTAAGGCTGGCACACTGAGTCTCGAA |
| | | GAAGTGAGGCGCAAATTCAATAACGGTGAAATCAACTTCCGGTCTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGAGCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 108 | Left ZFN-T2A- | GCCGCCATGGCAGAGAGACCCTTTCAATGTAGAATCTGTATGCAAAA |
| | Right ZFN (na) | TTTCTCTCAGAGTGGTAAGCTTGCAAGACACATGAGAACTCATACAG |
| | ZFN-L | GTGAGAAGCCGTTTGCATGTGACATTTGCGGTAGGAAATTTGCCTTG |
| | Codon diversified | AAACAGAATCTTTGTATGCACACAAAAATCCATACTGGTGAAAAGCC |
| | Version 2 | ATTCCAATGCCGCATCTGTATGCAAAAATTCGCGTGGCAGTCCAATT |
| | ZFN-R | TGCAGAACCATACCAAGATTCACACGGGAGAAAAACCATTTCAGTGC |
| | Not diversified | CGCATCTGCATGCGCAACTTTTCTAGATGAGGAAACCTTAGACGAGA |
| | | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCGAGGAGAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAAGGAGACTCGCGATAAGCACCTGAACCGAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGAGCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 109 | Left ZFN-T2A- | GCCGCGATGGCAGAGAGACCATTTGAGTGTAGAATCTGTATGCAGAA |
| | Right ZFN (na) | CTTTTCCCAATGAGGAAACCTGGCACGAGACATTAGAACCCATACTG |
| | ZFN-L | GAGAAAAGCCGTTCGCTTGCGACATTTGCGGTAGAAAATTTGCTTTG |
| | Codon diversified | AAACAGAACTTGTGTATGCATACCAAGATTCATACCGGCGAAAAACC |
| | Version 3 | ATTTCAATGCAGGATTTGTATGCAGAAGTTCGCCTGGCAATCCAATT |
| | ZFN-R | TGCAGAATCATACTAAAATTCATACCGGAGAAAAACCATTCCAATGC |
| | Not diversified | CGCATTTGTATGAGAAACTTTTCTACCTCTGGCAATCTCACCAGACA |
| | | TATGAGAACACACACAGGCGAGAAACCGTTCGCATGCGATATCTGTG |
| | | GGCGAAAGTTTGCCAGAAGATCCCATCTCACATCACATACTAAAATA |
| | | CATTTGCGAGGAAGTCAACTGGTCAAGTCCGAACTGGAGGAAAAAAA |
| | | AAGTGAGCTGCGAGACAAGTTGAAGTACGTACCACACGAATACATCG |
| | | AGCTGATTGAGATAGCACGGAAGTCTAGCCAGGATAGAATACTGGAG |
| | | ATGAAAGTTATGGAATTCTTTATGAAGGTGTACGGATACAGGGGGAA |
| | | GCATCTTGGCGGGAGCCGGAAACCAGACGGAGCAATCTATACCGTCG |
| | | GGTCACCTATAGACTATGGAGTTATTGTCGATACAAAGGCCTATTCA |
| | | GGAGGTTATAATCTGCCAATCGGCCAAGCCGACGAGATGGAGAGGTA |
| | | CGTGGAGGAAAATCAGACCAGAGACAAGCACCTGAAGCCTAATGAAT |
| | | GGTGGAAAGTGTACCCTAGCAGCGTCACTGAGTTCAAATTCCTGTTC |
| | | GTCAGCGGTCATTTTAAAGGAAATTATAAAGCCCAGCTCACTAGACT |
| | | CAACCATATTAGAAACTGCGAGGGAGCCGTACTTAGCGTTGAAGAGT |
| | | TGCTTATCGGAGGAGAGATGATGAAAGCCGGAAGCCTCACACTTGAA |
| | | GAAGTGCGAAGAAAATTCAATAACGGAGAGATAAATTTTAGGAGTGG |
| | | CAGCGGAGAGGGCAGAGGAAGCCTGCTCACCTGCGGTGACGTGGAGG |
| | | AAAACCCTGGCCCTGCCGCTATGGCTGAGAGGCCCTTCCAGTGTCGA |
| | | ATCTGCATGCAGAACTTCAGTCAGTCCGGCAACCTGGCCCGCCACAT |
| | | CCGCACCCACACCGGCGAGAAGCCTTTTGCCTGTGACATTTGTGGGA |
| | | GGAAATTTGCCCTGAAGGAGAACCTGTGTATGCATACCAAGATACAC |
| | | ACGGGCGAGAAGCCCTTCCAGTGTCGAATCTGCATGCAGAAGTTTGC |
| | | CTGGCAGTCCAACCTGCAGAACCATACCAAGATACACACGGGCGAGA |
| | | AGCCCTTCCAGTGTCGAATCTGCATGCGTAACTTCAGTACCTCCGGC |
| | | AACCTGACCCGCCACATCCGCACCCACACCGGCGAGAAGCCTTTTGC |
| | | CTGTGACATTTGTGGGAGGAAATTTGCCCGCCGCTCCCACCTGACCT |
| | | CCCATACCAAGATACACCTGCGGGGATCCCAGCTGGTGAAGAGCGAG |
| | | CTGGAGGAGAAGAAGTCCGAGCTGCGGCACAAGCTGAAGTACGTGCC |
| | | CCACGAGTACATCGAGCTGATCGAGATCGCCAGGAACAGCACCCAGG |
| | | ACCGCATCCTGGAGATGAAGGTGATGGAGTTCTTCATGAAGGTGTAC |
| | | GGCTACAGGGGAAAGCACCTGGGCGGAAGCAGAAAGCCTGACGGCGC |
| | | CATCTATACAGTGGGCAGCCCCATCGATTACGGCGTGATCGTGGACA |
| | | CAAAGGCCTACAGCGGCGGCTACAATCTGCCTATCGGCCAGGCCGAC |
| | | GAGATGGAGAGATACGTGGAGGAGAACCAGACCCGGGATAAGCACCT |
| | | CAACCCCAACGAGTGGTGGAAGGTGTACCCTAGCAGCGTGACCGAGT |
| | | TCAAGTTCCTGTTCGTGAGCGGCCACTTCAAGGGCAACTACAAGGCC |
| | | CAGCTGACCAGGCTGAACCACATCACCAACTGCGACGGCGCCGTGCT |
| | | GAGCGTGGAGGAGCTGCTGATCGGCGGCGAGATGATCAAAGCCGGCA |
| | | CCCTGACACTGGAGGAGGTGCGGCGCAAGTTCAACAACGGCGAGATC |
| | | AACTTCAGATCT |
| 110 | Left ZFN-T2A- | GCAGCAATGGCCGAGAGACCTTTTCAGTGCAGGATTTGTATGCAAAA |
| | Right ZFN (na) | CTTCTCTCAGTCCGGTAACCTGGCCCGGCACATACGAACACATACCG |
| | ZFN-L | GCGAAAAACCCTTTGCTTGCGACATCTGCGGAAGAAAGTTCGCTCTT |
| | Codon diversified | AAACAGAACCTGTGCATGCATACAAAAATTCATACAGGTGAGAAGCC |
| | Version 4 | ATTCCAATGCAGAATATGTATGCAGAAATTCGCCTGGCAAAGCAACC |
| | ZFN-R | TGCAAAACCACACTAAGATCCACACAGGGGAAAAGCCTTTTCAATGT |
| | Not diversified | AGAATCTGTATGAGAAACTTTAGTAGATCCGGAAATCTCACACGAGA |
| | | TATCAGAACCCACACTGGAGAAAAACCTTTTGCCTGCGAGATCTGCG |
| | | GAAGAAAATTCGCCCGAAGGTCCCACTTGAGTAGTCATACCAAAATC |
| | | CACTTGCGAGGCTCACAGCTGGTTAAATCCGAACTTGAAGAAAAAAA |
| | | AAGTGAACTGCGGCATAAACTGAAGTATGTCCCCCATGAATATATCG |
| | | AACTGATAGAAATCGCCCGAAATAGCACGCAAGATAGAATCCTCGAA |
| | | ATGAAGGTTATGGAATTTTTCATGAAGGTCTATGGATATAGGGGCAA |
| | | GCACCTTGGCGGATCCCGGAAACCTGATGGAGCTATCTACACAGTGG |
| | | GCTCACCAATAGACTATGGAGTTATCGTCGATACAAAAGCATACAGC |
| | | GGAGGATAGAATTTGCCAATAGGTCAAGCAGATGAGATGGAAAGATA |
| | | CGTGGAGGAAAAGCAAACAAGAGATAAGCATCTGAAGCCCAACGAAT |
| | | GGTGGAAAGTGTACCCCAGTTCTGTAACCGAATTTAAGTTCTTGTTC |
| | | GTTTCAGGTCACTTCAAGGGTAATTACAAGGCTCAACTGAGTAGACT |
| | | CAACCATATTACAAATTGCGATGGTGCTGTGCTTTCCGTGGAAGAAT |
| | | TGCTGATTGGTGGAGAGATGATAAAAGCTGGTACCCTCACCTTGGAA |
| | | GAAGTGCGCAGAAAATTCAATAATGGCGAGATCAACTTCCGAAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGAGCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAcATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 111 | Left ZFN-T2A- | GCAGCAATGGCAGAGAGACCATTTCAGTGCAGAATATGTATGCAAAA |
| | Right ZFN (na) | CTTCTCCCAGAGCGGTAATCTGGCTAGGCATATTAGAACACACACCG |
| | ZFN-L | GGGAAAAACCTTTCGCTTGCGATATATGTGGTAGAAAGTTCGCCCTC |
| | Codon diversified | AAACAGAATCTGTGCATGCACACTAAAATCCATACAGGAGAAAAGCC |
| | Version 5 | CTTTCAGTGTAGAATTTGTATGCAGAAATTTGCTTGGCAGTCAAATT |
| | ZFN-R | TGCAAAATCACACCAAAATACACACAGGAGAAAAACCATTTCAGTGT |
| | Not diversified | AGAATATGTATGAGAAATTTTTCCACTTCCGGAAATCTGAGCAGACA |
| | | TATACGGACACACACTGGGGAAAAGCCCTTCGCTTGCGACATCTGCG |
| | | GAAGAAAGTTCGCTAGACGGTCCCACTTGAGATCCCACACTAAGATA |
| | | CATCTTCGCGGTAGCCAACTGGTGAAAAGTGAACTGGAGGAAAAAAA |
| | | ATCTGAGCTGAGACATAAACTGAAATACGTACCACATGAATACATAG |
| | | AACTTATAGAAATAGCTAGGAACTCCACCCAGGACAGAATACTTGAA |
| | | ATGAAGGTCATGGAGTTTTTTATGAAAGTTTACGGATACAGGGGCAA |
| | | ACACCTTGGAGGGTCTCGGAAGCCTGATGGCGCAATTTATACCGTGG |
| | | GTAGCCCTATAGATTATGGAGTGATTGTGGATACAAAGGCTTACAGT |
| | | GGCGGCTATAATTTGCCTATCGGACAGGCCGATGAGATGGAAAGATA |
| | | CGTTGAAGAAAACCAAACACGAGATAAGCATCTGAACCCGAATGAAT |
| | | GGTGGAAAGTGTATCCTTCAAGCGTTACCGAGTTTAAGTTCCTCTTC |
| | | GTTTCTGGGCATTTCAAGGGCAACTACAAAGCTCAGCTTACAAGACT |
| | | CAACCACATAACGAATTGTGATGGAGGAGTCCTGAGCGTGGAAGAAC |
| | | TCCTTATTGGGGGTGAGATGATTAAAGCAGGGACCCTTACTCTTGAA |
| | | GAGGTTAGAAGAAAATTCAATAACGGAGAGATTAATTTTAGAAGTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGAGCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 112 | Left ZFN-T2A- | GCAGCCATGGCCGAACGCCCATTTCAATGTAGAATTTGTATGCAGAA |
| | Right ZFN (na) | TTTTTCACAATCAGGAAACCTGGCTAGAGATATCAGAACACATACTG |
| | ZFN-L | GAGAAAAGCCCTTTGCTTGTGATATCTGTGGAAGGAAATTCGCCCTG |
| | Codon diversified | AAACAAAACCTCTGTATGCACACAAAGATCCACACCGGCGAAAAGCC |
| | Version 6 | TTTCCAGTGTAGGATATGCATGCAAAAATTCGCCTGGCAGTCCAATC |
| | ZFN-R | TGCAGAACCATACCAAAATTCATACTGGTGAAAAGCCATTTCAGTGC |
| | Not diversified | AGAATATGTATGAGAAACTTTAGCACTTCAGGAAATCTCACAAGACA |
| | | TATAAGAACACATACAGGGGAAAAACCTTTTGCTTGCGATATCTGCG |
| | | GCAGGAAATTGGCTCGGAGAAGTCATCTCACAAGCCATACAAAAATC |
| | | CACCTGCGAGGAAGCCAGCTGGTCAAGTCTGAACTGGAAGAAAAAAA |
| | | AAGCGAACTGCGGCATAAACTCAAATACGTCCCACATGAATACATTG |
| | | AGCTCATCGAAATTGCTAGAAACTCTAGTCAAGATAGGATATTGGAG |
| | | ATGAAGGTAATGGAATTCTTCATGAAGGTTTATGGATATAGAGGAAA |
| | | ACATCTTGGAGGCAGTAGGAAACCCGATGGCGCTATCTACACCGTAG |
| | | GGAGTCCAATCGACTACGGCGTGATTGTTGACACCAAAGCCTATTCT |
| | | GGAGGGTATAATCTCCCAATTGGACAGGCAGATGAGATGGAAAGATA |
| | | TGTAGAAGAAAATCAGACAAGAGATAAGCACCTTAAGCCTAAGGAGT |
| | | GGTGGAAAGTGTACCCAAGCAGTGTTACTGAATTTAAATTTCTTTTT |
| | | GTATCAGGACACTTTAAAGGCAATTACAAAGCACAACTGACCAGACT |
| | | CAATCACATTACCAATTGCGACGGAGCCGTACTGAGCGTGGAGGAGT |
| | | TGCTGATCGGAGGCGAAATGATTAAAGCTGGCACTCTGACCCTGGAA |
| | | GAAGTAAGAAGAAAGTTCAATAATGGAGAAATAAACTTTCGCTCCGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGAGCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 113 | Left ZFN-T2A- | GCCGCTATGGCTGAGAGGCCCTTCCAGTGTCGAATCTGCATGCAGAA |
| | Right ZFN (na) | CTTCAGTCAGTCCGGCAACCTGGCCCGCCACATCCGCACCCACACCG |
| | ZFN-L | GCGAGAAGCCTTTTGCCTGTGACATTTGTGGGAGGAAATTTGCCCTG |
| | Not diversified | AAGCAGAACCTGTGTATGCATACCAAGATACACACGGGCGAGAAGCC |
| | ZFN-R | CTTCCAGTGTCGAATCTGCATGCAGAAGTTTGCCTGGCAGTCCAACC |
| | Not diversified | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGGGGATCCCAGCTGGTGAAGAGCGAGCTGGAGGAGAAGAA |
| | | GTCCGAGCTGCGGCACAAGCTGAAGTACGTGCCCCACGAGTACATCG |
| | | AGCTGATCGAGATCGCCAGGAACAGCACCCAGGACCGCATCCTGGAG |
| | | ATGAAGGTGATGGAGTTCTTCATGAAGGTGTACGGCTACAGGGGAAA |
| | | GCACCTGGGCGGAAGCAGAAAGCCTGACGGCGCCATCTATACAGTGG |
| | | GCAGCCCCATCGATTACGGCGTGATCGTGGACACAAAGGCCTACAGC |
| | | GGCGGCTACAATCTGCCTATCGGCCAGGCCGACGAGATGGAGAGATA |
| | | CGTGGAGGAGAACCAGACCCGGGATAAGCACCTCAACCCCAACGAGT |
| | | GGTGGAAGGTGTACCCTAGCAGCGTGACCGAGTTCAAGTTCCTGTTC |
| | | GTGAGCGGCCACTTCAAGGGCAACTACAAGGCCCAGCTGACCAGGCT |
| | | GAACCACATCACCAACTGCGACGGCGCCGTGCTGAGCGTGGAGGAGC |
| | | TGCTGATCGGCGGCGAGATGATCAAAGCCGGCACCCTGACACTGGAG |
| | | GAGGTGCGGCGCAAGTTCAACAACGGCGAGATCAACTTCAGATCTGG |
| | | CAGCGGAGAGGGCAGAGGAAGCCTGCTCACCTGCGGTGACGTGGAGG |
| | | AAAACCCTGGCCCTGTACCCGCCGCTATGGCTGAGAGGCCCTTCCAG |
| | | TGTCGAATCTGCATGCGTAACTTCAGTCAGTCCTCCGACCTGTCCCG |
| | | CCACATCCGCACCCACACCGGCGAGAAGCCTTTTGCCTGTGACATTT |
| | | GTGGGAGGAAATTTGCCCTGAAGCACAACCTGCTGACCCATACCAAG |
| | | ATACACACGGGCGAGAAGCCCTTCCAGTGTCGAATCTGCATGCAGAA |
| | | CTTCAGTGACCAGTCCAACCTGCGCGCCCACATCCGCACCCACACCG |
| | | GCGAGAAGCCTTTTGCCTGTGACATTTGTGGGAGGAAATTTGCCCGC |
| | | AACTTCTCCCTGACCATGCATACCAAGATACACACCGGAGAGCGCGG |
| | | CTTCCAGTGTCGAATCTGCATGCGTAACTTCAGTCTGCGCCACGACC |
| | | TGGAGCGCCACATCCGCACCCACACCGGCGAGAAGCCTTTTGCCTGT |
| | | GACATTTGTGGGAGGAAATTTGCCCACCGCTCCAACCTGAACAAGCA |
| | | TACCAAGATACACCTGCGGGGATCCCAGCTGGTGAAGAGCGAGCTGG |
| | | AGGAGAAGAAGTCCGAGCTGCGGCACAAGCTGAAGTACGTGCCCCAC |
| | | GAGTAGATCGAGCTGATCGAGATCGCGAGGAACAGCACCGAGGAGCG |
| | | CATCCTGGAGATGAAGGTGATGGAGTTCTTCATGAAGGTGTACGGCT |
| | | ACAGGGGAAAGCACCTGGGCGGAAGCAGAAAGCCTGACGGCGCCATC |
| | | TATACAGTGGGCAGCCCCATCGATTACGGCGTGATCGTGGACACAAA |
| | | GGCCTACAGCGGCGGCTACAATCTGAGCATCGGCCAGGCCGACGAGA |
| | | TGCAGAGATACGTGAAGGAGAACCAGACCCGGAATAAGCACATCAAC |
| | | CCCAACGAGTGGTGGAAGGTGTACCCTAGCAGCGTGACCGAGTTCAA |
| | | GTTCCTGTTCGTGAGCGGCCACTTCAAGGGCAACTACAAGGCCCAGC |
| | | TGACCAGGCTGAACCGCAAAACCAACTGCAATGGCGCCGTGCTGAGC |
| | | GTGGAGGAGCTGCTGATCGGCGGCGAGATGATCAAAGCCGGCACCCT |
| | | GACACTGGAGGAGGTGCGGCGCAAGTTCAACAACGGCGAGATCAACT |
| | | TC |
| 114 | Left ZFN-T2A- | GCCGCCATGGCAGAGAGACCCTTTCAATGTAGAATCTGTATGCAAAA |
| | Right ZFN (na) | TTTCTCTCAGAGTGGTAAGCTTGCAAGACACATGAGAACTCATACAG |
| | ZFN-L | GTGAGAAGCCGTTTGCATGTGACATTTGCGGTAGGAAATTTGCCTTG |
| | Codon diversified | AAACAGAATCTTTGTATGCACACAAAAATCCATACTGGTGAAAAGCC |
| | Version 2 | ATTCCAATGCCGCATCTGTATGCAAAAATTCGCGTGGCAGTCCAATT |
| | ZFN-R | TGGAGAACCATACCAAGATTCACACGGGAGAAAAACCATTTCAGTGC |
| | Codon diversified | CGCATCTGCATGCGCAACTTTTCTAGATGAGGAAACCTTAGACGAGA |
| | Version 4 | TATTCGGACGCACACTGGAGAAAAACCATTTGCTTGTGACATATGCG |
| | | GCCGAAAATTTGCCAGACGCTCTCATCTCACCTCACATACTAAGATT |
| | | CATTTGCGCGGAAGTCAGCTGGTGAAGAGTGAATTGGAAGAAAAAAA |
| | | GTCAGAGCTGAGACACAAACTGAAATATGTTCCACACGAGTACATCG |
| | | AGCTTATCGAGATAGCAAGAAACTCCACCGAGGAGAGAATTTTGGAA |
| | | ATGAAAGTTATGGAATTCTTTATGAAAGTGTATGGCTACAGGGGTAA |
| | | ACATCTGGGGGGATCAAGAAAGCCTGATGGTGCAATTTACACAGTGG |
| | | GCTCTCCTATCGACTACGGTGTGATCGTGGATACAAAGGCCTACTCT |
| | | GGAGGATATAATTTGCCTATTGGACAAGCCGATGAAATGGAAAGATA |
| | | TGTGGAGGAAAAGGAGACTCGCGATAAGCACCTGAACCGAAATGAAT |
| | | GGTGGAAAGTGTACCCTTCATCTGTTACCGAATTTAAATTTTTGTTC |
| | | GTTTCCGGGCATTTCAAGGGGAACTACAAGGCACAGCTGACGAGACT |
| | | GAATCACATCACGAACTGCGACGGCGCTGTACTGTCCGTGGAAGAGC |
| | | TTTTGATCGGGGGCGAAATGATTAAGGCCGGCACACTGACGCTGGAG |
| | | GAGGTGCGGCGAAAATTTAATAATGGCGAGATCAATTTTAGGAGTGG |
| | | CAGCGGAGAGGGCAGAGGAAGCCTGCTCACCTGCGGTGACGTGGAGG |
| | | AAAACCCTGGCCCTGTACCTGCCGCTATGGCTGAAAGACCTTTCCAG |
| | | TGTAGGATTTGCATGAGAAATTTTTCCCAATCATCCGACCTTTCAAG |
| | | GCATATTAGGACACACACCGGGGAAAAGCCATTTGCTTGTGATATCT |
| | | GCGGGCGCAAATTTGCTCTTAAGCACAATCTTCTTACCCACACCAAA |
| | | ATTCATACAGGAGAAAAACCTTTTCAATGTAGAATCTGCATGCAAAA |
| | | CTTTTCCGATGAGTGAAATCTTAGAGCTCATATGAGAACCCATACCG |
| | | GGGAGAAACCCTTTGCCTGCGACATATGCGGAAGAAAATTTGCTAGG |
| | | AACTTTAGTCTGACCATGCATACCAAAATTCATACCGGCGAACGCGG |
| | | TTTCCAGTGCAGGATTTGTATGAGAAATTTCTCACTGCGGCATGATC |
| | | TTGAAAGACACATACGAACTCATACCGGAGAAAAGCCATTCGCTTGC |
| | | GATATTTGTGGTAGAAAATTTGCCCACAGGTCTAACCTTAATAAGGA |
| | | CACCAAGATTCATCTCAGAGGATCTCAGCTGGTCAAATCAGAACTTG |
| | | AAGAGAAAAAAAGCGAACTGAGACATAAACTGAAGTACGTGCCTCAT |
| | | GAATACATAGAGCTCATTGAAATAGCTAGGAATAGTACACAGGACAG |
| | | GATACTTGAAATGAAGGTAATGGAATTTTTCATGAAGGTTTATGGAT |
| | | ACCGGGGGAAACATCTCGGGGGCAGCAGAAAACCAGACGGAGCAATT |
| | | TATACTGTCGGGAGTCCTATAGATTATGGCGTTATCGTCGATACAAA |
| | | GGCCTATTCCGGTGGGTACAACCTCTCAATTGGTCAGGCTGATGAGA |
| | | TGCAAAGATACGTCAAAGAAAACCAAACCAGAAATAAACATATAAAT |
| | | CCCAATGAATGGTGGAAAGTATACCCAAGTTCCGTGAGTGAATTCAA |
| | | GTTCCTTTTCGTGTCTGGCCACTTTAAAGGAAATTATAAAGCACAAT |
| | | TGACTAGACTGAATAGAAAAACAAACTGTAACGGCGGAGTGCTGTCA |
| | | GTGGAAGAACTGCTCATAGGTGGAGAGATGATCAAGGCCGGGACACT |
| | | TACTCTTGAGGAAGTTAGAAGGAAGTTCAACAACGGCGAAATCAACT |
| | | TT |
| 115 | Right ZFN-T2A- | GTACCTGCCGCTATGGCTGAAAGACCTTTCCAGTGTAGGATTTGCAT |
| | LeftZFN (na) | GAGAAATTTTTCCGAATCATCCGAGCTTTGAAGGCATATTAGGAGAC |
| | ZFN-R | ACACCGGGGAAAAGCCATTTGCTTGTGATATCTGCGGGCGCAAATTT |
| | Codon diversified | GCTCTTAAGCACAATCTTCTTAGCCACACCAAAATTCATACAGGAGA |
| | Version 4 | AAAACCTTTTCAATGTAGAATCTGCATGCAAAACTTTTCCGATCAGT |
| | ZFN-L | CAAATCTTAGAGCTCATATCAGAACCCATACCGGGGAGAAACCCTTT |
| | Codon diversified | GCCTGCGACATATGCGGAAGAAAATTTGCTAGGAACTTTAGTCTGAC |
| | Version 2 | CATGCATACCAAAATTCATACCGGCGAACGCGGTTTCCAGTGCAGGA |
| | | TTTGTATGAGAAATTTCTCACTGCGGCATGATCTTGAAAGACACATA |
| | | CGAACTCATACCGGAGAAAAGCCATTCGCTTGCGATATTTGTGGTAG |
| | | AAAATTTGCCCACAGGTCTAACCTTAATAAGCACACCAAGATTCATC |
| | | TCAGAGGATCTCAGCTGGTGAAATCAGAACTTGAAGAGAAAAAAAGC |
| | | GAAGTGAGACATAAACTGAAGTACGTGCCTCATGAATACATAGAGCT |
| | | CATTGAAATAGCTAGGAATAGTACACAGGACAGGATACTTGAAATGA |
| | | AGGTAATGGAATTTTTCATGAAGGTTTATGGATACCGGGGGAAACAT |
| | | CTCGGGGGCAGCAGAAAACCAGACGGAGCAATTTATACTGTCGGGAG |
| | | TCCTATAGATTATGGCGTTATCGTCGATACAAAGGCCTATTCCGGTG |
| | | GGTACAACCTCTCAATTGGTCAGGCTGATGAGATGCAAAGATACGTC |
| | | AAAGAAAACCAAACGAGAAATAAACATATAAATCCCAATGAATGGTG |
| | | GAAAGTATACCCAAGTTCCGTGACTGAATTCAAGTTCCTTTTCGTGT |
| | | CTGGCCACTTTAAAGGAAATTATAAAGCACAATTGAGTAGACTGAAT |
| | | AGAAAAACAAACTGTAACGGCGCAGTGCTGTCAGTGGAAGAACTGCT |
| | | CATAGGTGGAGAGATGATCAAGGCCGGGACACTTACTCTTGAGGAAG |
| | | TTAGAAGGAAGTTCAACAACGGCGAAATCAACTTTGGCAGCGGAGAG |
| | | GGCAGAGGAAGCCTGCTCACCTGCGGTGACGTGGAGGAAAACCCTGG |
| | | CCCTGCCGCCATGGCAGAGAGACCCTTTCAATGTAGAATCTGTATGC |
| | | AAAATTTCTCTCAGAGTGGTAACCTTGCAAGACACATCAGAACTCAT |
| | | ACAGGTGAGAAGCCGTTTGCATGTGACATTTGCGGTAGGAAATTTGC |
| | | CTTGAAACAGAATCTTTGTATGCACACAAAAATCCATACTGGTGAAA |
| | | AGCCATTCCAATGCCGCATCTGTATGCAAAAATTCGCGTGGCAGTCC |
| | | AATTTGCAGAACCATACCAAGATTCACACGGGAGAAAAACCATTTCA |
| | | GTGCCGCATCTGCATGCGCAACTTTTCTACATCAGGAAACCTTACAC |
| | | GACATATTCGGACGCACACTGGAGAAAAACCATTTGCTTGTGACATA |
| | | TGCGGCCGAAAATTTGCCAGACGCTCTCATCTCACCTCACATACTAA |
| | | GATTCATTTGCGCGGAAGTCAGCTGGTGAAGAGTGAATTGGAAGAAA |
| | | AAAAGTCAGAGCTGAGACACAAACTGAAATATGTTCGAGACGAGTAG |
| | | ATCGAGCTTATCGAGATAGCAAGAAACTCCACCGAGGAGAGAATTTT |
| | | GGAAATGAAAGTTATGGAATTCTTTATGAAAGTGTATGGCTACAGGG |
| | | GTAAACATCTGGGGGGATCAAGAAAGCCTGATGGTGCAATTTACACA |
| | | GTGGGCTCTCCTATCGACTACGGTGTGATCGTGGATACAAAGGCCTA |
| | | CTCTGGAGGATATAATTTGCCTATTGGACAAGCCGATGAAATGGAAA |
| | | GATATGTGGAGGAAAACGAGACTCGCGATAAGCACCTGAACCGAAAT |
| | | GAATGGTGGAAAGTGTACCCTTCATCTGTTACCGAATTTAAATTTTT |
| | | GTTCGTTTCCGGGCATTTCAAGGGGAACTACAAGGCACAGCTGACGA |
| | | GACTGAATCACATCACGAACTGCGACGGCGCTGTACTGTCCGTGGAA |
| | | GAGCTTTTGATCGGGGGCGAAATGATTAAGGCCGGCACACTGACGCT |
| | | GGAGGAGGTGCGGCGAAAATTTAATAATGGCGAGATCAATTTTAGGA |
| | | GT |
| 116 | Left ZFP | GCCGCTATGGCTGAGAGGCCCTTCCAGTGTCGAATCTGCATGCAGAA |
| | (ZFP-L) (na) | CTTCAGTCAGTCCGGCAACCTGGCCCGCCACATCCGCACCCACACCG |
| | Not diversified | GCGAGAAGCCTTTTGCCTGTGACATTTGTGGGAGGAAATTTGCCCTG |
| | | AAGCAGAACCTGTGTATGCATACCAAGATACACACGGGCGAGAAGCC |
| | | CTTCCAGTGTCGAATCTGCATGCAGAAGTTTGCCTGGCAGTCCAACC |
| | | TGCAGAACCATACCAAGATACACACGGGCGAGAAGCCCTTCCAGTGT |
| | | CGAATCTGCATGCGTAACTTCAGTACCTCCGGCAACCTGACCCGCCA |
| | | CATCCGCACCCACACCGGCGAGAAGCCTTTTGCCTGTGACATTTGTG |
| | | GGAGGAAATTTGCCCGCCGCTCCCACCTGACCTCCCATACCAAGATA |
| | | CACCTGCGG |
| 117 | Left ZFP | GCAGCAATGGCCGAACGACCCTTCCAATGCAGAATATGTATGCAGAA |
| | (ZFP-L) (na) | TTTTTCTCAGAGCGGGAACCTGGCGAGGCACATAAGAACCCATACAG |
| | Codon diversified | GAGAGAAGCCATTCGCATGCGATATTTGCGGTAGAAAATTTGCACTC |
| | Version 1 | AAACAAAATCTCTGTATGGAGACTAAAATCCATACAGGTGAAAAGCC |
| | | TTTTCAGTGCAGGATTTGTATGCAAAAATTTGCTTGGCAAAGTAACT |
| | | TGCAGAACCACACAAAGATACACACAGGAGAGAAACCCTTCCAATGC |
| | | CGAATCTGTATGCGCAACTTGAGTAGATCCGGAAATTTGAGTAGACA |
| | | TATTAGGACCCACACCGGCGAGAAGCCATTTGCCTGCGATATTTGTG |
| | | GAGGGAAATTCGCACGAGGGAGCCATCTGAGGAGTCATACTAAGATT |
| | | CATCTCCGC |
| 118 | Left ZFP | GCCGCCATGGCAGAGAGACCCTTTCAATGTAGAATCTGTATGCAAAA |
| | (ZFP-L) (na) | TTTCTCTCAGAGTGGTAACCTTGCAAGACACATCAGAACTCATACAG |
| | Codon diversified | GTGAGAAGCCGTTTGCATGTGACATTTGCGGTAGGAAATTTGCCTTG |
| | Version 2 | AAACAGAATCTTTGTATGCACACAAAAATCCATACTGGTGAAAAGCC |
| | | ATTCCAATGCCGCATCTGTATGCAAAAATTCGCGTGGCAGTCCAATT |
| | | TGCAGAACCATACCAAGATTGAGACGGGAGAAAAACCATTTCAGTGC |
| | | CGCATCTGCATGCGCAACTTTTCTAGATGAGGAAACCTTAGACGAGA |
| | | TATTCGGACGCACACTGGAGAAAAACCATTTGCTTGTGACATATGCG |
| | | GCCGAAAATTTGCGAGACGCTCTCATCTCACCTCACATACTAAGATT |
| | | CATTTGCGC |
| 119 | Left ZFP | GCCGCGATGGCAGAGAGACCATTTGAGTGTAGAATCTGTATGCAGAA |
| | (ZFP-L) (na) | CTTTTCCCAATGAGGAAACCTGGCACGAGACATTAGAACCCATACTG |
| | | GAGAAAAGCCGTTCGCTTGCGACATTTGCGGTAGAAAATTTGCTTTG |
| | Codon diversified | AAACAGAACTTGTGTATGCATACCAAGATTCATACCGGCGAAAAACC |
| | Version 3 | ATTTCAATGCAGGATTTGTATGCAGAAGTTCGCCTGGCAATCCAATT |
| | | TGCAGAATCATACTAAAATTCATACCGGAGAAAAACCATTCCAATGC |
| | | CGCATTTGTATGAGAAACTTTTCTACCTCTGGCAATCTCACCAGACA |
| | | TATCAGAACACACACAGGCGAGAAACCGTTCGCATGCGATATCTGTG |
| | | GGCGAAAGTTTGCCAGAAGATCCCATCTCACATCACATACTAAAATA |
| | | CATTTGCGA |
| 120 | Left ZFP | GCAGCAATGGCCGAGAGACCTTTTCAGTGCAGGATTTGTATGCAAAA |
| | (ZFP-L) (na) | CTTCTCTCAGTCCGGTAACCTGGCCCGGCACATACGAACACATACCG |
| | Codon diversified | GCGAAAAACCCTTTGCTTGCGACATCTGCGGAAGAAAGTTCGCTCTT |
| | Version 4 | AAACAGAACCTGTGCATGCATACAAAAATTCATACAGGTGAGAAGCC |
| | | ATTCCAATGCAGAATATGTATGCAGAAATTCGCCTGGCAAAGCAACC |
| | | TGCAAAACCACACTAAGATCCACACAGGGGAAAAGCCTTTTCAATGT |
| | | AGAATCTGTATGAGAAACTTTAGTAGATCCGGAAATCTCACACGAGA |
| | | TATCAGAACCCACACTGGAGAAAAACCTTTTGCCTGCGAGATCTGCG |
| | | GAAGAAAATTCGCCCGAAGGTCCCACTTGAGTAGTCATACCAAAATC |
| | | CACTTGCGA |
| 121 | Left ZFP | GCAGCAATGGCAGAGAGACCATTTCAGTGCAGAATATGTATGCAAAA |
| | (ZFP-L) (na) | CTTCTCCCAGAGCGGTAATCTGGCTAGGCATATTAGAACACACACCG |
| | Codon diversified | GGGAAAAACCTTTCGCTTGCGATATATGTGGTAGAAAGTTCGCCCTC |
| | Version 5 | AAACAGAATCTGTGCATGCACACTAAAATCCATACAGGAGAAAAGCC |
| | | CTTTCAGTGTAGAATTTGTATGCAGAAATTTGCTTGGCAGTCAAATT |
| | | TGCAAAATCACACCAAAATACACACAGGAGAAAAACCATTTCAGTGT |
| | | AGAATATGTATGAGAAATTTTTCCACTTCCGGAAATCTGAGCAGACA |
| | | TATACGGACACACACTGGGGAAAAGCCCTTCGCTTGCGACATCTGCG |
| | | GAAGAAAGTTCGCTAGACGGTCCCACTTGAGATCCCACACTAAGATA |
| | | CATCTTCGC |
| 122 | Left ZFP | GCAGCCATGGCCGAACGCCCATTTCAATGTAGAATTTGTATGCAGAA |
| | (ZFP-L) (na) | TTTTTCACAATCAGGAAACCTGGCTAGACATATCAGAACACATACTG |
| | Codon diversified | GAGAAAAGCCCTTTGCTTGTGATATCTGTGGAAGGAAATTCGCCCTG |
| | Version 6 | AAACAAAACCTCTGTATGCACACAAAGATCCACACCGGCGAAAAGCC |
| | | TTTCCAGTGTAGGATATGCATGCAAAAATTCGCCTGGCAGTCCAATC |
| | | TGCAGAACCATACCAAAATTCATACTGGTGAAAAGCCATTTCAGTGC |
| | | AGAATATGTATGAGAAACTTTAGCACTTCAGGAAATCTCACAAGACA |
| | | TATAAGAACACATACAGGGGAAAAACCTTTTGCTTGCGATATCTGCG |
| | | GCAGGAAATTGGCTCGGAGAAGTCATCTGAGAAGCCATACAAAAATC |
| | | CACCTGCGA |
| 123 | Right ZFP | GCCGCTATGGCTGAGAGGCCCTTCCAGTGTCGAATCTGCATGCGTAA |
| | (ZFP-L) (na) | CTTCAGTCAGTCCTCCGACCTGTCCCGCCACATCCGCACCCACACCG |
| | Not diversified | GCGAGAAGCCTTTTGCCTGTGACATTTGTGGGAGGAAATTTGCCCTG |
| | | AAGCACAACCTGCTGACCCATACCAAGATACACACGGGCGAGAAGCC |
| | | CTTCCAGTGTCGAATCTGCATGCAGAACTTCAGTGACCAGTCCAACC |
| | | TGCGCGCCCACATCCGCACCCACACCGGCGAGAAGCCTTTTGCCTGT |
| | | GACATTTGTGGGAGGAAATTTGCCCGCAACTTCTCCCTGACCATGCA |
| | | TACCAAGATACACACCGGAGAGCGCGGCTTCCAGTGTCGAATCTGCA |
| | | TGCGTAACTTCAGTCTGCGCCACGACCTGGAGCGCCACATCCGCACC |
| | | CACACCGGCGAGAAGCCTTTTGCCTGTGACATTTGTGGGAGGAAATT |
| | | TGCCCACCGCTCCAACCTGAACAAGCATACCAAGATACACCTGCGG |
| 124 | Right ZFP | GCTGCTATGGCTGAAAGACCTTTTCAATGTCGAATCTGCATGAGGAA |
| | (ZFP-L) (na) | TTTTAGTGAGTCATCCGACCTGAGCAGACACATTCGAACCCATACTG |
| | Codon diversified | GTGAAAAGCCATTTGCTTGCGATATATGTGGGAGAAAATTTGCGTTG |
| | Version 1 | AAACACAATCTGCTGACCCATACCAAGATTCATACCGGAGAAAAACC |
| | | ATTCCAATGCCGCATTTGTATGCAGAACTTTAGTGACGAGTCAAATC |
| | | TCCGCGCTCACATTCGAACCCACACTGGCGAAAAACCCTTTGCTTGT |
| | | GACATTTGCGGTCGGAAGTTTGCCCGAAATTTTTCTCTGACAATGCA |
| | | CACAAAAATCCACACCGGGGAACGCGGCTTTCAATGTAGGATCTGTA |
| | | TGAGAAATTTTAGCCTTAGACATGATTTGGAACGACATATCAGGACC |
| | | CATACAGGCGAGAAACCATTTGCGTGCGATATTTGTGGCAGGAAATT |
| | | CGCACATAGAAGTAATCTGAACAAGCATACAAAAATTCATCTGAGA |
| 125 | Right ZFP | GCTGCCATGGCCGAGAGACCATTTCAATGTCGGATTTGCATGCGCAA |
| | (ZFP-L) (na) | TTTTTCCGAGTCCTCTGACCTTAGCCGGCATATTCGGACACACACAG |
| | Codon diversified | GTGAAAAACCCTTCGCATGCGACATTTGCGGAAGAAAATTCGCTCTG |
| | Version 2 | AAACACAAGCTGCTTACCCATACAAAGATCGAGACCGGCGAGAAACC |
| | | GTTTCAATGCCGAATCTGTATGCAAAATTTTAGTGATCAAAGTAATC |
| | | TGAGAGCACATATTAGGACTCACACGGGCGAGAAGCCATTTGCGTGT |
| | | GATATCTGCGGCCGAAAATTCGCCCGGAATTTCTCTCTGACAATGCA |
| | | CACCAAAATCCACACTGGGGAACGAGGCTTTCAATGTAGAATATGTA |
| | | TGCGGAATTTCAGTCTGAGGCACGACCTGGAGCGGCACATCAGAACT |
| | | CACACCGGAGAAAAACCATTCGCTTGTGATATTTGCGGGAGGAAGTT |
| | | CGCCCATAGGAGCAATCTCAATAAACACACCAAAATACATCTTCGG |
| 126 | Right ZFP | GCCGCCATGGCCGAGCGCCCCTTCCAATGCCGCATATGCATGAGAAA |
| | (ZFP-L) (na) | TTTGAGCCAAAGTAGCGACCTGTGAGGAGACATTAGAACTCATACGG |
| | Codon diversified | GGGAGAAGCCATTTGCTTGCGATATTTGTGGCAGAAAATTCGCACTC |
| | Version 3 | AAACACAAGCTGCTGAGACACACCAAGATACACACGGGAGAGAAGCC |
| | | CTTCCAATGTAGAATATGTATGCAAAATTTGAGCGACCAAAGTAATT |
| | | TGAGAGCGCATATTCGAACTCACACCGGCGAAAAACCATTTGCCTGC |
| | | GATATTTGTGGGAGGAAATTTGCCAGGAATTTTTCACTCACCATGCA |
| | | CACTAAGATCCACACTGGCGAGCGCGGCTTCCAATGCAGAATCTGTA |
| | | TGCGAAACTTGAGTCTGCGGCATGACCTGGAAAGACATATAAGAACC |
| | | GAGACCGGAGAAAAACCCTTTGCCTGCGAGATATGTGGTAGAAAATT |
| | | CGCACATCGGAGTAAGCTTAAGAAACATACAAAGATCGAGTTGAGA |
| 127 | Right ZFP | GCCGCTATGGCTGAAAGACCTTTCCAGTGTAGGATTTGCATGAGAAA |
| | (ZFP-L) (na) | TTTTTCCCAATCATCCGACCTTTCAAGGCATATTAGGACACACACCG |
| | Codon diversified | GGGAAAAGCCATTTGCTTGTGATATCTGCGGGCGCAAATTTGCTCTT |
| | Version 4 | AAGGACAATCTTCTTACCGAGACCAAAATTCATACAGGAGAAAAACC |
| | | TTTTCAATGTAGAATCTGCATGCAAAACTTTTCCGATGAGTCAAATC |
| | | TTAGAGCTCATATCAGAACCCATACCGGGGAGAAACCCTTTGCCTGC |
| | | GACATATGCGGAAGAAAATTTGCTAGGAACTTTAGTCTGACCATGCA |
| | | TACCAAAATTCATACCGGCGAACGCGGTTTCCAGTGCAGGATTTGTA |
| | | TGAGAAATTTCTGAGTGCGGCATGATCTTGAAAGACACATACGAACT |
| | | CATACCGGAGAAAAGCCATTCGCTTGCGATATTTGTGGTAGAAAATT |
| | | TGCCCACAGGTCTAAGCTTAATAAGGAGACCAAGATTCATCTGAGA |
| 128 | Right ZFP | GCAGCTATGGCCGAACGCCCTTTTCAATGCAGAATATGTATGCGAAA |
| | (ZFP-L) (na) | CTTCTCCCAAAGCTCTGATCTGTCAAGGGAGATACGGACACACACCG |
| | Codon diversified | GCGAAAAACCCTTTGCATGTGACATTTGTGGAAGAAAATTCGCACTT |
| | Version 5 | AAACACAATCTCCTGACTCATACAAAAATACATACAGGCGAAAAACC |
| | | TTTCCAGTGCAGAATCTGTATGCAGAACTTTTCCGACCAATCCAATC |
| | | TTCGCGCCCACATTAGAACTCACACAGGGGAGAAACCTTTCGCTTGC |
| | | GACATATGCGGAAGAAAATTTGCCAGAAATTTTTCACTTACAATGCA |
| | | CACAAAAATACATACTGGGGAAAGAGGGTTTCAATGTCGAATCTGTA |
| | | TGAGAAATTTGAGTCTGCGCCATGATCTGGAGAGACATATAAGAACA |
| | | CACACAGGAGAGAAACCTTTTGCTTGTGACATATGCGGCCGAAAGTT |
| | | TGCTCATAGATCTAATCTTAACAAACATACAAAGATCCATCTTCGG |
| 129 | Right ZFP | GCTGCTATGGCTGAGAGACCTTTCCAATGTAGGATCTGTATGCGAAA |
| | (ZFP-L) (na) | CTTCTCCCAGAGCTCCGACCTGAGTCGCCATATAAGAACCCATACCG |
| | Codon diversified | GAGAAAAACCATTTGCTTGTGACATTTGTGGCAGAAAGTTCGCTCTT |
| | Version 6 | AAACACAACCTGCTTACACATACTAAAATACACACAGGGGAGAAACC |
| | | CTTTCAATGCCGGATCTGTATGCAAAACTTTAGCGATCAATCAAACT |
| | | TGCGAGCCCATATCCGCACTCACACCGGCGAGAAGCCTTTTGCATGC |
| | | GATATATGTGGACGGAAATTTGCTAGAAACTTCTCATTGAGCATGCA |
| | | TACAAAAATACACACCGGGGAACGAGGATTTCAATGTCGAATTTGTA |
| | | TGAGAAATTTTAGCCTTAGGCAGGACTTGGAACGGCACATAAGAACC |
| | | CACACCGGAGAGAAGCCTTTTGCTTGTGATATTTGCGGCAGAAAGTT |
| | | CGCCCATCGGAGCAATCTTAACAAGCACACCAAGATTCATTTGAGA |
| 136 | Left ZFP | AAMAERPFQCRICMQNFSQSGNLARHIRTHTGEKPFACDICGRKFAL |
| | (ZFP-L) | KQNLCMHTKIHTGEKPFQCRICMQKFAWQSNLQNHTKIHTGEKPFQC |
| | protein (aa) | RICMRNFSTSGNLTRHIRTHTGEKPFACDICGRKFARRSHLTSHTKI |
| | | HLR |
| 137 | Right ZFP | AAMAERPFQCRICMRNFSQSSDLSRHIRTHTGEKPFACDICGRKFAL |
| | (ZFP-R) | KHNLLTHTKIHTGEKPFQCRICMQNFSDQSNLRAHIRTHTGEKPFAC |
| | protein (aa) | DICGRKFARNFSLTMHTKIHTGERGFQCRICMRNFSLRHDLERHIRT |
| | | HTGEKPFACDICGRKFAHRSNLNKHTKIHLR |
| 138 | 2A peptide (T2A) | GSGEGRGSLLTCGDVEENPGP |
| 139 | Left ZFN | CCCAAGAAGAAGAGGAAGGTCGGCATTCATGGGGTACCCGCCGCTAT |
| | with N-terminal | GGCTGAGAGGCCCTTCCAGTGTCGAATCTGCATGCAGAACTTCAGTC |
| | modifications (na) | AGTCCGGCAACCTGGCCCGCCACATCCGCACCCACACCGGCGAGAAG |
| | (comprising NLS, | CCTTTTGCCTGTGACATTTGTGGGAGGAAATTTGCCCTGAAGCAGAA |
| | ZFP-L, and FokI) | CCTGTGTATGCATACCAAGATACACACGGGCGAGAAGCCCTTCCAGT |
| | Not diversified | GTCGAATCTGCATGCAGAAGTTTGCCTGGCAGTCCAACCTGCAGAAC |
| | | CATACCAAGATACACACGGGCGAGAAGCCCTTCGAGTGTCGAATCTG |
| | | CATGCGTAACTTCAGTACCTCCGGCAACCTGACCCGCCACATCCGCA |
| | | CCCACACCGGCGAGAAGCCTTTTGCCTGTGACATTTGTGGGAGGAAA |
| | | TTTGCCCGCCGCTCCCACCTGACCTCCCATACCAAGATACACCTGCG |
| | | GGGATCCCAGCTGGTGAAGAGCGAGCTGGAGGAGAAGAAGTCCGAGC |
| | | TGCGGCACAAGCTGAAGTACGTGCCCCACGAGTACATCGAGCTGATC |
| | | GAGATCGCCAGGAACAGCACCCAGGACCGCATCCTGGAGATGAAGGT |
| | | GATGGAGTTCTTCATGAAGGTGTACGGCTACAGGGGAAAGCACCTGG |
| | | GCGGAAGCAGAAAGCCTGACGGCGCCATCTATACAGTGGGCAGCCCC |
| | | ATCGATTACGGCGTGATCGTGGACACAAAGGCCTACAGCGGCGGCTA |
| | | CAATCTGCCTATCGGCCAGGCCGACGAGATGGAGAGATACGTGGAGG |
| | | AGAACCAGACCCGGGATAAGCACCTCAACCCCAACGAGTGGTGGAAG |
| | | GTGTACCCTAGCAGCGTGACCGAGTTCAAGTTCCTGTTCGTGAGCGG |
| | | CCACTTCAAGGGCAACTACAAGGCCCAGCTGACCAGGCTGAACCACA |
| | | TCACCAACTGCGACGGCGCCGTGCTGAGCGTGGAGGAGCTGCTGATC |
| | | GGCGGCGAGATGATCAAAGCCGGCACCCTGACACTGGAGGAGGTGCG |
| | | GCGCAAGTTCAACAACGGCGAGATCAACTTCAGATCT |
| 140 | Left ZFN | CCAAAGAAGAAAAGAAAAGTGGGGATCCATGGTGTACCCGCAGCAAT |
| | with N-terminal | GGCCGAACGACCCTTCCAATGCAGAATATGTATGCAGAATTTTTCTC |
| | modifications (na) | AGAGCGGGAACCTGGCGAGGCACATAAGAACCCATACAGGAGAGAAG |
| | (comprising NLS, | CCATTCGCATGCGATATTTGCGGTAGAAAATTTGCACTCAAACAAAA |
| | ZFP-L, and FokI) | TCTCTGTATGGAGACTAAAATCCATACAGGTGAAAAGCCTTTTGAGT |
| | Codon diversified | GCAGGATTTGTATGCAAAAATTTGCTTGGCAAAGTAACTTGCAGAAC |
| | Version 1 | CACACAAAGATACACACAGGAGAGAAACCCTTCCAATGCCGAATCTG |
| | | TATGCGCAACTTGAGTACATCCGGAAATTTGAGTAGACATATTAGGA |
| | | CCCACACCGGCGAGAAGCCATTTGCCTGCGATATTTGTGGACGGAAA |
| | | TTCGCACGAGGGAGCCATCTGAGGAGTCATACTAAGATTCATCTCCG |
| | | CGGCAGCCAGCTTGTGAAGTCCGAACTGGAGGAAAAGAAGAGCGAAC |
| | | TGCGCCACAAATTGAAATACGTTCCGCATGAGTACATAGAGCTCATT |
| | | GAAATCGCTAGAAACTCTACCCAAGACAGGATACTGGAAATGAAAGT |
| | | GATGGAATTTTTCATGAAAGTTTATGGTTATAGGGGCAAACATCTGG |
| | | GTGGCTCTCGCAAGCCCGATGGGGCCATTTATACTGTCGGCTCACCT |
| | | ATCGACTATGGCGTCATTGTGGATACCAAGGCTTATTCTGGAGGATA |
| | | CAACCTGCCCATCGGACAAGCAGACGAAATGGAAAGATACGTCGAGG |
| | | AGAATCAAAGCCGAGACAAGCATCTGAACCCAAAGGAGTGGTGGAAA |
| | | GTGTACCCGAGCAGCGTTACTGAGTTCAAATTTCTCTTTGTAAGCGG |
| | | ACATTTTAAAGGGAATTACAAAGCACAACTGAGTAGGCTGAACCATA |
| | | TAACCAACTGTGACGGGGCCGTATTGAGTGTGGAAGAGCTTCTGATT |
| | | GGAGGAGAGATGATTAAGGCTGGCACACTGAGTCTCGAAGAAGTGAG |
| | | GCGCAAATTCAATAACGGTGAAATCAACTTCCGGTCT |
| 141 | Left ZFN | CCTAAAAAAAAGCGGAAAGTGGGAATTCACGGCGTGCCCGCCGCCAT |
| | with N-terminal | GGCAGAGAGACCCTTTCAATGTAGAATCTGTATGCAAAATTTCTCTC |
| | modifications (na) | AGAGTGGTAAGCTTGCAAGACACATCAGAACTCATACAGGTGAGAAG |
| | (comprising NLS, | CCGTTTGCATGTGACATTTGCGGTAGGAAATTTGCCTTGAAACAGAA |
| | ZFP-L, and FokI) | TCTTTGTATGCACACAAAAATCCATACTGGTGAAAAGCCATTCCAAT |
| | Codon diversified | GCCGCATCTGTATGCAAAAATTCGCGTGGCAGTCCAATTTGCAGAAC |
| | Version 2 | CATACCAAGATTGAGACGGGAGAAAAACCATTTGAGTGCCGCATCTG |
| | | CATGCGCAACTTTTCTACATCAGGAAACCTTAGAGGACATATTCGGA |
| | | CGCACACTGGAGAAAAACCATTTGCTTGTGACATATGCGGCCGAAAA |
| | | TTTGCGAGACGCTCTCATCTCACCTCACATACTAAGATTCATTTGCG |
| | | CGGAAGTGAGCTGGTGAAGAGTGAATTGGAAGAAAAAAAGTCAGAGC |
| | | TGAGACACAAAGTGAAATATGTTCGAGACGAGTAGATCGAGCTTATC |
| | | GAGATAGCAAGAAACTCCACCGAGGAGAGAATTTTGGAAATGAAAGT |
| | | TATGGAATTCTTTATGAAAGTGTATGGCTACAGGGGTAAACATCTGG |
| | | GGGGATCAAGAAAGCCTGATGGTGCAATTTACACAGTGGGCTCTCCT |
| | | ATCGACTACGGTGTGATCGTGGATACAAAGGCCTACTCTGGAGGATA |
| | | TAATTTGCCTATTGGACAAGCCGATGAAATGGAAAGATATGTGGAGG |
| | | AAAACGAGACTCGCGATAAGCACCTGAACCGAAATGAATGGTGGAAA |
| | | GTGTACCCTTCATCTGTTACCGAATTTAAATTTTTGTTCGTTTCCGG |
| | | GCATTTCAAGGGGAACTACAAGGCACAGCTGAGGAGACTGAATGAGA |
| | | TCACGAACTGCGACGGCGCTGTACTGTCCGTGGAAGAGCTTTTGATC |
| | | GGGGGCGAAATGATTAAGGCCGGCACACTGACGCTGGAGGAGGTGCG |
| | | GCGAAAATTTAATAATGGCGAGATCAATTTTAGGAGT |
| 142 | Left ZFN | CCCAAAAAGAAGAGAAAAGTGGGAATCCACGGTGTACCGGCCGCGAT |
| | with N-terminal | GGCAGAGAGACCATTTGAGTGTAGAATCTGTATGCAGAACTTTTCCC |
| | modifications (na) | AATGAGGAAACCTGGCACGAGACATTAGAACCCATACTGGAGAAAAG |
| | (comprising NLS, | CCGTTCGCTTGCGACATTTGCGGTAGAAAATTTGCTTTGAAACAGAA |
| | ZFP-L, and FokI) | CTTGTGTATGCATACCAAGATTCATACCGGCGAAAAACCATTTCAAT |
| | Codon diversified | GCAGGATTTGTATGCAGAAGTTCGCCTGGCAATCCAATTTGCAGAAT |
| | Version 3 | CATACTAAAATTCATACCGGAGAAAAACCATTCCAATGCCGCATTTG |
| | | TATGAGAAACTTTTCTAGCTCTGGCAATCTCACCAGACATATCAGAA |
| | | CACACACAGGCGAGAAACCGTTCGCATGCGATATCTGTGGGCGAAAG |
| | | TTTGCCAGAAGATCCCATCTCACATCACATACTAAAATACATTTGCG |
| | | AGGAAGTCAACTGGTCAAGTCCGAACTGGAGGAAAAAAAAAGTGAGC |
| | | TGCGAGACAAGTTGAAGTACGTACCACACGAATACATCGAGCTGATT |
| | | GAGATAGCACGGAACTCTAGCCAGGATAGAATACTGGAGATGAAAGT |
| | | TATGGAATTCTTTATGAAGGTGTACGGATACAGGGGGAAGCATCTTG |
| | | GCGGGAGCCGGAAACCAGACGGAGCAATCTATACCGTCGGGTCACCT |
| | | ATAGACTATGGAGTTATTGTCGATACAAAGGCCTATTCAGGAGGTTA |
| | | TAATCTGCCAATCGGCCAAGCCGACGAGATGGAGAGGTACGTGGAGG |
| | | AAAATCAGACCAGAGACAAGCACCTGAAGCCTAATGAATGGTGGAAA |
| | | GTGTACCCTAGCAGCGTCACTGAGTTCAAATTCCTGTTCGTCAGCGG |
| | | TCATTTTAAAGGAAATTATAAAGCCCAGCTCACTAGACTCAACCATA |
| | | TTACAAACTGCGACGGAGCCGTACTTAGCGTTGAAGAGTTGCTTATC |
| | | GGAGGAGAGATGATCAAAGCCGGAACCCTCACACTTGAAGAAGTGCG |
| | | AAGAAAATTCAATAACGGAGAGATAAATTTTAGGAGT |
| 143 | Left ZFN | CCTAAGAAGAAGAGAAAAGTTGGAATACATGGAGTCCCCGCAGCAAT |
| | with N-terminal | GGCCGAGAGACCTTTTCAGTGCAGGATTTGTATGCAAAACTTCTCTC |
| | modifications (na) | AGTCCGGTAACCTGGCCCGGCACATACGAACACATACCGGCGAAAAA |
| | (comprising NLS, | CCCTTTGCTTGCGACATCTGCGGAAGAAAGTTCGCTCTTAAACAGAA |
| | ZFP-L, and FokI) | CCTGTGCATGCATACAAAAATTCATACAGGTGAGAAGCCATTCCAAT |
| | Codon diversified | GCAGAATATGTATGCAGAAATTCGCCTGGCAAAGCAACCTGCAAAAC |
| | Version 4 | CACACTAAGATCCACACAGGGGAAAAGCCTTTTCAATGTAGAATCTG |
| | | TATGAGAAACTTTAGTAGATCCGGAAATCTCACACGAGATATCAGAA |
| | | CCCACACTGGAGAAAAACCTTTTGCCTGCGACATCTGCGGAAGAAAA |
| | | TTCGCCCGAAGGTCCCACTTGACTAGTCATACCAAAATCCACTTGCG |
| | | AGGCTCACAGCTGGTTAAATCCGAAGTTGAAGAAAAAAAAAGTGAAG |
| | | TGCGGCATAAACTGAAGTATGTCCCCCATGAATATATCGAAGTGATA |
| | | GAAATCGCCCGAAATAGCACCCAAGATAGAATCCTCGAAATGAAGGT |
| | | TATGGAATTTTTCATGAAGGTCTATGGATATAGGGGCAAGCACCTTG |
| | | GCGGATCCCGGAAACCTGATGGAGCTATCTACACAGTGGGCTCACCA |
| | | ATAGACTATGGAGTTATCGTCGATACAAAAGCATACAGCGGAGGATA |
| | | CAATTTGCCAATAGGTCAAGCAGATGAGATGGAAAGATACGTGGAGG |
| | | AAAAGGAAACAAGAGATAAGCATCTGAAGCCCAACGAATGGTGGAAA |
| | | GTGTACCCCAGTTCTGTAACCGAATTTAAGTTCTTGTTCGTTTCAGG |
| | | TCACTTCAAGGGTAATTACAAGGCTCAACTGAGTAGACTCAACCATA |
| | | TTACAAATTGCGATGGTGCTGTGCTTTCCGTGGAAGAATTGCTGATT |
| | | GGTGGAGAGATGATAAAAGCTGGTACCCTCACCTTGGAAGAAGTGCG |
| | | CAGAAAATTCAATAATGGCGAGATCAACTTCCGAAGT |
| 144 | Left ZFN | CCCAAGAAGAAACGAAAAGTAGGAATCCATGGCGTGCCTGCAGCAAT |
| | with N-terminal | GGCAGAGAGACCATTTGAGTGCAGAATATGTATGCAAAACTTCTCCC |
| | modifications (na) | AGAGCGGTAATCTGGCTAGGCATATTAGAACACACACCGGGGAAAAA |
| | (comprising NLS, | CCTTTCGCTTGCGATATATGTGGTAGAAAGTTCGCCCTCAAACAGAA |
| | ZFP-L, and FokI) | TCTGTGCATGCACACTAAAATCCATACAGGAGAAAAGCCCTTTGAGT |
| | Codon diversified | GTAGAATTTGTATGCAGAAATTTGCTTGGCAGTCAAATTTGCAAAAT |
| | Version 5 | CACACCAAAATACACACAGGAGAAAAACCATTTGAGTGTAGAATATG |
| | | TATGAGAAATTTTTCCACTTCCGGAAATCTGAGCAGACATATACGGA |
| | | CACACACTGGGGAAAAGCCCTTCGCTTGCGACATCTGCGGAAGAAAG |
| | | TTCGCTAGACGGTCCCACTTGAGATCCCACACTAAGATACATCTTCG |
| | | CGGTAGCCAACTGGTGAAAAGTGAACTGGAGGAAAAAAAATCTGAGC |
| | | TGAGACATAAACTGAAATACGTACCACATGAATACATAGAACTTATA |
| | | GAAATAGCTAGGAACTCCACCCAGGACAGAATACTTGAAATGAAGGT |
| | | CATGGAGTTTTTTATGAAAGTTTACGGATACAGGGGCAAACACCTTG |
| | | GAGGGTCTCGGAAGCCTGATGGCGCAATTTATACCGTGGGTAGCCCT |
| | | ATAGATTATGGAGTGATTGTGGATACAAAGGCTTACAGTGGCGGCTA |
| | | TAATTTGCCTATCGGACAGGCCGATGAGATGGAAAGATACGTTGAAG |
| | | AAAACCAAACACGAGATAAGCATCTGAACCCCAATGAATGGTGGAAA |
| | | GTGTATCCTTCAAGCGTTACCGAGTTTAAGTTCCTCTTCGTTTCTGG |
| | | GCATTTCAAGGGCAACTACAAAGCTCAGGTTACAAGACTGAACGAGA |
| | | TAACCAATTGTGATGGAGCAGTCCTCAGCGTGGAAGAACTCCTTATT |
| | | GGGGGTGAGATGATTAAAGCAGGGACCCTTACTCTTGAAGAGGTTAG |
| | | AAGAAAATTCAATAAGGGAGAGATTAATTTTAGAAGT |
| 145 | Left ZFN | CCTAAGAAGAAAAGAAAGGTCGGCATTCATGGTGTGCCTGCAGCCAT |
| | with N-terminal | GGCCGAACGCCCATTTCAATGTAGAATTTGTATGCAGAATTTTTCAC |
| | modifications (na) | AATCAGGAAACCTGGCTAGACATATCAGAACACATACTGGAGAAAAG |
| | (comprising NLS, | CCCTTTGCTTGTGATATCTGTGGAAGGAAATTCGCCCTGAAACAAAA |
| | ZFP-L, and FokI) | CCTCTGTATGCACACAAAGATCCACACCGGCGAAAAGCCTTTCGAGT |
| | Codon diversified | GTAGGATATGCATGCAAAAATTCGCCTGGCAGTCCAATCTGCAGAAC |
| | Version 6 | CATACCAAAATTCATACTGGTGAAAAGCCATTTGAGTGCAGAATATG |
| | | TATGAGAAACTTTAGCACTTCAGGAAATCTCACAAGACATATAAGAA |
| | | CACATACAGGGGAAAAACCTTTTGCTTGCGATATCTGCGGCAGGAAA |
| | | TTCGCTCGGAGAAGTCATCTCACAAGCCATACAAAAATCCACCTGCG |
| | | AGGAAGCGAGCTGGTCAAGTCTGAACTGGAAGAAAAAAAAAGCGAAC |
| | | TGCGGCATAAACTGAAATACGTCCCACATGAATACATTGAGCTCATC |
| | | GAAATTGCTAGAAACTCTAGTCAAGATAGGATATTGGAGATGAAGGT |
| | | AATGGAATTCTTCATGAAGGTTTATGGATATAGAGGAAAACATCTTG |
| | | GAGGCAGTAGGAAACCCGATGGCGCTATCTACACCGTAGGGAGTCCA |
| | | ATCGACTACGGCGTGATTGTTGACACCAAAGCCTATTCTGGAGGGTA |
| | | TAATCTCCCAATTGGACAGGCAGATGAGATGGAAAGATATGTAGAAG |
| | | AAAATCAGACAAGAGATAAGCACCTTAAGCCTAAGGAGTGGTGGAAA |
| | | GTGTACCCAAGCAGTGTTACTGAATTTAAATTTCTTTTTGTATCAGG |
| | | AGACTTTAAAGGCAATTACAAAGCACAACTGACCAGACTCAATGAGA |
| | | TTACCAATTGCGACGGAGCCGTACTGAGCGTGGAGGAGTTGCTGATC |
| | | GGAGGCGAAATGATTAAAGCTGGCACTCTGACCCTGGAAGAAGTAAG |
| | | AAGAAAGTTCAATAATGGAGAAATAAACTTTCGCTCC |
| 146 | Right ZFN | CCCAAGAAGAAGAGGAAGGTCGGCATTCATGGGGTACCCGCCGCTAT |
| | with N-terminal | GGCTGAGAGGCCCTTCCAGTGTCGAATCTGCATGCGTAACTTCAGTC |
| | modifications (na) | AGTCCTCCGACCTGTCCCGCCACATCCGCACCCACACCGGCGAGAAG |
| | (comprising NLS, | CCTTTTGCCTGTGACATTTGTGGGAGGAAATTTGCCCTGAAGCACAA |
| | ZFP-R, and FokI) | CCTGCTGACCCATACCAAGATACACACGGGCGAGAAGCCCTTCCAGT |
| | Not diversified | GTCGAATCTGCATGCAGAACTTCAGTGACCAGTCCAACCTGCGCGCC |
| | | CACATCCGCACCCACACCGGCGAGAAGCCTTTTGCCTGTGACATTTG |
| | | TGGGAGGAAATTTGCCCGCAACTTCTCCCTGACCATGCATACCAAGA |
| | | TACACACCGGAGAGCGCGGCTTCCAGTGTCGAATCTGCATGCGTAAC |
| | | TTCAGTCTGCGCCACGACCTGGAGCGCCACATCCGCACCCACACCGG |
| | | CGAGAAGCCTTTTGCCTGTGACATTTGTGGGAGGAAATTTGCCCACC |
| | | GCTCCAACCTGAACAAGCATACCAAGATACACCTGCGGGGATCCCAG |
| | | CTGGTGAAGAGCGAGCTGGAGGAGAAGAAGTCCGAGCTGCGGCACAA |
| | | GCTGAAGTACGTGCCCCACGAGTACATCGAGCTGATCGAGATCGCCA |
| | | GGAACAGCACCCAGGACCGCATCCTGGAGATGAAGGTGATGGAGTTC |
| | | TTCATGAAGGTGTACGGCTACAGGGGAAAGCACCTGGGCGGAAGCAG |
| | | AAAGCCTGACGGCGCCATCTATACAGTGGGCAGCCCCATCGATTACG |
| | | GCGTGATCGTGGACACAAAGGCCTACAGCGGCGGCTACAATCTGAGC |
| | | ATCGGCCAGGCCGACGAGATGCAGAGATACGTGAAGGAGAACCAGAC |
| | | CCGGAATAAGCACATCAACCCCAACGAGTGGTGGAAGGTGTACCCTA |
| | | GCAGCGTGACCGAGTTCAAGTTCCTGTTCGTGAGCGGCCACTTCAAG |
| | | GGCAACTACAAGGCCCAGCTGACCAGGCTGAACCGCAAAACCAACTG |
| | | CAATGGCGCCGTGCTGAGCGTGGAGGAGCTGCTGATCGGCGGCGAGA |
| | | TGATCAAAGCCGGCACCCTGACACTGGAGGAGGTGCGGCGCAAGTTC |
| | | AACAACGGCGAGATCAACTTC |
| 147 | Right ZFN | CCTAAAAAGAAACGAAAAGTGGGCATTCACGGCGTACCTGCTGCTAT |
| | with N-terminal | GGCTGAAAGACCTTTTCAATGTCGAATCTGCATGAGGAATTTTAGTC |
| | modifications (na) | AGTCATCCGAGCTGAGCAGACACATTCGAACCCATACTGGTGAAAAG |
| | (comprising NLS, | CCATTTGCTTGCGATATATGTGGGAGAAAATTTGCGTTGAAACACAA |
| | ZFP-R, and FokI) | TCTGCTGAGCCATACCAAGATTCATACCGGAGAAAAACCATTCCAAT |
| | Codon diversified | GCCGCATTTGTATGCAGAACTTTAGTGACCAGTCAAATCTCCGCGCT |
| | Version 1 | CACATTCGAACCCACACTGGCGAAAAACCCTTTGCTTGTGACATTTG |
| | | CGGTCGGAAGTTTGCCCGAAATTTTTCTCTGACAATGCACACAAAAA |
| | | TCCACACCGGGGAACGCGGCTTTCAATGTAGGATCTGTATGAGAAAT |
| | | TTTAGCCTTAGACATGATTTGGAACGAGATATGAGGAGCCATACAGG |
| | | CGAGAAACCATTTGCGTGCGATATTTGTGGCAGGAAATTCGCACATA |
| | | GAAGTAATCTGAACAAGCATACAAAAATTCATCTCAGAGGAAGTGAG |
| | | CTGGTCAAAAGTGAACTGGAGGAAAAAAAGAGCGAACTGAGACACAA |
| | | ACTGAAGTACGTGCGAGACGAATATATTGAGCTGATTGAGATCGCGA |
| | | GGAACTCAACACAGGACCGCATTCTGGAGATGAAAGTGATGGAGTTT |
| | | TTCATGAAAGTATATGGATATAGAGGAAAACACCTTGGGGGTAGCCG |
| | | AAAGCCGGACGGGGCGATCTACACTGTGGGGTCACCAATTGATTATG |
| | | GCGTAATTGTCGATACCAAAGCCTACAGTGGGGGGTACAATCTGAGT |
| | | ATAGGACAGGCTGATGAAATGCAACGATACGTTAAGGAGAATCAGAC |
| | | TAGGAATAAACATATCAATCGAAATGAATGGTGGAAAGTCTATCCCA |
| | | GCAGCGTGACAGAATTTAAATTTTTGTTTGTCAGTGGACACTTCAAG |
| | | GGAAATTATAAGGCCCAGCTGAGTAGACTGAATAGGAAAACCAATTG |
| | | TAATGGCGCAGTGCTTTCAGTGGAGGAACTGCTCATTGGAGGTGAGA |
| | | TGATCAAGGCTGGAACCCTGACGCTGGAGGAGGTGCGGAGGAAGTTT |
| | | AACAATGGAGAAATTAACTTT |
| 148 | Right ZFN | CCTAAGAAAAAGAGAAAAGTCGGAATCCACGGTGTCCCAGCTGCCAT |
| | with N-terminal | GGCCGAGAGACCATTTCAATGTCGGATTTGCATGCGCAATTTTTCCC |
| | modifications (na) | AGTCCTCTGACCTTAGCCGGCATATTCGGACACACACAGGTGAAAAA |
| | (comprising NLS, | CCCTTCGCATGCGACATTTGCGGAAGAAAATTCGCTCTGAAACACAA |
| | ZFP-R, and FokI) | CCTGCTTACCCATACAAAGATCCACACCGGCGAGAAACCGTTTCAAT |
| | Codon diversified | GCCGAATCTGTATGCAAAATTTTAGTGATCAAAGTAATCTGAGAGGA |
| | Version 2 | CATATTAGGACTCACACGGGCGAGAAGCCATTTGCGTGTGATATCTG |
| | | CGGCCGAAAATTCGCCCGGAATTTCTCTCTGACAATGCACACCAAAA |
| | | TCCACACTGGGGAACGAGGCTTTCAATGTAGAATATGTATGCGGAAT |
| | | TTCAGTCTGAGGCACGACCTGGAGCGGCACATCAGAACTCACACCGG |
| | | AGAAAAACCATTCGCTTGTGATATTTGCGGGAGGAAGTTCGCCCATA |
| | | GGAGCAATCTCAATAAAGACACCAAAATACATCTTCGGGGTTCTCAA |
| | | CTGGTGAAATCCGAACTGGAAGAAAAGAAATCAGAATTGCGGCATAA |
| | | ACTGAAGTATGTGCCCCATGAGTACATAGAACTGATCGAGATCGCAA |
| | | GGAACTCTACCCAGGACAGAATACTTGAAATGAAGGTCATGGAATTT |
| | | TTTATGAAAGTGTACGGCTACAGAGGAAAACATTTGGGAGGCAGTCG |
| | | AAAACCAGATGGCGCAATCTATACAGTCGGGTCCCCCATAGATTACG |
| | | GAGTGATTGTCGAGACAAAAGCCTATTCCGGAGGATATAACCTTAGT |
| | | ATCGGCCAGGCCGACGAGATGCAACGCTATGTGAAAGAAAACCAAAC |
| | | AAGAAATAAAGATATCAATCCAAAGGAGTGGTGGAAGGTATATCCAA |
| | | GCAGTGTCACAGAATTCAAATTCCTCTTCGTGAGTGGGCACTTTAAA |
| | | GGCAACTACAAAGCTCAATTGACCAGGCTCAATCGGAAAACTAATTG |
| | | CAATGGCGCAGTCCTTAGCGTCGAAGAATTGCTGATTGGCGGGGAAA |
| | | TGATTAAAGCAGGAACTTTGACCTTGGAGGAAGTAGGGAGAAAGTTT |
| | | AACAACGGCGAGATTAATTTT |
| 149 | Right ZFN | CCCAAGAAGAAAAGAAAAGTAGGAATTCACGGAGTCCCTGCCGCCAT |
| | with N-terminal | GGCCGAGCGCCCCTTCCAATGCCGCATATGCATGAGAAATTTCAGCC |
| | modifications (na) | AAAGTAGCGACCTGTCACGACACATTAGAACTCATACGGGGGAGAAG |
| | (comprising NLS, | CCATTTGCTTGCGATATTTGTGGCAGAAAATTCGCACTCAAACACAA |
| | ZFP-R, and FokI) | CCTGCTCACACACACCAAGATACACACGGGAGAGAAGCCCTTCCAAT |
| | Codon diversified | GTAGAATATGTATGCAAAATTTGAGCGAGCAAAGTAATTTGAGAGCG |
| | Version 3 | CATATTCGAACTCACACCGGCGAAAAACCATTTGCCTGCGATATTTG |
| | | TGGGAGGAAATTTGCCAGGAATTTTTCACTCACCATGCACACTAAGA |
| | | TCCACACTGGCGAGCGCGGCTTCCAATGCAGAATCTGTATGCGAAAC |
| | | TTCAGTCTGCGGCATGACCTGGAAAGACATATAAGAACCCACACCGG |
| | | AGAAAAACCCTTTGCCTGCGAGATATGTGGTAGAAAATTCGCACATC |
| | | GGAGTAACCTTAACAAAGATACAAAGATCCACTTGAGAGGCAGTGAG |
| | | CTGGTGAAATCTGAGCTGGAAGAGAAGAAATCTGAAGTGCGACATAA |
| | | ATTGAAGTACGTCCCACACGAGTAGATCGAGTTGATCGAAATTGCCC |
| | | GGAATAGCACCCAGGATAGAATATTGGAAATGAAAGTAATGGAGTTT |
| | | TTTATGAAGGTTTATGGTTACAGAGGCAAGCACCTTGGAGGAAGCAG |
| | | GAAACCAGATGGGGCGATTTACACCGTTGGGAGTCCCATCGATTACG |
| | | GAGTCATCGTGGACACAAAGGCCTATTCCGGAGGCTACAACCTCAGT |
| | | ATCGGGCAAGCCGATGAGATGCAGAGATATGTTAAAGAAAATCAGAC |
| | | GCGAAACAAGCACATTAACCCAAAGGAATGGTGGAAAGTTTACCCTA |
| | | GCTCAGTGACAGAATTTAAGTTTCTGTTTGTCAGCGGCCACTTCAAG |
| | | GGGAATTATAAAGCACAACTGAGCCGCCTGAAGCGAAAAACCAACTG |
| | | TAACGGTGCTGTGCTGAGTGTCGAAGAGTTGCTTATCGGAGGAGAGA |
| | | TGATAAAGGCCGGCACACTGACGCTTGAAGAGGTACGGCGAAAATTC |
| | | AATAACGGAGAGATTAATTTT |
| 150 | Right ZFN | CCAAAAAAAAAACGCAAGGTTGGAATACACGGTGTACCTGCCGCTAT |
| | with N-terminal | GGCTGAAAGACCTTTCCAGTGTAGGATTTGCATGAGAAATTTTTCCC |
| | modifications (na) | AATCATCCGACCTTTCAAGGCATATTAGGACACACACCGGGGAAAAG |
| | (comprising NLS, | CCATTTGCTTGTGATATCTGCGGGCGCAAATTTGCTCTTAAGCACAA |
| | ZFP-R, and FokI) | TCTTCTTACCCACACCAAAATTCATACAGGAGAAAAACCTTTTCAAT |
| | Codon diversified | GTAGAATCTGCATGCAAAACTTTTCCGATCAGTCAAATCTTAGAGCT |
| | Version 4 | CATATCAGAACCCATACCGGGGAGAAACCCTTTGCCTGCGACATATG |
| | | CGGAAGAAAATTTGCTAGGAACTTTAGTCTGAGCATGCATACCAAAA |
| | | TTCATACCGGCGAACGCGGTTTCCAGTGCAGGATTTGTATGAGAAAT |
| | | TTCTCACTGCGGCATGATCTTGAAAGACACATACGAACTCATACCGG |
| | | AGAAAAGCCATTCGCTTGCGATATTTGTGGTAGAAAATTTGCCCACA |
| | | GGTCTAACCTTAATAAGCACACCAAGATTCATCTCAGAGGATCTGAG |
| | | CTGGTCAAATCAGAACTTGAAGAGAAAAAAAGCGAACTGAGACATAA |
| | | ACTGAAGTACGTGCCTCATGAATACATAGAGCTCATTGAAATAGCTA |
| | | GGAATAGTACACAGGACAGGATACTTGAAATGAAGGTAATGGAATTT |
| | | TTCATGAAGGTTTATGGATACCGGGGGAAACATCTCGGGGGCAGCAG |
| | | AAAACCAGAGGGAGGAATTTATACTGTCGGGAGTCCTATAGATTATG |
| | | GCGTTATCGTCGATACAAAGGCCTATTCCGGTGGGTACAACCTCTCA |
| | | ATTGGTCAGGCTGATGAGATGCAAAGATACGTCAAAGAAAACCAAAC |
| | | CAGAAATAAACATATAAATCCGAATGAATGGTGGAAAGTATACCCAA |
| | | GTTCCGTGACTGAATTCAAGTTCCTTTTCGTGTCTGGCCACTTTAAA |
| | | GGAAATTATAAAGCACAATTGAGTAGACTGAATAGAAAAACAAACTG |
| | | TAACGGCGCAGTGCTGTCAGTGGAAGAACTGCTCATAGGTGGAGAGA |
| | | TGATCAAGGCCGGGACACTTACTCTTGAGGAAGTTAGAAGGAAGTTC |
| | | AACAACGGCGAAATCAACTTT |
| 151 | Right ZFN | CCAAAGAAAAAGAGGAAGGTGGGAATACATGGAGTACCAGCAGCTAT |
| | with N-terminal | GGCCGAACGCCCTTTTCAATGCAGAATATGTATGCGAAACTTCTCCC |
| | modifications (na) | AAAGCTCTGATCTGTCAAGGCACATACGGACACACACCGGCGAAAAA |
| | (comprising NLS, | CCCTTTGCATGTGAGATTTGTGGAAGAAAATTCGCACTTAAACACAA |
| | ZFP-R, and FokI) | TCTCCTGAGTCATACAAAAATACATACAGGCGAAAAACCTTTCGAGT |
| | Codon diversified | GCAGAATCTGTATGCAGAACTTTTCCGACCAATCCAATCTTCGCGCC |
| | Version 5 | CACATTAGAACTCACACAGGGGAGAAACCTTTCGCTTGCGACATATG |
| | | CGGAAGAAAATTTGCCAGAAATTTTTCACTTAGAATGCACACAAAAA |
| | | TACATACTGGGGAAAGAGGGTTTGAATGTCGAATCTGTATGAGAAAT |
| | | TTCAGTCTGCGCCATGATCTGGAGAGACATATAAGAACACACACAGG |
| | | AGAGAAACCTTTTGCTTGTGACATATGCGGCCGAAAGTTTGCTCATA |
| | | GATCTAATCTTAACAAACATACAAAGATCCATCTTCGGGGTTCACAA |
| | | CTGGTCAAGTCAGAATTGGAAGAGAAAAAATCTGAGCTGAGGCACAA |
| | | ATTGAAATACGTTCCTCACGAGTATATTGAACTTATCGAGATAGCGC |
| | | GCAATAGTACACAAGATAGAATCTTGGAGATGAAAGTTATGGAATTC |
| | | TTTATGAAAGTCTATGGCTATAGGGGAAAACACCTGGGGGGTAGCAG |
| | | GAAAGCTGATGGAGCTATCTATACCGTAGGATCACCTATTGATTATG |
| | | GAGTAATTGTGGACACTAAGGCATATTCCGGAGGATATAATTTGAGT |
| | | ATTGGTCAGGCCGACGAAATGCAACGATACGTGAAGGAAAATCAGAC |
| | | CCGGAACAAACACATTAATCCGAATGAATGGTGGAAGGTATACCCTA |
| | | GTAGCGTTACAGAGTTTAAATTCCTTTTCGTCAGCGGCCACTTTAAA |
| | | GGAAATTATAAAGGACAACTCACCAGACTTAATCGAAAAACTAACTG |
| | | TAACGGCGCCGTACTGTCAGTGGAGGAGCTGCTCATTGGAGGCGAGA |
| | | TGATCAAGGCCGGTACTCTCAGACTGGAAGAAGTTAGAAGAAAGTTC |
| | | AACAACGGGGAAATTAATTTC |
| 152 | Right ZFN | CCCAAAAAGAAAAGAAAGGTGGGTATTCACGGAGTTCCCGCTGCTAT |
| | with N-terminal | GGCTGAGAGACCTTTCCAATGTAGGATCTGTATGCGAAACTTCTCCC |
| | modifications (na) | AGAGCTCCGAGCTGAGTCGCCATATAAGAACCCATACCGGAGAAAAA |
| | (comprising NLS, | CCATTTGCTTGTGACATTTGTGGCAGAAAGTTCGCTCTTAAACACAA |
| | ZFP-R, and FokI) | CCTGCTTACACATACTAAAATACACACAGGGGAGAAACCCTTTCAAT |
| | Codon diversified | GCCGGATCTGTATGCAAAACTTTAGCGATCAATCAAACTTGCGAGCC |
| | Version 6 | CATATCCGCACTCACACCGGCGAGAAGCCTTTTGCATGCGATATATG |
| | | TGGAGGGAAATTTGCTAGAAACTTCTCATTGAGCATGCATACAAAAA |
| | | TAGACACCGGGGAACGAGGATTTCAATGTCGAATTTGTATGAGAAAT |
| | | TTTAGCCTTAGGCACGACTTGGAACGGCACATAAGAACCCACACCGG |
| | | AGAGAAGCCTTTTGCTTGTGATATTTGCGGCAGAAAGTTCGCCCATC |
| | | GCAGCAATCTTAACAAGCAGACCAAGATTCATTTGAGAGGTTCCGAG |
| | | CTGGTCAAAAGCGAACTTGAAGAAAAGAAATCCGAGCTTAGACACAA |
| | | ACTGAAATACGTGCCTCACGAGTATATTGAGCTGATTGAAATAGCAA |
| | | GGAATTCAACACAAGACAGGATCCTCGAAATGAAGGTTATGGAGTTT |
| | | TTCATGAAAGTTTACGGCTACAGAGGGAAGCATCTGGGCGGATCAAG |
| | | AAAAGCAGACGGCGCAATCTAGACAGTTGGATCCCCAATAGATTACG |
| | | GAGTGATTGTTGACACCAAGGCTTATTGAGGAGGTTAGAATCTGTCC |
| | | ATTGGTCAGGCCGATGAAATGCAAAGATATGTTAAGGAAAATCAAAC |
| | | TCGAAACAAACACATTAATCCAAACGAATGGTGGAAAGTATATCCAA |
| | | GCTCCGTCACTGAATTTAAATTTTTGTTTGTATCCGGACATTTTAAG |
| | | GGCAACTATAAGGCTGAACTGAGCAGACTGAATAGGAAGACCAATTG |
| | | TAACGGAGCTGTACTCAGCGTGGAAGAACTGCTTATTGGAGGCGAAA |
| | | TGATTAAGGCTGGCACACTTACACTCGAAGAAGTTAGAAGAAAATTC |
| | | AACAATGGTGAGATAAACTTC |
| 157 | FokI | CAGCTGGTGAAGAGCGAGCTGGAGGAGAAGAAGTCCGAGCTGCGGCA |
| | (Right ZFN) | CAAGCTGAAGTACGTGCCCCACGAGTAGATCGAGCTGATCGAGATCG |
| | Not diversified | CCAGGAACAGCACCCAGGACCGCATCCTGGAGATGAAGGTGATGGAG |
| | (na) | TTCTTCATGAAGGTGTACGGCTACAGGGGAAAGCACCTGGGCGGAAG |
| | | CAGAAAGCCTGACGGCGCCATCTATACAGTGGGCAGCCCCATCGATT |
| | | ACGGCGTGATCGTGGACACAAAGGCCTACAGCGGCGGCTACAATCTG |
| | | AGCATCGGCCAGGCCGACGAGATGCAGAGATACGTGAAGGAGAACCA |
| | | GACCCGGAATAAGCACATCAACCCCAACGAGTGGTGGAAGGTGTACC |
| | | CTAGCAGCGTGACCGAGTTCAAGTTCCTGTTCGTGAGCGGCCACTTC |
| | | AAGGGCAACTACAAGGCCCAGCTGACCAGGCTGAACCGCAAAACCAA |
| | | CTGCAATGGCGCCGTGCTGAGCGTGGAGGAGCTGCTGATCGGCGGCG |
| | | AGATGATCAAAGCCGGCACCCTGACACTGGAGGAGGTGCGGCGCAAG |
| | | TTGAACAACGGCGAGATGAACTTC |
| 158 | FokI | CAGCTGGTCAAAAGTGAACTGGAGGAAAAAAAGAGCGAACTGAGACA |
| | (Right ZFN) | CAAACTGAAGTACGTGCCAGACGAATATATTGAGCTGATTGAGATCG |
| | Codon diversified | CGAGGAACTCAACACAGGACCGCATTCTGGAGATGAAAGTGATGGAG |
| | (na) | TTTTTCATGAAAGTATATGGATATAGAGGAAAACACCTTGGGGGTAG |
| | Version 1 | CCGAAAGCCGGACGGGGCGATCTACACTGTGGGGTCACCAATTGATT |
| | | ATGGCGTAATTGTCGATACCAAAGCCTACAGTGGGGGGTACAATCTG |
| | | AGTATAGGACAGGCTGATGAAATGCAACGATACGTTAAGGAGAATCA |
| | | GACTAGGAATAAACATATCAATCGAAATGAATGGTGGAAAGTCTATC |
| | | CCAGCAGCGTGACAGAATTTAAATTTTTGTTTGTCAGTGGACACTTC |
| | | AAGGGAAATTATAAGGCCCAGCTGAGTAGACTGAATAGGAAAACCAA |
| | | TTGTAATGGCGCAGTGCTTTCAGTGGAGGAACTGCTCATTGGAGGTG |
| | | AGATGATCAAGGCTGGAACCCTGACGCTGGAGGAGGTGCGGAGGAAG |
| | | TTTAACAATGGAGAAATTAACTTT |
| 159 | FokI | CAACTGGTGAAATCCGAACTGGAAGAAAAGAAATCAGAATTGCGGCA |
| | (Right ZFN) | TAAACTGAAGTATGTGCCCCATGAGTACATAGAACTGATCGAGATCG |
| | Codon diversified | CAAGGAACTCTAGCGAGGAGAGAATACTTGAAATGAAGGTCATGGAA |
| | (na) | TTTTTTATGAAAGTGTACGGCTACAGAGGAAAACATTTGGGAGGCAG |
| | Version 2 | TCGAAAACCAGATGGCGCAATCTATACAGTCGGGTCCCCCATAGATT |
| | | ACGGAGTGATTGTCGAGACAAAAGCCTATTCCGGAGGATATAACCTT |
| | | AGTATCGGCCAGGCCGACGAGATGCAACGCTATGTGAAAGAAAACCA |
| | | AACAAGAAATAAACATATCAATCCAAACGAGTGGTGGAAGGTATATC |
| | | CAAGCAGTGTCACAGAATTCAAATTCCTCTTCGTGAGTGGGCACTTT |
| | | AAAGGCAACTACAAAGCTCAATTGAGCAGGCTCAATCGGAAAACTAA |
| | | TTGCAATGGCGCAGTCCTTAGCGTCGAAGAATTGCTGATTGGCGGGG |
| | | AAATGATTAAAGCAGGAACTTTGAGCTTGGAGGAAGTAGGGAGAAAG |
| | | TTTAACAACGGCGAGATTAATTTT |
| 160 | FokI | CAGCTGGTGAAATCTGAGCTGGAAGAGAAGAAATCTGAAGTGCGACA |
| | (Right ZFN) | TAAATTGAAGTACGTCCGAGACGAGTAGATCGAGTTGATCGAAATTG |
| | Codon diversified | CCCGGAATAGCACCCAGGATAGAATATTGGAAATGAAAGTAATGGAG |
| | (na) | TTTTTTATGAAGGTTTATGGTTACAGAGGCAAGCACCTTGGAGGAAG |
| | Version 3 | CAGGAAACCAGATGGGGCGATTTACACCGTTGGGAGTCCCATCGATT |
| | | ACGGAGTCATCGTGGACACAAAGGCCTATTCCGGAGGCTACAACCTC |
| | | AGTATCGGGCAAGCCGATGAGATGCAGAGATATGTTAAAGAAAATCA |
| | | GACGCGAAACAAGGAGATTAACCCAAACGAATGGTGGAAAGTTTACC |
| | | CTAGCTCAGTGACAGAATTTAAGTTTCTGTTTGTCAGCGGCCACTTC |
| | | AAGGGGAATTATAAAGCACAACTGACCCGCCTGAACCGAAAAACCAA |
| | | CTGTAACGGTGCTGTGCTGAGTGTCGAAGAGTTGCTTATCGGAGGAG |
| | | AGATGATAAAGGCCGGCACACTGACGCTTGAAGAGGTACGGCGAAAA |
| | | TTCAATAACGGAGAGATTAATTTT |
| 161 | FokI | CAGCTGGTGAAATCAGAACTTGAAGAGAAAAAAAGCGAACTGAGACA |
| | (Right ZFN) | TAAACTGAAGTACGTGCCTCATGAATACATAGAGCTCATTGAAATAG |
| | Codon diversified | CTAGGAATAGTACACAGGACAGGATACTTGAAATGAAGGTAATGGAA |
| | (na) | TTTTTCATGAAGGTTTATGGATACCGGGGGAAACATCTCGGGGGCAG |
| | Version 4 | CAGAAAACCAGACGGAGCAATTTATACTGTCGGGAGTGCTATAGATT |
| | | ATGGCGTTATCGTCGATACAAAGGCCTATTCCGGTGGGTACAACCTC |
| | | TCAATTGGTCAGGCTGATGAGATGCAAAGATACGTCAAAGAAAACGA |
| | | AACCAGAAATAAACATATAAATCCCAATGAATGGTGGAAAGTATACC |
| | | CAAGTTCCGTGACTGAATTCAAGTTCCTTTTCGTGTCTGGCCACTTT |
| | | AAAGGAAATTATAAAGCACAATTGAGTAGACTGAATAGAAAAACAAA |
| | | CTGTAACGGCGCAGTGCTGTCAGTGGAAGAACTGCTCATAGGTGGAG |
| | | AGATGATCAAGGCCGGGACACTTAGTCTTGAGGAAGTTAGAAGGAAG |
| | | TTCAACAACGGCGAAATCAACTTT |
| 162 | FokI | CAACTGGTCAAGTCAGAATTGGAAGAGAAAAAATCTGAGCTGAGGCA |
| | (Right ZFN) | GAAATTGAAATACGTTCCTGAGGAGTATATTGAAGTTATCGAGATAG |
| | Codon diversified | CCCGCAATAGTACACAAGATAGAATCTTGGAGATGAAAGTTATGGAA |
| | (na) | TTCTTTATGAAAGTCTATGGCTATAGGGGAAAACACCTGGGGGGTAG |
| | Version 5 | CAGGAAACCTGATGGAGCTATCTATACCGTAGGATCACCTATTGATT |
| | | ATGGAGTAATTGTGGACACTAAGGCATATTCCGGAGGATATAATTTG |
| | | AGTATTGGTCAGGCCGACGAAATGCAACGATACGTGAAGGAAAATCA |
| | | GACCCGCAACAAACACATTAATCCCAATGAATGGTGGAAGGTATACC |
| | | CTAGTAGCGTTACAGAGTTTAAATTCCTTTTCGTCAGCGGCCACTTT |
| | | AAAGGAAATTATAAAGCACAACTCACCAGACTTAATCGAAAAACTAA |
| | | CTGTAACGGCGCCGTACTGTCAGTGGAGGAGCTGCTCATTGGAGGCG |
| | | AGATGATCAAGGCCGGTACTCTCAGACTGGAAGAAGTTAGAAGAAAG |
| | | TTCAACAACGGGGAAATTAATTTC |
| 163 | FokI | CAGCTGGTCAAAAGCGAAGTTGAAGAAAAGAAATCCGAGCTTAGACA |
| | (Right ZFN) | CAAACTGAAATACGTGCCTCACGAGTATATTGAGCTGATTGAAATAG |
| | Codon diversified | CAAGGAATTCAACACAAGACAGGATCCTCGAAATGAAGGTTATGGAG |
| | (na) | TTTTTCATGAAAGTTTACGGCTACAGAGGGAAGCATCTGGGCGGATC |
| | Version 6 | AAGAAAACCAGACGGCGCAATCTAGACAGTTGGATCCCGAATAGATT |
| | | ACGGAGTGATTGTTGACACCAAGGCTTATTCAGGAGGTTAGAATCTG |
| | | TCCATTGGTCAGGCCGATGAAATGCAAAGATATGTTAAGGAAAATCA |
| | | AACTCGAAAGAAACACATTAATCGAAAGGAATGGTGGAAAGTATATC |
| | | CAAGCTCCGTCACTGAATTTAAATTTTTGTTTGTATCCGGACATTTT |
| | | AAGGGCAACTATAAGGCTCAACTGAGCAGACTGAATAGGAAGACCAA |
| | | TTGTAACGGAGCTGTACTCAGCGTGGAAGAACTGCTTATTGGAGGCG |
| | | AAATGATTAAGGCTGGCACACTTACACTGGAAGAAGTTAGAAGAAAA |
| | | TTCAACAATGGTGAGATAAACTTC |
| 164 | FokI | CAGCTGGTGAAGAGCGAGCTGGAGGAGAAGAAGTCCGAGCTGCGGCA |
| | (Left ZFN) | CAAGCTGAAGTACGTGCCCCACGAGTAGATCGAGCTGATCGAGATCG |
| | Not diversified | CCAGGAACAGCACCCAGGACCGCATCCTGGAGATGAAGGTGATGGAG |
| | (na) | TTCTTCATGAAGGTGTACGGCTACAGGGGAAAGCACCTGGGCGGAAG |
| | | CAGAAAGCCTGACGGCGCCATCTATACAGTGGGCAGCCCCATCGATT |
| | | ACGGCGTGATCGTGGACACAAAGGCCTACAGCGGCGGCTACAATCTG |
| | | CCTATCGGCCAGGCCGACGAGATGGAGAGATACGTGGAGGAGAACCA |
| | | GACCCGGGATAAGCACCTCAACCCCAACGAGTGGTGGAAGGTGTACC |
| | | CTAGCAGCGTGACCGAGTTCAAGTTCCTGTTCGTGAGCGGCCACTTC |
| | | AAGGGCAACTACAAGGCCCAGCTGACCAGGCTGAACCACATCACCAA |
| | | CTGCGACGGCGCCGTGCTGAGCGTGGAGGAGCTGCTGATCGGCGGCG |
| | | AGATGATCAAAGCCGGCACCCTGACACTGGAGGAGGTGCGGCGCAAG |
| | | TTCAACAACGGCGAGATCAACTTGAGATCT |
| 165 | FokI | CAGCTTGTGAAGTCCGAACTGGAGGAAAAGAAGAGCGAACTGCGCCA |
| | (Left ZFN) | CAAATTGAAATACGTTCCGCATGAGTACATAGAGCTCATTGAAATCG |
| | Codon diversified | CTAGAAACTCTAGCCAAGACAGGATACTGGAAATGAAAGTGATGGAA |
| | (na) | TTTTTCATGAAAGTTTATGGTTATAGGGGCAAACATCTGGGTGGCTC |
| | Version 1 | TCGCAAGCCCGATGGGGCCATTTATACTGTCGGCTCACCTATCGACT |
| | | ATGGCGTCATTGTGGATACCAAGGCTTATTCTGGAGGATACAACCTG |
| | | CCCATCGGAGAAGCAGACGAAATGGAAAGATACGTCGAGGAGAATCA |
| | | AACCCGAGACAAGCATCTGAAGCGAAAGGAGTGGTGGAAAGTGTACC |
| | | CGAGCAGCGTTACTGAGTTCAAATTTCTCTTTGTAAGCGGACATTTT |
| | | AAAGGGAATTACAAAGCACAACTGAGTAGGCTGAAGCATATAACCAA |
| | | CTGTGACGGGGCCGTATTGAGTGTGGAAGAGCTTCTGATTGGAGGAG |
| | | AGATGATTAAGGCTGGCACACTGACTCTCGAAGAAGTGAGGCGCAAA |
| | | TTCAATAACGGTGAAATCAACTTCCGGTCT |
| 166 | FokI | CAGCTGGTGAAGAGTGAATTGGAAGAAAAAAAGTCAGAGCTGAGACA |
| | (Right ZFN) | CAAACTGAAATATGTTCGAGACGAGTAGATCGAGCTTATCGAGATAG |
| | Codon diversified | CAAGAAACTCGAGCGAGGACAGAATTTTGGAAATGAAAGTTATGGAA |
| | (na) | TTCTTTATGAAAGTGTATGGCTACAGGGGTAAACATCTGGGGGGATC |
| | Version 2 | AAGAAAGCCTGATGGTGCAATTTACACAGTGGGCTCTCCTATCGACT |
| | | ACGGTGTGATCGTGGATACAAAGGCCTACTCTGGAGGATATAATTTG |
| | | CCTATTGGACAAGCCGATGAAATGGAAAGATATGTGGAGGAAAACCA |
| | | GACTCGCGATAAGGACCTGAACCGAAATGAATGGTGGAAAGTGTAGC |
| | | CTTCATCTGTTACCGAATTTAAATTTTTGTTCGTTTCCGGGCATTTC |
| | | AAGGGGAACTACAAGGCACAGCTGACGAGACTGAATCACATGAGGAA |
| | | CTGCGACGGCGCTGTACTGTCCGTGGAAGAGCTTTTGATCGGGGGCG |
| | | AAATGATTAAGGCCGGCACACTGACGCTGGAGGAGGTGCGGCGAAAA |
| | | TTTAATAATGGCGAGATGAATTTTAGGAGT |
| 167 | FokI | CAACTGGTCAAGTCCGAACTGGAGGAAAAAAAAAGTGAGCTGCGACA |
| | (Right ZFN) (na) | CAAGTTGAAGTACGTACGAGACGAATACATCGAGCTGATTGAGATAG |
| | Codon diversified | CACGGAACTCTAGCCAGGATAGAATACTGGAGATGAAAGTTATGGAA |
| | Version 3 | TTCTTTATGAAGGTGTACGGATACAGGGGGAAGCATCTTGGCGGGAG |
| | | CCGGAAACCAGACGGAGCAATCTATACCGTCGGGTCACCTATAGACT |
| | | ATGGAGTTATTGTCGATACAAAGGCCTATTCAGGAGGTTATAATCTG |
| | | CCAATCGGCCAAGCCGACGAGATGGAGAGGTACGTGGAGGAAAATCA |
| | | GACCAGAGACAAGGAGCTGAACCCTAATGAATGGTGGAAAGTGTACC |
| | | CTAGCAGCGTCACTGAGTTCAAATTCCTGTTCGTCAGCGGTCATTTT |
| | | AAAGGAAATTATAAAGCCCAGCTGAGTAGACTGAACCATATTACAAA |
| | | CTGCGACGGAGCCGTACTTAGCGTTGAAGAGTTGCTTATCGGAGGAG |
| | | AGATGATGAAAGCCGGAACGGTGAGACTTGAAGAAGTGCGAAGAAAA |
| | | TTCAATAACGGAGAGATAAATTTTAGGAGT |
| 168 | FokI | CAGCTGGTTAAATCCGAACTTGAAGAAAAAAAAAGTGAACTGCGGCA |
| | (Right ZFN) | TAAACTGAAGTATGTCCCCCATGAATATATCGAACTGATAGAAATCG |
| | Codon diversified | CCCGAAATAGGAGGCAAGATAGAATCCTCGAAATGAAGGTTATGGAA |
| | (na) | TTTTTCATGAAGGTCTATGGATATAGGGGCAAGCACCTTGGCGGATC |
| | Version 4 | CCGGAAACCTGATGGAGCTATCTACACAGTGGGCTCACCAATAGACT |
| | | ATGGAGTTATCGTCGATACAAAAGCATACAGCGGAGGATACAATTTG |
| | | CCAATAGGTCAAGCAGATGAGATGGAAAGATACGTGGAGGAAAACCA |
| | | AACAAGAGATAAGCATCTGAACCCGAACGAATGGTGGAAAGTGTACC |
| | | CCAGTTCTGTAACCGAATTTAAGTTCTTGTTCGTTTCAGGTCACTTC |
| | | AAGGGTAATTACAAGGCTGAACTGACTAGACTGAACCATATTACAAA |
| | | TTGCGATGGTGCTGTGCTTTCCGTGGAAGAATTGCTGATTGGTGGAG |
| | | AGATGATAAAAGCTGGTACCCTCACCTTGGAAGAAGTGCGCAGAAAA |
| | | TTCAATAATGGCGAGATCAACTTCCGAAGT |
| 169 | FokI | CAACTGGTGAAAAGTGAACTGGAGGAAAAAAAATCTGAGCTGAGACA |
| | (Right ZFN) | TAAACTGAAATACGTACCACATGAATACATAGAACTTATAGAAATAG |
| | Codon diversified | CTAGGAACTCGAGCGAGGACAGAATACTTGAAATGAAGGTCATGGAG |
| | (na) | TTTTTTATGAAAGTTTACGGATACAGGGGCAAACACCTTGGAGGGTC |
| | Version 5 | TCGGAAGCCTGATGGCGCAATTTATACCGTGGGTAGCCCTATAGATT |
| | | ATGGAGTGATTGTGGATACAAAGGCTTACAGTGGCGGCTATAATTTG |
| | | CCTATCGGACAGGCCGATGAGATGGAAAGATACGTTGAAGAAAACGA |
| | | AACACGAGATAAGCATCTGAACCCCAATGAATGGTGGAAAGTGTATC |
| | | CTTCAAGCGTTACCGAGTTTAAGTTCCTCTTCGTTTCTGGGCATTTC |
| | | AAGGGCAACTACAAAGCTCAGCTTACAAGACTCAACCACATAACCAA |
| | | TTGTGATGGAGCAGTCCTCAGCGTGGAAGAACTCCTTATTGGGGGTG |
| | | AGATGATTAAAGCAGGGACCCTTAGTCTTGAAGAGGTTAGAAGAAAA |
| | | TTCAATAACGGAGAGATTAATTTTAGAAGT |
| 170 | FokI | CAGCTGGTCAAGTCTGAACTGGAAGAAAAAAAAAGCGAACTGCGGCA |
| | (Right ZFN) | TAAACTCAAATACGTCCCACATGAATACATTGAGCTCATCGAAATTG |
| | Codon diversified | CTAGAAACTCTAGTCAAGATAGGATATTGGAGATGAAGGTAATGGAA |
| | (na) | TTCTTCATGAAGGTTTATGGATATAGAGGAAAACATCTTGGAGGCAG |
| | Version 6 | TAGGAAACCCGATGGCGCTATCTACACCGTAGGGAGTCCAATCGACT |
| | | ACGGCGTGATTGTTGACACCAAAGCCTATTCTGGAGGGTATAATCTC |
| | | GCAATTGGACAGGCAGATGAGATGGAAAGATATGTAGAAGAAAATCA |
| | | GAGAAGAGATAAGGAGCTTAACCCTAACGAGTGGTGGAAAGTGTACC |
| | | CAAGCAGTGTTACTGAATTTAAATTTCTTTTTGTATCAGGACACTTT |
| | | AAAGGCAATTACAAAGCACAACTGACCAGACTCAATGAGATTACCAA |
| | | TTGCGACGGAGCCGTACTGAGCGTGGAGGAGTTGCTGATCGGAGGCG |
| | | AAATGATTAAAGCTGGCACTCTGACCCTGGAAGAAGTAAGAAGAAAG |
| | | TTCAATAATGGAGAAATAAACTTTCGCTCC |
| 171 | FokI | QLVKSELEEKKSELRHKLKYVPHEYIELIEIARNSTQDRILEMKVME |
| | (Right ZFN) (aa) | FFMKVYGYRGKHLGGSRKPDGAIYTVGSPIDYGVIVDTKAYSGGYNL |
| | | SIGQADEMQRYVKENQTRNKHINPNEWWKVYPSSVTEFKFLFVSGHF |
| | | KGNYKAQLTRLNRKTNCNGAVLSVEELLIGGEMIKAGTLTLEEVRRK |
| | | FNNGEINF |
| 172 | FokI | QLVKSELEEKKSELRHKLKYVPHEYIELIEIARNSTQDRILEMKVME |
| | (Left ZFN) (aa) | FFMKVYGYRGKHLGGSRKPDGAIYTVGSPIDYGVIVDTKAYSGGYNL |
| | | PIGQADEMERYVEENQTRDKHLNPNEWWKVYPSSVTEFKFLFVSGHF |
| | | KGNYKAQLTRLNHITNCDGAVLSVEELLIGGEMIKAGTLTLEEVRRK |
| | | FNNGEINFRS |

TABLE 3
Exemplary 2-in-1 Constructs

| SEQ | | |
|---|---|---|
| ID | Feature/ | |
| NO | Description | Annotated Nucleic Acid (na) Sequence |
| 35 | GUS130-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R1-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 1 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCG{CCTAAAAAGAAACGAAAAGTGGGCA |
| | | TTCAC}GGCGTACCT [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 36 | GUS131-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R2-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 2 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] AGGGCC{CCTAAGAAAAAGAGAAAAGTCGGAA |
| | | TCCAC}GGTGTCCCA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 37 | GUS132-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R3-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 3 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] AAGGCA{CCCAAGAAGAAAAGAAAAGTAGGAA |
| | | TTCAC}GGAGTCCCT [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 38 | GUS133-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R1_HL-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 4 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCT{CCAAAAAAAAAACGCAAGGTTGGAA |
| | | TACAC}GGTGTACCT [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 39 | GUS134-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R2_HL-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 5 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCT{CCAAAGAAAAAGAGGAAGGTGGGAA |
| | | TACAT}GGAGTACCA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 40 | GUS135-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R3_HL-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 6 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCA{CCCAAAAAGAAAAGAAAGGTGGGTA |
| | | TTCAC}GGAGTTCCCGCT [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 41 | GUS136-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R-L) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Not diversified | TCCTCCAGCAGCTGTTTGTGTGCTGCCTCTGAAGTCCACACTGA |
| | ZFN-L | ACAAACTTCAGCCTAGTCATGTCCCTAAAATGGGCAAACATTGC |
| | Not diversified | AAGCAGCAAACAGCAAACACACAGCCCTCCCTGCCTGCTGACCT |
| | | TGGAGCTGGGGCAGAGGTCAGAGACCTCTCTGGGCCCATGCCAC |
| | | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCC{CCCAAGAAGAAGAGGAAGGTCGGCA |
| | | TTCAT}GGGGTACCC [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] (GGCAGCGGAGAGGGCAGA |
| | | GGAAGCCTGCTCACCTGCGGTGACGTGGAGGAAAACCCTGGCCC |
| | | T)ACGCGTGCCATGG [symbol] [symbol] |
| | | [symbol] [symbol] ATGGCC{C |
| | | CCAAGAAGAAGAGGAAGGTCGGCATTCAT}GGGGTACCCgccgc |
| | | tatggctgagaggcccttccagtgtcgaatctgcatgcagaact |
| | | tcagtcagtccggcaacctggcccgccacatccgcacccacacc |
| | | ggcgagaagccttttgcctgtgacatttgtgggaggaaatttgc |
| | | cctgaagcagaacctgtgtatgcataccaagatacacacgggcg |
| | | agaagcccttccagtgtcgaatctgcatgcagaagtttgcctgg |
| | | cagtccaacctgcagaaccataccaagatacacacgggcgagaa |
| | | gcccttccagtgtcgaatctgcatgcgtaacttcagtacctccg |
| | | gcaacctgacccgccacatccgcacccacaccggcgagaagcct |
| | | tttgcctgtgacatttgtgggaggaaatttgcccgccgctccca |
| | | cctgacctcccataccaagatacacctgcggggatcccagctgg |
| | | tgaagagcgagctggaggagaagaagtccgagctgcggcacaag |
| | | ctgaagtacgtgccccacgagtacatcgagctgatcgagatcgc |
| | | caggaacagcacccaggaccgcatcctggagatgaaggtgatgg |
| | | agttcttcatgaaggtgtacggctacaggggaaagcacctgggc |
| | | ggaagcagaaagcctgacggcgccatctatacagtgggcagccc |
| | | catcgattacggcgtgatcgtggacacaaaggcctacagcggcg |
| | | gctacaatctgcctatcggccaggccgacgagatggagagatac |
| | | gtggaggagaaccagacccgggataagcacctcaaccccaacga |
| | | gtggtggaaggtgtaccctagcagcgtgaccgagttcaagttcc |
| | | tgttcgtgagcggccacttcaagggcaactacaaggcccagctg |
| | | accaggctgaaccacatcaccaactgcgacggcgccgtgctgag |
| | | cgtggaggagctgctgatcggcggcgagatgatcaaagccggca |
| | | ccctgacactggaggaggtgcggcgcaagttcaacaacggcgag |
| | | atcaacttcagatcttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 42 | GUS140-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L1-R) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 1 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCT{CCAAAGAAGAAAAGAAAAGTGGGGA |
| | | TCCAT}GGTGTACCCgcagcaatggccgaacgacccttccaatg |
| | | cagaatatgtatgcagaatttttctcagagcgggaacctggcga |
| | | ggcacataagaacccatacaggagagaagccattcgcatgcgat |
| | | atttgcggtagaaaatttgcactcaaacaaaatctctgtatgca |
| | | cactaaaatccatacaggtgaaaagccttttcagtgcaggattt |
| | | gtatgcaaaaatttgcttggcaaagtaacttgcagaaccacaca |
| | | aagatacacacaggagagaaacccttccaatgccgaatctgtat |
| | | gcgcaacttcagtacatccggaaatttgactagacatattagga |
| | | cccacaccggcgagaagccatttgcctgcgatatttgtggacgg |
| | | aaattcgcacgacgcagccatctgaccagtcatactaagattca |
| | | tctccgcggcagccagcttgtgaagtccgaactggaggaaaaga |
| | | agagcgaactgcgccacaaattgaaatacgttccgcatgagtac |
| | | atagagctcattgaaatcgctagaaactctacccaagacaggat |
| | | actggaaatgaaagtgatggaatttttcatgaaagtttatggtt |
| | | ataggggcaaacatctgggtggctctcgcaagcccgatggggcc |
| | | atttatactgtcggctcacctatcgactatggcgtcattgtgga |
| | | taccaaggcttattctggaggatacaacctgcccatcggacaag |
| | | cagacgaaatggaaagatacgtcgaggagaatcaaacccgagac |
| | | aagcatctgaacccaaacgagtggtggaaagtgtacccgagcag |
| | | cgttactgagttcaaatttctctttgtaagcggacattttaaag |
| | | ggaattacaaagcacaactgactaggctgaaccatataaccaac |
| | | tgtgacggggccgtattgagtgtggaagagcttctgattggagg |
| | | agagatgattaaggctggcacactgactctcgaagaagtgaggc |
| | | gcaaattcaataacggtgaaatcaacttccggtct(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 49 | GUS141-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L2-R) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 2 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] AAGGCA{CCTAAAAAAAAGCGGAAAGTGGGAA |
| | | TTCAC}GGCGTGCCCgccgccatggcagagagaccctttcaatg |
| | | tagaatctgtatgcaaaatttctctcagagtggtaaccttgcaa |
| | | gacacatcagaactcatacaggtgagaagccgtttgcatgtgac |
| | | atttgcggtaggaaatttgccttgaaacagaatctttgtatgca |
| | | cacaaaaatccatactggtgaaaagccattccaatgccgcatct |
| | | gtatgcaaaaattcgcgtggcagtccaatttgcagaaccatacc |
| | | aagattcacacgggagaaaaaccatttcagtgccgcatctgcat |
| | | gcgcaacttttctacatcaggaaaccttacacgacatattcgga |
| | | cgcacactggagaaaaaccatttgcttgtgacatatgcggccga |
| | | aaatttgccagacgctctcatctcacctcacatactaagattca |
| | | tttgcgcggaagtcagctggtgaagagtgaattggaagaaaaaa |
| | | agtcagagctgagacacaaactgaaatatgttccacacgagtac |
| | | atcgagcttatcgagatagcaagaaactccacccaggacagaat |
| | | tttggaaatgaaagttatggaattctttatgaaagtgtatggct |
| | | acaggggtaaacatctggggggatcaagaaagcctgatggtgca |
| | | atttacacagtgggctctcctatcgactacggtgtgatcgtgga |
| | | tacaaaggcctactctggaggatataatttgcctattggacaag |
| | | ccgatgaaatggaaagatatgtggaggaaaaccagactcgcgat |
| | | aagcacctgaacccaaatgaatggtggaaagtgtacccttcatc |
| | | tgttaccgaatttaaatttttgttcgtttccgggcatttcaagg |
| | | ggaactacaaggcacagctgacgagactgaatcacatcacgaac |
| | | tgcgacggcgctgtactgtccgtggaagagcttttgatcggggg |
| | | cgaaatgattaaggccggcacactgacgctggaggaggtgcggc |
| | | gaaaatttaataatggcgagatcaattttaggagt(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 43 | GUS143-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L1_HL-R) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 4 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] AGGGCA{CCTAAGAAGAAGAGAAAAGTTGGAA |
| | | TACAT}GGAGTCCCCgcagcaatggccgagagaccttttcagtg |
| | | caggatttgtatgcaaaacttctctcagtccggtaacctggccc |
| | | ggcacatacgaacacataccggcgaaaaaccctttgcttgcgac |
| | | atctgcggaagaaagttcgctcttaaacagaacctgtgcatgca |
| | | tacaaaaattcatacaggtgagaagccattccaatgcagaatat |
| | | gtatgcagaaattcgcctggcaaagcaacctgcaaaaccacact |
| | | aagatccacacaggggaaaagccttttcaatgtagaatctgtat |
| | | gagaaactttagtacatccggaaatctcacacgacatatcagaa |
| | | cccacactggagaaaaaccttttgcctgcgacatctgcggaaga |
| | | aaattcgcccgaaggtcccacttgactagtcataccaaaatcca |
| | | cttgcgaggctcacagctggttaaatccgaacttgaagaaaaaa |
| | | aaagtgaactgcggcataaactgaagtatgtcccccatgaatat |
| | | atcgaactgatagaaatcgcccgaaatagcacccaagatagaat |
| | | cctcgaaatgaaggttatggaatttttcatgaaggtctatggat |
| | | ataggggcaagcaccttggcggatcccggaaacctgatggagct |
| | | atctacacagtgggctcaccaatagactatggagttatcgtcga |
| | | tacaaaagcatacagcggaggatacaatttgccaataggtcaag |
| | | cagatgagatggaaagatacgtggaggaaaaccaaacaagagat |
| | | aagcatctgaaccccaacgaatggtggaaagtgtaccccagttc |
| | | tgtaaccgaatttaagttcttgttcgtttcaggtcacttcaagg |
| | | gtaattacaaggctcaactgactagactcaaccatattacaaat |
| | | tgcgatggtgctgtgctttccgtggaagaattgctgattggtgg |
| | | agagatgataaaagctggtaccctcaccttggaagaagtgcgca |
| | | gaaaattcaataatggcgagatcaacttccgaagt(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 44 | GUS144-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L2_HL-R) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 5 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCC{CCCAAGAAGAAACGAAAAGTAGGAA |
| | | TCCAT}GGCGTGCCTgcagcaatggcagagagaccatttcagtg |
| | | cagaatatgtatgcaaaacttctcccagagcggtaatctggcta |
| | | ggcatattagaacacacaccggggaaaaacctttcgcttgcgat |
| | | atatgtggtagaaagttcgccctcaaacagaatctgtgcatgca |
| | | cactaaaatccatacaggagaaaagccctttcagtgtagaattt |
| | | gtatgcagaaatttgcttggcagtcaaatttgcaaaatcacacc |
| | | aaaatacacacaggagaaaaaccatttcagtgtagaatatgtat |
| | | gagaaatttttccacttccggaaatctgaccagacatatacgga |
| | | cacacactggggaaaagcccttcgcttgcgacatctgcggaaga |
| | | aagttcgctagacggtcccacttgacatcccacactaagataca |
| | | tcttcgcggtagccaactggtgaaaagtgaactggaggaaaaaa |
| | | aatctgagctgagacataaactgaaatacgtaccacatgaatac |
| | | atagaacttatagaaatagctaggaactccacccaggacagaat |
| | | acttgaaatgaaggtcatggagttttttatgaaagtttacggat |
| | | acaggggcaaacaccttggagggtctcggaagcctgatggcgca |
| | | atttataccgtgggtagccctatagattatggagtgattgtgga |
| | | tacaaaggcttacagtggcggctataatttgcctatcggacagg |
| | | ccgatgagatggaaagatacgttgaagaaaaccaaacacgagat |
| | | aagcatctgaaccccaatgaatggtggaaagtgtatccttcaag |
| | | cgttaccgagtttaagttcctcttcgtttctgggcatttcaagg |
| | | gcaactacaaagctcagcttacaagactcaaccacataaccaat |
| | | tgtgatggagcagtcctcagcgtggaagaactccttattggggg |
| | | tgagatgattaaagcagggacccttactcttgaagaggttagaa |
| | | gaaaattcaataacggagagattaattttagaagt(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 45 | GUS145-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L3_HL-R) | GCGCGCAGAGAGGGAGTGGCCAACTCCATCACTAGGGGTTCCT] |
| | (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 6 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Not diversified | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCA{CCTAAGAAGAAAAGAAAGGTCGGCA |
| | | TTCAT}GGTGTGCCTgcagccatggccgaacgcccatttcaatg |
| | | tagaatttgtatgcagaatttttcacaatcaggaaacctggcta |
| | | gacatatcagaacacatactggagaaaagccctttgcttgtgat |
| | | atctgtggaaggaaattcgccctgaaacaaaacctctgtatgca |
| | | cacaaagatccacaccggcgaaaagcctttccagtgtaggatat |
| | | gcatgcaaaaattcgcctggcagtccaatctgcagaaccatacc |
| | | aaaattcatactggtgaaaagccatttcagtgcagaatatgtat |
| | | gagaaactttagcacttcaggaaatctcacaagacatataagaa |
| | | cacatacaggggaaaaaccttttgcttgcgatatctgcggcagg |
| | | aaattcgctcggagaagtcatctcacaagccatacaaaaatcca |
| | | cctgcgaggaagccagctggtcaagtctgaactggaagaaaaaa |
| | | aaagcgaactgcggcataaactcaaatacgtcccacatgaatac |
| | | attgagctcatcgaaattgctagaaactctactcaagataggat |
| | | attggagatgaaggtaatggaattcttcatgaaggtttatggat |
| | | atagaggaaaacatcttggaggcagtaggaaacccgatggcgct |
| | | atctacaccgtagggagtccaatcgactacggcgtgattgttga |
| | | caccaaagcctattctggagggtataatctcccaattggacagg |
| | | cagatgagatggaaagatatgtagaagaaaatcagacaagagat |
| | | aagcaccttaaccctaacgagtggtggaaagtgtacccaagcag |
| | | tgttactgaatttaaatttctttttgtatcaggacactttaaag |
| | | gcaattacaaagcacaactgaccagactcaatcacattaccaat |
| | | tgcgacggagccgtactgagcgtggaggagttgctgatcggagg |
| | | cgaaatgattaaagctggcactctgaccctggaagaagtaagaa |
| | | gaaagttcaataatggagaaataaactttcgctcc(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 46 | GUS146-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L-R) (na) | GCGCGCAGAGAGGGAGTGGCCAACTCCATCACTAGGGGTTCCT] |
| | ZFN-R | GCGGCCTAAGCTTGAGCTCTTCGAAAGGCTCAGAGGCACACAGG |
| | Not diversified | AGTTTCTGGGCTCACCCTGCCCCCTTCCAACCCCTCAGTTCCCA |
| | ZFN-L | TCCTCCAGCAGCTGTTTGTGTGCTGCCTCTGAAGTCCACACTGA |
| | Not diversified | ACAAACTTCAGCCTAGTCATGTCCCTAAAATGGGCAAACATTGC |
| | | AAGCAGCAAACAGCAAACACACAGCCCTCCCTGCCTGCTGACCT |
| | | TGGAGCTGGGGCAGAGGTCAGAGACCTCTCTGGGCCCATGCCAC |
| | | CTCCAACATCCACTCGACCCCTTGGAATTTCGGTGGAGAGGAGC |
| | | AGAGGTTGTCCTGGCGTGGTTTAGGTAGTGTGAGAGGGGTCCCG |
| | | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCC{CCCAAGAAGAAGAGGAAGGTCGGCA |
| | | TTCAT}GGGGTACCCgccgctatggctgagaggcccttccagtg |
| | | tcgaatctgcatgcagaacttcagtcagtccggcaacctggccc |
| | | gccacatccgcacccacaccggcgagaagccttttgcctgtgac |
| | | atttgtgggaggaaatttgccctgaagcagaacctgtgtatgca |
| | | taccaagatacacacgggcgagaagcccttccagtgtcgaatct |
| | | gcatgcagaagtttgcctggcagtccaacctgcagaaccatacc |
| | | aagatacacacgggcgagaagcccttccagtgtcgaatctgcat |
| | | gcgtaacttcagtacctccggcaacctgacccgccacatccgca |
| | | cccacaccggcgagaagccttttgcctgtgacatttgtgggagg |
| | | aaatttgcccgccgctcccacctgacctcccataccaagataca |
| | | cctgcggggatcccagctggtgaagagcgagctggaggagaaga |
| | | agtccgagctgcggcacaagctgaagtacgtgccccacgagtac |
| | | atcgagctgatcgagatcgccaggaacagcacccaggaccgcat |
| | | cctggagatgaaggtgatggagttcttcatgaaggtgtacggct |
| | | acaggggaaagcacctgggcggaagcagaaagcctgacggcgcc |
| | | atctatacagtgggcagccccatcgattacggcgtgatcgtgga |
| | | cacaaaggcctacagcggcggctacaatctgcctatcggccagg |
| | | ccgacgagatggagagatacgtggaggagaaccagacccgggat |
| | | aagcacctcaaccccaacgagtggtggaaggtgtaccctagcag |
| | | cgtgaccgagttcaagttcctgttcgtgagcggccacttcaagg |
| | | gcaactacaaggcccagctgaccaggctgaaccacatcaecaac |
| | | tgcgacggcgccgtgctgagcgtggaggagctgetgateggegg |
| | | egagatgatcaaagccggcaccctgacactggaggaggtgcggc |
| | | gcaagttcaacaacggcgagatcaacttcagatct(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCC{CCCAAGAAGAAGAGGAAGGTCGGCATTCAT}GGGG |
| | | TACCC [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 47 | GUS150-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (L2- | GCGCGCAGAGAGGGAGTGGCCAACTCCATCACTAGGGGTTCCT] |
| | R1_HL) (na) | GCGGCCTAAGCTTGAGCTCTTCGAAAGGCTCAGAGGCACACAGG |
| | ZFN-L | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 2 | AAGCAGCAAACAGCAAACACACAGCCCTCCCTGCCTGCTGACCT |
| | ZFN-R | TGGAGCTGGGGCAGAGGTCAGAGACCTCTCTGGGCCCATGCCAC |
| | Codon | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | Version 4 | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTGAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCA{CCTAAAAAAAAGCGGAAAGTGGGAA |
| | | TTCAC}GGCGTGCCCgccgccatggcagagagaccctttcaatg |
| | | tagaatctgtatgcaaaatttctctcagagtggtaaccttgcaa |
| | | gacacatcagaactcatacaggtgagaagccgtttgcatgtgac |
| | | atttgcggtaggaaatttgccttgaaacagaatctttgtatgca |
| | | cacaaaaatccatactggtgaaaagccattccaatgccgcatct |
| | | gtatgcaaaaattcgcgtggcagtccaatttgcagaaccatacc |
| | | aagattcacacgggagaaaaaccatttcagtgccgcatctgcat |
| | | gcgcaacttttctacatcaggaaaccttacacgacatattcgga |
| | | cgcacactggagaaaaaccatttgcttgtgacatatgcggccga |
| | | aaatttgccagacgctctcatctcacctcacatactaagattca |
| | | tttgcgcggaagtcagctggtgaagagtgaattggaagaaaaaa |
| | | agtcagagctgagacacaaactgaaatatgttccacacgagtac |
| | | atcgagcttatcgagatagcaagaaactccacccaggacagaat |
| | | tttggaaatgaaagttatggaattctttatgaaagtgtatggct |
| | | acaggggtaaacatctggggggatcaagaaagcctgatggtgca |
| | | atttacacagtgggctctcctatcgactacggtgtgatcgtgga |
| | | tacaaaggcctactctggaggatataatttgcctattggacaag |
| | | ccgatgaaatggaaagatatgtggaggaaaaccagactcgcgat |
| | | aagcacctgaacccaaatgaatggtggaaagtgtacccttcatc |
| | | tgttaccgaatttaaatttttgttcgtttccgggcatttcaagg |
| | | ggaactacaaggcacagctgacgagactgaatcacatcacgaac |
| | | tgcgacggcgctgtactgtccgtggaagagcttttgatcggggg |
| | | cgaaatgattaaggccggcacactgacgctggaggaggtgcggc |
| | | gaaaatttaataatggcgagatcaattttaggagt(GGCAGCGG |
| | | AGAGGGCAGAGGAAGCCTGCTCACCTGCGGTGACGTGGAGGAAA |
| | | ACCCTGGCCCT)ACGCGTGCCATG [symbol] |
| | | [symbol] [symbol] |
| | | [symbol] ATGGCT{CCAAAAAAAAAACGCAAGGTTGGAATACAC}GGTG |
| | | TACCT [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] CTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |
| 48 | GUS151-pAAV- | [CTGCGCGCTCGCTCGCTCACTGAGGCCGCCCGGGCAAAGCCCG |
| | hZFN-2-in-1 | GGCGTCGGGCGACCTTTGGTCGCCCGGCCTCAGTGAGCGAGCGA |
| | vector (R1-_HL- | GCGCGCAGAGAGGGAGTGGCCAACTCCATCACTAGGGGTTCCT] |
| | L2) (na) | GCGGCCTAAGCTTGAGCTCTTCGAAAGGCTCAGAGGCACACAGG |
| | ZFN-R | AGTTTCTGGGCTCACCCTGCCCCCTTCCAACCCCTCAGTTCCCA |
| | Codon | TCCTCCAGCAGCTGTTTGTGTGCTGCCTCTGAAGTCCACACTGA |
| | diversified | ACAAACTTCAGCCTAGTCATGTCCCTAAAATGGGCAAACATTGC |
| | Version 4 | AAGCAGCAAACAGCAAACACACAGCCCTCCCTGCCTGCTGACCT |
| | ZFN-L | TGGAGCTGGGGCAGAGGTCAGAGACCTCTCTGGGCCCATGCCAC |
| | Codon | CTCCAACATCCACTCGACCCCTTGGAATTTCGGTGGAGAGGAGC |
| | diversified | AGAGGTTGTCCTGGCGTGGTTTAGGTAGTGTGAGAGGGGTCCCG |
| | Version 2 | GGGATCTTGCTACCAGTGGAACAGCCACTAAGGATTCTGCAGTG |
| | | AGAGCAGAGGGCCAGCTAAGTGGTACTCTCCCAGAGACTGTCTG |
| | | ACTCACGCCACCCCCTCCACCTTGGACACAGGACGCTGTGGTTT |
| | | CTGAGCCAGGTACAATGACTCCTTTCGGTAAGTGCAGTGGAAGC |
| | | TGTACACTGCCCAGGCAAAGCGTCCGGGCAGCGTAGGCGGGCGA |
| | | CTCAGATCCCAGCCAGTGGACTTAGCCCCTGTTTGCTCCTCCGA |
| | | TAACTGGGGTGACCTTGGTTAATATTCACCAGCAGCCTCCCCCG |
| | | TTGCCCCTCTGGATCCACTGCTTAAATACGGACGAGGACAGGGC |
| | | CCTGTCTCCTCAGCTTCAGGCACCACCACTGACCTGGGACAGTC |
| | | CTAGGTGCTTGTTCTTTTTGCAGAAGCTCAGAATAAACGCTCAA |
| | | CTTTGGCAGATACTAGTCAGGTAAGTATCAAGGTTACAAGACAG |
| | | GTTTAAGGAGACCAATAGAAACTGGGCTTGTCGAGACAGAGAAG |
| | | ACTCTTGCGTTTCTGATAGGCACCTATTGGTCTTACTGACATCC |
| | | ACTTTGCCTTTCTCTCCACAGGACCGGTGCCATG [symbol] |
| | | [symbol] [symbol] |
| | | [symbol] ATGGCT{CCAAAAAAAAAACGCAAGGTTGGAA |
| | | TACAC}GGTGTACCTG [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | GITCAACAACGGCGAAATCAACTTT(GGCAGCGGAGAGGGCAGA |
| | | GGAAGCCTGCTCACCTGCGGTGACGTGGAGGAAAACCCTGGCCC |
| | | T)ACGCGTGCCATG [symbol] |
| | | [symbol] [symbol] ATGGCA{C |
| | | CTAAAAAAAAGCGGAAAGTGGGAATTCAC}GGCGTGCCCgccgc |
| | | catggcagagagaccctttcaatgtagaatctgtatgcaaaatt |
| | | tctctcagagtggtaaccttgcaagacacatcagaactcataca |
| | | ggtgagaagccgtttgcatgtgacatttgcggtaggaaatttgc |
| | | cttgaaacagaatctttgtatgcacacaaaaatccatactggtg |
| | | aaaagccattccaatgccgcatctgtatgcaaaaattcgcgtgg |
| | | cagtccaatttgcagaaccataccaagattcacacgggagaaaa |
| | | accatttcagtgccgcatctgcatgcgcaacttttctacatcag |
| | | gaaaccttacacgacatattcggacgcacactggagaaaaacca |
| | | tttgcttgtgacatatgcggccgaaaatttgccagacgctctca |
| | | tctcacctcacatactaagattcatttgcgcggaagtcagctgg |
| | | tgaagagtgaattggaagaaaaaaagtcagagctgagacacaaa |
| | | ctgaaatatgttccacacgagtacatcgagcttatcgagatagc |
| | | aagaaactccacccaggacagaattttggaaatgaaagttatgg |
| | | aattctttatgaaagtgtatggctacaggggtaaacatctgggg |
| | | ggatcaagaaagcctgatggtgcaatttacacagtgggctctcc |
| | | tatcgactacggtgtgatcgtggatacaaaggcctactctggag |
| | | gatataatttgcctattggacaagccgatgaaatggaaagatat |
| | | gtggaggaaaaccagactcgcgataagcacctgaacccaaatga |
| | | atggtggaaagtgtacccttcatctgttaccgaatttaaatttt |
| | | tgttcgtttccgggcatttcaaggggaactacaaggcacagctg |
| | | acgagactgaatcacatcacgaactgcgacggcgctgtactgtc |
| | | cgtggaagagcttttgatcgggggcgaaatgattaaggccggca |
| | | cactgacgctggaggaggtgcggcgaaaatttaataatggcgag |
| | | atcaattttaggagttgataaCTCGAGTCTAGA [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] [symbol] |
| | | [symbol] GCTAGC [symbol] [symbol] |
| | | [symbol] [symbol] |
| | | [symbol] [symbol] |
| | | [symbol] [symbol] |
| | | [symbol] [symbol] |
| | | [symbol] GCGGCCGCGTCGAGCGC[AGGAACCC |
| | | CTAGTGATGGAGTTGGCCACTCCCTCTCTGCGCGCTCGCTCGCT |
| | | CACTGAGGCCGCCCGGGCTTTGCCCGGGCGGCCTCAGTGAGCGA |
| | | GCGAGCGCGCAG] |

Legend:
5′ITR = [plain text in brackets]
ApoE (Enhancer) = underline
hAAT (Promoter) = italics
5′UTR = boldHuman β-globin/IgG chimeric intron = double underline3xFLAG = bold italicsNLS = {plain text in curly brackets}
ZFN-L = lower case
2A peptide = (plain text in parentheses)
ZFN-R = Dashed underline
WPREmut6 = Dotted underline
Polyadenylation signal = Wavy underline
3′ITR = [bold text in brackets]

### NUMBERED EMBODIMENTS

Particular embodiments of the disclosure are set forth in the following numbered paragraphs:
1. A method for treating or preventing a lysosomal storage disorder in a subject, the method comprising modifying a target sequence in the genome of a cell of said subject by introducing into the cell a nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      or a vector comprising said nucleic acid encoding a 2-in-1 zinc finger nuclease variant;
      wherein the polynucleotide encoding the 2A self-cleaving peptide is positioned between the polynucleotide encoding the first zinc finger nuclease and the polynucleotide encoding the second zinc finger nuclease.
2. A method for correcting a lysosomal storage disease-causing mutation in the genome of a cell, the method comprising modifying a target sequence in the genome of the cell by introducing into the cell a nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      or a vector comprising said nucleic acid encoding a 2-in-1 zinc finger nuclease variant;
      wherein the polynucleotide encoding the 2A self-cleaving peptide is positioned between the polynucleotide encoding the first zinc finger nuclease and the polynucleotide encoding the second zinc finger nuclease.
3. A method for modifying the genome of a cell comprising a mutation in a gene associated with a lysosomal storage disease, the method comprising introducing into a cell a nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      or a vector comprising said nucleic acid encoding a 2-in-1 zinc finger nuclease variant;
      wherein the polynucleotide encoding the 2A self-cleaving peptide is positioned between the polynucleotide encoding the first zinc finger nuclease and the polynucleotide encoding the second zinc finger nuclease.
4. A method for integrating an exogenous nucleotide sequence into a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      or a vector comprising said nucleic acid encoding a 2-in-1 zinc finger nuclease variant;
      wherein the polynucleotide encoding the 2A self-cleaving peptide is positioned between the polynucleotide encoding the first zinc finger nuclease and the polynucleotide encoding the second zinc finger nuclease.
5. A method for disrupting a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      or a vector comprising said nucleic acid encoding a 2-in-1 zinc finger nuclease variant;
      wherein the polynucleotide encoding the 2A self-cleaving peptide is positioned between the polynucleotide encoding the first zinc finger nuclease and the polynucleotide encoding the second zinc finger nuclease.
6. The method according to any one of paragraphs 1-5, further comprising introducing into the cell a donor nucleic acid or a vector comprising said donor nucleic acid, wherein said donor nucleic acid comprises a polynucleotide encoding a corrective lysosomal storage disease-associated protein or enzyme or portion thereof
7. The method according to paragraph 6, wherein the donor nucleic acid is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.
8. The method according to paragraph 6, wherein corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.
9. The method according to any one of paragraph 1-8, wherein the nucleic acid encoding a 2-in-1 zinc finger nuclease variant further comprises a polynucleotide sequence selected from one or more of:
   a. a polynucleotide sequence encoding a nuclear localization sequence;
   b. a 5′ITR polynucleotide sequence;
   c. an enhancer polynucleotide sequence;
   d. a promoter polynucleotide sequence;
   e. a 5′UTR polynucleotide sequence;
   f. a chimeric intron polynucleotide sequence;
   g. a polynucleotide sequences encoding an epitope tag;
   h. a polynucleotide sequence encoding a Fok I cleavage domain;
   i. a post-transcriptional regulatory element polynucleotide sequence;
   j. a polyadenylation signal sequence; and
   k. a 3′ITR polynucleotide sequence.
10. The method according to paragraph 9, wherein the nucleic acid encoding a 2-in-1 zinc finger nuclease variant comprises two independent polynucleotide sequences encoding two nuclear localization sequences.
11. The method according to paragraph 9, wherein the nucleic acid encoding a 2-in-1 zinc finger nuclease variant comprises two or more independent polynucleotide sequences encoding two or more epitope tags.
12. The method according to paragraph 9, wherein the nucleic acid encoding a 2-in-1 zinc finger nuclease variant comprises two or more independent polynucleotide sequences encoding two or more Fok I cleavage domains.
13. The method according to any one of paragraphs 1-12, wherein the polynucleotide encoding the first zinc finger nuclease is codon diversified.
14. The method according to any one of paragraphs 1-12, wherein the polynucleotide encoding the second zinc finger nuclease is codon diversified.
15. The method according to any one of paragraphs 1-12, wherein the polynucleotide encoding the first zinc finger nuclease is codon diversified and the polynucleotide encoding the second zinc finger nuclease is codon diversified.
16. The method according to any one of paragraphs 1-12, wherein the polynucleotide encoding the first zinc finger nuclease comprises the nucleotide sequence of any one of SEQ ID NOs: 116-129.
17. The method according to any one of paragraphs 1-12 or 16, wherein the polynucleotide encoding the second zinc finger nuclease comprises the nucleotide sequence of any one of SEQ ID NOs: 116-129.
18. The method according to any one of paragraphs 1-12, wherein the polynucleotide encoding the first zinc finger nuclease comprises a nucleotide sequence encoding the amino acid sequence of SEQ ID NOs: 136 or 137.
19. The method according to any one of paragraphs 1-12 or 18, wherein the polynucleotide encoding the second zinc finger nuclease comprises a nucleotide sequence encoding the amino acid sequence of SEQ ID NOs: 136 or 137.
20. The method according to any one of paragraphs 1-12, wherein the polynucleotide sequence encoding the first zinc finger nuclease comprises the nucleotide sequence of any one of SEQ ID NOs: 71-84.
21. The method according to any one of paragraphs 1-12 or 20, wherein the polynucleotide sequence encoding the second zinc finger nuclease comprises the nucleotide sequence of any one of SEQ ID NOs: 71-84.
22. The method according to any one of paragraphs 1-12, wherein the polynucleotide encoding the first zinc finger nuclease comprises a nucleotide sequence encoding the amino acid sequence of SEQ ID NOs: 130 or 131.
23. The method according to any one of paragraphs 1-12 or 22, wherein the polynucleotide encoding the second zinc finger nuclease comprises a nucleotide sequence encoding the amino sequence of SEQ ID NOs: 130 or 131.
24. The method according to any one of paragraphs 1-12, wherein the polynucleotide sequence encoding the first zinc finger nuclease comprises the nucleotide sequence of any one of SEQ ID NOs: 139-152.
25. The method according to any one of paragraphs 1-12 or 24, wherein the polynucleotide sequence encoding the second zinc finger nuclease comprises the nucleotide sequence of any one of SEQ ID NOs: 139-152.
26. The method according to any one of paragraphs 1-12, wherein the polynucleotide sequence encoding the first zinc finger nuclease comprises the nucleotide sequence of any one of SEQ ID NOs: 17-23 and 25-31.
27. The method according to any one of paragraphs 1-12 or 26, wherein the polynucleotide sequence encoding the second zinc finger nuclease comprises the nucleotide sequence of any one of SEQ ID NOs: 17-23 and 25-31.
28. The method according to any one of paragraphs 1-27, wherein the nucleic acid encoding a 2-in-1 zinc finger nuclease variant comprises a nucleotide sequence selected from any one of SEQ ID NO: 85-115.
29. The method according to any one of paragraphs 1-27, wherein the nucleic acid encoding a 2-in-1 zinc finger nuclease variant comprises a nucleotide sequence selected from any one of SEQ ID NO: 35-49.
30. The method according to any one of paragraphs 1-29, wherein the vector is an AAV vector.
31. A method for treating or preventing a lysosomal storage disorder in a subject, the method comprising modifying a target sequence in the genome of a cell of said subject by introducing into the cell a 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and the second zinc finger nuclease.
32. A method for correcting a lysosomal storage disease-causing mutation in the genome of a cell, the method comprising modifying a target sequence in the genome of the cell by introducing into the cell a 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and second zinc finger nuclease.
33. A method for modifying the genome of a cell comprising a mutation in a gene associated with a lysosomal storage disease, the method comprising introducing into a cell a 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and second zinc finger nuclease.
34. A method for integrating an exogenous nucleotide sequence into a target nucleotide sequence in a gene of a cell, wherein said gene comprises a mutation associated with a lysosomal storage disease, the method comprising introducing into the cell a 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and second zinc finger nuclease.
35. A method for disrupting a target nucleotide sequence in a gene of a cell, wherein said gene comprises a mutation associated with a lysosomal storage disease, the method comprising introducing into the cell a 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and second zinc finger nuclease.
36. The method according to any one of paragraphs 31-35, further comprising introducing into the cell a donor nucleic acid or a vector comprising said donor nucleic acid, wherein said donor nucleic acid comprises a polynucleotide encoding a corrective lysosomal storage disease-associated protein or enzyme or portion thereof
37. The method according to paragraph 36, wherein the donor nucleic acid is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.
38. The method according to paragraph 36, wherein corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.
39. The method according to any one of paragraphs 31-38, wherein the 2-in-1 zinc finger nuclease variant further comprises one or more of:
   a. a nuclear localization sequence;
   b. an epitope tag; and
   c. a Fok I cleavage domain.
40. The method according to paragraph 39, wherein the 2-in-1 zinc finger nuclease variant comprises two independent nuclear localization sequences.
41. The method according to paragraph 39, wherein the 2-in-1 zinc finger nuclease variant comprises two or more independent epitope tags.
42. The method according to paragraph 39, wherein the 2-in-1 zinc finger nuclease variant comprises two or more independent Fok I cleavage domains.
43. The method according to any one of paragraphs 31-42, wherein the first zinc finger nuclease is codon diversified.
44. The method according to any one of paragraphs 31-42, wherein the second zinc finger nuclease is codon diversified.
45. The method according to any one of paragraphs 31-42, wherein the first zinc finger nuclease is codon diversified and the second zinc finger nuclease is codon diversified.
46. The method according to any one of paragraphs 31-42, wherein the first zinc finger nuclease is encoded by a polynucleotide comprising the nucleotide sequence of any one of SEQ ID NOs: 116-129.
47. The method according to any one of paragraphs 31-42 or 46, wherein the second zinc finger nuclease is encoded by a polynucleotide comprising the nucleotide sequence of any one of SEQ ID NOs: 116-129.
48. The method according to any one of paragraphs 31-42 wherein the first zinc finger nuclease comprises the amino acid sequence of SEQ ID NOs: 136 or 137.
49. The method according to any one of paragraphs 31-42 or 48, wherein the second zinc finger nuclease comprises the amino acid sequence of SEQ ID NOs: 136 or 137.
50. The method according to any one of paragraphs 31-42, wherein the first zinc finger nuclease is encoded by a polynucleotide sequence comprising the nucleotide sequence of any one of SEQ ID NOs: 71-84.
51. The method according to any one of paragraphs 31-42 or 50, wherein the second zinc finger nuclease is encoded by a polynucleotide sequence comprising the nucleotide sequence of any one of SEQ ID NOs: 71-84.
52. The method according to any one of paragraphs 31-42, wherein the first zinc finger nuclease comprises the amino acid sequence of SEQ ID NOs: 130 or 131.
53. The method according to any one of paragraphs 31-42 or 52, wherein the second zinc finger nuclease comprises the amino sequence of SEQ ID NOs: 130 or 131.
54. The method according to any one of paragraphs 31-42, wherein the first zinc finger nuclease is encoded by a polynucleotide comprising the nucleotide sequence of any one of SEQ ID NOs: 139-152.
55. The method according to any one of paragraphs 31-42 or 54, wherein the second zinc finger nuclease is encoded by a polynucleotide comprising the nucleotide sequence of any one of SEQ ID NOs: 139-152.
56. The method according to any one of paragraphs 31-42, wherein the first zinc finger nuclease is encoded by a polynucleotide comprising the nucleotide sequence of any one of SEQ ID NOs: 17-23 and 25-31.
57. The method according to any one of paragraphs 31-42 or 56, wherein the second zinc finger nuclease is encoded by a polynucleotide comprising the nucleotide sequence of any one of SEQ ID NOs: 17-23 and 25-31.
58. The method according to any one of paragraphs 1-57, wherein the 2-in-1 zinc finger nuclease variant is encoded by a nucleotide sequence selected from any one of SEQ ID NO: 85-115.
59. The method according to any one of paragraphs 1-57, wherein the 2-in-1 zinc finger nuclease variant is encoded by a nucleotide sequence selected from any one of SEQ ID NO: 35-49.
60. The method according to any one of paragraphs 1-59, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
61. The method according to any one of paragraphs 1-59, wherein the lysosomal storage disease is selected from MPSI and MPSII.
62. The method according to paragraph 61, wherein the lysosomal storage disease is selected from the group consisting of MPS I—Hurler Syndrome, MPS I—Scheie Syndrome, and MPS I—Hurler-Scheie Syndrome.
63. The method according to paragraph 61, wherein the lysosomal storage disease is MPSII Hunter Syndrome.
64. A nucleic acid encoding a 2-in-1 zinc finger nuclease variant comprising:
   a. a polynucleotide encoding a first zinc finger nuclease;
   b. a polynucleotide encoding a second zinc finger nuclease; and
   c. a polynucleotide encoding a 2A self-cleaving peptide;
      wherein the polynucleotide encoding the 2A self-cleaving peptide is positioned between the polynucleotide encoding the first zinc finger nuclease and the polynucleotide encoding the second zinc finger nuclease.
65. The nucleic acid according to paragraph 64, wherein the nucleic acid encoding a 2-in-1 zinc finger nuclease variant further comprises a polynucleotide sequence selected from one or more of:
   a. a polynucleotide sequence encoding a nuclear localization sequence;
   b. a 5′ITR polynucleotide sequence;
   c. an enhancer polynucleotide sequence;
   d. a promoter polynucleotide sequence;
   e. a 5′UTR polynucleotide sequence;
   f. a chimeric intron polynucleotide sequence;
   g. a polynucleotide sequences encoding an epitope tag;
   h. a polynucleotide sequence encoding a Fok I cleavage domain;
   i. a post-transcriptional regulatory element polynucleotide sequence;
   j. a polyadenylation signal sequence; and
   k. a 3′ITR polynucleotide sequence.
66. The nucleic acid according to paragraph 65, wherein the nucleic acid encoding a 2-in-1 zinc finger nuclease variant comprises two independent polynucleotide sequences encoding two nuclear localization sequences.
67. The nucleic acid according to paragraph 65, wherein the nucleic acid encoding a 2-in-1 zinc finger nuclease variant comprises two or more independent polynucleotide sequences encoding two or more epitope tags.
68. The nucleic acid according to paragraph 65, wherein the nucleic acid encoding a 2-in-1 zinc finger nuclease variant comprises two or more independent polynucleotide sequences encoding two or more Fok I cleavage domains.
69. The nucleic acid according to any one of paragraphs 64-68, wherein the polynucleotide encoding the first zinc finger nuclease is codon diversified.
70. The nucleic acid according to any one of paragraphs 64-68, wherein the polynucleotide encoding the second zinc finger nuclease is codon diversified.
71. The nucleic acid according any one of paragraphs 64-68, wherein the polynucleotide encoding the first zinc finger nuclease is codon diversified and the polynucleotide encoding the second zinc finger nuclease is codon diversified.
72. The nucleic acid according to any one of paragraphs 64-69, wherein the polynucleotide encoding the first zinc finger nuclease comprises the nucleotide sequence of any one of SEQ ID NOs: 116-129.
73. The nucleic acid according to any one of paragraphs 64-68 or 72, wherein the polynucleotide encoding the second zinc finger nuclease comprises the nucleotide sequence of any one of SEQ ID NOs: 116-129.
74. The nucleic acid according to any one of paragraphs 64-68, wherein the polynucleotide encoding the first zinc finger nuclease comprises a nucleotide sequence encoding the amino acid sequence of SEQ ID NOs: 136 or 137.
75. The nucleic acid according to any one of paragraphs 64-68 or 74, wherein the polynucleotide encoding the second zinc finger nuclease comprises a nucleotide sequence encoding the amino acid sequence of SEQ ID NOs: 136 or 137.
76. The nucleic acid according to any one of paragraphs 64-68, wherein the polynucleotide sequence encoding the first zinc finger nuclease comprises the nucleotide sequence of any one of SEQ ID NOs: 71-84.
77. The nucleic acid according to any one of paragraphs 64-68 or 76, wherein the polynucleotide sequence encoding the second zinc finger nuclease comprises the nucleotide sequence of any one of SEQ ID NOs: 71-84.
78. The nucleic acid according to any one of paragraphs 64-68, wherein the polynucleotide encoding the first zinc finger nuclease comprises a nucleotide sequence encoding the amino acid sequence of SEQ ID NOs: 130 or 131.
79. The nucleic acid according to any one of paragraphs 64-68 or 78, wherein the polynucleotide encoding the second zinc finger nuclease comprises a nucleotide sequence encoding the amino sequence of SEQ ID NOs: 130 or 131.
80. The nucleic acid according to any one of paragraphs 64-68, wherein the polynucleotide sequence encoding the first zinc finger nuclease comprises the nucleotide sequence of any one of SEQ ID NOs: 139-152.
81. The nucleic acid according to any one of paragraphs 64-68 or 80, wherein the polynucleotide sequence encoding the second zinc finger nuclease comprises the nucleotide sequence of any one of SEQ ID NOs: 139-152.
82. The nucleic acid according to any one of paragraphs 64-68, wherein the polynucleotide sequence encoding the first zinc finger nuclease comprises the nucleotide sequence of any one of SEQ ID NOs: 17-23 and 25-31.
83. The nucleic acid according to any one of paragraphs 64-68 or 82, wherein the polynucleotide sequence encoding the second zinc finger nuclease comprises the nucleotide sequence of any one of SEQ ID NOs: 17-23 and 25-31.
84. The nucleic acid according to any one of paragraphs 64-83, wherein the nucleic acid encoding a 2-in-1 zinc finger nuclease variant comprises a nucleotide sequence selected from any one of SEQ ID NO: 85-115.
85. The nucleic acid according to any one of paragraphs 641-83, wherein the nucleic acid encoding a 2-in-1 zinc finger nuclease variant comprises a nucleotide sequence selected from any one of SEQ ID NO: 35-49.
86. A 2-in-1 zinc finger nuclease variant comprising:
   a. a first zinc finger nuclease;
   b. a second zinc finger nuclease; and
   c. a 2A self-cleaving peptide;
      wherein the 2A self-cleaving peptide is positioned between the first zinc finger nuclease and second zinc finger nuclease.
87. The 2-in-1 zinc finger nuclease variant according to paragraph 86, further comprising one or more of:
   a. a nuclear localization sequence;
   b. an epitope tag; and
   c. a Fok I cleavage domain.
88. The 2-in-1 zinc finger nuclease variant according to paragraph 87, wherein the 2-in-1 zinc finger nuclease variant comprises two independent nuclear localization sequences.
89. The 2-in-1 zinc finger nuclease variant according to paragraph 87, wherein the 2-in-1 zinc finger nuclease variant comprises two or more independent epitope tags.
90. The 2-in-1 zinc finger nuclease variant according to paragraph 87, wherein the 2-in-1 zinc finger nuclease variant comprises two or more independent Fok I cleavage domains.
91. The 2-in-1 zinc finger nuclease variant according to any one of paragraphs 86-90, wherein the first zinc finger nuclease is codon diversified.
92. The 2-in-1 zinc finger nuclease variant according to any one of paragraphs 86-90, wherein the second zinc finger nuclease is codon diversified.
93. The 2-in-1 zinc finger nuclease variant according any one of paragraphs 86-90, wherein the first zinc finger nuclease is codon diversified and the second zinc finger nuclease is codon diversified.
94. The 2-in-1 zinc finger nuclease variant according to any one of paragraphs 86-90, wherein the first zinc finger nuclease is encoded by a polynucleotide comprising the nucleotide sequence of any one of SEQ ID NOs: 116-129.
95. The 2-in-1 zinc finger nuclease variant according to any one of paragraphs 86-90, wherein the second zinc finger nuclease is encoded by a polynucleotide comprising the nucleotide sequence of any one of SEQ ID NOs: 116-129.
96. The 2-in-1 zinc finger nuclease variant according to any one of paragraphs 86-90, wherein the first zinc finger nuclease comprises the amino acid sequence of SEQ ID NOs: 136 or 137.
97. The 2-in-1 zinc finger nuclease variant according to any one of paragraphs 86-90 or 96, wherein the second zinc finger nuclease comprises the amino acid sequence of SEQ ID NOs: 136 or 137.
98. The 2-in-1 zinc finger nuclease variant according to any one of paragraphs 86-90, wherein the first zinc finger nuclease is encoded by a polynucleotide sequence comprising the nucleotide sequence of any one of SEQ ID NOs: 71-84.
99. The 2-in-1 zinc finger nuclease variant according to any one of paragraphs 86-90 or 98, wherein the second zinc finger nuclease is encoded by a polynucleotide sequence comprising the nucleotide sequence of any one of SEQ ID NOs: 71-84.
100. The 2-in-1 zinc finger nuclease variant according to any one of paragraphs 86-90, wherein the first zinc finger nuclease comprises the amino acid sequence of SEQ ID NOs: 130 or 131.
101. The 2-in-1 zinc finger nuclease variant according to any one of paragraphs 86-90 or 100, wherein the second zinc finger nuclease comprises the amino sequence of SEQ ID NOs: 130 or 131.
102. The 2-in-1 zinc finger nuclease variant according to any one of paragraphs 86-90, wherein the first zinc finger nuclease is encoded by a polynucleotide comprising the nucleotide sequence of any one of SEQ ID NOs: 139-152.
103. The 2-in-1 zinc finger nuclease variant according to any one of paragraphs 86-90 or 102, wherein the second zinc finger nuclease is encoded by a polynucleotide comprising the nucleotide sequence of any one of SEQ ID NOs: 139-152.
104. The 2-in-1 zinc finger nuclease variant according to any one of paragraphs 86-90, wherein the first zinc finger nuclease is encoded by a polynucleotide comprising the nucleotide sequence of any one of SEQ ID NOs: 17-23 and 25-31.
105. The 2-in-1 zinc finger nuclease variant according to any one of paragraphs 86-90 or 104, wherein the second zinc finger nuclease is encoded by a polynucleotide comprising the nucleotide sequence of any one of SEQ ID NOs: 17-23 and 25-31.
106. The 2-in-1 zinc finger nuclease variant according to any one of paragraphs 86-105, wherein the 2-in-1 zinc finger nuclease variant is encoded by a nucleotide sequence selected from any one of SEQ ID NO: 85-115.
107. A vector comprising the nucleic acid according to any one of paragraphs 64-85.
108. A cell comprising the nucleic acid according to any one of paragraphs 64-85 or the vector according to paragraph 107.
109. A pharmaceutical composition comprising a nucleic acid according to any one of paragraphs 64-85, a vector according to paragraph 104 or a 2-in-1 zinc finger nuclease variant according to any one of paragraphs 86-106.
110. The pharmaceutical composition according to paragraph 109, further comprising a donor nucleic acid.
111. The nucleic acid according to any one of paragraphs 64-85, for use in treating or preventing a lysosomal storage disorder.
112. The 2-in-1 zinc finger nuclease variant according to any one of paragraphs 86-106, for use in treating or preventing a lysosomal storage disorder.
113. The vector according to paragraph 107, for use in treating or preventing a lysosomal storage disorder.
114. The cell according to paragraph 108, for use in treating or preventing a lysosomal storage disorder.
115. The nucleic acid according to any one of paragraphs 64-85, for use in correcting a lysosomal storage disease-causing mutation in the genome of a cell.
116. The nucleic acid for use according to paragraph 115, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I— Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
117. The nucleic acid for use according to paragraph 115, wherein the lysosomal storage disease is selected from MPSI and MPSII.
118. The nucleic acid for use according to paragraph 117, wherein the lysosomal storage disease is selected from the group consisting of MPS I—Hurler Syndrome, MPS I—Scheie Syndrome, and MPS I—Hurler-Scheie Syndrome.
119. The nucleic acid for use according to paragraph 117, wherein the lysosomal storage disease is MPSII Hunter Syndrome.
120. The 2-in-1 zinc finger nuclease variant according to any one of paragraphs 86-106, for use in correcting a lysosomal storage disease-causing mutation in the genome of a cell.
121. The zinc finger nuclease variant for use according to paragraph 120, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
122. The zinc finger nuclease variant for use according to paragraph 120, wherein the lysosomal storage disease is selected from MPSI and MPSII.
123. The zinc finger nuclease variant for use according to paragraph 122, wherein the lysosomal storage disease is selected from the group consisting of MPS I—Hurler Syndrome, MPS I—Scheie Syndrome, and MPS I—Hurler-Scheie Syndrome.
124. The zinc finger nuclease variant for use according to paragraph 122, wherein the lysosomal storage disease is MPSII Hunter Syndrome.
125. The vector according to paragraph 107, for use in correcting a lysosomal storage disease-causing mutation in the genome of a cell.
126. The vector for use according to paragraph 125, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I— Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
127. The vector for use according to paragraph 125, wherein the lysosomal storage disease is selected from MPSI and MPSII.
128. The vector for use according to paragraph 127, wherein the lysosomal storage disease is selected from the group consisting of MPS I—Hurler Syndrome, MPS I—Scheie Syndrome, and MPS I—Hurler-Scheie Syndrome.
129. The vector for use according to paragraph 127, wherein the lysosomal storage disease is MPSII Hunter Syndrome.
130. The cell according to paragraph 108, for use in correcting a lysosomal storage disease-causing mutation in the genome of a cell.
131. The cell for use according to paragraph 130, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
132. The cell for use according to paragraph 130, wherein the lysosomal storage disease is selected from MPSI and MPSII.
133. The cell for use according to paragraph 132, wherein the lysosomal storage disease is selected from the group consisting of MPS I—Hurler Syndrome, MPS I—Scheie Syndrome, and MPS I—Hurler-Scheie Syndrome.
134. The cell for use according to paragraph 132, wherein the lysosomal storage disease is MPSII Hunter Syndrome.
135. The nucleic acid according to any one of paragraphs 64-85, for use in integrating an exogenous nucleotide sequence into a target nucleotide sequence in a gene of a cell.
136. The 2-in-1 zinc finger nuclease variant according to any one of paragraphs 86-106, for use in integrating an exogenous nucleotide sequence into a target nucleotide sequence in a gene of a cell.
137. The vector according to paragraph 107, for use in integrating an exogenous nucleotide sequence into a target nucleotide sequence in a gene of a cell.
138. The cell according to paragraph 108, for use in integrating an exogenous nucleotide sequence into a target nucleotide sequence in a gene of a cell.
139. The nucleic acid according to any one of paragraphs 64-85, for use in disrupting a target nucleotide sequence in a gene of a cell, wherein said gene comprises a mutation associated with a lysosomal storage disease.
140. The nucleic acid for use according to paragraph 139, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
141. The nucleic acid for use according to paragraph 139, wherein the lysosomal storage disease is selected from MPSI and MPSII.
142. The nucleic acid for use according to paragraph 141, wherein the lysosomal storage disease is selected from the group consisting of MPS I—Hurler Syndrome, MPS I—Scheie Syndrome, and MPS I—Hurler-Scheie Syndrome.
143. The nucleic acid for use according to paragraph 142, wherein the lysosomal storage disease is MPSII Hunter Syndrome.
144. The 2-in-1 zinc finger nuclease variant according to any one of paragraphs 86-106, for use in disrupting a target nucleotide sequence in a gene of a cell, wherein said gene comprises a mutation associated with a lysosomal storage disease.
145. The 2-in-1 zinc finger nuclease variant for use according to paragraph 144, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
146. The 2-in-1 zinc finger nuclease variant for use according to paragraph 144, wherein the lysosomal storage disease is selected from MPSI and MPSII.
147. The 2-in-1 zinc finger nuclease variant for use according to paragraph 146, wherein the lysosomal storage disease is selected from the group consisting of MPS I—Hurler Syndrome, MPS I—Scheie Syndrome, and MPS I—Hurler-Scheie Syndrome.
148. The 2-in-1 zinc finger nuclease variant for use according to paragraph 146, wherein the lysosomal storage disease is MPSII Hunter Syndrome.
149. The vector according to paragraph 107, for use in disrupting a target nucleotide sequence in a gene of a cell, wherein said gene comprises a mutation associated with a lysosomal storage disease.
150. The vector for use according to paragraph 149, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
151. The vector for use according to paragraph 149, wherein the lysosomal storage disease is selected from MPSI and MPSII.
152. The vector for use according to paragraph 151, wherein the lysosomal storage disease is selected from the group consisting of MPS I—Hurler Syndrome, MPS I—Scheie Syndrome, and MPS I—Hurler-Scheie Syndrome.
153. The vector for use according to paragraph 151, wherein the lysosomal storage disease is MPSII Hunter Syndrome.
154. The cell according to paragraph 108, for use in disrupting a target nucleotide sequence in a gene of a cell, wherein said gene comprises a mutation associated with a lysosomal storage disease.
155. The cell for use according to paragraph 154, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS TUB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
156. The cell for use according to paragraph 154, wherein the lysosomal storage disease is selected from MPSI and MPSII.
157. The cell for use according to paragraph 156, wherein the lysosomal storage disease is selected from the group consisting of MPS I—Hurler Syndrome, MPS I—Scheie Syndrome, and MPS I—Hurler-Scheie Syndrome.
158. The cell for use according to paragraph 156, wherein the lysosomal storage disease is MPSII Hunter Syndrome.
159. Use of a nucleic acid according to any one of paragraph 64-85, for the preparation of a medicament for treating or preventing a lysosomal storage disorder.
160. Use of a 2-in-1 zinc finger nuclease variant according to any one of paragraphs 86-106, for the preparation of a medicament for treating or preventing a lysosomal storage disorder.
161. Use of a vector according to paragraph 107, for the preparation of a medicament for treating or preventing a lysosomal storage disorder.
162. Use of a cell according to paragraph 108, for the preparation of a medicament for treating or preventing a lysosomal storage disorder.
163. Use of a nucleic acid according to any one of paragraph 64-85, for the preparation of a medicament for correcting a lysosomal storage disease-causing mutation in the genome of a cell.
164. Use of a 2-in-1 zinc finger nuclease variant according to any one of paragraphs 86-106, for the preparation of a medicament for correcting a lysosomal storage disease-causing mutation in the genome of a cell.
165. Use of a vector according to paragraph 107, for the preparation of a medicament for correcting a lysosomal storage disease-causing mutation in the genome of a cell.
166. Use of a cell according to paragraph 108, for the preparation of a medicament for correcting a lysosomal storage disease-causing mutation in the genome of a cell.
167. The use according to any one of paragraphs 159-166, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.
168. The use according to any one of paragraphs 159-166, wherein the lysosomal storage disease is selected from MPSI and MPSII.
169. The use according to paragraph 168, wherein the lysosomal storage disease is selected from the group consisting of MPS I—Hurler Syndrome, MPS I—Scheie Syndrome, and MPS I—Hurler-Scheie Syndrome.
170. The use according to paragraph 168, wherein the lysosomal storage disease is MPSII Hunter Syndrome.
171. Use of a nucleic acid according to any one of paragraph 64-85, for the preparation of a medicament for integrating an exogenous nucleotide sequence into a target nucleotide sequence in a gene of a cell.
172. Use of a 2-in-1 zinc finger nuclease variant according to any one of paragraphs 86-106, for the preparation of a medicament for integrating an exogenous nucleotide sequence into a target nucleotide sequence in a gene of a cell.
173. Use of a vector according to paragraph 107, for the preparation of a medicament for integrating an exogenous nucleotide sequence into a target nucleotide sequence in a gene of a cell.
174. Use of a cell according to paragraph 108, for the preparation of a medicament for integrating an exogenous nucleotide sequence into a target nucleotide sequence in a gene of a cell.
175. Use of a nucleic acid according to any one of paragraph 64-85, for the preparation of a medicament for disrupting a target nucleotide sequence in a gene of a cell, wherein said gene comprises a mutation associated with a lysosomal storage disease.
176. Use of a 2-in-1 zinc finger nuclease variant according to any one of paragraphs 86-106, for the preparation of a medicament for disrupting a target nucleotide sequence in a gene of a cell, wherein said gene comprises a mutation associated with a lysosomal storage disease.
177. Use of a vector according to paragraph 107, for the preparation of a medicament for disrupting a target nucleotide sequence in a gene of a cell, wherein said gene comprises a mutation associated with a lysosomal storage disease.
178. Use of a cell according to paragraph 108, for the preparation of a medicament for disrupting a target nucleotide sequence in a gene of a cell, wherein said gene comprises a mutation associated with a lysosomal storage disease.

The following Examples relate to exemplary embodiments of the present disclosure in which the nuclease comprises a zinc finger nuclease (ZFN). It will be appreciated that these examples are included merely for the purpose of illustration of certain features and embodiments of the present disclosure and are not intended to be limiting. Those skilled in the art will also recognize, or be able to ascertain using no more than routine experimentation numerous equivalents to the methods, nucleic acids, proteins, vectors and cells described herein. Such equivalents are considered to be within the scope of the present disclosure.

### EXAMPLES

### Example 1: Nuclease Constructs

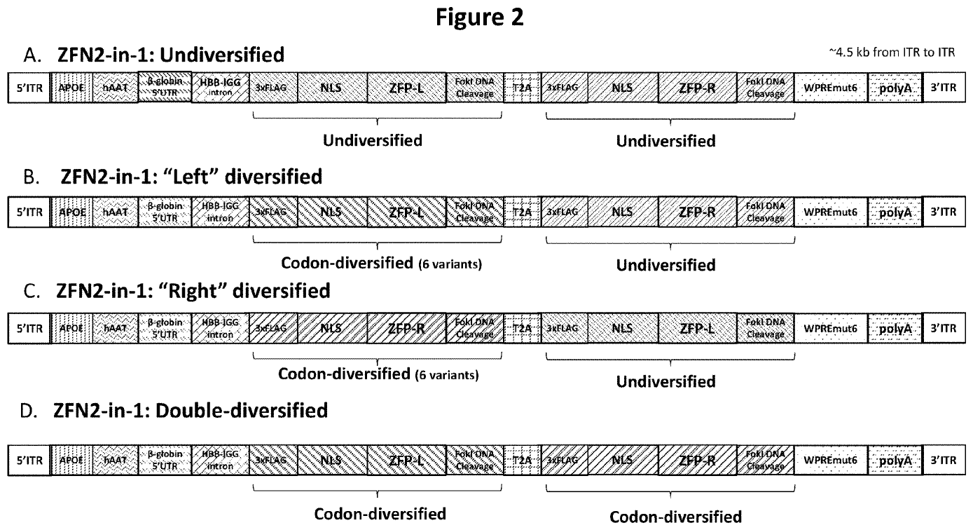

AAV (including AAV2/6) vector particles comprising separate left and right ZFNs; 2-in-1 nuclease constructs in which neither left nor right ZFNs were codon diversified; 2-in-1 constructs in which either the left or right ZFN was codon diversified (single diversified); or 2-in-1 constructs in which both the left and right ZFNs were codon diversified (dual diversified) were generated using standard techniques. The ZFN2-in-1 constructs were designed to comprise of 5′ and 3′ inverted terminal repeat (ITR) regions, an enhancer (APOE), a promoter (hAAT), a 5′ untranslated region (UTR) (β-globin), a chimeric intron (HBB-IGG), an epitope tag (3×FLAG), nuclear localization signal sequences (NLS), zinc-finger DNA binding domain (ZFP), Fok I DNA cleavage domains, a 2A peptide (T2A), a posttranscriptional regulatory element (WPREmut6), and polyadenylation sequence (bGH polyA). See, FIG. 2, Table 1, Table 2 and Table 3 for constructs used.

### Example 2: Assessment of Recombination in ZFN 2-In-1 Constructs

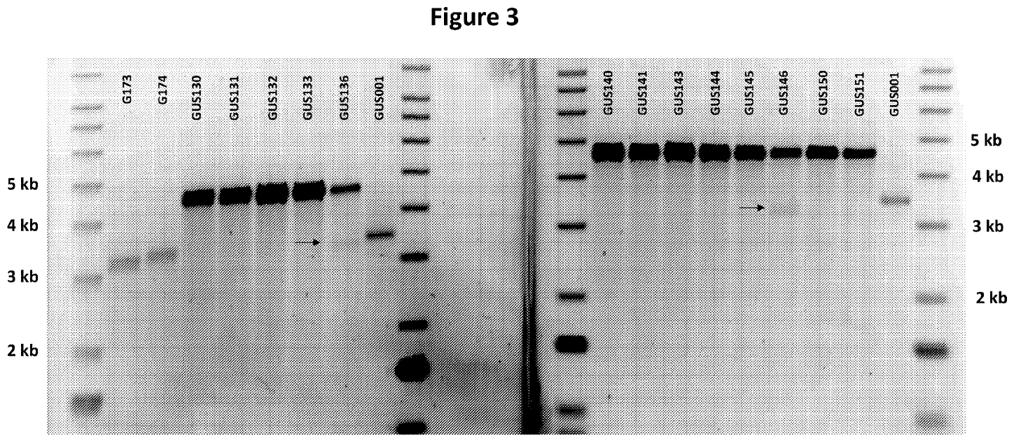

The ZFN 2-in-1 constructs in AAV2/6-HEK293 cells as described in Example 1 were produced. DNA was purified from the AAV particles and evaluated for recombination (inter- and intra-finger) and/or packaging errors by alkaline agarose gel and by Nextera sequence. The ZFN-2-in-1 constructs having single-diversified ZFNs (GUS130, GUS131, GUS132, GUS133, GUS134, GUS140, GUS141, GUS143, GUS144, and GUS145) and double diversified ZFNs (GUS150 and GUS151) resulted in DNA bands of an expected size of approximately 4.5 kilobases (kb). Recombination and/or packaging errors were observed (inter- or intra-sequence) 2-in-1 ZFN constructs in which neither left nor right ZFN was codon diversified (GUS136 and GUS146) (band marked with arrow). See FIG. 3.

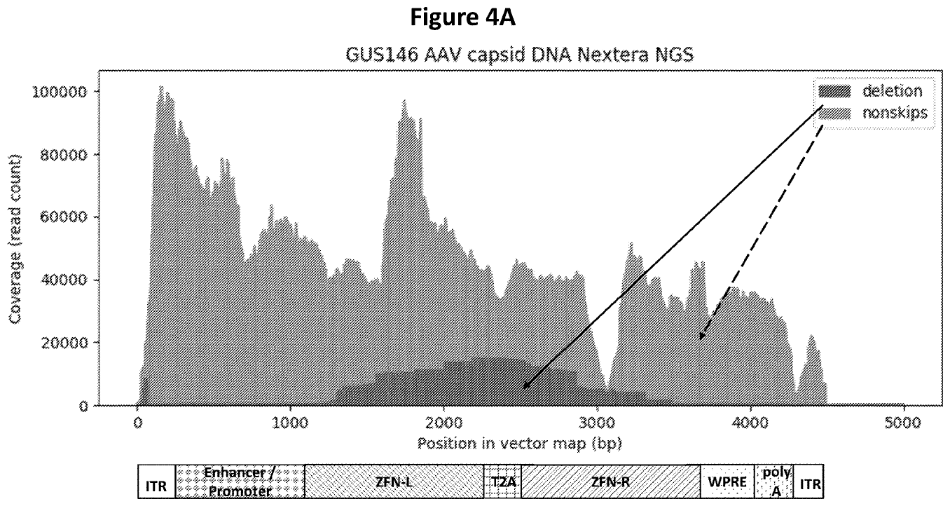

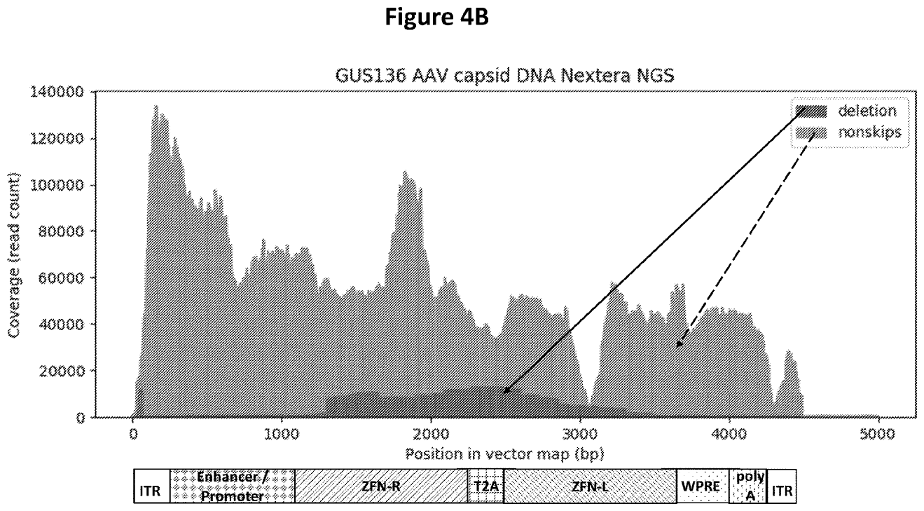

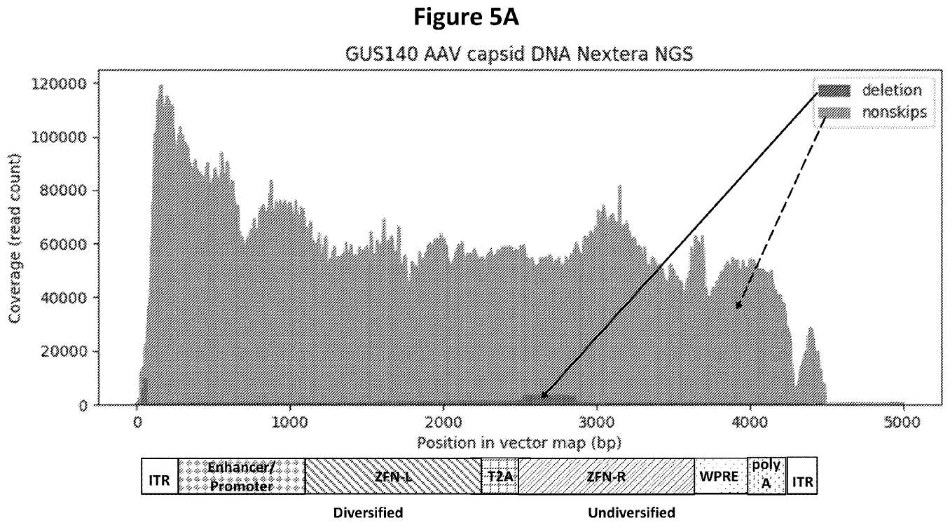

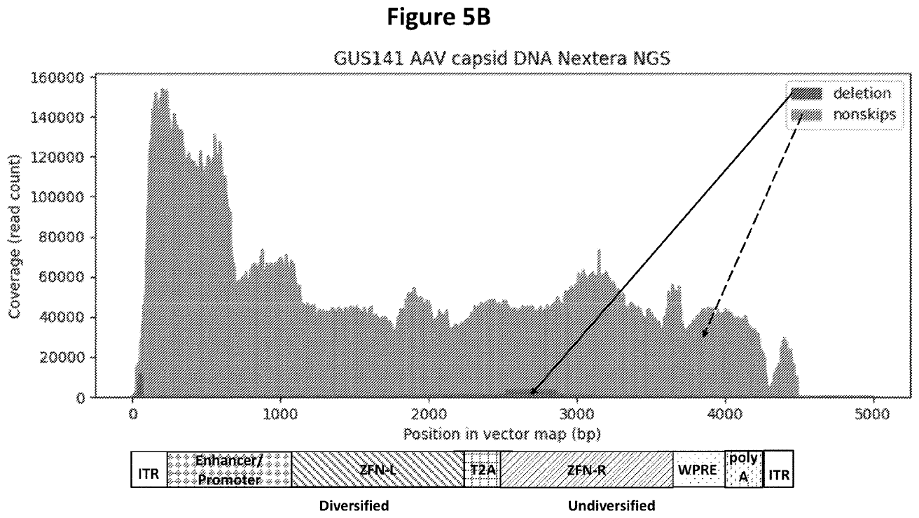

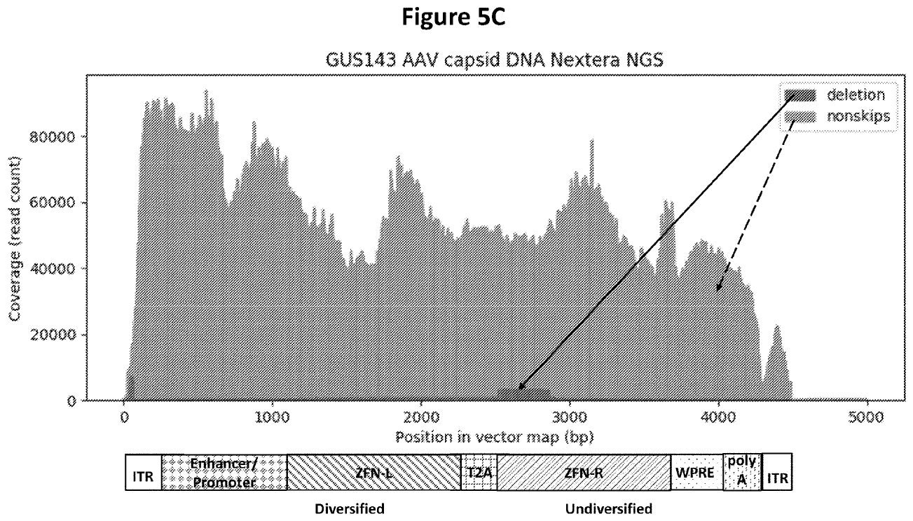

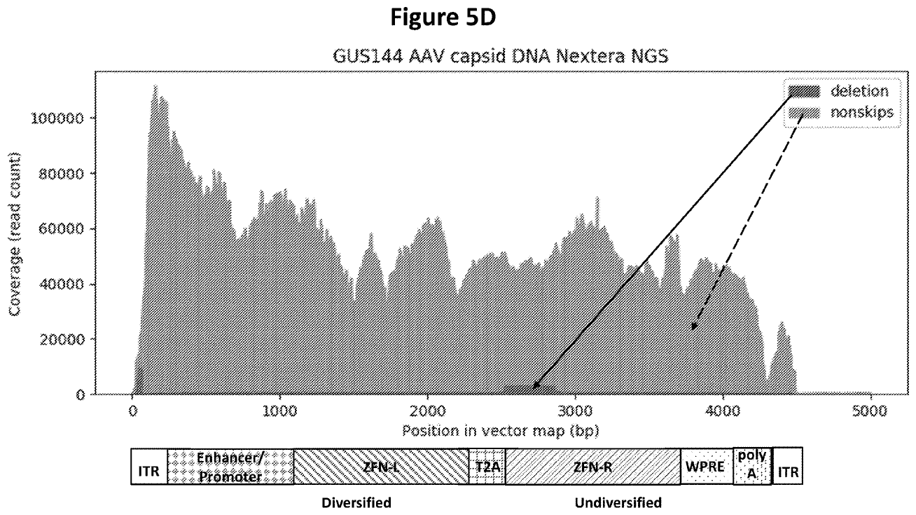

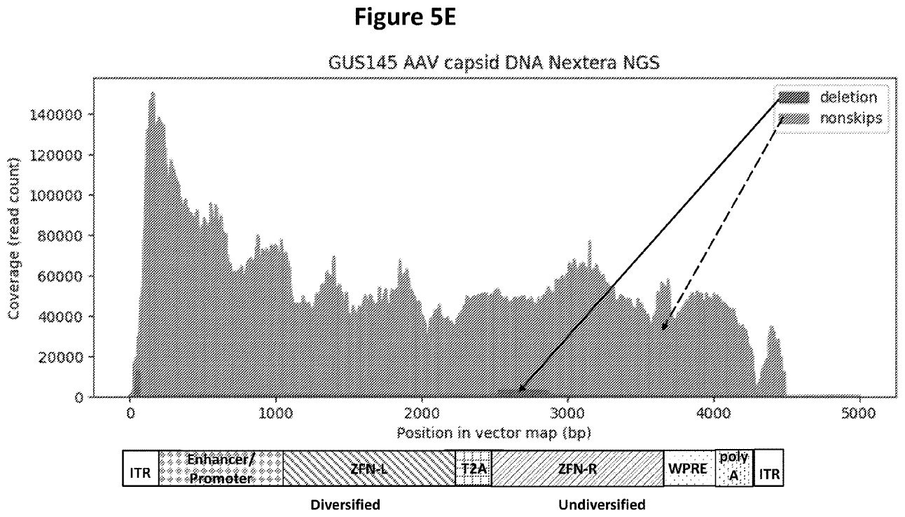

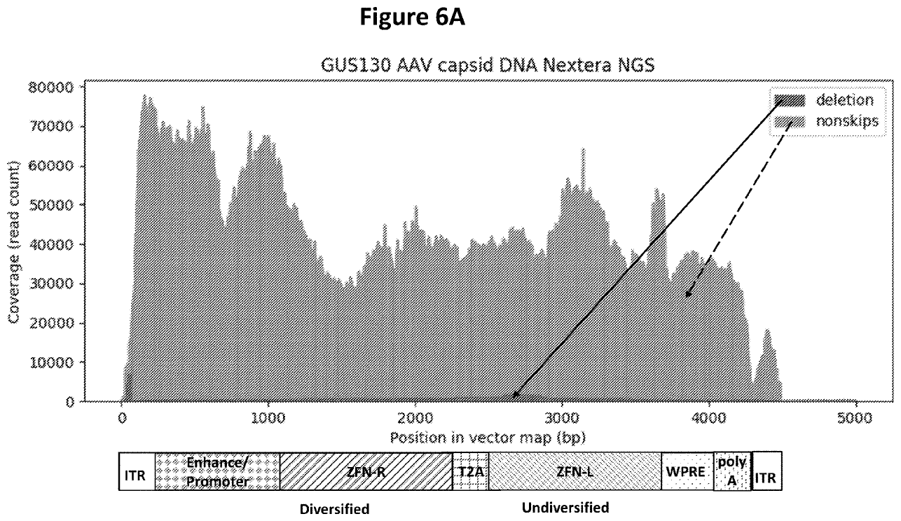

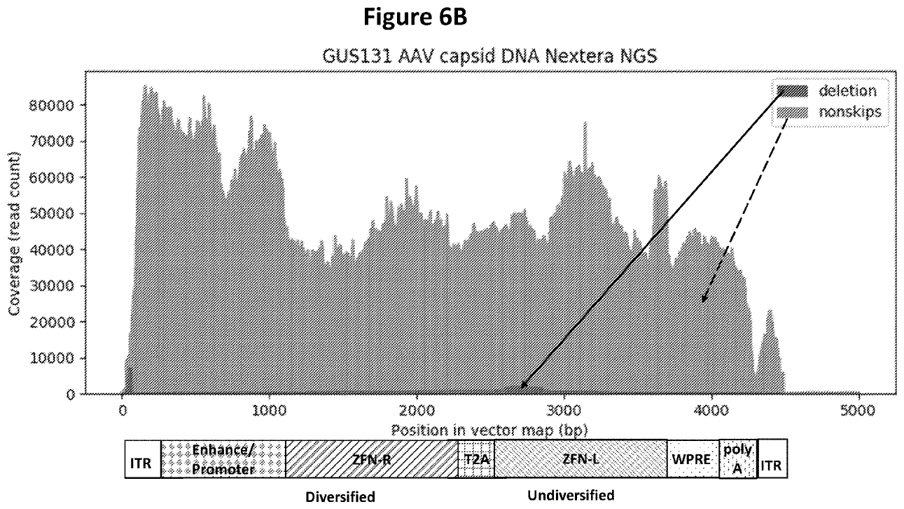

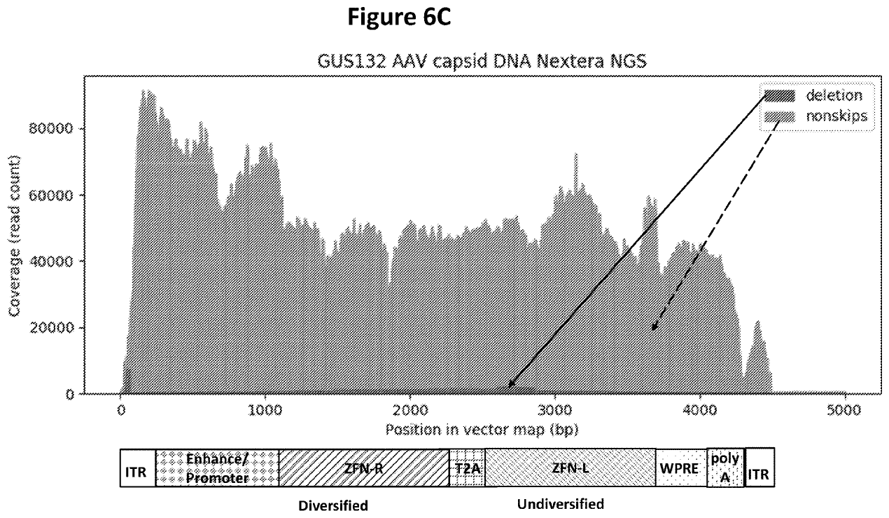

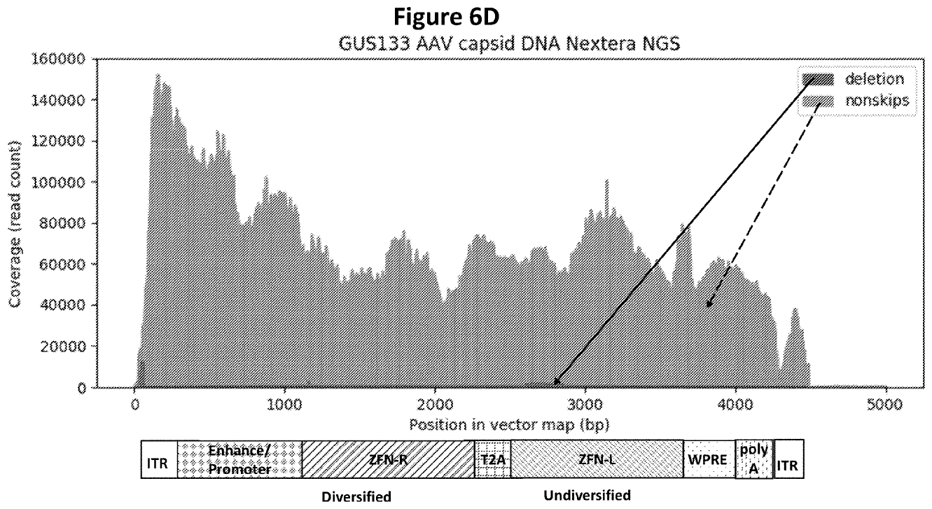

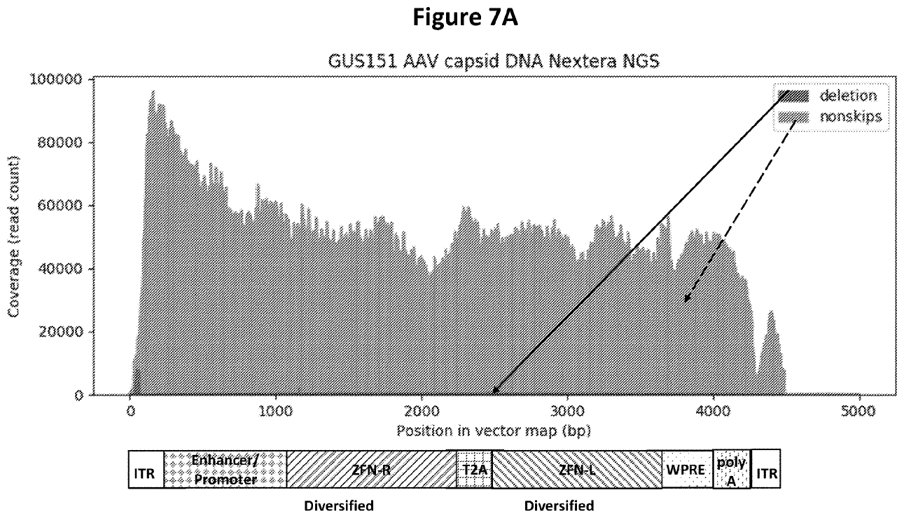

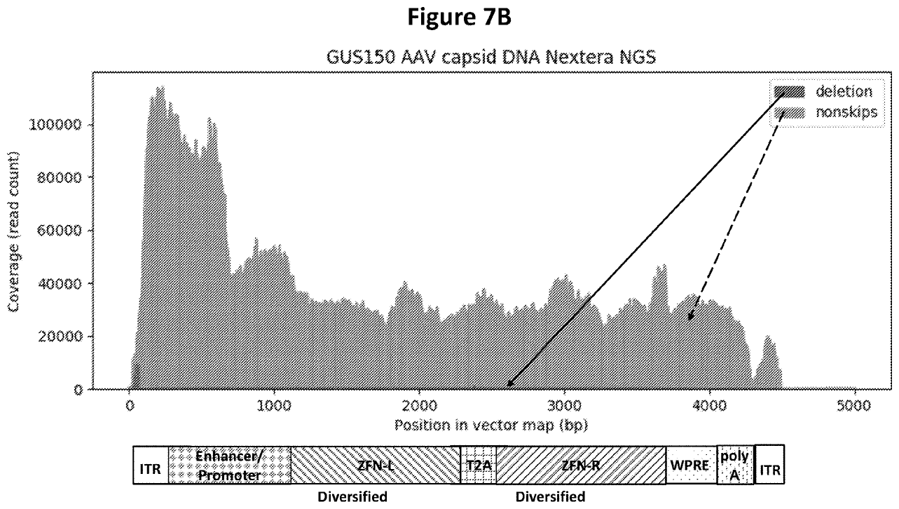

Nextera deletion plots were also used to assess recombinations. DNA was fragmented with transposase, PCR amplified and next generation sequencing (NGS) was performed. NGS reads were aligned to construct maps with a large-deletion tolerant aligner (i.e., GSNAP: Genomic Short-Read Nucleotide Alignment Program). Within the program, deletions larger than 3 kb were removed. Results are presented in FIGS. 4A-4B, 5A-5E, 6A-6D and 7A-7B. The graphs depict NGS coverage (read counts) over the span of the sequence length of the ZFN2-in-1 constructs, and show regions where recombination and deletions occur. FIG. 4A-4B are the results for undiversified ZFN2-in-1 constructs, GUS146 and GUS136. FIG. 5A-5E are the results for ZFN2-in-1 constructs with diversified left ZFNs and undiversified right ZFNs, GUS140, GUS141, GUS143, GUS144 and GUS145. FIG. 6A-6D are the results for ZFN2-in-1 constructs with diversified right ZFNs and undiversified left ZFNs, GUS130, GUS131, GUS132, and GUS133. FIG. 7A-7B are the results for double-diversified ZFN2-in-1 constructs, GUS150 and GUS151, which detected very few or no deletion events occurring in the ZFN regions of the constructs. Detection of a deletion event in an ITR region may indicate poor coverage due to secondary structures, internal duplications, and/or GC-richness. Overall, ZFN2-in-1 constructs with undiversified ZFNs leads to an increased rate of recombination events (e.g. deletions), while codon diversification of ZFNs have very few or no recombination events occurring.

### Example 3: Zinc Finger Nuclease Construct Activity and Expression in Cells

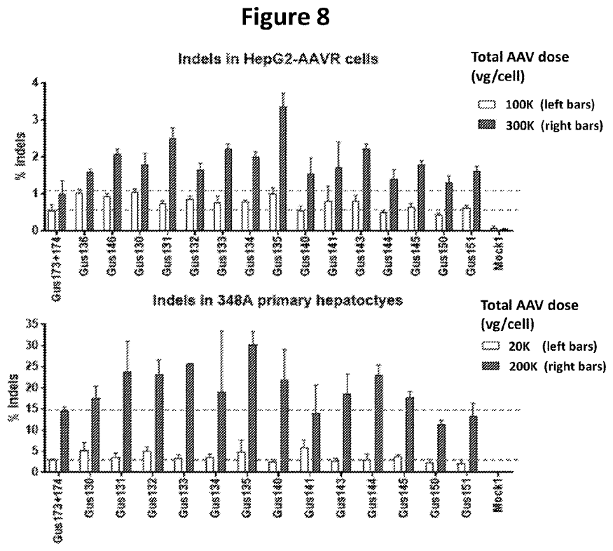

The AAV vectors were also evaluated for activity as measured by the percentage of insertions or deletions (% indels), essentially as described in in U.S. Patent Publication No. 2019/0241877. Briefly, HepG2 cells and 348A primary hepatocytes were transduced AAV ZFN vectors as described above at 100,000 vg/cell or 300,000 vg/cell (HepG2 cells) and at 20,000 vg/cell or 200,000 vg/cell (348 cells). As shown in FIG. 8, Panels A and B, single- and double-diversified ZFN2-in-1 constructs exhibit activity comparable to 2 separate ZFN vectors.

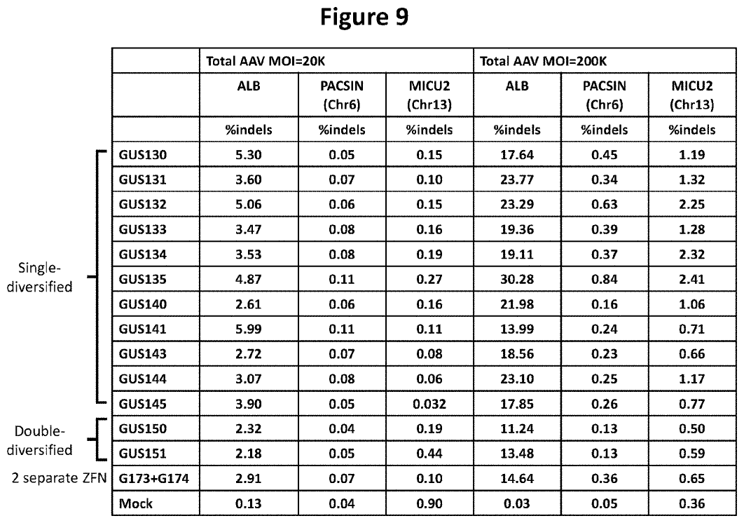

Activity of the ZFN constructs for on-target (ALB) and off-target (MICU2 and PACSIN1) genes was also measured (% indel) in 348A primary human hepatocytes. The activity for the ZFN2-in-1 constructs were comparable activity to the activity of two separate ZFN control constructs for the on-target Albumin (ALB) and off-target (MICU2 and PACSIN1) genes. FIG. 9.

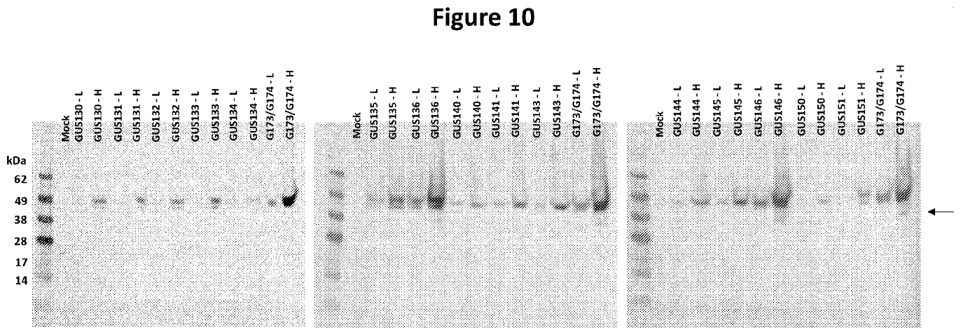

Western Blot analysis was also performed to evaluate ZFN expression from AAV nuclease constructs introduced into HepG2 cells. Briefly, HepG2 cells were transfected with 50,000 vg/cell (low “L”) or 150,000 (high “H”) vg/cell of each separate AAV ZFN construct (G173/G174) or with 100,000 vg/cell (low “L”) or 300,000 vg/cell (high “H”) with undiversified, single diversified or double diversified 2-in-1 constructs as described above. Protein expression was detected via the Flag-M2 Protein. The expected band size is 45-50 kDa (size varies based on ZFN length and position relative to T2A). As shown in FIG. 10, ZFN expression was detectable from all constructs.

Thus, the 2-in-1 constructs described herein are expressed and active and both single and dual diversified 2-in-1 constructs reduce recombination rates as compared to undiversified 2-in-1 constructs.

What is claimed is:

1. A method for treating or preventing a lysosomal storage disorder in a subject, the method comprising modifying a target sequence in the genome of a cell of said subject by introducing into the cell a nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
or a vector comprising said nucleic acid encoding a 2-in-1 zinc finger nuclease variant;
wherein the polynucleotide encoding the 2A self-cleaving peptide is positioned between the polynucleotide encoding the first zinc finger nuclease and the polynucleotide encoding the second zinc finger nuclease.

2. A method for correcting a lysosomal storage disease-causing mutation in the genome of a cell, the method comprising modifying a target sequence in the genome of the cell by introducing into the cell a nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
or a vector comprising said nucleic acid encoding a 2-in-1 zinc finger nuclease variant;
wherein the polynucleotide encoding the 2A self-cleaving peptide is positioned between the polynucleotide encoding the first zinc finger nuclease and the polynucleotide encoding the second zinc finger nuclease.

3. A method for modifying the genome of a cell comprising a mutation in a gene associated with a lysosomal storage disease, the method comprising introducing into a cell a nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
or a vector comprising said nucleic acid encoding a 2-in-1 zinc finger nuclease variant;
wherein the polynucleotide encoding the 2A self-cleaving peptide is positioned between the polynucleotide encoding the first zinc finger nuclease and the polynucleotide encoding the second zinc finger nuclease.

4. A method for integrating an exogenous nucleotide sequence into a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
or a vector comprising said nucleic acid encoding a 2-in-1 zinc finger nuclease variant;
wherein the polynucleotide encoding the 2A self-cleaving peptide is positioned between the polynucleotide encoding the first zinc finger nuclease and the polynucleotide encoding the second zinc finger nuclease.

5. A method for disrupting a target nucleotide sequence in a gene of a cell, wherein said gene comprises a mutation associated with a lysosomal storage disease, the method comprising introducing into the cell a nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
or a vector comprising said nucleic acid encoding a 2-in-1 zinc finger nuclease variant;
wherein the polynucleotide encoding the 2A self-cleaving peptide is positioned between the polynucleotide encoding the first zinc finger nuclease and the polynucleotide encoding the second zinc finger nuclease.

6. The method according to any one of claims 1-5, further comprising introducing into the cell a donor nucleic acid or a vector comprising said donor nucleic acid, wherein said donor nucleic acid comprises a polynucleotide encoding a corrective lysosomal storage disease-associated protein or enzyme or portion thereof.

7. The method according to claim 6, wherein the donor nucleic acid is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.

8. The method according to claim 6, wherein corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.

9. The method according to any one of claim 1-8, wherein the nucleic acid encoding a 2-in-1 zinc finger nuclease variant further comprises a polynucleotide sequence selected from one or more of:
a. a polynucleotide sequence encoding a nuclear localization sequence;
b. a 5′ITR polynucleotide sequence;
c. an enhancer polynucleotide sequence;
d. a promoter polynucleotide sequence;
e. a 5′UTR polynucleotide sequence;
f. a chimeric intron polynucleotide sequence;
g. a polynucleotide sequences encoding an epitope tag;
h. a polynucleotide sequence encoding a Fok I cleavage domain;
i. a post-transcriptional regulatory element polynucleotide sequence;
j. a polyadenylation signal sequence; and
k. a 3′ITR polynucleotide sequence.

10. The method according to claim 9, wherein the nucleic acid encoding a 2-in-1 zinc finger nuclease variant comprises two independent polynucleotide sequences encoding two nuclear localization sequences.

11. The method according to claim 9, wherein the nucleic acid encoding a 2-in-1 zinc finger nuclease variant comprises two or more independent polynucleotide sequences encoding two or more epitope tags.

12. The method according to claim 9, wherein the nucleic acid encoding a 2-in-1 zinc finger nuclease variant comprises two or more independent polynucleotide sequences encoding two or more Fok I cleavage domains.

13. The method according to any one of claims 1-12, wherein the polynucleotide encoding the first zinc finger nuclease is codon diversified.

14. The method according to any one of claims 1-12, wherein the polynucleotide encoding the second zinc finger nuclease is codon diversified.

15. The method according to any one of claims 1-12, wherein the polynucleotide encoding the first zinc finger nuclease is codon diversified and the polynucleotide encoding the second zinc finger nuclease is codon diversified.

16. The method according to any one of claims 1-12, wherein the polynucleotide encoding the first zinc finger nuclease comprises the nucleotide sequence of any one of SEQ ID NOs: 116-129.

17. The method according to any one of claim 1-12 or 16, wherein the polynucleotide encoding the second zinc finger nuclease comprises the nucleotide sequence of any one of SEQ ID NOs: 116-129.

18. The method according to any one of claims 1-12, wherein the polynucleotide encoding the first zinc finger nuclease comprises a nucleotide sequence encoding the amino acid sequence of SEQ ID NOs: 136 or 137.

19. The method according to any one of claim 1-12 or 18, wherein the polynucleotide encoding the second zinc finger nuclease comprises a nucleotide sequence encoding the amino acid sequence of SEQ ID NOs: 136 or 137.

20. The method according to any one of claims 1-12, wherein the polynucleotide sequence encoding the first zinc finger nuclease comprises the nucleotide sequence of any one of SEQ ID NOs: 71-84.

21. The method according to any one of claim 1-12 or 20, wherein the polynucleotide sequence encoding the second zinc finger nuclease comprises the nucleotide sequence of any one of SEQ ID NOs: 71-84.

22. The method according to any one of claims 1-12, wherein the polynucleotide encoding the first zinc finger nuclease comprises a nucleotide sequence encoding the amino acid sequence of SEQ ID NOs: 130 or 131.

23. The method according to any one of claim 1-12 or 22, wherein the polynucleotide encoding the second zinc finger nuclease comprises a nucleotide sequence encoding the amino sequence of SEQ ID NOs: 130 or 131.

24. The method according to any one of claims 1-12, wherein the polynucleotide sequence encoding the first zinc finger nuclease comprises the nucleotide sequence of any one of SEQ ID NOs: 139-152.

25. The method according to any one of claim 1-12 or 24, wherein the polynucleotide sequence encoding the second zinc finger nuclease comprises the nucleotide sequence of any one of SEQ ID NOs: 139-152.

26. The method according to any one of claims 1-12, wherein the polynucleotide sequence encoding the first zinc finger nuclease comprises the nucleotide sequence of any one of SEQ ID NOs: 17-23 and 25-31.

27. The method according to any one of claim 1-12 or 26, wherein the polynucleotide sequence encoding the second zinc finger nuclease comprises the nucleotide sequence of any one of SEQ ID NOs: 17-23 and 25-31.

28. The method according to any one of claims 1-27, wherein the nucleic acid encoding a 2-in-1 zinc finger nuclease variant comprises a nucleotide sequence selected from any one of SEQ ID NO: 85-115.

29. The method according to any one of claims 1-27, wherein the nucleic acid encoding a 2-in-1 zinc finger nuclease variant comprises a nucleotide sequence selected from any one of SEQ ID NO: 35-49.

30. The method according to any one of claims 1-29, wherein the vector is an AAV vector.

31. A method for treating or preventing a lysosomal storage disorder in a subject, the method comprising modifying a target sequence in the genome of a cell of said subject by introducing into the cell a 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and the second zinc finger nuclease.

32. A method for correcting a lysosomal storage disease-causing mutation in the genome of a cell, the method comprising modifying a target sequence in the genome of the cell by introducing into the cell a 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and second zinc finger nuclease.

33. A method for modifying the genome of a cell comprising a mutation in a gene associated with a lysosomal storage disease, the method comprising introducing into a cell a 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and second zinc finger nuclease.

34. A method for integrating an exogenous nucleotide sequence into a target nucleotide sequence in a gene of a cell, wherein said gene comprises a mutation associated with a lysosomal storage disease, the method comprising introducing into the cell a 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and second zinc finger nuclease.

35. A method for disrupting a target nucleotide sequence in a gene of a cell, wherein said gene comprises a mutation associated with a lysosomal storage disease, the method comprising introducing into the cell a 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and second zinc finger nuclease.

36. The method according to any one of claims 31-35, further comprising introducing into the cell a donor nucleic acid or a vector comprising said donor nucleic acid, wherein said donor nucleic acid comprises a polynucleotide encoding a corrective lysosomal storage disease-associated protein or enzyme or portion thereof.

37. The method according to claim 36, wherein the donor nucleic acid is selected from the group consisting of MAN2B1, AGA, LIPA, CTNS, LAMP2, GLA, ASAH1, FUCA1, CTSA, GBA, GLB1, HEXB, HEXA, GM2A, GNPTAB, GALC, ARSA, IDUA, IDS, SGSH, NAGLU, GSNAT, GNS, GALNS, GLB1, ARSB, GUSB, HYAL1, NEU1, GNPTG, MCOLN1, SUMF1, PPT1, TPP1, CLN3, DNAJC5, CLN5, CLN6, CLN7, CLN8, SMPD1, SMPD1, NPC1, NPC2, PAH, GAA, CTSK, SLC17A5, and NAGA.

38. The method according to claim 36, wherein corrective lysosomal storage disease-associated protein or enzyme is selected from the group consisting of Alpha-D-mannosidase, N-aspartyl-beta-glucosaminidase, Lysosomal acid lipase, Cystinosin, Lysosomal associated membrane protein 2, Alpha-galactosidase A, Acid ceramidase, Alpha fucosidase, Cathepsin A, Acid beta-glucocerebrosidase, Beta galactosidase, Beta hexosaminidase A, Beta hexosaminidase B, Beta-hexosaminidase, GM2 ganglioside activator (GM2A), GLcNAc-1-phosphotransferase, Beta-galactosylceramidase, Lysosomal acid lipase, Arylsulfatase A, Alpha-L-iduronidase, Iduronate-2-sulphatase, Heparan N-sulfatase, Alpha-N-acetylglucosaminidase, acetyl CoA:alpha-glucosaminide acetyltransferase, N-acetyl glucosamine-6-sulfatase, Galactosamine-6-sulfate sulfatase, Beta-galactosidase, Arylsulfatase B, Beta-glucuronidase, Hyaluronidase, Neuraminidase, GlcNAc-1-phosphotransferase, Mucolipin-1, Formylglycine-generating enzyme (FGE), Palmitoyl-protein thioesterase 1, tripeptidyl peptidase 1, CLN3 protein, Cysteine string protein alpha, CLN5 protein, CLN6 protein, CLN7 protein, CLN8 protein, Acid sphingomyelinase, NPC 1/NPC 2, Phenylalanine hydroxylase, Acid alpha-glucosidase, cathepsin K, Sialin (sialic acid transporter), and Alpha-N-acetylgalactosaminidase.

39. The method according to any one of claims 31-38, wherein the 2-in-1 zinc finger nuclease variant further comprises one or more of:
a. a nuclear localization sequence;
b. an epitope tag; and
c. a Fok I cleavage domain.

40. The method according to claim 39, wherein the 2-in-1 zinc finger nuclease variant comprises two independent nuclear localization sequences.

41. The method according to claim 39, wherein the 2-in-1 zinc finger nuclease variant comprises two or more independent epitope tags.

42. The method according to claim 39, wherein the 2-in-1 zinc finger nuclease variant comprises two or more independent Fok I cleavage domains.

43. The method according to any one of claims 31-42, wherein the first zinc finger nuclease is codon diversified.

44. The method according to any one of claims 31-42, wherein the second zinc finger nuclease is codon diversified.

45. The method according to any one of claims 31-42, wherein the first zinc finger nuclease is codon diversified and the second zinc finger nuclease is codon diversified.

46. The method according to any one of claims 31-42, wherein the first zinc finger nuclease is encoded by a polynucleotide comprising the nucleotide sequence of any one of SEQ ID NOs: 116-129.

47. The method according to any one of claim 31-42 or 46, wherein the second zinc finger nuclease is encoded by a polynucleotide comprising the nucleotide sequence of any one of SEQ ID NOs: 116-129.

48. The method according to any one of claims 31-42 wherein the first zinc finger nuclease comprises the amino acid sequence of SEQ ID NOs: 136 or 137.

49. The method according to any one of claim 31-42 or 48, wherein the second zinc finger nuclease comprises the amino acid sequence of SEQ ID NOs: 136 or 137.

50. The method according to any one of claims 31-42, wherein the first zinc finger nuclease is encoded by a polynucleotide sequence comprising the nucleotide sequence of any one of SEQ ID NOs: 71-84.

51. The method according to any one of claim 31-42 or 50, wherein the second zinc finger nuclease is encoded by a polynucleotide sequence comprising the nucleotide sequence of any one of SEQ ID NOs: 71-84.

52. The method according to any one of claims 31-42, wherein the first zinc finger nuclease comprises the amino acid sequence of SEQ ID NOs: 130 or 131.

53. The method according to any one of claim 31-42 or 52, wherein the second zinc finger nuclease comprises the amino sequence of SEQ ID NOs: 130 or 131.

54. The method according to any one of claims 31-42, wherein the first zinc finger nuclease is encoded by a polynucleotide comprising the nucleotide sequence of any one of SEQ ID NOs: 139-152.

55. The method according to any one of claim 31-42 or 54, wherein the second zinc finger nuclease is encoded by a polynucleotide comprising the nucleotide sequence of any one of SEQ ID NOs: 139-152.

56. The method according to any one of claims 31-42, wherein the first zinc finger nuclease is encoded by a polynucleotide comprising the nucleotide sequence of any one of SEQ ID NOs: 17-23 and 25-31.

57. The method according to any one of claim 31-42 or 56, wherein the second zinc finger nuclease is encoded by a polynucleotide comprising the nucleotide sequence of any one of SEQ ID NOs: 17-23 and 25-31.

58. The method according to any one of claims 1-57, wherein the 2-in-1 zinc finger nuclease variant is encoded by a nucleotide sequence selected from any one of SEQ ID NO: 85-115.

59. The method according to any one of claims 1-57, wherein the 2-in-1 zinc finger nuclease variant is encoded by a nucleotide sequence selected from any one of SEQ ID NO: 35-49.

60. The method according to any one of claims 1-59, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I— Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

61. The method according to any one of claims 1-59, wherein the lysosomal storage disease is selected from MPSI and MPSII.

62. The method according to claim 61, wherein the lysosomal storage disease is selected from the group consisting of MPS I—Hurler Syndrome, MPS I—Scheie Syndrome, and MPS I—Hurler-Scheie Syndrome.

63. The method according to claim 61, wherein the lysosomal storage disease is MPSII Hunter Syndrome.

64. A nucleic acid encoding a 2-in-1 zinc finger nuclease variant comprising:
a. a polynucleotide encoding a first zinc finger nuclease;
b. a polynucleotide encoding a second zinc finger nuclease; and
c. a polynucleotide encoding a 2A self-cleaving peptide;
wherein the polynucleotide encoding the 2A self-cleaving peptide is positioned between the polynucleotide encoding the first zinc finger nuclease and the polynucleotide encoding the second zinc finger nuclease.

65. The nucleic acid according to claim 64, wherein the nucleic acid encoding a 2-in-1 zinc finger nuclease variant further comprises a polynucleotide sequence selected from one or more of:
a. a polynucleotide sequence encoding a nuclear localization sequence;
b. a 5′ITR polynucleotide sequence;
c. an enhancer polynucleotide sequence;
d. a promoter polynucleotide sequence;
e. a 5′UTR polynucleotide sequence;
f. a chimeric intron polynucleotide sequence;
g. a polynucleotide sequences encoding an epitope tag;
h. a polynucleotide sequence encoding a Fok I cleavage domain;
i. a post-transcriptional regulatory element polynucleotide sequence;
j. a polyadenylation signal sequence; and
k. a 3′ITR polynucleotide sequence.

66. The nucleic acid according to claim 65, wherein the nucleic acid encoding a 2-in-1 zinc finger nuclease variant comprises two independent polynucleotide sequences encoding two nuclear localization sequences.

67. The nucleic acid according to claim 65, wherein the nucleic acid encoding a 2-in-1 zinc finger nuclease variant comprises two or more independent polynucleotide sequences encoding two or more epitope tags.

68. The nucleic acid according to claim 65, wherein the nucleic acid encoding a 2-in-1 zinc finger nuclease variant comprises two or more independent polynucleotide sequences encoding two or more Fok I cleavage domains.

69. The nucleic acid according to any one of claims 64-68, wherein the polynucleotide encoding the first zinc finger nuclease is codon diversified.

70. The nucleic acid according to any one of claims 64-68, wherein the polynucleotide encoding the second zinc finger nuclease is codon diversified.

71. The nucleic acid according any one of claims 64-68, wherein the polynucleotide encoding the first zinc finger nuclease is codon diversified and the polynucleotide encoding the second zinc finger nuclease is codon diversified.

72. The nucleic acid according to any one of claims 64-69, wherein the polynucleotide encoding the first zinc finger nuclease comprises the nucleotide sequence of any one of SEQ ID NOs: 116-129.

73. The nucleic acid according to any one of claim 64-68 or 72, wherein the polynucleotide encoding the second zinc finger nuclease comprises the nucleotide sequence of any one of SEQ ID NOs: 116-129.

74. The nucleic acid according to any one of claims 64-68, wherein the polynucleotide encoding the first zinc finger nuclease comprises a nucleotide sequence encoding the amino acid sequence of SEQ ID NOs: 136 or 137.

75. The nucleic acid according to any one of claim 64-68 or 74, wherein the polynucleotide encoding the second zinc finger nuclease comprises a nucleotide sequence encoding the amino acid sequence of SEQ ID NOs: 136 or 137.

76. The nucleic acid according to any one of claims 64-68, wherein the polynucleotide sequence encoding the first zinc finger nuclease comprises the nucleotide sequence of any one of SEQ ID NOs: 71-84.

77. The nucleic acid according to any one of claim 64-68 or 76, wherein the polynucleotide sequence encoding the second zinc finger nuclease comprises the nucleotide sequence of any one of SEQ ID NOs: 71-84.

78. The nucleic acid according to any one of claims 64-68, wherein the polynucleotide encoding the first zinc finger nuclease comprises a nucleotide sequence encoding the amino acid sequence of SEQ ID NOs: 130 or 131.

79. The nucleic acid according to any one of claim 64-68 or 78, wherein the polynucleotide encoding the second zinc finger nuclease comprises a nucleotide sequence encoding the amino sequence of SEQ ID NOs: 130 or 131.

80. The nucleic acid according to any one of claims 64-68, wherein the polynucleotide sequence encoding the first zinc finger nuclease comprises the nucleotide sequence of any one of SEQ ID NOs: 139-152.

81. The nucleic acid according to any one of claim 64-68 or 80, wherein the polynucleotide sequence encoding the second zinc finger nuclease comprises the nucleotide sequence of any one of SEQ ID NOs: 139-152.

82. The nucleic acid according to any one of claims 64-68, wherein the polynucleotide sequence encoding the first zinc finger nuclease comprises the nucleotide sequence of any one of SEQ ID NOs: 17-23 and 25-31.

83. The nucleic acid according to any one of claim 64-68 or 82, wherein the polynucleotide sequence encoding the second zinc finger nuclease comprises the nucleotide sequence of any one of SEQ ID NOs: 17-23 and 25-31.

84. The nucleic acid according to any one of claims 64-83, wherein the nucleic acid encoding a 2-in-1 zinc finger nuclease variant comprises a nucleotide sequence selected from any one of SEQ ID NO: 85-115.

85. The nucleic acid according to any one of claims 641-83, wherein the nucleic acid encoding a 2-in-1 zinc finger nuclease variant comprises a nucleotide sequence selected from any one of SEQ ID NO: 35-49.

86. A 2-in-1 zinc finger nuclease variant comprising:
a. a first zinc finger nuclease;
b. a second zinc finger nuclease; and
c. a 2A self-cleaving peptide;
wherein the 2A self-cleaving peptide is positioned between the first zinc finger nuclease and second zinc finger nuclease.

87. The 2-in-1 zinc finger nuclease variant according to claim 86, further comprising one or more of:
a. a nuclear localization sequence;
b. an epitope tag; and
c. a Fok I cleavage domain.

88. The 2-in-1 zinc finger nuclease variant according to claim 87, wherein the 2-in-1 zinc finger nuclease variant comprises two independent nuclear localization sequences.

89. The 2-in-1 zinc finger nuclease variant according to claim 87, wherein the 2-in-1 zinc finger nuclease variant comprises two or more independent epitope tags.

90. The 2-in-1 zinc finger nuclease variant according to claim 87, wherein the 2-in-1 zinc finger nuclease variant comprises two or more independent Fok I cleavage domains.

91. The 2-in-1 zinc finger nuclease variant according to any one of claims 86-90, wherein the first zinc finger nuclease is codon diversified.

92. The 2-in-1 zinc finger nuclease variant according to any one of claims 86-90, wherein the second zinc finger nuclease is codon diversified.

93. The 2-in-1 zinc finger nuclease variant according any one of claims 86-90, wherein the first zinc finger nuclease is codon diversified and the second zinc finger nuclease is codon diversified.

94. The 2-in-1 zinc finger nuclease variant according to any one of claims 86-90, wherein the first zinc finger nuclease is encoded by a polynucleotide comprising the nucleotide sequence of any one of SEQ ID NOs: 116-129.

95. The 2-in-1 zinc finger nuclease variant according to any one of claims 86-90, wherein the second zinc finger nuclease is encoded by a polynucleotide comprising the nucleotide sequence of any one of SEQ ID NOs: 116-129.

96. The 2-in-1 zinc finger nuclease variant according to any one of claims 86-90, wherein the first zinc finger nuclease comprises the amino acid sequence of SEQ ID NOs: 136 or 137.

97. The 2-in-1 zinc finger nuclease variant according to any one of claim 86-90 or 96, wherein the second zinc finger nuclease comprises the amino acid sequence of SEQ ID NOs: 136 or 137.

98. The 2-in-1 zinc finger nuclease variant according to any one of claims 86-90, wherein the first zinc finger nuclease is encoded by a polynucleotide sequence comprising the nucleotide sequence of any one of SEQ ID NOs: 71-84.

99. The 2-in-1 zinc finger nuclease variant according to any one of claim 86-90 or 98, wherein the second zinc finger nuclease is encoded by a polynucleotide sequence comprising the nucleotide sequence of any one of SEQ ID NOs: 71-84.

100. The 2-in-1 zinc finger nuclease variant according to any one of claims 86-90, wherein the first zinc finger nuclease comprises the amino acid sequence of SEQ ID NOs: 130 or 131.

101. The 2-in-1 zinc finger nuclease variant according to any one of claim 86-90 or 100, wherein the second zinc finger nuclease comprises the amino sequence of SEQ ID NOs: 130 or 131.

102. The 2-in-1 zinc finger nuclease variant according to any one of claims 86-90, wherein the first zinc finger nuclease is encoded by a polynucleotide comprising the nucleotide sequence of any one of SEQ ID NOs: 139-152.

103. The 2-in-1 zinc finger nuclease variant according to any one of claim 86-90 or 102, wherein the second zinc finger nuclease is encoded by a polynucleotide comprising the nucleotide sequence of any one of SEQ ID NOs: 139-152.

104. The 2-in-1 zinc finger nuclease variant according to any one of claims 86-90, wherein the first zinc finger nuclease is encoded by a polynucleotide comprising the nucleotide sequence of any one of SEQ ID NOs: 17-23 and 25-31.

105. The 2-in-1 zinc finger nuclease variant according to any one of claim 86-90 or 104, wherein the second zinc finger nuclease is encoded by a polynucleotide comprising the nucleotide sequence of any one of SEQ ID NOs: 17-23 and 25-31.

106. The 2-in-1 zinc finger nuclease variant according to any one of claims 86-105, wherein the 2-in-1 zinc finger nuclease variant is encoded by a nucleotide sequence selected from any one of SEQ ID NO: 85-115.

107. A vector comprising the nucleic acid according to any one of claims 64-85.

108. A cell comprising the nucleic acid according to any one of claims 64-85 or the vector according to claim 107.

109. A pharmaceutical composition comprising a nucleic acid according to any one of claims 64-85, a vector according to claim 104 or a 2-in-1 zinc finger nuclease variant according to any one of claims 86-106.

110. The pharmaceutical composition according to claim 109, further comprising a donor nucleic acid.

111. The nucleic acid according to any one of claims 64-85, for use in treating or preventing a lysosomal storage disorder.

112. The 2-in-1 zinc finger nuclease variant according to any one of claims 86-106, for use in treating or preventing a lysosomal storage disorder.

113. The vector according to claim 107, for use in treating or preventing a lysosomal storage disorder.

114. The cell according to claim 108, for use in treating or preventing a lysosomal storage disorder.

115. The nucleic acid according to any one of claims 64-85, for use in correcting a lysosomal storage disease-causing mutation in the genome of a cell.

116. The nucleic acid for use according to claim 115, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

117. The nucleic acid for use according to claim 115, wherein the lysosomal storage disease is selected from MPSI and MPSII.

118. The nucleic acid for use according to claim 117, wherein the lysosomal storage disease is selected from the group consisting of MPS I—Hurler Syndrome, MPS I—Scheie Syndrome, and MPS I—Hurler-Scheie Syndrome.

119. The nucleic acid for use according to claim 117, wherein the lysosomal storage disease is MPSII Hunter Syndrome.

120. The 2-in-1 zinc finger nuclease variant according to any one of claims 86-106, for use in correcting a lysosomal storage disease-causing mutation in the genome of a cell.

121. The zinc finger nuclease variant for use according to claim 120, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

122. The zinc finger nuclease variant for use according to claim 120, wherein the lysosomal storage disease is selected from MPSI and MPSII.

123. The zinc finger nuclease variant for use according to claim 122, wherein the lysosomal storage disease is selected from the group consisting of MPS I—Hurler Syndrome, MPS I—Scheie Syndrome, and MPS I—Hurler-Scheie Syndrome.

124. The zinc finger nuclease variant for use according to claim 122, wherein the lysosomal storage disease is MPSII Hunter Syndrome.

125. The vector according to claim 107, for use in correcting a lysosomal storage disease-causing mutation in the genome of a cell.

126. The vector for use according to claim 125, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

127. The vector for use according to claim 125, wherein the lysosomal storage disease is selected from MPSI and MPSII.

128. The vector for use according to claim 127, wherein the lysosomal storage disease is selected from the group consisting of MPS I—Hurler Syndrome, MPS I—Scheie Syndrome, and MPS I—Hurler-Scheie Syndrome.

129. The vector for use according to claim 127, wherein the lysosomal storage disease is MPSII Hunter Syndrome.

130. The cell according to claim 108, for use in correcting a lysosomal storage disease-causing mutation in the genome of a cell.

131. The cell for use according to claim 130, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

132. The cell for use according to claim 130, wherein the lysosomal storage disease is selected from MPSI and MPSII.

133. The cell for use according to claim 132, wherein the lysosomal storage disease is selected from the group consisting of MPS I—Hurler Syndrome, MPS I—Scheie Syndrome, and MPS I—Hurler-Scheie Syndrome.

134. The cell for use according to claim 132, wherein the lysosomal storage disease is MPSII Hunter Syndrome.

135. The nucleic acid according to any one of claims 64-85, for use in integrating an exogenous nucleotide sequence into a target nucleotide sequence in a gene of a cell.

136. The 2-in-1 zinc finger nuclease variant according to any one of claims 86-106, for use in integrating an exogenous nucleotide sequence into a target nucleotide sequence in a gene of a cell.

137. The vector according to claim 107, for use in integrating an exogenous nucleotide sequence into a target nucleotide sequence in a gene of a cell.

138. The cell according to claim 108, for use in integrating an exogenous nucleotide sequence into a target nucleotide sequence in a gene of a cell.

139. The nucleic acid according to any one of claims 64-85, for use in disrupting a target nucleotide sequence in a gene of a cell, wherein said gene comprises a mutation associated with a lysosomal storage disease.

140. The nucleic acid for use according to claim 139, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

141. The nucleic acid for use according to claim 139, wherein the lysosomal storage disease is selected from MPSI and MPSII.

142. The nucleic acid for use according to claim 141, wherein the lysosomal storage disease is selected from the group consisting of MPS I—Hurler Syndrome, MPS I—Scheie Syndrome, and MPS I—Hurler-Scheie Syndrome.

143. The nucleic acid for use according to claim 142, wherein the lysosomal storage disease is MPSII Hunter Syndrome.

144. The 2-in-1 zinc finger nuclease variant according to any one of claims 86-106, for use in disrupting a target nucleotide sequence in a gene of a cell, wherein said gene comprises a mutation associated with a lysosomal storage disease.

145. The 2-in-1 zinc finger nuclease variant for use according to claim 144, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

146. The 2-in-1 zinc finger nuclease variant for use according to claim 144, wherein the lysosomal storage disease is selected from MPSI and MPSII.

147. The 2-in-1 zinc finger nuclease variant for use according to claim 146, wherein the lysosomal storage disease is selected from the group consisting of MPS I—Hurler Syndrome, MPS I—Scheie Syndrome, and MPS I—Hurler-Scheie Syndrome.

148. The 2-in-1 zinc finger nuclease variant for use according to claim 146, wherein the lysosomal storage disease is MPSII Hunter Syndrome.

149. The vector according to claim 107, for use in disrupting a target nucleotide sequence in a gene of a cell, wherein said gene comprises a mutation associated with a lysosomal storage disease.

150. The vector for use according to claim 149, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

151. The vector for use according to claim 149, wherein the lysosomal storage disease is selected from MPSI and MPSII.

152. The vector for use according to claim 151, wherein the lysosomal storage disease is selected from the group consisting of MPS I—Hurler Syndrome, MPS I—Scheie Syndrome, and MPS I—Hurler-Scheie Syndrome.

153. The vector for use according to claim 151, wherein the lysosomal storage disease is MPSII Hunter Syndrome.

154. The cell according to claim 108, for use in disrupting a target nucleotide sequence in a gene of a cell, wherein said gene comprises a mutation associated with a lysosomal storage disease.

155. The cell for use according to claim 154, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (PRA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS TUB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

156. The cell for use according to claim 154, wherein the lysosomal storage disease is selected from MPSI and MPSII.

157. The cell for use according to claim 156, wherein the lysosomal storage disease is selected from the group consisting of MPS I—Hurler Syndrome, MPS I—Scheie Syndrome, and MPS I—Hurler-Scheie Syndrome.

158. The cell for use according to claim 156, wherein the lysosomal storage disease is MPSII Hunter Syndrome.

159. Use of a nucleic acid according to any one of claim 64-85, for the preparation of a medicament for treating or preventing a lysosomal storage disorder.

160. Use of a 2-in-1 zinc finger nuclease variant according to any one of claims 86-106, for the preparation of a medicament for treating or preventing a lysosomal storage disorder.

161. Use of a vector according to claim 107, for the preparation of a medicament for treating or preventing a lysosomal storage disorder.

162. Use of a cell according to claim 108, for the preparation of a medicament for treating or preventing a lysosomal storage disorder.

163. Use of a nucleic acid according to any one of claim 64-85, for the preparation of a medicament for correcting a lysosomal storage disease-causing mutation in the genome of a cell.

164. Use of a 2-in-1 zinc finger nuclease variant according to any one of claims 86-106, for the preparation of a medicament for correcting a lysosomal storage disease-causing mutation in the genome of a cell.

165. Use of a vector according to claim 107, for the preparation of a medicament for correcting a lysosomal storage disease-causing mutation in the genome of a cell.

166. Use of a cell according to claim 108, for the preparation of a medicament for correcting a lysosomal storage disease-causing mutation in the genome of a cell.

167. The use according to any one of claims 159-166, wherein the lysosomal storage disease is selected from the group consisting of Alpha-mannosidosis, Aspartylglucosaminuria, Cholesteryl ester storage disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis (Types I, II and III), GM2 Sandhoff Disease (I/FA), GM2 Tay-Sachs disease, GM2 Gangliosidosis AB variant, I-Cell Disease/Mucolipidosis II, Krabbe Disease, Lysosomal acid lipase deficiency, Metachromatic Leukodystrophy, MPS I—Hurler Syndrome, MPS I—Scheie Syndrome, MPS I—Hurler-Scheie Syndrome, MPS II Hunter Syndrome, MPS IIIA—Sanfilippo Syndrome Type A, MPS IIIB—Sanfilippo Syndrome Type B, MPS IIIC—Sanfilippo Syndrome Type C, MPSIIID—Sanfilippo Syndrome Type D, MPS IV—Morquio Type A, MPS IV—Morquio Type B, MPS VI—Maroteaux-Lamy, MPS VII—Sly Syndrome, MPS IX—Hyaluronidase Deficiency, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Niemann-Pick Disease Type A, Niemann-Pick Disease Type B, Niemann-Pick Disease Type C, Phenylketonuria, Pompe Disease, Pycnodysostosis, Sialic Acid Storage Disease, Schindler Disease, and Wolman Disease.

168. The use according to any one of claims 159-166, wherein the lysosomal storage disease is selected from MPSI and MPSII.

169. The use according to claim 168, wherein the lysosomal storage disease is selected from the group consisting of MPS I—Hurler Syndrome, MPS I—Scheie Syndrome, and MPS I—Hurler-Scheie Syndrome.

170. The use according to claim 168, wherein the lysosomal storage disease is MPSII Hunter Syndrome.

171. Use of a nucleic acid according to any one of claim 64-85, for the preparation of a medicament for integrating an exogenous nucleotide sequence into a target nucleotide sequence in a gene of a cell.

172. Use of a 2-in-1 zinc finger nuclease variant according to any one of claims 86-106, for the preparation of a medicament for integrating an exogenous nucleotide sequence into a target nucleotide sequence in a gene of a cell.

173. Use of a vector according to claim 107, for the preparation of a medicament for integrating an exogenous nucleotide sequence into a target nucleotide sequence in a gene of a cell.

174. Use of a cell according to claim 108, for the preparation of a medicament for integrating an exogenous nucleotide sequence into a target nucleotide sequence in a gene of a cell.

175. Use of a nucleic acid according to any one of claim 64-85, for the preparation of a medicament for disrupting a target nucleotide sequence in a gene of a cell, wherein said gene comprises a mutation associated with a lysosomal storage disease.

176. Use of a 2-in-1 zinc finger nuclease variant according to any one of claims 86-106, for the preparation of a medicament for disrupting a target nucleotide sequence in a gene of a cell, wherein said gene comprises a mutation associated with a lysosomal storage disease.

177. Use of a vector according to claim 107, for the preparation of a medicament for disrupting a target nucleotide sequence in a gene of a cell, wherein said gene comprises a mutation associated with a lysosomal storage disease.

178. Use of a cell according to claim 108, for the preparation of a medicament for disrupting a target nucleotide sequence in a gene of a cell, wherein said gene comprises a mutation associated with a lysosomal storage disease.
